(19) United States
(12) Patent Application Publication
(10) Pub. No.: US 2024/0058425 A1
(43) Pub. Date: Feb. 22, 2024

(54) SYSTEMS AND METHODS FOR GENOME-WIDE ANNOTATION OF GENE REGULATORY ELEMENTS LINKED TO CELL FITNESS

(71) Applicant: Duke University, Durham, NC (US)

(72) Inventors: Charles A. Gersbach, Chapel Hill, NC (US); Alejandro Barrera, Durham, NC (US); Tyler S. Klann, Durham, NC (US); Maria ter Weele, Durham, NC (US); Gregory E. Crawford, Chapel Hill, NC (US); Timothy E. Reddy, Durham, NC (US)

(21) Appl. No.: 18/180,718

(22) Filed: Mar. 8, 2023

Related U.S. Application Data
(60) Provisional application No. 63/317,847, filed on Mar. 8, 2022.
(60) Provisional application No. 63/372,373, filed on Mar. 8, 2022.

(51) Int. Cl. A61K 38/46; C12N 15/11; C12N 9/22; C12N 15/90; C12N 15/86; C12N 15/10; C12N 5/09; A61K 31/7088; A61P 35/02

(52) U.S. Cl. CPC A61K 38/465; C12N 15/11; C12N 9/22; C12N 15/907; C12N 15/86; C12N 15/1082; C12N 5/0694; A61K 31/7088; A61P 35/02; C12N 2310/20; C12N 2800/80; C12N 2740/15043

(57) ABSTRACT

Disclosed herein are compositions and methods for targeting a novel regulatory element of a gene. The compositions may be used in methods of modifying growth of a cell, decreasing cell fitness, increasing cell fitness, and/or treating cancer such as leukemia.

### CROSS-REFERENCE TO RELATED APPLICATIONS

This application claims priority to U.S. Provisional Patent Application No. 63/317,847 filed Mar. 8, 2022, and U.S. Provisional Patent Application No. 63/372,373 filed Mar. 8, 2022, the entire contents of each of which are hereby incorporated by reference.

### STATEMENT REGARDING FEDERALLY SPONSORED RESEARCH

This invention was made with government support under grants UM1HG009428, RO1HG010741, RM1HG011123, DP20D008586, and R01DA036865 awarded by the National Institutes of Health. The government has certain rights in the invention.

### REFERENCE TO AN ELECTRONIC SEQUENCE LISTING

The contents of the electronic sequence listing (028193-9363-US04 Sequence Listing.xml, 509,861 bytes, and created on Jul. 25, 2023) is herein incorporated by reference in its entirety.

### FIELD

This disclosure relates to targeting gene regulatory elements that affect cell fitness. The disclosure further relates to compositions and methods for treating leukemia.

### INTRODUCTION

Human gene regulatory elements control gene expression and orchestrate many biological processes including cell differentiation, proliferation, and environmental responses. Genetic and epigenetic variation that alters gene regulatory element function is a primary contributor to human traits and susceptibility to common disease. Studies of chromatin state and transcription factor occupancy have identified millions of putative human gene regulatory elements. The biological importance and large number of putative human gene regulatory elements have motivated the development of high-throughput technologies to measure regulatory element activity genome-wide. Examples include genome-wide assays that measure putative regulatory element activity on reporter gene expression, and targeted CRISPR-based methods to measure the effects of genetic or epigenetic perturbation of up to thousands of regulatory elements in their native chromosomal context.

One measure of gene or regulatory element function is its contribution to overall cell fitness, comprising the balance of cell survival and proliferation. Genome-wide technologies, such as RNAi and CRISPR-based screens, have identified genes involved in diverse cellular processes. CRISPR-based genetic or epigenetic perturbation of noncoding regulatory elements within specific genomic loci have identified target genes and downstream effects on cell phenotypes. However, these perturbation screens of distal regulatory elements have generally been limited to small regions of the genome or loci encoding oncogenes. Consequently, functional understanding of the millions of predicted human gene regulatory elements remains sparse, making it difficult to routinely establish gene regulatory contributions to human traits and disease.

### SUMMARY

In an aspect, the disclosure relates to a composition for treating leukemia. The composition may include a Cas9 protein or a fusion protein, wherein the fusion protein comprises two heterologous polypeptide domains, wherein the first polypeptide domain comprises a Cas9 protein and the second polypeptide domain has an activity selected from the group consisting of transcription activation activity, transcription repression activity, transcription release factor activity, histone modification activity, nuclease activity, nucleic acid association activity, methylase activity, and demethylase activity; and at least one guide RNA (gRNA) that targets the Cas9 protein to a regulatory element of a target gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR.

In some embodiments, the gRNA targets the Cas9 protein to a polynucleotide sequence comprising a sequence selected from SEQ ID NOs: 339-479. In some embodiments, the gRNA is encoded by a polynucleotide sequence comprising a sequence selected from SEQ ID NOs: 57-197 or comprises a polynucleotide sequence comprising a sequence selected from SEQ ID NOs: 198-338. In some embodiments, the composition inhibits cell viability. In some embodiments, the target gene is selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1. In some embodiments, the gRNA targets the Cas9 protein to a polynucleotide sequence comprising a sequence selected from SEQ ID NOs: 339-473. In some embodiments, the gRNA is encoded by a polynucleotide sequence comprising a sequence selected from SEQ ID NOs: 57-191 or comprises a polynucleotide sequence comprising a sequence selected from SEQ ID NOs: 198-332. In some embodiments, the composition increases cell viability. In some embodiments, the target gene is selected from FADS3, RPAP1, SLC25A39, RP13-20L14.6, FOXA2, and GMPR. In some embodiments, the gRNA targets the Cas9 protein to a polynucleotide sequence comprising a sequence selected from SEQ ID NOs: 474-479. In some embodiments, the gRNA is encoded by a polynucleotide sequence comprising a sequence selected from SEQ ID NOs: 192-197 or comprises a polynucleotide sequence comprising a sequence selected from SEQ ID NOs: 333-338. In some embodiments, the Cas protein comprises a Streptococcus pyogenes Cas9 protein, a Staphylococcus aureus Cas9 protein, or any fragment thereof. In some embodiments, the Cas9 protein comprises an amino acid sequence having at least 90% or greater identity to a sequence selected from SEQ ID NOs: 20-23, or any fragment thereof, or is encoded by a polynucleotide comprising a sequence having at least 90% or greater identity to a sequence selected from SEQ ID NOs: 24-26, or any fragment thereof. In some embodiments, the Cas9 protein comprises an amino acid sequence having one, two, three, four, five or more changes selected from amino acid substitutions, insertions, or deletions, relative to a sequence selected from SEQ ID NOs: 20-23, or any fragment thereof, or is encoded by a polynucleotide comprising a sequence having one, two, three, four, five or more changes selected from nucleotide substitutions, insertions, or deletions, relative to a sequence selected from SEQ ID NOs: 24-26, or any fragment thereof. In some embodiments, the Cas9 protein comprises the amino acid sequence of SEQ ID NO: 20 or 21 or 22 or 23, or any fragment thereof, or is encoded by a polynucleotide comprising a sequence selected from SEQ ID NOs: 24 or 25 or 26. In some embodiments, the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3. In some embodiments, the second polypeptide domain has transcription repression activity. In some embodiments, the second polypeptide domain comprises KRAB. In some embodiments, KRAB comprises an amino acid sequence having at least 90% or greater identity to SEQ ID NO: 55, or any fragment thereof. In some embodiments, KRAB comprises an amino acid sequence having one, two, three, four, five or more changes selected from amino acid substitutions, insertions, or deletions, relative to SEQ ID NO: 55, or any fragment thereof. In some embodiments, KRAB comprises the amino acid sequence of SEQ ID NO: 55, or any fragment thereof. In some embodiments, fusion protein comprises an amino acid sequence having at least 90% or greater identity to SEQ ID NO: 40 or 42, or any fragment thereof. In some embodiments, fusion protein comprises an amino acid sequence having one, two, three, four, five or more changes selected from amino acid substitutions, insertions, or deletions, relative to SEQ ID NO: 40 or 42, or any fragment thereof. In some embodiments, fusion protein comprises the amino acid sequence of SEQ ID NO: 40 or 42, or any fragment thereof. In some embodiments, the leukemia is chronic myeloid leukemia (CML). In some embodiments, the leukemia is acute myeloid leukemia (AML).

In a further aspect, the disclosure relates to an isolated polynucleotide sequence comprising a sequence selected from SEQ ID NOs: 57-338. In a further aspect, the disclosure relates to an isolated polynucleotide sequence encoding a composition as detailed herein. In a further aspect, the disclosure relates to a vector comprising an isolated polynucleotide sequence as detailed herein. In a further aspect, the disclosure relates to a vector encoding a composition as detailed herein. In a further aspect, the disclosure relates to a cell comprising a composition as detailed herein, an isolated polynucleotide sequence as detailed herein, or a vector as detailed herein, or a combination thereof. In a further aspect, the disclosure relates to a pharmaceutical composition comprising a composition as detailed herein, an isolated polynucleotide sequence as detailed herein, a vector as detailed herein, or a cell as detailed herein, or a combination thereof.

Another aspect of the disclosure provides method of treating leukemia in a subject. The method may include targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the subject. In some embodiments, modifying the expression of the gene comprises reducing expression of the gene. In some embodiments, the method includes administering to the subject a composition as detailed herein, an isolated polynucleotide sequence as detailed herein, a vector as detailed herein, a cell as detailed herein, or a pharmaceutical composition as detailed herein, or a combination thereof. In some embodiments, the leukemia is chronic myeloid leukemia (CML). In some embodiments, the leukemia is acute myeloid leukemia (AML).

Another aspect of the disclosure provides a method of modifying growth of a cell. The method may include targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR in the cell. In some embodiments, the method includes administering to the cell a composition as detailed herein, an isolated polynucleotide sequence as detailed herein, or a vector as detailed herein, or a combination thereof.

Another aspect of the disclosure provides a method of decreasing cell fitness. The method may include targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, ILI ORB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the cell. In some embodiments, the targeting includes administering to a cell a composition as detailed herein, an isolated polynucleotide sequence as detailed herein, or a vector as detailed herein, or a combination thereof. In some embodiments, decreasing cell fitness comprises decreasing cell growth rate, decreasing cell growth duration, decreasing cell size, increasing cell death, or a combination thereof.

Another aspect of the disclosure provides a method of increasing cell fitness. The method may include targeting a regulatory element of, or modifying the expression of, a gene selected from FADS3, RPAP1, SLC25A39, RP13-20L14.6, FOXA2, and GMPR in the cell. In some embodiments, the targeting comprises administering to a cell a composition as detailed herein, an isolated polynucleotide sequence as detailed herein, or a vector as detailed herein, or a combination thereof. In some embodiments, increasing cell fitness comprises increasing cell growth rate, increasing cell growth duration, increasing cell size, or a combination thereof.

Another aspect of the disclosure provides all that is disclosed in any of TABLES S1-S17, 18A, 18B, 19A, and 19B of Klann et al. 2021, “Genome-wide annotation of gene regulatory elements linked to cell fitness” bioRxiv doi: 10.1101/2021.03.08.434470, which is incorporated herein by reference in its entirety. Another aspect of the disclosure provides any and all methods, and/or processes, and/or devices, and/or systems, and/or devices, and/or kits, and/or products, and/or materials, and/or compositions, and/or uses shown and/or described expressly or by implication in the information provided herewith, including but not limited to features that may be apparent and/or understood by those of skill in the art.

The disclosure provides for other aspects and embodiments that will be apparent in light of the following detailed description and accompanying figures.

### BRIEF DESCRIPTION OF THE DRAWINGS

FIG. 1A is an overall schematic of (i) discovery wgCERES screen, (ii) secondary validation screen of regulatory elements, (iii) cell-type specificity, and (iv) single-cell (scCERES) readout to connect cell fitness-associated regulatory elements to target genes. FIG. 1B is a schematic of wgCERES approach. gRNAs are designed to all DHSs in the K562 cell line and synthesized as a pool for lentiviral delivery. K562 cells either constitutively expressing or not expressing dCas9^{KRAB} are treated with the lentiviral gRNA library at a low MOI and cultured for 14 population doublings. Genomic DNA is then harvested and the gRNA abundance is quantified by Illumina sequencing. FIG. 1C is a schematic describing four levels of gRNA grouping analyses, including individual gRNA, sliding windows of 2 or 3 gRNAs, and averaging all gRNAs within a DHS. FIG. 1D is a summary of DHS hits identified by significant changes to individual gRNAs or grouped gRNAs. FIG. 1E is a volcano plot of significance of gRNA changes relative to log 2 (fold-change). FIG. 1F is a distribution of significant gRNAs relative to transcriptional start sites of nearest genes. FIG. 1G shows representative examples of significant distal DHS hits (blue boxes) that also have a significant DHS hit at a TSS of the nearest gene. ChromHMM tracks indicate promoters (red), putative enhancers (yellow), and polycomb repressed regions (gray). FIG. 1H shows a UMAP dimensionality reduction plot showing different ChromHMM chromatin state informed classes of significant (FDR<0.1) DHS hits. Histone modifications as well as several epigenetic modifying proteins were included as input for dimensionality reduction. FIG. 1I shows relative abundances of significantly depleted or enriched gRNAs relative to ChromHMM classes of genome annotations.

FIG. 2A is a volcano plot showing 9,833 significantly depleted or enriched gRNAs. Depleted gRNAs were more abundant and show larger effect size than enriched gRNAs. FIG. 2B shows a comparison of log 2 (fold-change) between the first wgCERES screen and the second sub-library screen. Only gRNAs that overlap both studies are represented (N=50,021 common gRNAs). FIG. 2C shows a distribution of number of significant gRNAs per DHS between the first wgCERES screen and the second sub-library screen. Inset shows combined counts for more than 1 gRNA per DHS. FIG. 2D is screenshot examples of DHS hits (blue boxes) that displayed a smaller number significant gRNA hits (red lines) in the wgCERES discovery screen, and additional significant gRNAs in the more densely tiled distal sub-library validation screen.

FIG. 3A shows individual gRNAs that were tested in K562 cells constitutively expressing dCas9^{KRAB}, and mRNA changes were detected through qRT-PCR. Dark gray and light gray bars indicate validation gRNAs that were depleted or enriched, respectively, in the screen. Black bars indicate non-targeting gRNA control. Bars with diagonal lines indicate cells only expressing dCas9^{KRAB}, without gRNA transduction. Significance was determined by one-way analysis of variance followed by Dunnett's test (n=3 biological replicates, mean±s.e.m.): ****P<0.0001, ***P<0.001, **P<0.01, *P<0.05 versus dCas9^{KRAB} only control. All fold enrichments are relative to the transduction of a control gRNA and normalized to TBP. FIG. 3B shows validation of a gRNA by RNA-seq shows that the largest effect is the nearest gene (GALM) and the “ABC linked gene” indicates GALM as the predicted target gene. FIG. 3C shows validation of a gRNA by RNA-seq that shows the nearest gene (COMMD8) is not differentially expressed, and therefore is not the likely target gene. FIG. 3D shows validation of a gRNA that has no predicted ABC target gene, but displays many differentially expressed genes. Genes with significant differences in gene expression are shown in dark gray (padj≤0.05). Gene ontologies show top 10 enriched categories for each RNA-seq analysis.

FIG. 4A shows individual gRNAs that were delivered to cells in a GFP expressing vector and co-seeded with equal numbers of cells transduced with a non-targeting gRNA in an mCherry expressing vector. Proportion of cells were assayed at day 1 post-seeding, and day 7 or day 14 post-seeding to determine the fold-change in the proportion of GFP vs mCherry positive cells. FIG. 4B shows individual validations for gRNAs that were depleted in the second sub-library screen. FIG. 4C shows individual validations for gRNAs that were enriched in the sub-library screen (n=3 biological replicates, mean±s.e.m.). Gene names represent putative target genes identified for each gRNA hit by ABC method (Fulco et al., Science. 2016, 354, 769-773, which is incorporated herein by reference in its entirety; Fulco et al., Nature Genetics. 2019, 51, 1664-1669, which is incorporated herein by reference in its entirety). Statistics indicate significance by two-way ANOVA with Dunnett's multiple comparisons test relative to non-targeting gRNA at day 7 or day 14: *P<0.05, ***P<0.001, ****P<0.0001.

FIG. 5A shows the characterization of promoter and distal DHS hits relative to chromatin accessibility from 53 diverse cell types. Specificity index of 1 indicates DHS that is unique to K562, while specificity index of 0 represents ubiquitous DHS site across all cell types. All DHS sites identified in K562 cells are shown as a comparison. FIG. 5B is a volcano plot showing 31,193 significant gRNAs either depleted or enriched following 14 population doublings of OCI-AML2 cells. Depleted gRNAs were more abundant and show larger effect size than enriched gRNAs. Dark gray points indicate significant gRNAs (FDR<0.1), mid-gray points indicate non-targeting control gRNAs, and light gray points indicate non-significant gRNAs. FIG. 5C is a comparison of the sub-library screen in K562 cells versus OCI-AML2 cells. Log 2 (fold-change) is plotted for every gRNA in each screen. Black points indicate gRNAs significant in both cell types. Dark gray points indicate gRNAs significant only in the OCI-AML2 cells, while light gray points indicate gRNAs only significant in K562 cells. Midgray points indicate gRNAs not significant in either cell type. Legend shows the number of gRNAs that are significant in either direction. FIG. 5D is a representative example of gRNA hits that are significantly depleted in both K562 and OCI-AML2 cell types. Blue box highlights the region of interest and red gRNAs indicate significant depletion. Note this region is marked by open chromatin in both cell types. FIG. 5E is a representative example of gRNAs that are significantly depleted in K562 cells, but not significant (black gRNAs, note Y-axis difference) in OCI-AML2 cells. Blue boxes highlight regions of interest, and the left region represents a DHS that is uniquely accessible in K562 cells.

FIG. 6A shows the relationship between distance of regulatory element-gene link and significance. Perturbations closer to the transcriptional start site of genes tend to be more significant overall. FIG. 6B shows the number of regulatory elements per individual gene detected. FIG. 6C shows the number of genes affected by individual regulatory elements. FIG. 6D shows DHS hit with seven gene connections listed in FIG. 6C. Blue box represents DHS targeted for silencing, as well as gRNA log 2 (fold-change) depletion through CERES, and chromatin accessibility. The seven target genes are shown as yellow connectors. Inset shows this DHS hit directly overlaps a CTCF binding site (also marked by CTCF ChromHMM chromatin state). FIG. 6E shows genome browser tracks of the LMO2 locus including links of enhancers to genes, and CERES depletion of three downstream regions listed at Target 1, 2, and 3. FIG. 6F is a single cell expression analysis showing significant depletion of LMO2 expression in cells containing gRNAs for Target 1 and Target 2, but not Target 3. Asterisks indicate empirical p-values <0.01. FIG. 6G shows LMO2 mRNA fold-change by qRT-PCR in response to individual gRNA perturbations for LMO2 Target 1, 2, and 3. ****p-value <0.0001 versus control, one-way analysis of variance followed by Dunnett's test (n=3 biological replicates; mean±s.e.m.).

FIG. 7 is an overview of gRNA design for discovery wgCERES screen, validation screen, and single cell scCERES screen. Shown are the number of gRNAs that are used for each screen, the number of DHS sites that are detected as significant, and the ones that were included in subsequent screens.

FIGS. 8A-8B shows significant DHS hits identified by using different gRNA groupings. “UpSet” plots showing the different sets of significant DHSs hits identified by each of the four gRNA grouping analyses shown in FIGS. 1C-1D. Significance was determined by assessing changes in abundance of single gRNAs (gRNA) or changes in averaged abundance of clusters of 2 adjacent gRNAs (Bin2), clusters of 3 adjacent gRNAs (Bin3), or all gRNAs within the entire DHS (DHS). FIG. 8A shows significant DHS hits that were depleted in the wgCERES screen by one or more of the four analyses. FIG. 8B shows significant DHS hits that were enriched in the wgCERES screen. FIG. 8C shows significant DHS hits that contained enriched and depleted gRNAs in the wgCERES screen.

FIG. 9 shows analysis of gRNA attributes in the screen. Various numerical features were collected for individually significant gRNAs and their means were plotted stratified by significance (FDR=0.05, plotted mean±s.e.m.). “True” represents gRNAs that were significantly changed in abundance (depleted or enriched) in the wgCERES screen (FDR<0.05). “False” represents gRNAs that were not significantly changed (FDR≥0.05). Statistics indicate significance by Wilcoxon tests: ***P<0.001.

FIGS. 10A-10B are representative screenshots of significant DHS hits that are distant from a TSS. Light blue boxes represent significant DHS hits from the wgCERES screen. wgCERES plots show enrichment or depletion of gRNAs (significant gRNAs are red, and nonsignificant gRNAs are gray). DNase-seq shows regions of chromatin accessibility. ChromHMM shows predicted chromatin state based on histone modifications, including promoter (red), putative enhancer (yellow), and polycomb repressed regions (gray). FIG. 10A shows three DHSs˜75 kb upstream of the LMO2 oncogene had significant depletion of gRNAs. Note that while the majority of DHS sites around LMO2 show a depletion in the assay, consistent with its well-characterized oncogenic potential, there is one intronic DHS site that shows enrichment, and is possibly indicative of a repressor for LMO2. FIG. 10B shows a representative example of significant DHS sites at the promoter and ˜20 kb upstream of the RPL39 gene. FIG. 10C shows a representative example of significant DHS sites at the promoter, immediately downstream, and ˜25 kb downstream of the CEBPB gene.

FIG. 11 is a UMAP dimensionality reduction with input factor distributions. Each panel shows UMAP dimensionality reduction overlaid with counts per million (CPM) enrichment for different histone modifications, CTCF, POL2, and p300 binding. Darker grays indicate more enriched signal for each ChIP-seq factor. In the last two panels, the composite wgCERES top3 score is overlaid. Darker grays for depleted DHSs indicate more negative values while darker grays for enriched scores indicate more positive values.

FIGS. 12A-12B are representative screenshots of DHS hits in polycomb-repressed regions. Shown are a number of significant DHS hits that are depleted in the wgCERES screen (red bars in wgCERES track). H3K27me3 ChIP-seq and ChromHMM track indicates polycomb repressed regions (gray). FIG. 12A is data from single-cell CERES (scCERES) screen and shows that perturbing the DHS hit highlighted in blue box impacts gene expression for both GPER and ZFAND2A genes. FIG. 12B is data from scCERES that shows that perturbing the DHS hit in blue box significantly impacts gene expression of both ADARB1 and LSS genes.

FIG. 13A shows a dark gray line that represents distances between significant promoter DHS hits (<3 kb from TSS) and the nearest significant DHS hit. To assess if these distances are different than chance, non-significant DHSs were randomly sampled 1,000 times. Each permutation had the same number of non-significant DHS sites as the total number of significant promoter DHSs hits. Light gray line represents distances between significant promoter DHS hits to permuted non-significant DHS. FIG. 13B is the same as FIG. 13A but for significant distal DHS hits (>3 kb from TSSs). Light gray line represents distances between significant distal DHS hits to permuted non-significant DHS. FIG. 13C shows deciles of equal sized bins for FIG. 13A showing box plots of distances for significant DHS hits vs. permuted data. Significant differences were determined by t-test. FIG. 13D is the same as FIG. 13C but for significant distal DHS hits. ****P 0.0001, *P 0.05, ns=not significant. FIG. 13E is a representative example of clustered hits that had 7 significant DHS hits in close proximity around the HDAC7/VDR locus.

FIGS. 14A-14B show a comparison of promoter wgCERES DHS hits to other published essentiality studies. Three previous studies have performed essentiality studies in K562 cells, including one study that performed CRISPRi targeting promoters (Horlbeck et al., eLife. 2016, 5, doi:10.7554/elife.19760, which is incorporated herein by reference in its entirety) and 2 studies using CRISPR/Cas9 targeting exons (Lenoir et al., Nucleic Acids Res. 2018, 46, D776-D780, which is incorporated herein by reference in its entirety; Wang et al., Cell. 2017, 168, 890-903.e15, which is incorporated herein by reference in its entirety). FIG. 14A is a scatter plot of effect sizes for promoters identified by wgCERES (Y-axis) vs. effect size for promoters targeted by CRISPRi (X-axis). Note that there is general correspondence between assays. FIG. 14B is an “UpSet” plot showing overlap of significant promoter/gene hits identified by all 4 studies. Note that in addition to these comparisons, the majority of regions identified and characterized herein were in distal non-promoter regulatory elements that were not identified in previous studies.

FIGS. 15A-15H show a distribution of hits across analyses in K562 distal sub screen. “UpSet” plots showing distribution statistically significant DHSs across each analysis type in the K562 distal sub-library. Distribution of DHSs that were statistically significant in both the K562 validation library and the first wgCERES discovery screen from the (FIG. 15A) gRNA-level, (FIG. 15B) bins of 2 gRNAs, (FIG. 15C) bins of 3 gRNAs, and (FIG. 15D) DHS-level analysis. Distributions of gRNAs present in both the K562 distal sub-library and only found in the first K562 wgCERES (FIG. 15E) gRNA-level analysis, (FIG. 15F) bins of 2 gRNAs, (FIG. 15G) bins of 3 gRNAs, and (FIG. 15H) DHS-level analyses. Targeting DHSs significant only in the grouped analyses from the discovery screen yielded significant DHSs (across multiple analysis types) in the validation screen, demonstrating the utility of these grouped analyses. Also, tiling DHSs more densely with gRNAs yields more DHSs that are significant across multiple analyses.

FIG. 16 is individual gRNA validations on mRNA abundance for predicted gene interactions. Individual gRNAs were tested in K562 cells constitutively expressing dCas9^{KRAB}. mRNA changes were detected through qRT-PCR. Black bars indicate validation gRNAs that were depleted in the screen. Checkered bars indicate gRNAs that were enriched in the screen. Bars with diagonal lines indicate non-targeting gRNA control. Gray bars indicate cells only expressing dCas9^{KRAB}, without gRNA transduction. Statistics indicate significance by one-way analysis of variance followed by Dunnett's test (n=3 biological replicates, mean±s.e.m.): ****P<0.0001, **P<0.01, *P<0.05 versus dCas9^{KRAB} only control. All fold enrichments are relative to the transduction of a control gRNA and normalized to TBP.

FIGS. 17A-17E show validation of DHS hits around SLC4A1 locus with individual gRNA perturbation. For significant DHS hits identified in the wgCERES discovery screen, a subset of individual gRNAs were tested in a separate validation experiment. After transducing a single gRNA into K562 cells that express dCas9^{KRAB} cells were expanded for ˜14 doublings and then harvested for RNA-seq. As a control, K562 cells not expressing dCas9^{KRAB} were transduced with the same gRNAs. FIG. 17A is a screenshot of a region around SLC4A1 that was targeted with 4 individual gRNAs (highlighted with blue boxes). FIG. 17B-17E are volcano plots and gene ontology enrichments for each of the 4 regions targeted, as shown in FIG. 17A. Genes with significant differences in gene expression are shown in dark gray (padj 0.05). Note that the SLC4A1 gene was the most depleted gene for each experiment.

FIGS. 18A-180 show validation of DHS hits around GMPR locus with individual gRNA perturbation. For significant DHS hits identified in wgCERES screen, a subset of individual gRNAs were tested in a separate validation experiment. After transducing a single gRNA into K562 cells that express dCas9^{KRAB}, cells were expanded for ˜14 doublings and then harvested for RNA-seq. As a control, K562 cells not expressing dCas9^{KRAB} were transduced with the same gRNAs. FIG. 18A is a screenshot of a region in the GMPR gene that was targeted with 2 individual gRNAs (highlighted with blue boxes). FIG. 18B-18C are volcano plots and gene ontology enrichments for each of the 4 regions targeted, as shown in FIG. 18A. Genes with significant differences in gene expression are shown in dark gray (padj 0.05). Note that the GMPR gene did not show any significant changes in gene expression. However, a number of histone genes were differentially expressed 8 Mb away in both experiments. FIG. 18D is a zoomed out screenshot of this region (blue highlight=GMPR, orange highlight=histones cluster 8 Mb away).

FIGS. 19A-19I show validation of DHS hits with individual gRNA perturbation. For significant DHS hits identified in wgCERES screen, a subset of individual gRNAs were tested in a separate validation experiment. After transducing a single gRNA into K562 cells that express dCas9^{KRAB}, cells were expanded for ˜14 doublings and then harvested for RNA-seq. As a control, K562 cells not expressing dCas9^{KRAB} were transduced with the same gRNAs. FIGS. 19A-19I show volcano plots and gene ontology enrichments for each region targeted. Genes with significant differences in gene expression are shown in dark gray (padj≤0.05).

FIGS. 20A-20C show a single cell CERES-seq gRNA distribution and analysis schematic. Cells constitutively expressing dCas9^{KRAB} were transduced with a lentiviral library of 3,201 gRNAs targeting 3,051 DNase-I hypersensitive sites and 150 non-targeting gRNAs. 56,882 cells were barcoded for single-cell RNA-seq at 5 days post-transduction. FIG. 20A is a distribution of the number of gRNAs per cell. Blue dashed line indicates the mean number of eight gRNAs per cell. FIG. 20B is a distribution of the number of cells containing each gRNA in the library. The dashed line indicates the mean number of 111 cells containing each individual gRNA. FIG. 20C is a schematic of scCERES analysis pipeline.

### DETAILED DESCRIPTION

As detailed herein, thousands of human gene regulatory elements were identified that functionally contribute to cell fitness, using a genome-wide CRISPR-based epigenome editing screen that individually targeted each of the >100,000 putative gene regulatory elements defined by open chromatin sites in human K562 leukemia cells for their role in regulating essential cellular processes. In an initial screen containing more than 1 million gRNAs, 12,000 regulatory elements with evidence of impact on cell fitness were discovered. The properties, distribution, cell-type specificity, and target genes of the identified regulatory elements were further characterized, including evaluating cell-type specificity in a second cancer cell line and identifying target genes of the regulatory elements using CERES perturbations combined with single cell RNA-seq. The identified regulatory elements and target genes confirmed and complemented results from gene-based screens and indicated new pathways and molecular processes that contribute to cell fitness. The comprehensive and quantitative genome-wide map of essential regulatory elements and function detailed herein represents a framework for extensive characterization of noncoding regulatory elements and variants that drive complex cell phenotypes and contribute to human traits, diseases, and disease risk. Further detailed herein are compositions and methods for targeting newly discovered gene regulatory elements affecting cell fitness to treat diseases such as leukemia.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medullablastoma, non-small cell lung cancer, and/or meothioma. In embodiments detailed herein, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated.

“Target region” as used herein refers to the region of the target gene to which the CRISPR/Cas9-based gene editing or targeting system is designed to bind.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132, which is incorporated herein by reference in its entirety). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. AGENTS TO MODIFY CELLULAR FITNESS

The compositions and methods detailed herein may be used, for example, to modify or modulate cellular fitness and/or treat disease. Modifying or modulating may include increasing or decreasing, for example. In some embodiments, the compositions and methods comprise an agent that modifies or modulates cellular fitness. The agent may comprise, for example, a polynucleotide, a polypeptide, a small molecule, a lipid, a carbohydrate, or a combination thereof. In some embodiments, the agent comprises a protein. In some embodiments, the agent comprises an antibody. In some embodiments, the agent comprises siRNA. In some embodiments, the agent comprises a DNA targeting composition as detailed herein or at least one component thereof.

The agent, or the composition or the method comprising the agent, may target a gene or a regulatory element thereof. Regulatory elements include, for example, promoters and enhancers. Regulatory elements may be within 1000 base pairs of the transcription start site. Regulatory elements may be within 600 base pairs of the transcription start site. The agent, or the composition or the method comprising the agent, may modify the expression of a gene. For example, the agent, or the composition or the method comprising the agent, may reduce, inhibit, increase, or enhance the expression of a gene. The agent, or the composition or the method comprising the agent, may directly or indirectly modulate the activity of the gene's protein product. For example, the agent, or the composition or the method comprising the agent, may increase or decrease the binding or enzymatic activity of the gene's protein product, inhibit the binding of the gene's protein product to another molecule or ligand, increase the binding of the gene's protein product to another molecule or ligand, increase or decrease the degradation of the gene's protein product, or a combination thereof. The gene, or a regulatory element thereof, or a region thereof, may be as listed in any one of TABLES 18A, 18B, 19A, and/or 19B. The gene, or a regulatory element thereof, or a region thereof, may be as listed in any one of TABLES S1-S17. TABLES S1-S17 are as in Klann et al. 2021, “Genome-wide annotation of gene regulatory elements linked to cell fitness” bioRxiv doi: 10.1101/2021.03.08.434470, which is incorporated herein by reference in its entirety, and which is referred to herein as “Klann et al.” TABLE S1 of Klann et al. is incorporated herein by reference in its entirety. TABLE S2 of Klann et al. is incorporated herein by reference in its entirety. TABLE S3 of Klann et al. is incorporated herein by reference in its entirety. TABLE S4 of Klann et al. is incorporated herein by reference in its entirety. TABLE S5 of Klann et al. is incorporated herein by reference in its entirety. TABLE S6 of Klann et al. is incorporated herein by reference in its entirety. TABLE S7 of Klann et al. is incorporated herein by reference in its entirety. TABLE S8 of Klann et al. is incorporated herein by reference in its entirety. TABLE S9 of Klann et al. is incorporated herein by reference in its entirety. TABLE S10 of Klann et al. is incorporated herein by reference in its entirety. TABLE S11 of Klann et al. is incorporated herein by reference in its entirety. TABLE S12 of Klann et al. is incorporated herein by reference in its entirety. TABLE S13 of Klann et al. is incorporated herein by reference in its entirety. TABLE S14 of Klann et al. is incorporated herein by reference in its entirety. TABLE S15 of Klann et al. is incorporated herein by reference in its entirety. TABLE S16 of Klann et al. is incorporated herein by reference in its entirety. TABLE S17 of Klann et al. is incorporated herein by reference in its entirety.

### 3. DNA TARGETING SYSTEMS

A “DNA Targeting System” as used herein is a system capable of specifically targeting a particular region of DNA and modulating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a guide RNA, a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

### 4. CRISPR/CAS-BASED GENE EDITING SYSTEM

Provided herein are CRISPR/Cas9-based gene editing systems. The CRISPR/Cas-based gene editing system may be used to modulate cellular fitness. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 20. In certain embodiments, the Cas9 molecule is a Staphylococcus aureus Cas9 molecule (also referred herein as “SaCas9”). SaCas9 may comprise an amino acid sequence of SEQ ID NO: 21. The Cas9 protein may comprise an amino acid sequence having at least 75%, 80%, 85%, 90%, 95%, 97%, 98%, 99% or greater identity to SEQ ID NO: 20 or 21, or any fragment thereof. The Cas9 protein may comprise an amino acid sequence having one, two, three, four, five or more changes selected from amino acid substitutions, insertions, or deletions, relative to SEQ ID NO: 20 or 21, or any fragment thereof.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647, which is incorporated herein by reference in its entirety). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681, which is incorporated herein by reference in its entirety). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 49).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 22. A S. pyogenes Cas9 protein with D10A and H849A mutations may comprise an amino acid sequence of SEQ ID NO: 23. The Cas9 protein may comprise an amino acid sequence having at least 75%, 80%, 85%, 90%, 95%, 97%, 98%, 99% or greater identity to SEQ ID NO: 22 or 23, or any fragment thereof. The Cas9 protein may comprise an amino acid sequence having one, two, three, four, five or more changes selected from amino acid substitutions, insertions, or deletions, relative to SEQ ID NO: 22 or 23, or any fragment thereof. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 24. In certain embodiments, the mutant S. aureus Cas9 molecule comprises a N580A mutation. The nucleotide sequence encoding this mutant S. aureus Cas9 molecule is set forth in SEQ ID NO: 25. The Cas9 protein may be encoded by a polynucleotide comprising a sequence having at least 75%, 80%, 85%, 90%, 95%, 97%, 98%, 99% or greater identity to SEQ ID NO: 24 or 25, or any fragment thereof. The Cas9 protein may be encoded by a polynucleotide comprising a sequence having one, two, three, four, five or more changes selected from nucleotide substitutions, insertions, or deletions, relative to SEQ ID NO: 24 or 25, or any fragment thereof.

In some embodiments, the Cas9 protein is a VQR variant. The VQR variant of Cas9 is a mutant with a different PAM recognition, as detailed in Kleinstiver, et al. (Nature 2015, 523, 481-485, which is incorporated herein by reference in its entirety).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 26. Exemplary codon optimized nucleic acid sequences encoding a Cas9 molecule of S. aureus, and optionally containing nuclear localization sequences (NLSs), are set forth in SEQ ID NOs: 27-33. Another exemplary codon optimized nucleic acid sequence encoding a Cas9 molecule of S. aureus comprises the nucleotides 1293-4451 of SEQ ID NO: 34.

b. Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)ₙ, wherein n is an integer between 0 and 10 (SEQ ID NO: 50). In a GS linker, n can be adjusted to optimize the linker length and achieve appropriate separation of the functional domains. Other examples of linkers may include, for example, Gly-Gly-Gly-Gly-Gly (SEQ ID NO: 51), Gly-Gly-Ala-Gly-Gly (SEQ ID NO: 52), Gly/Ser rich linkers such as Gly-Gly-Gly-Gly-Ser-Ser-Ser (SEQ ID NO: 53), or Gly/Ala rich linkers such as Gly-Gly-Gly-Gly-Ala-Ala-Ala (SEQ ID NO: 54).

In some embodiments, the Cas protein and/or the Cas fusion protein and/or gRNAs detailed herein may be used in compositions and methods for modulating expression of gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 35 or SEQ ID NO: 36. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 37, encoded by the polynucleotide of SEQ ID NO: 38. VPH may comprise a polypeptide having the amino acid sequence of SEQ ID NO: 45, encoded by the polynucleotide of SEQ ID NO: 46. VPR may comprise a polypeptide having the amino acid sequence of SEQ ID NO: 47, encoded by the polynucleotide of SEQ ID NO: 48.

ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. KRAB may comprise a polypeptide having an amino acid sequence of SEQ ID NO: 55, encoded by a polynucleotide comprising the sequence of SEQ ID NO: 56. For example, the fusion protein may be S. pyogenes dCas9-KRAB (polynucleotide sequence SEQ ID NO: 39; protein sequence SEQ ID NO: 40). The fusion protein may be S. aureus dCas9-KRAB (polynucleotide sequence SEQ ID NO: 41; protein sequence SEQ ID NO: 42).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 35 or SEQ ID NO: 36.

v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Teti, also known as Teti CD (Ten-eleven translocation methylcytosine dioxygenase 1; polynucleotide sequence SEQ ID NO: 43; amino acid sequence SEQ ID NO: 44). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target a gene, or a regulatory element thereof, or a region thereof, as listed in any one of TABLES 18A, 18B, 19A, and/or 19B. The gRNA may comprise a sequence, and/or be encoded by a sequence, and/or target a sequence, and/or correspond to a gene region, and/or bind to a gene region listed in any one of TABLES 18A, 18B, 19A, and/or 19B. The gRNA may target a gene, or a regulatory element thereof, or a region thereof, as listed in any one of TABLES S1-S17. The gRNA may comprise a sequence, and/or be encoded by a sequence, and/or target a sequence, and/or correspond to a gene region, and/or bind to a gene region listed in any one of TABLES S1-S17. TABLES S1-S17 are as in Klann et al. 2021, “Genome-wide annotation of gene regulatory elements linked to cell fitness” bioRxiv doi: 10.1101/2021.03.08.434470, which is incorporated herein by reference in its entirety, and which is referred to herein as “Klann et al.” TABLE S1 of Klann et al. is incorporated herein by reference in its entirety. TABLE S2 of Klann et al. is incorporated herein by reference in its entirety. TABLE S3 of Klann et al. is incorporated herein by reference in its entirety. TABLE S4 of Klann et al. is incorporated herein by reference in its entirety. TABLE S5 of Klann et al. is incorporated herein by reference in its entirety. TABLE S6 of Klann et al. is incorporated herein by reference in its entirety. TABLE S7 of Klann et al. is incorporated herein by reference in its entirety. TABLE S8 of Klann et al. is incorporated herein by reference in its entirety. TABLE S9 of Klann et al. is incorporated herein by reference in its entirety. TABLE S10 of Klann et al. is incorporated herein by reference in its entirety. TABLE S11 of Klann et al. is incorporated herein by reference in its entirety. TABLE S12 of Klann et al. is incorporated herein by reference in its entirety. TABLE S13 of Klann et al. is incorporated herein by reference in its entirety. TABLE S14 of Klann et al. is incorporated herein by reference in its entirety. TABLE S15 of Klann et al. is incorporated herein by reference in its entirety. TABLE S16 of Klann et al. is incorporated herein by reference in its entirety. TABLE S17 of Klann et al. is incorporated herein by reference in its entirety.

The gRNA may target a gene regulatory element. The gRNA may target a regulatory element of a gene selected from those listed in TABLE 18A or TABLE 19A. The gRNA may target a regulatory element of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR. In some embodiments, the gRNA targets a regulatory element of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 and may be used to decrease cell fitness. In some embodiments, the gRNA targets a regulatory element of a gene selected from FADS3, RPAP1, SLC25A39, RP13-20L14.6, FOXA2, and GMPR and may be used to increase cell fitness.

The gRNA may be selected from the gRNAs listed in TABLE 18A or TABLE 19A. The gRNA may comprise a polynucleotide sequence comprising at least one of SEQ ID NOs: 198-338, or a complement thereof, or a variant thereof, or a truncation thereof. The gRNA may be encoded by a polynucleotide sequence comprising at least one of SEQ ID NOs: 57-197, or a complement thereof, or a variant thereof, or a truncation thereof. The gRNA may bind and target a polynucleotide sequence comprising at least one of SEQ ID NOs: 339-479, or a complement thereof, or a variant thereof, or a truncation thereof. A truncation may be 1, 2, 3, 4, 5, 6, 7, 8, or 9 nucleotides shorter than the reference sequence.

In some embodiments, the gRNA targets a regulatory element and may be used to decrease cell fitness. For example, the gRNA may target a regulatory element associated with a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1. In some embodiments, the gRNA is selected from the gRNAs listed in TABLE 18A. The gRNA may comprise a polynucleotide sequence comprising at least one of SEQ ID NOs: 198-332, or a complement thereof, or a variant thereof, or a truncation thereof. The gRNA may be encoded by a polynucleotide sequence comprising at least one of SEQ ID NOs: 57-191, or a complement thereof, or a variant thereof, or a truncation thereof. The gRNA may bind and target a polynucleotide sequence comprising at least one of SEQ ID NOs: 339-473, or a complement thereof, or a variant thereof, or a truncation thereof. A truncation may be 1, 2, 3, 4, 5, 6, 7, 8, or 9 nucleotides shorter than the reference sequence. Decreasing cell fitness may include, for example, decreasing cell growth, decreasing cell growth rate, decreasing cell growth duration, decreasing cell size, increasing cell death, or a combination thereof.

In some embodiments, the gRNA targets a regulatory element and may be used to increase cell fitness. For example, the gRNA may target a regulatory element associated with a gene selected from FADS3, RPAP1, SLC25A39, RP13-20L14.6, FOXA2, and GMPR. In some embodiments, the gRNA is selected from the gRNAs listed in TABLE 19A. The gRNA may comprise a polynucleotide sequence comprising at least one of SEQ ID NOs: 333-338, or a complement thereof, or a variant thereof, or a truncation thereof. The gRNA may be encoded by a polynucleotide sequence comprising at least one of SEQ ID NOs: 192-197, or a complement thereof, or a variant thereof, or a truncation thereof. The gRNA may bind and target a polynucleotide sequence comprising at least one of SEQ ID NOs: 474-479, or a complement thereof, or a variant thereof, or a truncation thereof. A truncation may be 1, 2, 3, 4, 5, 6, 7, 8, or 9 nucleotides shorter than the reference sequence. Increasing cell fitness may include, for example, increasing cell growth, increasing cell growth rate, increasing cell growth duration, increasing cell size, or a combination thereof.

TABLE 18A
gRNAs for use in decreasing cell fitness.

| | | | | | log2Fold |
|---|---|---|---|---|---|
| # | DNA encoding gRNA | gRNA | Target | Gene | Change |
| 1 | CTCAAGGGAGAAGGTTAAT | CUCAAGGGAGAAGGUUAA | CCACTCAAGGGAGAAGGTTA | SCD | −1.499927588 |
| | T (SEQ ID NO: 57) | UU (SEQ ID NO: 198) | ATT (SEQ ID NO: 339) | | |
| 2 | GGCCTACCAGATAACCAGT | GGCCUACCAGAUAACCAG | GGCCTACCAGATAACCAGTTG | LDB1 | −1.773061245 |
| | T (SEQ ID NO: 58) | UU (SEQ ID NO: 199) | GG (SEQ ID NO: 340) | | |
| 3 | GGCCTACCAGATAACCAGT | GGCCUACCAGAUAACCAG | GGCCTACCAGATAACCAGTTG | NOLC1 | −1.773061245 |
| | T (SEQ ID NO: 59) | UU (SEQ ID NO: 200) | GG (SEQ ID NO: 341) | | |
| 4 | TGCGTCACATGAGAGGAA | UGCGUCACAUGAGAGGAA | TGCGTCACATGAGAGGAAGTT | CASP7 | −1.710885183 |
| | GT (SEQ ID NO:60) | GU (SEQ ID NO: 201) | GG (SEQ ID NO: 342) | | |
| 5 | AGTGACAGTGGATGCCATA | AGUGACAGUGGAUGCCAU | AGTGACAGTGGATGCCATAAC | EIF3A | −2.269982229 |
| | A (SEQ ID NO: 61) | AA (SEQ ID NO: 202) | GG (SEQ ID NO: 343) | | |
| 6 | AGTGACAGTGGATGCCATA | AGUGACAGUGGAUGCCAU | AGTGACAGTGGATGCCATAAC | FAM45A | −2.269982229 |
| | A (SEQ ID NO: 62) | AA (SEQ ID NO: 203) | GG (SEQ ID NO: 344) | | |
| 7 | GAGGGGGAGCGGGGCGA | GAGGGGGAGCGGGGCGA | CCCGAGGGGGAGCGGGGCG | BNIP3 | −0.763455908 |
| | GAG (SEQ ID NO: 63) | GAG (SEQ ID NO: 204) | AGAG (SEQ ID NO: 345) | | |
| 8 | CGGGAGTGGCTGCTCGCG | CGGGAGUGGCUGCUCGC | CGGGAGTGGCTGCTCGCGGA | MASTL | −3.669938146 |
| | GA (SEQ ID NO: 64) | GGA (SEQ ID NO: 205) | GGG (SEQ ID NO: 346) | | |
| 9 | GGAGGTGAGGAAGGAGGG | GGAGGUGAGGAAGGAGG | CCAGGAGGTGAGGAAGGAGG | AKR1E2 | −0.297415894 |
| | AA (SEQ ID NO: 65) | GAA (SEQ ID NO: 206) | GAA (SEQ ID NO: 347) | | |
| 10 | GCAGTGCCTCCGGCGGGG | GCAGUGCCUCCGGCGGG | GCAGTGCCTCCGGCGGGGGT | CRTAM | −0.382928483 |
| | GT (SEQ ID NO: 66) | GGU (SEQ ID NO: 207) | AGG (SEQ ID NO: 348) | | |
| 11 | TGATTAGGGACAGTTCCCC | UGAUUAGGGACAGUUCCC | CCTTGATTAGGGACAGTTCCC | LMO2 | −2.299987034 |
| | G (SEQ ID NO: 67) | CG (SEQ ID NO: 208) | CG (SEQ ID NO: 349) | | |
| 12 | TAACTGTTACATGAAGACA | UAACUGUUACAUGAAGAC | CCCTAACTGTTACATGAAGAC | LMO2 | −4.187824104 |
| | A (SEQ ID NO: 68) | AA (SEQ ID NO: 209) | AA (SEQ ID NO: 350) | | |
| 13 | GGCGCCCAGAAAACCTAG | GGCGCCCAGAAAACCUAG | GGCGCCCAGAAAACCTAGGT | GAB2 | −2.6002839 |
| | GT (SEQ ID NO: 69) | GU (SEQ ID NO: 210) | GGG (SEQ ID NO: 351) | | |
| 14 | TCTATCTTCTGCCCTGACT | UCUAUCUUCUGCCCUGAC | CCATCTATCTTCTGCCCTGAC | GAB2 | −2.409253932 |
| | T (SEQ ID NO: 70) | UU (SEQ ID NO: 211) | TT (SEQ ID NO: 352) | | |
| 15 | AGGGGCGAGCGGAGAGGA | AGGGGCGAGCGGAGAGG | CCTAGGGGCGAGCGGAGAG | PGAM5 | −0.871448583 |
| | GG (SEQ ID NO: 71) | AGG (SEQ ID NO: 212) | GAGG (SEQ ID NO: 353) | | |
| 16 | GCGCCTCGCGTTCCTTGG | GCGCCUCGCGUUCCUUG | GCGCCTCGCGTTCCTTGGTA | YARS2 | −2.920738928 |
| | TA (SEQ ID NO: 72) | GUA (SEQ ID NO: 213) | CGG (SEQ ID NO: 354) | | |
| 17 | TTAAGCTAGGGAGAATATT | UUAAGCUAGGGAGAAUAU | CCATTAAGCTAGGGAGAATAT | KLHDC1 | −2.482482045 |
| | G (SEQ ID NO: 73) | UG (SEQ ID NO: 214) | TG (SEQ ID NO: 355) | | |
| 18 | TCCCTCTGGGGTTGAGGA | UCCCUCUGGGGUUGAGG | TCCCTCTGGGGTTGAGGAGG | PDCD7 | −0.660746748 |
| | GG (SEQ ID NO: 74) | AGG (SEQ ID NO: 215) | AGG (SEQ ID NO: 356) | | |
| 19 | TCCCTCTGGGGTTGAGGA | UCCCUCUGGGGUUGAGG | TCCCTCTGGGGTTGAGGAGG | ZNF609 | −0.660746748 |
| | GG (SEQ ID NO: 75) | AGG (SEQ ID NO: 216) | AGG (SEQ ID NO: 357) | | |
| 20 | TGAGACCAGCCTGGGTGA | UGAGACCAGCCUGGGUGA | CCGTGAGACCAGCCTGGGTG | NR2F2 | −0.584098352 |
| | CA (SEQ ID NO: 76) | CA (SEQ ID NO: 217) | ACA (SEQ ID NO: 358) | | |
| 21 | TGAGACCAGCCTGGGTGA | UGAGACCAGCCUGGGUGA | CCGTGAGACCAGCCTGGGTG | NR2F2- | −0.584098352 |
| | CA (SEQ ID NO: 77) | CA (SEQ ID NO: 218) | ACA (SEQ ID NO: 359) | AS1 | |
| 22 | GGGAACTGGAACTGCCTG | GGGAACUGGAACUGCCUG | GGGAACTGGAACTGCCTGCG | PLK1 | −4.746174907 |
| | CG (SEQ ID NO: 78) | CG (SEQ ID NO: 219) | GGG (SEQ ID NO: 360) | | |
| 23 | GGCGAAAGCCGACTCGAG | GGCGAAAGCCGACUCGAG | GGCGAAAGCCGACTCGAGGG | ZG16B | −0.219671275 |
| | GG (SEQ ID NO: 79) | GG (SEQ ID NO: 220) | TGG (SEQ ID NO: 361) | | |
| 24 | CCGGTCAGGGATGTTAGG | CCGGUCAGGGAUGUUAG | CCGGTCAGGGATGTTAGGAG | CBFA2T3 | −4.224370075 |
| | AG (SEQ ID NO: 80) | GAG (SEQ ID NO: 221) | CGG (SEQ ID NO: 362) | | |
| 25 | CCGGTCAGGGATGTTAGG | CCGGUCAGGGAUGUUAG | CCGGTCAGGGATGTTAGGAG | MVD | −4.224370075 |
| | AG (SEQ ID NO: 81) | GAG (SEQ ID NO: 222) | CGG (SEQ ID NO: 363) | | |
| 26 | CCGGTCAGGGATGTTAGG | CCGGUCAGGGAUGUUAG | CCGGTCAGGGATGTTAGGAG | SPATA33 | −4.224370075 |
| | AG (SEQ ID NO: 82) | GAG (SEQ ID NO: 223) | CGG (SEQ ID NO: 364) | | |
| 27 | TGCTCCTGGAAGCCCCAC | UGCUCCUGGAAGCCCCAC | TGCTCCTGGAAGCCCCACCC | SREBF1 | −0.797359326 |
| | CC (SEQ ID NO: 83) | CC (SEQ ID NO: 224) | CGG (SEQ ID NO: 365) | | |
| 28 | TGATAGAGCTGGGGGACC | UGAUAGAGCUGGGGGACC | CCCTGATAGAGCTGGGGGAC | CTD- | −0.53313193 |
| | CG (SEQ ID NO: 84) | CG (SEQ ID NO: 225) | CCG (SEQ ID NO: 366) | 2008P7.1 | |
| 29 | TTACAGCAGGAACAAGACT | UUACAGCAGGAACAAGAC | TTACAGCAGGAACAAGACTCA | CCR10 | −1.843687278 |
| | C (SEQ ID NO: 85) | UC (SEQ ID NO: 226) | GG (SEQ ID NO: 367) | | |
| 30 | TTACAGCAGGAACAAGACT | UUACAGCAGGAACAAGAC | TTACAGCAGGAACAAGACTCA | HAP1 | −1.843687278 |
| | C (SEQ ID NO: 86) | UC (SEQ ID NO: 227) | GG (SEQ ID NO: 368) | | |
| 31 | TTACAGCAGGAACAAGACT | UUACAGCAGGAACAAGAC | TTACAGCAGGAACAAGACTCA | PTRF | −1.843687278 |
| | C (SEQ ID NO: 87) | UC (SEQ ID NO: 228) | GG (SEQ ID NO: 369) | | |
| 32 | TTACAGCAGGAACAAGACT | UUACAGCAGGAACAAGAC | TTACAGCAGGAACAAGACTCA | STAT3 | −1.843687278 |
| | C (SEQ ID NO: 88) | UC (SEQ ID NO: 229) | GG (SEQ ID NO: 370) | | |
| 33 | TTACAGCAGGAACAAGACT | UUACAGCAGGAACAAGAC | TTACAGCAGGAACAAGACTCA | STAT5A | −1.843687278 |
| | C (SEQ ID NO: 89) | UC (SEQ ID NO: 230) | GG (SEQ ID NO: 371) | | |
| 34 | TTACAGCAGGAACAAGACT | UUACAGCAGGAACAAGAC | TTACAGCAGGAACAAGACTCA | STAT5B | −1.843687278 |
| | C (SEQ ID NO: 90) | UC (SEQ ID NO: 231) | GG (SEQ ID NO: 372) | | |
| 35 | AGGGATGGACCCCAGCTC | AGGGAUGGACCCCAGCUC | AGGGATGGACCCCAGCTCCA | CAMKK1 | −1.155590004 |
| | CA (SEQ ID NO: 91) | CA (SEQ ID NO: 232) | GGG (SEQ ID NO: 373) | | |
| 36 | ACTAGCAGAAGGCCCTGA | ACUAGCAGAAGGCCCUGA | CCAACTAGCAGAAGGCCCTG | RSAD1 | −0.793709187 |
| | AG (SEQ ID NO: 92) | AG (SEQ ID NO: 233) | AAG (SEQ ID NO: 374) | | |
| 37 | ACTAGCAGAAGGCCCTGA | ACUAGCAGAAGGCCCUGA | CCAACTAGCAGAAGGCCCTG | XYLT2 | −0.793709187 |
| | AG (SEQ ID NO: 93) | AG (SEQ ID NO: 234) | AAG (SEQ ID NO: 375) | | |
| 38 | AGCTGGACTGGGCCAGAG | AGCUGGACUGGGCCAGAG | AGCTGGACTGGGCCAGAGCG | ERN1 | −1.456150356 |
| | CG (SEQ ID NO: 94) | CG (SEQ ID NO: 235) | GGG (SEQ ID NO: 376) | | |
| 39 | GCCCAGTTGGGGGATTCG | GCCCAGUUGGGGGAUUC | CCAGCCCAGTTGGGGGATTC | CARD14 | −4.195662865 |
| | GG (SEQ ID NO: 95) | GGG (SEQ ID NO: 236) | GGG (SEQ ID NO: 377) | | |
| 40 | ACGTGGAGGGGCGGCTCC | ACGUGGAGGGGCGGCUC | ACGTGGAGGGGCGGCTCCGT | KLF1 | −1.316522962 |
| | GT (SEQ ID NO: 96) | CGU (SEQ ID NO: 237) | GGG (SEQ ID NO: 378) | | |
| 41 | ACGTGGAGGGGCGGCTCC | ACGUGGAGGGGCGGCUC | ACGTGGAGGGGCGGCTCCGT | TNPO2 | −1.316522962 |
| | GT (SEQ ID NO: 97) | CGU (SEQ ID NO: 238) | GGG (SEQ ID NO: 379) | | |
| 42 | GATTCCTGCGGGAACCGG | GAUUCCUGCGGGAACCGG | GATTCCTGCGGGAACCGGGG | RASAL3 | −0.401754515 |
| | GG (SEQ ID NO: 98) | GG (SEQ ID NO: 239) | CGG (SEQ ID NO: 380) | | |
| 43 | GGTCGACAGCTTGGGTCC | GGUCGACAGCUUGGGUC | GGTCGACAGCTTGGGTCCCT | AC005256.1 | −0.757866457 |
| | CT (SEQ ID NO: 99) | CCU (SEQ ID NO: 240) | CGG (SEQ ID NO: 381) | | |
| 44 | GGTCGACAGCTTGGGTCC | GGUCGACAGCUUGGGUC | GGTCGACAGCTTGGGTCCCT | GIPC3 | −0.757866457 |
| | CT (SEQ ID NO: 100) | CCU (SEQ ID NO: 241) | CGG (SEQ ID NO: 382) | | |
| 45 | GGTCGACAGCTTGGGTCC | GGUCGACAGCUUGGGUC | GGTCGACAGCTTGGGTCCCT | MKNK2 | −0.757866457 |
| | CT (SEQ ID NO: 101) | CCU (SEQ ID NO: 242) | CGG (SEQ ID NO: 383) | | |
| 46 | ACTTGAGCCTGGGGGTGT | ACUUGAGCCUGGGGGUG | ACTTGAGCCTGGGGGTGTCC | PDCD5 | −2.45026283 |
| | CC (SEQ ID NO: 102) | UCC (SEQ ID NO: 243) | AGG (SEQ ID NO: 384) | | |
| 47 | ACTAACCCGCTGGCCCTC | ACUAACCCGCUGGCCCUC | CCTACTAACCCGCTGGCCCT | CTC- | −0.585672828 |
| | CC (SEQ ID NO: 103) | CC (SEQ ID NO: 244) | CCC (SEQ ID NO: 385) | 273B12.10 | |
| 48 | TCTGAGGTGTGACCACACA | UCUGAGGUGUGACCACAC | TCTGAGGTGTGACCACACAG | CTD- | −2.700478104 |
| | G (SEQ ID NO: 104) | AG (SEQ ID NO: 245) | AGG (SEQ ID NO: 386) | 3073N11.9 | |
| 49 | GAGTGAGGGGAGGTGGAG | GAGUGAGGGGAGGUGGA | CCTGAGTGAGGGGAGGTGGA | AC008440.5 | −2.203212712 |
| | AG (SEQ ID NO: 105) | GAG (SEQ ID NO: 246) | GAG (SEQ ID NO: 387) | | |
| 50 | TCATCGACCTAGCTCCCAG | UCAUCGACCUAGCUCCCA | CCATCATCGACCTAGCTCCCA | SARS | −2.55293289 |
| | A (SEQ ID NO: 106) | GA (SEQ ID NO: 247) | GA (SEQ ID NO: 388) | | |
| 51 | GGGAGTGGCACACCCTGA | GGGAGUGGCACACCCUGA | CCTGGGAGTGGCACACCCTG | SARS | −0.711547573 |
| | TA (SEQ ID NO: 107) | UA (SEQ ID NO: 248) | ATA (SEQ ID NO: 389) | | |
| 52 | TCCCTACACGGGCAGGAG | UCCCUACACGGGCAGGAG | CCTTCCCTACACGGGCAGGA | RP5- | −1.667165813 |
| | GA (SEQ ID NO: 108) | GA (SEQ ID NO: 249) | GGA (SEQ ID NO: 390) | 1065J22.8 | |
| 53 | TCCCTACACGGGCAGGAG | UCCCUACACGGGCAGGAG | CCTTCCCTACACGGGCAGGA | SARS | −1.667165813 |
| | GA (SEQ ID NO: 109) | GA (SEQ ID NO: 250) | GGA (SEQ ID NO: 391) | | |
| 54 | GTCCCTCCCGTGGGGCTG | GUCCCUCCCGUGGGGCU | CCCGTCCCTCCCGTGGGGCT | DFFA | −1.059035361 |
| | AT (SEQ ID NO: 110) | GAU (SEQ ID NO: 251) | GAT (SEQ ID NO: 392) | | |
| 55 | GTCCCTCCCGTGGGGCTG | GUCCCUCCCGUGGGGCU | CCCGTCCCTCCCGTGGGGCT | KIAA2013 | −1.059035361 |
| | AT (SEQ ID NO: 111) | GAU (SEQ ID NO: 252) | GAT (SEQ ID NO: 393) | | |
| 56 | TCATAGTCACACCCAGGAG | UCAUAGUCACACCCAGGA | TCATAGTCACACCCAGGAGGT | RP11- | −5.360362095 |
| | G (SEQ ID NO: 112) | GG (SEQ ID NO: 253) | GG (SEQ ID NO: 394) | 196G18.24 | |
| 57 | GAGGCACAGCCTGCTCTC | GAGGCACAGCCUGCUCUC | CCTGAGGCACAGCCTGCTCT | THEM4 | −0.304275846 |
| | TC (SEQ ID NO: 113) | UC (SEQ ID NO: 254) | CTC (SEQ ID NO: 395) | | |
| 58 | GGGGAGCTGGGGAGGATG | GGGGAGCUGGGGAGGAU | GGGGAGCTGGGGAGGATGG | SLAMF1 | −0.202557574 |
| | GA (SEQ ID NO: 114) | GGA (SEQ ID NO: 255) | ATGG (SEQ ID NO: 396) | | |
| 59 | CCGATGGGAGGAGGCAGG | CCGAUGGGAGGAGGCAG | CCGATGGGAGGAGGCAGGG | snoU13 | −0.312253516 |
| | GG (SEQ ID NO: 115) | GGG (SEQ ID NO: 256) | GTGG (SEQ ID NO: 397) | | |
| 60 | AGCGCCCTGAGAGCCCTG | AGCGCCCUGAGAGCCCUG | CCCAGCGCCCTGAGAGCCCT | PPP1R15B | −1.858478461 |
| | AA (SEQ ID NO: 116) | AA (SEQ ID NO: 257) | GAA (SEQ ID NO: 398) | | |
| 61 | GCTGTACAGGGCGAGAGA | GCUGUACAGGGCGAGAGA | CCGGCTGTACAGGGCGAGAG | RP5- | −2.523650892 |
| | AG (SEQ ID NO: 117) | AG (SEQ ID NO: 258) | AAG (SEQ ID NO: 399) | 1092A3.4 | |
| 62 | TGTGACCGGACCACCCTC | UGUGACCGGACCACCCUC | CCTTGTGACCGGACCACCCT | MEGF6 | −1.098391527 |
| | CA (SEQ ID NO: 118) | CA (SEQ ID NO: 259) | CCA (SEQ ID NO: 400) | | |
| 63 | TGTGACCGGACCACCCTC | UGUGACCGGACCACCCUC | CCTTGTGACCGGACCACCCT | WRAP73 | −1.098391527 |
| | CA (SEQ ID NO: 119) | CA (SEQ ID NO: 260) | CCA (SEQ ID NO: 401) | | |
| 64 | GCCAGAACGTGAACCACT | GCCAGAACGUGAACCACU | CCTGCCAGAACGTGAACCAC | CDC20 | −1.990500496 |
| | GG (SEQ ID NO: 120) | GG (SEQ ID NO: 261) | TGG (SEQ ID NO: 402) | | |
| 65 | GCCAGAACGTGAACCACT | GCCAGAACGUGAACCACU | CCTGCCAGAACGTGAACCAC | TIE1 | −1.990500496 |
| | GG (SEQ ID NO: 121) | GG (SEQ ID NO: 262) | TGG (SEQ ID NO: 403) | | |
| 66 | CTGAGACCCAGGAGGTGC | CUGAGACCCAGGAGGUGC | CCCCTGAGACCCAGGAGGTG | DNAJC11 | −1.568794977 |
| | TG (SEQ ID NO: 122) | UG (SEQ ID NO: 263) | CTG (SEQ ID NO: 404) | | |
| 67 | AGGCCTAGCAGTGCGAGT | AGGCCUAGCAGUGCGAGU | AGGCCTAGCAGTGCGAGTGT | BCL2L1 | −2.930273816 |
| | GT (SEQ ID NO: 123) | GU (SEQ ID NO: 264) | GGG (SEQ ID NO: 405) | | |
| 68 | GGCCACAGGATGTCAAGA | GGCCACAGGAUGUCAAGA | CCTGGCCACAGGATGTCAAG | TPX2 | −2.724166279 |
| | AA (SEQ ID NO: 124) | AA (SEQ ID NO: 265) | AAA (SEQ ID NO: 406) | | |
| 69 | GGAGGGCACCCAGGTCCT | GGAGGGCACCCAGGUCCU | GGAGGGCACCCAGGTCCTCA | OSBPL2 | −4.046949933 |
| | CA (SEQ ID NO: 125) | CA (SEQ ID NO: 266) | TGG (SEQ ID NO: 407) | | |
| 70 | GGAGGGCACCCAGGTCCT | GGAGGGCACCCAGGUCCU | GGAGGGCACCCAGGTCCTCA | SS18L1 | −4.046949933 |
| | CA (SEQ ID NO: 126) | CA (SEQ ID NO: 267) | TGG (SEQ ID NO: 408) | | |
| 71 | GGGCGTGAGGGGGCCAAC | GGGCGUGAGGGGGCCAA | GGGCGTGAGGGGGCCAACCA | AP000265.1 | −4.090254293 |
| | CA (SEQ ID NO: 127) | CCA (SEQ ID NO: 268) | TGG (SEQ ID NO:409 ) | | |
| 72 | GGGCGTGAGGGGGCCAAC | GGGCGUGAGGGGGCCAA | GGGCGTGAGGGGGCCAACCA | IL10RB | −4.090254293 |
| | CA (SEQ ID NO: 128) | CCA (SEQ ID NO: 269) | TGG (SEQ ID NO: 410) | | |
| 73 | GGGCGTGAGGGGGCCAAC | GGGCGUGAGGGGGCCAA | GGGCGTGAGGGGGCCAACCA | MIS18A | −4.090254293 |
| | CA (SEQ ID NO: 129) | CCA (SEQ ID NO: 270) | TGG (SEQ ID NO: 411) | | |
| 74 | GGGCGTGAGGGGGCCAAC | GGGCGUGAGGGGGCCAA | GGGCGTGAGGGGGCCAACCA | MRPS6 | −4.090254293 |
| | CA (SEQ ID NO: 130) | CCA (SEQ ID NO: 271) | TGG (SEQ ID NO: 412) | | |
| 75 | AGAGGACAGAGGCAGGAG | AGAGGACAGAGGCAGGAG | AGAGGACAGAGGCAGGAGGG | AP001476.4 | −0.683911933 |
| | GG (SEQ ID NO: 131) | GG (SEQ ID NO: 272) | AGG (SEQ ID NO: 413) | | |
| 76 | TCCCCTCTAGCGGTAAGG | UCCCCUCUAGCGGUAAGG | CCATCCCCTCTAGCGGTAAG | USP18 | −0.726414208 |
| | CC (SEQ ID NO: 132) | CC (SEQ ID NO: 273) | GCC (SEQ ID NO: 414) | | |
| 77 | CTCATCCATCGGCCATGTG | CUCAUCCAUCGGCCAUGU | CTCATCCATCGGCCATGTGCA | AC004463.6 | −0.240173353 |
| | C (SEQ ID NO: 133) | GC (SEQ ID NO: 274) | GG (SEQ ID NO: 415) | | |
| 78 | TTAACCTGGGGGGAACCC | UUAACCUGGGGGGAACCC | CCTTTAACCTGGGGGGAACC | ERCC3 | −0.880872443 |
| | AC (SEQ ID NO: 134) | AC (SEQ ID NO: 275) | CAC (SEQ ID NO: 416) | | |
| 79 | GCGCTCAGTAACCGGAGG | GCGCUCAGUAACCGGAGG | CCTGCGCTCAGTAACCGGAG | SRBD1 | −3.510953718 |
| | AA (SEQ ID NO: 135) | AA (SEQ ID NO: 276) | GAA (SEQ ID NO: 417) | | |
| 80 | GGAGAGCTGGGGCCACAG | GGAGAGCUGGGGCCACA | CCCGGAGAGCTGGGGCCACA | BCYRN1 | −0.669374994 |
| | CT (SEQ ID NO: 136) | GCU (SEQ ID NO: 277) | GCT (SEQ ID NO: 418) | | |
| 81 | GGAGAGCTGGGGCCACAG | GGAGAGCUGGGGCCACA | CCCGGAGAGCTGGGGCCACA | EPCAM | −0.669374994 |
| | CT (SEQ ID NO: 137) | GCU (SEQ ID NO: 278) | GCT (SEQ ID NO: 419) | | |
| 82 | GGGCCCAGCCAGTCCCAA | GGGCCCAGCCAGUCCCAA | CCCGGGCCCAGCCAGTCCCA | FOXN2 | −1.056757668 |
| | CT (SEQ ID NO: 138) | CU (SEQ ID NO: 279) | ACT (SEQ ID NO: 420) | | |
| 83 | GCCCGAGGAGTGGGACGT | GCCCGAGGAGUGGGACG | GCCCGAGGAGTGGGACGTGG | PNPT1 | −3.787114905 |
| | GG (SEQ ID NO: 139) | UGG (SEQ ID NO: 280) | GGG (SEQ ID NO: 421) | | |
| 84 | GTGGGACCTCTCCGATTCA | GUGGGACCUCUCCGAUUC | CCTGTGGGACCTCTCCGATTC | HK2 | −1.659673132 |
| | C (SEQ ID NO: 140) | AC (SEQ ID NO: 281) | AC (SEQ ID NO: 422) | | |
| 85 | TCCTCGAGCCCACCCCCG | UCCUCGAGCCCACCCCCG | CCCTCCTCGAGCCCACCCCC | INO80B | −1.707593267 |
| | CA (SEQ ID NO: 141) | CA (SEQ ID NO: 282) | GCA (SEQ ID NO: 423) | | |
| 86 | GTCCCTATGGACCAGCAC | GUCCCUAUGGACCAGCAC | GTCCCTATGGACCAGCACCA | GHRLOS | −1.182500906 |
| | CA (SEQ ID NO: 142) | CA (SEQ ID NO: 283) | GGG (SEQ ID NO: 424) | | |
| 87 | GGAGGTAGCAAAAACCCT | GGAGGUAGCAAAAACCCU | GGAGGTAGCAAAAACCCTGG | ATP6V1A | −3.139614024 |
| | GG (SEQ ID NO: 143) | GG (SEQ ID NO: 284) | AGG (SEQ ID NO: 425) | | |
| 88 | TTCAGGCCATGAAGGGAA | UUCAGGCCAUGAAGGGAA | TTCAGGCCATGAAGGGAAGT | RP11- | −1.067410009 |
| | GT (SEQ ID NO: 144) | GU (SEQ ID NO: 285) | GGG (SEQ ID NO: 426) | 53616.2 | |
| 89 | ACTCCCCTCCGAGAGCCG | ACUCCCCUCCGAGAGCCG | ACTCCCCTCCGAGAGCCGGG | DROSHA | −1.775504833 |
| | GG (SEQ ID NO: 145) | GG (SEQ ID NO: 286) | CGG (SEQ ID NO: 427) | | |
| 90 | CTGCCAGCGGGAACTGTG | CUGCCAGCGGGAACUGUG | CTGCCAGCGGGAACTGTGTA | PELO | −5.486691788 |
| | TA (SEQ ID NO: 146) | UA (SEQ ID NO: 287) | GGG (SEQ ID NO: 428) | | |
| 91 | AGGTGAACATCCCTAGGAA | AGGUGAACAUCCCUAGGA | AGGTGAACATCCCTAGGAAAA | PELO | −5.468864005 |
| | A (SEQ ID NO: 147) | AA (SEQ ID NO: 288) | GG (SEQ ID NO: 429) | | |
| 92 | TCACAGCTGGTCGGAAGC | UCACAGCUGGUCGGAAGC | TCACAGCTGGTCGGAAGCTC | RIOK2 | −2.456552248 |
| | TC (SEQ ID NO: 148) | UC (SEQ ID NO: 289) | AGG (SEQ ID NO: 430) | | |
| 93 | GCATGCCCGGGACCAGCT | GCAUGCCCGGGACCAGCU | CCCGCATGCCCGGGACCAGC | PHACTR1 | −1.64365509 |
| | GT (SEQ ID NO: 149) | GU (SEQ ID NO: 290) | TGT (SEQ ID NO: 431) | | |
| 94 | TTAGGGCACTTGAGAGACT | UUAGGGCACUUGAGAGAC | TTAGGGCACTTGAGAGACTG | AHI1 | −1.396274517 |
| | G (SEQ ID NO: 150) | UG (SEQ ID NO: 291) | GGG (SEQ ID NO: 432) | | |
| 95 | TTAGGGCACTTGAGAGACT | UUAGGGCACUUGAGAGAC | TTAGGGCACTTGAGAGACTG | MYB | −1.396274517 |
| | G (SEQ ID NO: 151) | UG (SEQ ID NO: 292) | GGG (SEQ ID NO: 433) | | |
| 96 | CTGAAACCAAAGCAGTACT | CUGAAACCAAAGCAGUAC | CTGAAACCAAAGCAGTACTTT | MYB | −2.406371188 |
| | T (SEQ ID NO: 152) | UU (SEQ ID NO: 293) | GG (SEQ ID NO: 434) | | |
| 97 | TGAGAGCAGGCGGAGGGA | UGAGAGCAGGCGGAGGG | CCATGAGAGCAGGCGGAGGG | ULBP1 | −0.274041877 |
| | AG (SEQ ID NO: 153) | AAG (SEQ ID NO: 294) | AAG (SEQ ID NO: 435) | | |
| 98 | GAGGGCGGCTGGGTGTCG | GAGGGCGGCUGGGUGUC | CCCGAGGGCGGCTGGGTGTC | FBXO5 | −4.044506266 |
| | GG (SEQ ID NO: 154) | GGG (SEQ ID NO: 295) | GGG (SEQ ID NO: 436) | | |
| 99 | CTGTGACTCGCGTAGCAG | CUGUGACUCGCGUAGCAG | CCACTGTGACTCGCGTAGCA | HIST1H1D | −4.084054163 |
| | AG (SEQ ID NO: 155) | AG (SEQ ID NO: 296) | GAG (SEQ ID NO: 437) | | |
| 100 | CTGTGACTCGCGTAGCAG | CUGUGACUCGCGUAGCAG | CCACTGTGACTCGCGTAGCA | HIST1H1T | −4.084054163 |
| | AG (SEQ ID NO: 156) | AG (SEQ ID NO: 297) | GAG (SEQ ID NO: 438) | | |
| 101 | CTGTGACTCGCGTAGCAG | CUGUGACUCGCGUAGCAG | CCACTGTGACTCGCGTAGCA | HIST1H2AC | −4.084054163 |
| | AG (SEQ ID NO: 157) | AG (SEQ ID NO: 298) | GAG (SEQ ID NO: 439) | | |
| 102 | GGGCGGCCCTAAGCTCAG | GGGCGGCCCUAAGCUCAG | GGGCGGCCCTAAGCTCAGGA | NFKBIL1 | −4.623945627 |
| | GA (SEQ ID NO: 158) | GA (SEQ ID NO: 299) | TGG (SEQ ID NO: 440) | | |
| 103 | GGGCGGCCCTAAGCTCAG | GGGCGGCCCUAAGCUCAG | GGGCGGCCCTAAGCTCAGGA | PPP1R10 | −4.623945627 |
| | GA (SEQ ID NO: 159) | GA (SEQ ID NO: 300) | TGG (SEQ ID NO: 441) | | |
| 104 | GGGCGGCCCTAAGCTCAG | GGGCGGCCCUAAGCUCAG | GGGCGGCCCTAAGCTCAGGA | XXbac- | −4.623945627 |
| | GA (SEQ ID NO: 160) | GA (SEQ ID NO: 301) | TGG (SEQ ID NO: 442) | BPG252P9. | |
| | | | | 10 | |
| 105 | CCCGACGGTGGTTCACGA | CCCGACGGUGGUUCACGA | CCCCCCGACGGTGGTTCACG | ATP6V1G2 | −3.387411471 |
| | AA (SEQ ID NO: 161) | AA (SEQ ID NO: 302) | AAA (SEQ ID NO: 443) | | |
| 106 | CCCGACGGTGGTTCACGA | CCCGACGGUGGUUCACGA | CCCCCCGACGGTGGTTCACG | TUBB | −3.387411471 |
| | AA (SEQ ID NO: 162) | AA (SEQ ID NO: 303) | AAA (SEQ ID NO: 444) | | |
| 107 | AGGAGCAGCCCGGGAACC | AGGAGCAGCCCGGGAACC | CCCAGGAGCAGCCCGGGAAC | DHX16 | −1.185735621 |
| | CA (SEQ ID NO: 163) | CA (SEQ ID NO: 304) | CCA (SEQ ID NO: 445) | | |
| 108 | AGGAGCAGCCCGGGAACC | AGGAGCAGCCCGGGAACC | CCCAGGAGCAGCCCGGGAAC | MICA | −1.185735621 |
| | CA (SEQ ID NO: 164) | CA (SEQ ID NO: 305) | CCA (SEQ ID NO: 446) | | |
| 109 | AGGAGCAGCCCGGGAACC | AGGAGCAGCCCGGGAACC | CCCAGGAGCAGCCCGGGAAC | MICB | −1.185735621 |
| | CA (SEQ ID NO: 165) | CA (SEQ ID NO: 306) | CCA (SEQ ID NO: 447) | | |
| 110 | AGGAGCAGCCCGGGAACC | AGGAGCAGCCCGGGAACC | CCCAGGAGCAGCCCGGGAAC | PPP1R10 | −1.185735621 |
| | CA (SEQ ID NO: 166) | CA (SEQ ID NO: 307) | CCA (SEQ ID NO: 448) | | |
| 111 | CGCTGCCGGCCAGCGTCC | CGCUGCCGGCCAGCGUC | CGCTGCCGGCCAGCGTCCTC | RP11- | −2.516398914 |
| | TC (SEQ ID NO: 167) | CUC (SEQ ID NO: 308) | TGG (SEQ ID NO: 449) | 140K17.3 | |
| 112 | CGCTAACAGAGCCGCCAC | CGCUAACAGAGCCGCCAC | CCACGCTAACAGAGCCGCCA | FRS3 | −0.928362359 |
| | AT (SEQ ID NO: 168) | AU (SEQ ID NO: 309) | CAT (SEQ ID NO: 450) | | |
| 113 | TAAGAAGTCGGCAGGGGC | UAAGAAGUCGGCAGGGGC | CCTTAAGAAGTCGGCAGGGG | CDHR3 | −5.132244174 |
| | AG (SEQ ID NO: 169) | AG (SEQ ID NO: 310) | CAG (SEQ ID NO: 451) | | |
| 114 | TAAGAAGTCGGCAGGGGC | UAAGAAGUCGGCAGGGGC | CCTTAAGAAGTCGGCAGGGG | RP4-5 | −5.132244174 |
| | AG (SEQ ID NO: 170) | AG (SEQ ID NO: 311) | CAG (SEQ ID NO: 452) | 93H12.1 | |
| 115 | TAAGAAGTCGGCAGGGGC | UAAGAAGUCGGCAGGGGC | CCTTAAGAAGTCGGCAGGGG | RP5- | −5.132244174 |
| | AG (SEQ ID NO: 171) | AG (SEQ ID NO: 312) | CAG (SEQ ID NO: 453) | 884M6.1 | |
| 116 | CCAGCGCTGGAGGGCAGC | CCAGCGCUGGAGGGCAG | CCAGCGCTGGAGGGCAGCG | PSMG3 | −1.952802165 |
| | GG (SEQ ID NO: 172) | CGG (SEQ ID NO: 313) | GGGG (SEQ ID NO: 454) | | |
| 117 | CAGCCCTCGCGTGTACCT | CAGCCCUCGCGUGUACCU | CAGCCCTCGCGTGTACCTGA | DDX56 | −4.133673733 |
| | GA (SEQ ID NO: 173) | GA (SEQ ID NO: 314) | AGG (SEQ ID NO: 455) | | |
| 118 | TGCGAAAGGCACAGGATC | UGCGAAAGGCACAGGAUC | TGCGAAAGGCACAGGATCCC | MSRA | −0.179998978 |
| | CC (SEQ ID NO: 174) | CC (SEQ ID NO: 315 | CGG (SEQ ID NO: 456) | | |
| 119 | GTGCTGGCTGTAGAGGTTA | GUGCUGGCUGUAGAGGU | GTGCTGGCTGTAGAGGTTAAA | CDC26 | −3.962930049 |
| | A (SEQ ID NO: 175) | UAA (SEQ ID NO: 316) | GG (SEQ ID NO: 457) | | |
| 120 | GTGCTGGCTGTAGAGGTTA | GUGCUGGCUGUAGAGGU | GTGCTGGCTGTAGAGGTTAAA | RNF183 | −3.962930049 |
| | A (SEQ ID NO: 176) | UAA (SEQ ID NO: 317) | GG (SEQ ID NO: 458) | | |
| 121 | CCTCACAACGGGGAGGAA | CCUCACAACGGGGAGGAA | CCACCTCACAACGGGGAGGA | ENG | −0.78733016 |
| | AC (SEQ ID NO: 177) | AC (SEQ ID NO: 318) | AAC (SEQ ID NO: 459) | | |
| 122 | GGGCTTGCTGAGCACTCG | GGGCUUGCUGAGCACUC | CCCGGGCTTGCTGAGCACTC | RP11- | −0.790115359 |
| | CG (SEQ ID NO: 178) | GCG (SEQ ID NO: 319) | GCG (SEQ ID NO: 460) | 545E17.3 | |
| 123 | GAGCACTGAGAGGAGCGG | GAGCACUGAGAGGAGCGG | CCGGAGCACTGAGAGGAGCG | C9orf171 | −1.533863746 |
| | GG (SEQ ID NO: 179) | GG (SEQ ID NO: 320) | GGG (SEQ ID NO: 461) | | |
| 124 | CCATGCTCATGAGCACTGG | CCAUGCUCAUGAGCACUG | CCTCCATGCTCATGAGCACTG | INPP5E | −0.904211923 |
| | A (SEQ ID NO: 180) | GA (SEQ ID NO: 321) | GA (SEQ ID NO: 462) | | |
| 125 | CCATGCTCATGAGCACTGG | CCAUGCUCAUGAGCACUG | CCTCCATGCTCATGAGCACTG | PTGDS | −0.904211923 |
| | A (SEQ ID NO: 181) | GA (SEQ ID NO: 322) | GA (SEQ ID NO: 463) | | |
| 126 | GGCCGAGCGCCCCAGGTC | GGCCGAGCGCCCCAGGU | CCCGGCCGAGCGCCCCAGGT | RAB33A | −2.144598052 |
| | GG (SEQ ID NO: 182) | CGG (SEQ ID NO: 323) | CGG (SEQ ID NO: 464) | | |
| 127 | TCAGGGATGGGAGAGGAG | UCAGGGAUGGGAGAGGA | TCAGGGATGGGAGAGGAGGA | DUSP9 | −2.318799109 |
| | GA (SEQ ID NO: 183) | GGA (SEQ ID NO: 324) | GGG (SEQ ID NO: 465) | | |
| 128 | TCTACCGGTACCCTCTCCC | UCUACCGGUACCCUCUCC | CCATCTACCGGTACCCTCTCC | GATA1 | −0.266425485 |
| | C (SEQ ID NO: 184) | CC (SEQ ID NO: 325) | CC (SEQ ID NO: 466) | | |
| 129 | TCTACCGGTACCCTCTCCC | UCUACCGGUACCCUCUCC | CCATCTACCGGTACCCTCTCC | GLOD5 | −0.266425485 |
| | C (SEQ ID NO: 185) | CC (SEQ ID NO: 326) | CC (SEQ ID NO: 467) | | |
| 130 | TCTACCGGTACCCTCTCCC | UCUACCGGUACCCUCUCC | CCATCTACCGGTACCCTCTCC | HDAC6 | −0.266425485 |
| | C (SEQ ID NO: 186) | CC (SEQ ID NO: 327) | CC (SEQ ID NO: 468) | | |
| 131 | TCTACCGGTACCCTCTCCC | UCUACCGGUACCCUCUCC | CCATCTACCGGTACCCTCTCC | PLP2 | −0.266425485 |
| | C (SEQ ID NO: 187) | CC (SEQ ID NO: 328) | CC (SEQ ID NO: 469) | | |
| 132 | TCTACCGGTACCCTCTCCC | UCUACCGGUACCCUCUCC | CCATCTACCGGTACCCTCTCC | SUV39H1 | −0.266425485 |
| | C (SEQ ID NO: 188) | CC (SEQ ID NO: 329) | CC (SEQ ID NO: 470) | | |
| 133 | TCTACCGGTACCCTCTCCC | UCUACCGGUACCCUCUCC | CCATCTACCGGTACCCTCTCC | WAS | −0.266425485 |
| | C (SEQ ID NO: 189) | CC (SEQ ID NO: 330) | CC (SEQ ID NO: 471) | | |
| 134 | TGAGCCTCAGAGGTATCCT | UGAGCCUCAGAGGUAUCC | CCATGAGCCTCAGAGGTATC | PIM2 | −1.630300488 |
| | G (SEQ ID NO: 190) | UG (SEQ ID NO: 331) | CTG (SEQ ID NO: 472) | | |
| 135 | TCGTAAGATATCAACCATC | UCGUAAGAUAUCAACCAU | CCCTCGTAAGATATCAACCAT | IGBP1 | −2.649530558 |
| | T (SEQ ID NO: 191) | CU (SEQ ID NO: 332) | CT (SEQ ID NO: 473) | | |

DNA encoding gRNA = Protospacer = gRNA protospacer sequence (20 nt).
Target = gRNA protospacer + PAM for guides in the ′+′ strand; reversed complement of PAM+gRNA protospacer for guides in the ′−′ strand.
Gene = HUGO Gene Symbol.
log2FoldChange = log2 fold-change of gRNA enrichment when comparing K562 cells with dCas9-KRAB vs K562 WT cells. A positive value corresponds with gRNAs increasing cells fitness; a negative value indicates gRNAs decreasing cell fitness.

TABLE 18B
gRNAs for use in decreasing cell fitness.

| | Chr | Start | End | | | | Chr | Start | End | log2Fold | | |
|---|---|---|---|---|---|---|---|---|---|---|---|---|
| # | grna | grna | grna | strand | PAM | Gene | gene | gene | gene | Change | pvalue | padj |
| 1 | chr10 | 102119098 | 102119117 | − | TGG | SCD | chr10 | 102106881 | 102124591 | −1.499927588 | 1.16589E−10 | 5.82358E−08 |
| 2 | chr10 | 103872287 | 103872306 | + | GGG | LDB1 | chr10 | 103867317 | 103880210 | −1.773061245 | 3.52298E−20 | 5.34419E−17 |
| 3 | chr10 | 103872287 | 103872306 | + | GGG | NOLC1 | chr10 | 103911933 | 103923627 | −1.773061245 | 3.52298E−20 | 5.34419E−17 |
| 4 | chr10 | 115478924 | 115478943 | + | TGG | CASP7 | chr10 | 115438942 | 115490662 | −1.710885183 | 2.54989E−09 | 6.96152E−07 |
| 5 | chr10 | 120826758 | 120826777 | + | CGG | EIF3A | chr10 | 120794356 | 120840316 | −2.269982229 | 9.14925E−12 | 4.648E−09 |
| 6 | chr10 | 120826758 | 120826777 | + | CGG | FAM45A | chr10 | 120863598 | 120897496 | −2.269982229 | 9.14925E−12 | 4.648E−09 |
| 7 | chr10 | 134649517 | 134649536 | − | GGG | BNIP3 | chr10 | 133781578 | 133795435 | −0.763455908 | 4.31827E−10 | 4.67533E−08 |
| 8 | chr10 | 27444298 | 27444317 | + | GGG | MASTL | chr10 | 27443753 | 27475853 | −3.669938146 | 4.64718E−13 | 9.02762E−11 |
| 9 | chr10 | 4869655 | 4869674 | − | TGG | AKR1E2 | chr10 | 4828821 | 4890254 | −0.297415894 | 0.000287781 | 0.008685744 |
| 10 | chr11 | 123431984 | 123432003 | + | AGG | CRTAM | chr11 | 122709208 | 122743347 | −0.382928483 | 0.000175874 | 0.014548598 |
| 11 | chr11 | 33963334 | 33963353 | − | AGG | LMO2 | chr11 | 33880122 | 33913836 | −2.299987034 | 1.84878E−10 | 1.27592E−07 |
| 12 | chr11 | 33966584 | 33966603 | − | GGG | LMO2 | chr11 | 33880122 | 33913836 | −4.187824104 | 8.33352E−38 | 5.18445E−34 |
| 13 | chr11 | 78001597 | 78001616 | + | GGG | GAB2 | chr11 | 77926343 | 78129394 | −2.6002839 | 1.37404E−11 | 2.22661E−09 |
| 14 | chr11 | 78003476 | 78003495 | − | TGG | GAB2 | chr11 | 77926343 | 78129394 | −2.409253932 | 1.09221E−14 | 1.3886E−11 |
| 15 | chr12 | 133174674 | 133174693 | − | AGG | PGAM5 | chr12 | 133287405 | 133299228 | −0.871448583 | 1.55777E−10 | 1.00041E−08 |
| 16 | chr12 | 32908415 | 32908434 | + | CGG | YARS2 | chr12 | 32880424 | 32908836 | −2.920738928 | 3.14594E−19 | 4.22503E−16 |
| 17 | chr14 | 50066320 | 50066339 | − | TGG | KLHDC1 | chr14 | 50159823 | 50219870 | −2.482482045 | 2.6692E−43 | 1.02718E−39 |
| 18 | chr15 | 64753743 | 64753762 | + | AGG | PDCD7 | chr15 | 65409717 | 65426174 | −0.660746748 | 5.21377E−06 | 0.000417837 |
| 19 | chr15 | 64753743 | 64753762 | + | AGG | ZNF609 | chr15 | 64752941 | 64978264 | −0.660746748 | 5.21377E−06 | 0.000417837 |
| 20 | chr15 | 96816767 | 96816786 | − | CGG | NR2F2 | chr15 | 96869167 | 96883492 | −0.584098352 | 3.86556E−07 | 4.78026E−05 |
| 21 | chr15 | 96816767 | 96816786 | − | CGG | NR2F2- | chr15 | 96670598 | 96870590 | −0.584098352 | 3.86556E−07 | 4.78026E−05 |
| | | | | | | AS1 | | | | | | |
| 22 | chr16 | 23690678 | 23690697 | + | GGG | PLK1 | chr16 | 23688977 | 23701688 | −4.746174907 | 1.12222E−17 | 3.92398E−15 |
| 23 | chr16 | 3156431 | 3156450 | + | TGG | ZG16B | chr16 | 2880170 | 2888967 | −0.219671275 | 0.000106666 | 0.002449228 |
| 24 | chr16 | 89059487 | 89059506 | + | CGG | CBFA2T3 | chr16 | 88941266 | 89043612 | −4.224370075 | 1.82402E−23 | 1.16325E−20 |
| 25 | chr16 | 89059487 | 89059506 | + | CGG | MVD | chr16 | 88718343 | 88729569 | 4.224370075 | 1.82402E−23 | 1.16325E−20 |
| 26 | chr16 | 89059487 | 89059506 | + | CGG | SPATA33 | chr16 | 89724210 | 89737680 | −4.224370075 | 1.82402E−23 | 1.16325E−20 |
| 27 | chr17 | 17723351 | 17723370 | + | CGG | SREBF1 | chr17 | 17713713 | 17740325 | −0.797359326 | 1.59025E−06 | 0.000179597 |
| 28 | chr17 | 26075388 | 26075407 | − | GGG | CTD- | chr17 | 26590660 | 26593395 | −0.53313193 | 1.30879E−06 | 0.000157631 |
| | | | | | | 2008P7.1 | | | | | | |
| 29 | chr17 | 40472220 | 40472239 | + | AGG | CCR10 | chr17 | 40830907 | 40835935 | −1.843687278 | 1.08941E−05 | 0.000601642 |
| 30 | chr17 | 40472220 | 40472239 | + | AGG | HAP1 | chr17 | 39873994 | 39890896 | −1.843687278 | 1.08941E−05 | 0.000601642 |
| 31 | chr17 | 40472220 | 40472239 | + | AGG | PTRF | chr17 | 40554470 | 40575535 | −1.843687278 | 1.08941E−05 | 0.000601642 |
| 32 | chr17 | 40472220 | 40472239 | + | AGG | STAT3 | chr17 | 40465342 | 40540586 | −1.843687278 | 1.08941E−05 | 0.000601642 |
| 33 | chr17 | 40472220 | 40472239 | + | AGG | STAT5A | chr17 | 40439565 | 40463961 | −1.843687278 | 1.08941E−05 | 0.000601642 |
| 34 | chr17 | 40472220 | 40472239 | + | AGG | STAT5B | chr17 | 40351186 | 40428725 | −1.843687278 | 1.08941E−05 | 0.000601642 |
| 35 | chr17 | 4447479 | 4447498 | + | GGG | CAMKK1 | chr17 | 3763609 | 3798185 | −1.155590004 | 3.15936E−11 | 1.11288E−08 |
| 36 | chr17 | 48961477 | 48961496 | − | TGG | RSAD1 | chr17 | 48556161 | 48563336 | 0.793709187 | 3.60648E−09 | 5.17199E−07 |
| 37 | chr17 | 48961477 | 48961496 | − | TGG | XYLT2 | chr17 | 48423453 | 48440499 | −0.793709187 | 3.60648E−09 | 5.17199E−07 |
| 38 | chr17 | 62015741 | 62015760 | + | GGG | ERN1 | chr17 | 62116502 | 62208179 | −1.456150356 | 5.83823E−09 | 1.65323E−06 |
| 39 | chr17 | 78222801 | 78222820 | − | TGG | CARD14 | chr17 | 78143791 | 78183130 | −4.195662865 | 1.33839E−20 | 6.23076E−18 |
| 40 | chr19 | 12996886 | 12996905 | + | GGG | KLF1 | chr19 | 12995237 | 12997995 | −1.316522962 | 7.40827E−07 | 5.189E−05 |
| 41 | chr19 | 12996886 | 12996905 | + | GGG | TNPO2 | chr19 | 12810008 | 12834825 | −1.316522962 | 7.40827E−07 | 5.189E−05 |
| 42 | chr19 | 16188145 | 16188164 | + | CGG | RASAL3 | chr19 | 15562435 | 15575382 | −0.401754515 | 0.001333663 | 0.035342875 |
| 43 | chr19 | 2730264 | 2730283 | + | CGG | AC0052561 | chr19 | 1748055 | 1748744 | −0.757866457 | 1.99532E−06 | 0.000233659 |
| 44 | chr19 | 2730264 | 2730283 | + | CGG | GIPC3 | chr19 | 3585551 | 3593539 | −0.757866457 | 1.99532E−06 | 0.000233659 |
| 45 | chr19 | 2730264 | 2730283 | + | CGG | MKNK2 | chr19 | 2037470 | 2051243 | −0.757866457 | 1.99532E−06 | 0.000233659 |
| 46 | chr19 | 33178821 | 33178840 | + | AGG | PDCD5 | chr19 | 33071974 | 33078358 | −2.45026283 | 5.07478E−07 | 3.66374E−05 |
| 47 | chr19 | 48269449 | 48269468 | − | AGG | CTC- | chr19 | 49016464 | 49016917 | −0.585672828 | 6.20258E−07 | 3.74213E−05 |
| | | | | | | 273B12.10 | | | | | | |
| 48 | chr19 | 51479221 | 51479240 | + | AGG | CTD- | chr19 | 52022985 | 52023514 | −2.700478104 | 1.99815E−14 | 1.30766E−11 |
| | | | | | | 3073N11.9 | | | | | | |
| 49 | chr19 | 54637756 | 54637775 | − | AGG | AC0084405 | chr19 | 54377880 | 54379303 | −2.203212712 | 3.24701E−06 | 0.000200551 |
| 50 | chr1 | 109757641 | 109757660 | − | TGG | SARS | chr1 | 109756540 | 109780791 | −2.55293289 | 8.47606E−18 | 1.57051E−14 |
| 51 | chr1 | 109761346 | 109761365 | − | AGG | SARS | chr1 | 109756540 | 109780791 | −0.711547573 | 0.000122219 | 0.014735573 |
| 52 | chr1 | 109782410 | 109782429 | − | AGG | RP5- | chr1 | 109630593 | 109633480 | −1.667165813 | 2.57063E−12 | 1.4157E−09 |
| | | | | | | 1065J22.8 | | | | | | |
| 53 | chr1 | 109782410 | 109782429 | − | AGG | SARS | chr1 | 109756540 | 109780791 | −1.667165813 | 2.57063E−12 | 1.4157E−09 |
| 54 | chr1 | 11369670 | 11369689 | − | GGG | DFFA | chr1 | 10516579 | 10532583 | −1.059035361 | 1.80175E−08 | 1.98494E−06 |
| 55 | chr1 | 11369670 | 11369689 | − | GGG | KIAA2013 | chr1 | 11979648 | 11986485 | −1.059035361 | 1.80175E−08 | 1.98494E−06 |
| 56 | chr1 | 149046669 | 149046688 | + | TGG | RP11- | chr1 | 149817383 | 149818053 | −5.360362095 | 7.79536E−45 | 3.30712E−41 |
| | | | | | | 196G18.24 | | | | | | |
| 57 | chr1 | 151117362 | 151117381 | − | AGG | THEM4 | chr1 | 151846060 | 151882284 | −0.304275846 | 7.32627E−05 | 0.002894576 |
| 58 | chr1 | 160054752 | 160054771 | + | TGG | SLAMF1 | chr1 | 160577890 | 160617085 | −0.202557574 | 0.001683471 | 0.040916866 |
| 59 | chr1 | 202561511 | 202561530 | + | TGG | snoU13 | chr1 | 202200792 | 202200892 | −0.312253516 | 0.00144774 | 0.041984789 |
| 60 | chr1 | 204380211 | 204380230 | − | GGG | PPP1R15B | chr1 | 204372515 | 204380919 | −1.858478461 | 7.42176E−17 | 3.64283E−14 |
| 61 | chr1 | 28422902 | 28422921 | − | CGG | RP5- | chr1 | 28566020 | 28567964 | −2.523650892 | 5.10562E−12 | 2.98551E−09 |
| | | | | | | 1092A3.4 | | | | | | |
| 62 | chr1 | 2890481 | 2890500 | − | AGG | MEGF6 | chr1 | 3406484 | 3528059 | −1.098391527 | 2.88244E−15 | 8.14035E−13 |
| 63 | chr1 | 2890481 | 2890500 | − | AGG | WRAP73 | chr1 | 3547331 | 3569325 | −1.098391527 | 2.88244E−15 | 8.14035E−13 |
| 64 | chr1 | 43826400 | 43826419 | − | AGG | CDC20 | chr1 | 43824626 | 43828874 | −1.990500496 | 9.414E−19 | 9.68984E−16 |
| 65 | chr1 | 43826400 | 43826419 | − | AGG | TIE1 | chr1 | 43766664 | 43788779 | −1.990500496 | 9.414E−19 | 9.68984E−16 |
| 66 | chr1 | 5698888 | 5698907 | − | GGG | DNAJC11 | chr1 | 6694228 | 6761984 | −1.568794977 | 2.04716E−14 | 6.79146E−12 |
| 67 | chr20 | 30263861 | 30263880 | + | GGG | BCL2L1 | chr20 | 30252255 | 30311792 | −2.930273816 | 3.30308E−15 | 1.61492E−12 |
| 68 | chr20 | 30328119 | 30328138 | − | AGG | TPX2 | chr20 | 30327074 | 30389608 | −2.724166279 | 1.56366E−26 | 4.0341E−23 |
| 69 | chr20 | 59904474 | 59904493 | + | TGG | OSBPL2 | chr20 | 60813580 | 60871268 | −4.046949933 | 2.50203E−27 | 7.13776E−24 |
| 70 | chr20 | 59904474 | 59904493 | + | TGG | SS18L1 | chr20 | 60718822 | 60757540 | −4.046949933 | 2.50203E−27 | 7.13776E−24 |
| 71 | chr21 | 34473691 | 34473710 | + | TGG | AP000265.1 | chr21 | 33632115 | 33633896 | −4.090254293 | 3.02773E−26 | 3.16557E−23 |
| 72 | chr21 | 34473691 | 34473710 | + | TGG | IL10RB | chr21 | 34638663 | 34669539 | −4.090254293 | 3.02773E−26 | 3.16557E−23 |
| 73 | chr21 | 34473691 | 34473710 | + | TGG | MIS18A | chr21 | 33640530 | 33651380 | −4.090254293 | 3.02773E−26 | 3.16557E−23 |
| 74 | chr21 | 34473691 | 34473710 | + | TGG | MRPS6 | chr21 | 35445524 | 35515334 | −4.090254293 | 3.02773E−26 | 3.16557E−23 |
| 75 | chr21 | 47027472 | 47027491 | + | AGG | AP001476.4 | chr21 | 47472510 | 47473019 | −0.683911933 | 8.94094E−09 | 6.67722E−07 |
| 76 | chr22 | 18371792 | 18371811 | − | TGG | USP18 | chr22 | 18632666 | 18660164 | −0.726414208 | 2.0014E−05 | 0.000793051 |
| 77 | chr22 | 19733132 | 19733151 | + | AGG | AC004463.6 | chr22 | 19158908 | 19160352 | −0.240173353 | 0.00181737 | 0.036788733 |
| 78 | chr2 | 127955643 | 127955662 | − | AGG | ERCC3 | chr2 | 128014866 | 128051752 | −0.880872443 | 7.8799E−10 | 1.42295E−07 |
| 79 | chr2 | 45837776 | 45837795 | − | AGG | SRBD1 | chr2 | 45615819 | 45839304 | −3.510953718 | 1.19959E−10 | 1.67597E−08 |
| 80 | chr2 | 47563629 | 47563648 | − | GGG | BCYRN1 | chr2 | 47558199 | 47571656 | −0.669374994 | 1.62525E−07 | 1.83626E−05 |
| 81 | chr2 | 47563629 | 47563648 | − | GGG | EPCAM | chr2 | 47572297 | 47614740 | −0.669374994 | 1.62525E−07 | 1.83626E−05 |
| 82 | chr2 | 48542453 | 48542472 | − | GGG | FOXN2 | chr2 | 48541776 | 48606433 | −1.056757668 | 2.6411E−10 | 6.45332E−08 |
| 83 | chr2 | 55920328 | 55920347 | + | GGG | PNPT1 | chr2 | 55861400 | 55921045 | −3.787114905 | 2.61895E−16 | 7.84163E−14 |
| 84 | chr2 | 75061352 | 75061371 | − | AGG | HK2 | chr2 | 75061108 | 75120486 | −1.659673132 | 1.60267E−07 | 1.30231E−05 |
| 85 | chr2 | 75062270 | 75062289 | − | GGG | INO80B | chr2 | 74682150 | 74688011 | −1.707593267 | 1.50193E−19 | 9.22056E−17 |
| 86 | chr3 | 10492368 | 10492387 | + | GGG | GHRLOS | chr3 | 10327438 | 10335133 | −1.182500906 | 1.05326E−05 | 0.000872859 |
| 87 | chr3 | 113466355 | 113466374 | + | AGG | ATP6V1A | chr3 | 113465866 | 113530903 | −3.139614024 | 2.52123E−10 | 3.36716E−08 |
| 88 | chr3 | 13629109 | 13629128 | + | GGG | RP11- | chr3 | 14313873 | 14345345 | −1.067410009 | 5.44202E−14 | 1.71401E−11 |
| | | | | | | 53616.2 | | | | | | |
| 89 | chr5 | 31531835 | 31531854 | + | CGG | DROSHA | chr5 | 31400604 | 31532303 | −1.775504833 | 1.58118E−10 | 4.70048E−08 |
| 90 | chr5 | 52095954 | 52095973 | + | GGG | PELO | chr5 | 52083774 | 52099880 | −5.486691788 | 5.78604E−21 | 2.77084E−18 |
| 91 | chr5 | 52096719 | 52096738 | + | AGG | PELO | chr5 | 52083774 | 52099880 | −5.468864005 | 8.8166E−52 | 1.44721E−47 |
| 92 | chr5 | 96518508 | 96518527 | + | AGG | RIOK2 | chr5 | 96496571 | 96518964 | −2.456552248 | 1.89182E−12 | 1.04632E−09 |
| 93 | chr6 | 13273676 | 13273695 | − | GGG | PHACTR1 | chr6 | 12717893 | 13288645 | −1.64365509 | 3.44083E−14 | 2.28113E−11 |
| 94 | chr6 | 135642599 | 135642618 | + | GGG | AHI1 | chr6 | 135604670 | 135818914 | −1.396274517 | 1.15575E−11 | 4.50697E−09 |
| 95 | chr6 | 135642599 | 135642618 | + | GGG | MYB | chr6 | 135502453 | 135540311 | −1.396274517 | 1.15575E−11 | 4.50697E−09 |
| 96 | chr6 | 135644551 | 135644570 | + | TGG | MYB | chr6 | 135502453 | 135540311 | −2.406371188 | 7.60863E−12 | 3.97888E−09 |
| 97 | chr6 | 150849962 | 150849981 | − | TGG | ULBP1 | chr6 | 150285143 | 150294846 | −0.274041877 | 0.000105494 | 0.003097959 |
| 98 | chr6 | 153303811 | 153303830 | − | GGG | FBXO5 | chr6 | 153291664 | 153304714 | −4.044506266 | 1.16332E−13 | 2.50561E−11 |
| 99 | chr6 | 26330594 | 26330613 | − | TGG | HIST1H1D | chr6 | 26234440 | 26235216 | −4.084054163 | 3.3717E−16 | 9.85392E−14 |
| 100 | chr6 | 26330594 | 26330613 | − | TGG | HIST1H1T | chr6 | 26107640 | 26108364 | −4.084054163 | 3.3717E−16 | 9.85392E−14 |
| 101 | chr6 | 26330594 | 26330613 | − | TGG | HIST1H2AC | chr6 | 26124373 | 26139344 | −4.084054163 | 3.3717E−16 | 9.85392E−14 |
| 102 | chr6 | 30582859 | 30582878 | + | TGG | NFKBIL1 | chr6 | 31514647 | 31526606 | −4.623945627 | 7.0866E−31 | 2.36483E−27 |
| 103 | chr6 | 30582859 | 30582878 | + | TGG | PPP1R10 | chr6 | 30568177 | 30586389 | −4.623945627 | 7.0866E−31 | 2.36483E−27 |
| 104 | chr6 | 30582859 | 30582878 | + | TGG | XXbac- | chr6 | 30710706 | 30711369 | −4.623945627 | 7.0866E−31 | 2.36483E−27 |
| | | | | | | BPG252P9.10 | | | | | | |
| 105 | chr6 | 30690539 | 30690558 | − | GGG | ATP6V1G2 | chr6 | 31512239 | 31516204 | −3.387411471 | 3.56752E−24 | 5.43426E−21 |
| 106 | chr6 | 30690539 | 30690558 | − | GGG | TUBB | chr6 | 30687978 | 30693203 | −3.387411471 | 3.56752E−24 | 5.43426E−21 |
| 107 | chr6 | 31466673 | 31466692 | − | GGG | DHX16 | chr6 | 30620896 | 30640814 | −1.185735621 | 2.31405E−08 | 3.31259E−06 |
| 108 | chr6 | 31466673 | 31466692 | − | GGG | MICA | chr6 | 31371356 | 31383092 | −1.185735621 | 2.31405E−08 | 3.31259E−06 |
| 109 | chr6 | 31466673 | 31466692 | − | GGG | MICB | chr6 | 31462658 | 31478901 | −1.185735621 | 2.31405E−08 | 3.31259E−06 |
| 110 | chr6 | 31466673 | 31466692 | − | GGG | PPP1R10 | chr6 | 30568177 | 30586389 | −1.185735621 | 2.31405E−08 | 3.31259E−06 |
| 111 | chr6 | 33850892 | 33850911 | + | TGG | RP11- | chr6 | 34664094 | 34665247 | −2.516398914 | 5.90832E−13 | 5.92749E−10 |
| | | | | | | 140K17.3 | | | | | | |
| 112 | chr6 | 41736949 | 41736968 | − | TGG | FRS3 | chr6 | 41737914 | 41754280 | −0.928362359 | 2.31747E−07 | 2.74453E−05 |
| 113 | chr7 | 106415349 | 106415368 | − | AGG | CDHR3 | chr7 | 105517242 | 105676877 | −5.132244174 | 5.64046E−24 | 3.83788E−21 |
| 114 | chr7 | 106415349 | 106415368 | − | AGG | RP4- | chr7 | 107220002 | 107220502 | −5.132244174 | 5.64046E−24 | 3.83788E−21 |
| | | | | | | 593H12.1 | | | | | | |
| 115 | chr7 | 106415349 | 106415368 | − | AGG | RP5- | chr7 | 106415457 | 106436010 | −5.132244174 | 5.64046E−24 | 3.83788E−21 |
| | | | | | | 884M6.1 | | | | | | |
| 116 | chr7 | 2511388 | 2511407 | + | GGG | PSMG3 | chr7 | 1606966 | 1610641 | −1.952802165 | 2.77116E−07 | 3.90355E−05 |
| 117 | chr7 | 44613419 | 44613438 | + | AGG | DDX56 | chr7 | 44605016 | 44614650 | −4.133673733 | 1.07448E−91 | 7.28359E−87 |
| 118 | chr8 | 10653800 | 10653819 | + | CGG | MSRA | chr8 | 9911778 | 10286401 | −0.179998978 | 0.004196968 | 0.101464575 |
| 119 | chr9 | 116036899 | 116036918 | + | AGG | CDC26 | chr9 | 116018115 | 116037869 | −3.962930049 | 1.08239E−58 | 2.52042E−54 |
| 120 | chr9 | 116036899 | 116036918 | + | AGG | RNF183 | chr9 | 116059373 | 116065656 | −3.962930049 | 1.08239E−58 | 2.52042E−54 |
| 121 | chr9 | 130470076 | 130470095 | − | TGG | ENG | chr9 | 130577291 | 130617035 | −0.78733016 | 7.37096E−05 | 0.009765999 |
| 122 | chr9 | 132482478 | 132482497 | − | GGG | RP11- | chr9 | 131486724 | 131495473 | −0.790115359 | 2.99498E−07 | 2.46879E−05 |
| | | | | | | 545E17.3 | | | | | | |
| 123 | chr9 | 134929647 | 134929666 | − | CGG | C9orf171 | chr9 | 135285430 | 135448704 | −1.533863746 | 5.21377E−09 | 7.70369E−07 |
| 124 | chr9 | 139087909 | 139087928 | − | AGG | INPP5E | chr9 | 139323071 | 139334274 | −0.904211923 | 2.35996E−07 | 2.10281E−05 |
| 125 | chr9 | 139087909 | 139087928 | − | AGG | PTGDS | chr9 | 139871956 | 139879887 | −0.904211923 | 2.35996E−07 | 2.10281E−05 |
| 126 | chrX | 129254624 | 129254643 | − | GGG | RAB33A | chrX | 129305623 | 129318844 | −2.144598052 | 1.84255E−08 | 4.75306E−06 |
| 127 | chrX | 152841252 | 152841271 | + | GGG | DUSP9 | chrX | 152907946 | 152916781 | −2.318799109 | 1.01668E−07 | 2.43354E−05 |
| 128 | chrX | 48648021 | 48648040 | − | TGG | GATA1 | chrX | 48644962 | 48652716 | −0.266425485 | 0.000180701 | 0.005905845 |
| 129 | chrX | 48648021 | 48648040 | − | TGG | GLOD5 | chrX | 48620154 | 48632064 | −0.266425485 | 0.000180701 | 0.005905845 |
| 130 | chrX | 48648021 | 48648040 | − | TGG | HDAC6 | chrX | 48659784 | 48683392 | −0.266425485 | 0.000180701 | 0.005905845 |
| 131 | chrX | 48648021 | 48648040 | − | TGG | PLP2 | chrX | 49028273 | 49031588 | −0.266425485 | 0.000180701 | 0.005905845 |
| 132 | chrX | 48648021 | 48648040 | − | TGG | SUV39H1 | chrX | 48553945 | 48567403 | −0.266425485 | 0.000180701 | 0.005905845 |
| 133 | chrX | 48648021 | 48648040 | − | TGG | WAS | chrX | 48534985 | 48549818 | −0.266425485 | 0.000180701 | 0.005905845 |
| 134 | chrX | 48798027 | 48798046 | − | TGG | PIM2 | chrX | 48770459 | 48776301 | −1.630300488 | 5.53268E−07 | 0.000113843 |
| 135 | chrX | 69354151 | 69354170 | − | GGG | IGBP1 | chrX | 69353299 | 69386174 | −2.649530558 | 4.33867E−13 | 1.05411E−10 |

Chr grna = gRNA chromosome.
Start grna = gRNA start coodinate (in hg19).
End grna = gRNA end coodinate (in hg19).
strand = gRNA strand.
PAM = SpCas9 PAM (NGG).
Gene = HUGO Gene Symbol.
Chr gene = Gene chromosome.
Start gene = Gene end coordinate of the longest isoform annotated in Gencode v22 (in hg19).
End gene = Gene start coordinate of the longest isoform annotated in Gencode v22 (in hg19).
log2FoldChange = log2 fold-change of gRNA enrichment when comparing K562 cells with dCas9-KRAB vs K562 WT cells. A positive value corresponds with gRNAs increasing cells fitness; a negative value indicates gRNAs decreasing cell fitness.
pvalue = gRNA enrichment p-values corresponding to the Wald test performed by DESeq2.
padj = gRNA enrichment adjusted p-values corresponding to the Wald test performed by DESeq2, after correcting multiple hypothesis testing with the Independent Hypothesis Weighting method.

TABLE 19A
gRNAs for use in increasing cell fitness.

| | | | | | log2Fold |
|---|---|---|---|---|---|
| # | DNA encoding gRNA | gRNA | Target | Gene | Change |
| 136 | GAACTAGGATCCCACAGGGT | GAACUAGGAUCCCACAGGGU | GAACTAGGATCCCACAGGGTTGG | FADS3 | 0.311438728 |
| | (SEQ ID NO: 192) | (SEQ ID NO: 333) | (SEQ ID NO: 474) | | |
| 137 | GCTTCCTCCTCTCCACTCCT | GCUUCCUCCUCUCCACUCCU | CCCGCTTCCTCCTCTCCACTCCT | RPAP1 | 0.21770682 |
| | (SEQ ID NO: 193) | (SEQ ID NO: 334) | (SEQ ID NO: 475) | | |
| 138 | CAGGTCCTCTCCTATCTCTT | CAGGUCCUCUCCURUCUCUU | CAGGTCCTCTCCTATCTCTTTGG | SLC25A39 | 0.402017292 |
| | (SEQ ID NO: 194) | (SEQ ID NO: 335) | (SEQ ID NO: 476) | | |
| 139 | AACAGTTTAATCAATTAGCG | AACAGUUUAAUCAAUUAGCG | CCTAACAGTTTAATCAATTAGCG | RP13- | 0.286876456 |
| | (SEQ ID NO: 195) | (SEQ ID NO: 336) | (SEQ ID NO: 477) | 20L14.6 | |
| 140 | TACTGAGTCATACCAATGTT | UACUGAGUCAUACCAAUGUU | CCGTACTGAGTCATACCAATGTT | FOXA2 | 0.209612865 |
| | (SEQ ID NO: 196) | (SEQ ID NO: 337) | (SEQ ID NO: 478) | | |
| 141 | TCCATTTCAGTTTATACCAG | UCCAUUUCAGUUUAUACCAG | CCTTCCATTTCAGTTTATACCAG | GMPR | 0.236732414 |
| | (SEQ ID NO: 197) | (SEQ ID NO: 338) | (SEQ ID NO: 479) | | |

DNA encoding gRNA = Protospacer = gRNA protospacer sequence (20 nt).
Target = gRNA protospacer + PAM for guides in the ′+′ strand; reversed complement of PAM + gRNA protospacer for guides in the ′−′ strand.
Gene = HUGO Gene Symbol.
log2FoldChange = log2 fold-change of gRNA enrichment when comparing K562 cells with dCas9-KRAB vs K562 WT cells. A positive value corresponds with gRNAs increasing cells fitness; a negative value indicates gRNAs decreasing cell fitness.

TABLE 19B
gRNAs for use in increasing cell fitness.

| | Chr | Start | End | | | | Chr | Start | End | log2Fold | | |
|---|---|---|---|---|---|---|---|---|---|---|---|---|
| # | grna | grna | grna | strand | PAM | Gene | gene | gene | gene | Change | pvalue | padj |
| 136 | chr11 | 61299861 | 61299880 | + | TGG | FADS3 | chr11 | 61640991 | 61659523 | 0.311438728 | 0.000734608 | 0.02349938 |
| 137 | chr15 | 42273690 | 42273709 | − | GGG | RPAP1 | chr15 | 41809374 | 41836467 | 0.21770682 | 0.000250826 | 0.00560094 |
| 138 | chr17 | 42365788 | 42365807 | + | TGG | SLC25A39 | chr17 | 42396993 | 42402238 | 0.402017292 | 7.08604E−05 | 0.002744997 |
| 139 | chr17 | 81056702 | 81056721 | − | AGG | RP13- | chr17 | 80412149 | 80416397 | 0.286876456 | 0.001252739 | 0.036378654 |
| | | | | | | 20L14.6 | | | | | | |
| 140 | chr20 | 22392272 | 22392291 | − | CGG | FOXA2 | chr20 | 22561643 | 22566093 | 0.209612865 | 0.001406984 | 0.040222606 |
| 141 | chr6 | 16215985 | 16216004 | − | AGG | GMPR | chr6 | 16238811 | 16295780 | 0.236732414 | 0.00549195 | 0.126641266 |

Chr grna = gRNA chromosome.
Start grna = gRNA start coodinate (in hg19).
End grna = gRNA end coodinate (in hg19).
strand = gRNA strand.
PAM = SpCas9 PAM (NGG).
Gene = HUGO Gene Symbol.
Chr gene = Gene chromosome.
Start gene = Gene end coordinate of the longest isoform annotated in Gencode v22 (in hg19).
End gene = Gene start coordinate of the longest isoform annotated in Gencode v22 (in hg19).
log2FoldChange = log2 fold-change of gRNA enrichment when comparing K562 cells with dCas9-KRAB vs K562 WT cells. A positive value corresponds with gRNAs increasing cells fitness; a negative value indicates gRNAs decreasing cell fitness.
pvalue = gRNA enrichment p-values corresponding to the Wald test performed by DESeq2.
padj = gRNA enrichment adjusted p-values corresponding to the Wald test performed by DESeq2, after correcting multiple hypothesis testing with the Independent Hypothesis Weighting method.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Repair Pathways

The CRISPR/Cas9-based gene editing system may be used to introduce site-specific double strand breaks at targeted genomic loci, such as at a gene regulatory element affecting cellular fitness. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

i) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

ii) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 5. GENETIC CONSTRUCTS

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a M HCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human 8-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646, which is incorporated herein by reference in its entirety). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151, which is incorporated herein by reference in its entirety). The modified AAV vector may be AAV2i8G9 (Shen et al. J. Biol. Chem. 2013, 288, 28814-28823, which is incorporated herein by reference in its entirety).

### 6. PHARMACEUTICAL COMPOSITIONS

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 7. ADMINISTRATION

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. The cell may be a cancer cell.

### 8. KITS

Provided herein is a kit, which may be used to modulate cellular fitness. The kit may be used to treat cancer such as leukemia. The kit comprises genetic constructs or a composition comprising the same, as described above, and instructions for using said composition. In some embodiments, the kit comprises at least one gRNA comprising a polynucleotide sequence selected from SEQ ID NO: 198-338, a complement thereof, a variant thereof, or fragment thereof, or at least one gRNA encoded by a polynucleotide comprising a sequence selected from SEQ ID NO: 57-197, a complement thereof, a variant thereof, or fragment thereof, or at least one gRNA targeting a polynucleotide comprising a sequence selected from SEQ ID NO: 339-479, a complement thereof, a variant thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for modifying cellular fitness and/or for treating cancer such as leukemia may include a modified AAV vector that includes a gRNA molecule(s) and a Cas9 protein or fusion protein, as described above, that specifically binds a gene regulatory element as detailed herein.

### 9. METHODS

a. Methods of Treating Leukemia

Provided herein are methods of treating leukemia in a subject. The methods may include comprising targeting a regulatory element of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 or modifying (for example, reducing) the expression of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the subject. The method may include administering to the subject an agent as detailed herein, a DNA targeting composition as detailed herein, a polynucleotide sequence as detailed herein, a vector as detailed herein, a cell as detailed herein, or a pharmaceutical composition as detailed herein, or a combination thereof.

b. Methods of Modifying Growth of a Cell

Provided herein are methods of modifying growth of a cell. The methods may include targeting a regulatory element of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR or modifying the expression of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-536I6.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR in the cell. The method may include administering to the subject an agent as detailed herein, a DNA targeting composition as detailed herein, a polynucleotide sequence as detailed herein, a vector as detailed herein, a cell as detailed herein, or a pharmaceutical composition as detailed herein, or a combination thereof.

c. Methods of Decreasing Cell Fitness

Provided herein are methods of decreasing cell fitness. The methods may include administering to a cell an agent as detailed herein, a DNA targeting composition as detailed herein, a polynucleotide sequence as detailed herein, or a vector as detailed herein, or a combination thereof. In some embodiments, the expression of a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 is reduced to decrease the cell fitness. In some embodiments, decreasing cell fitness comprises decreasing cell growth rate, decreasing cell growth duration, decreasing cell size, increasing cell death, or a combination thereof.

d. Methods of Increasing Cell Fitness

Provided herein are methods of increasing cell fitness. The methods may include administering to a cell an agent as detailed herein, a DNA targeting composition as detailed herein, a polynucleotide sequence as detailed herein, or a vector as detailed herein, or a combination thereof. In some embodiments, the expression of a gene selected from FADS3, RPAP1, SLC25A39, RP13-20L14.6, FOXA2, and GMPR is reduced to increase the cell fitness. In some embodiments, increasing cell fitness comprises increasing cell growth rate, increasing cell growth duration, increasing cell size, or a combination thereof.

### 10. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Plasmids. The lentiviral dCas9-KRAB plasmid (Addgene #83890) was generated by cloning in a P2A-HygroR (APH) cassette after dCas9-KRAB using Gibson assembly (NEB, E2611L). The lentiviral gRNA expression plasmid was cloned by combining a U6-gRNA cassette containing the gRNA-(F+E)-combined scaffold sequence (Chen et al., Cell. 2013, 155, 1479-1491, which is incorporated herein by reference in its entirety) with an EGFP-P2A-PAC or mCherry-P2A-PAC cassette into a lentiviral expression backbone (Addgene #83925) using Gibson assembly. Individual gRNAs were ordered as oligonucleotides (IDT-DNA), phosphorylated, hybridized, and ligated into the EGFP gRNA plasmid or the mCherry gRNA plasmid using BsmBI sites.

Cell Culture. K562 and HEK293T (for lentiviral packaging) cells were obtained from the American Tissue Collection Center (ATCC) via the Duke University Cancer Center Facilities. OCI-AML2 cells were gifted from Anthony Letai at Dana Farber Cancer Institute. K562 and OCIAML2 cells were maintained in RPMI 1640 media supplemented with 10% FBS and 1% penicillin-streptomycin. HEK293T cells were maintained in DMEM High Glucose supplemented with 10% FBS and 1% penicillin-streptomycin. All cell lines were cultured at 37° C. and 5% CO₂.

For the genome-wide discovery screen, a clonal K562-dCas9^{KRAB} cell line was used, and generated by transduction of dCas9-KRAB-P2A-HygroR lentivirus with polybrene at a concentration of 8 μg/mL. Cells were selected 2 days post-transduction with Hygromycin B (600 μg/mL, ThermoFisher, 10687010) for 10 days followed by sorting single-cells into 96-well plates with a SH800 sorter (Sony Biotechnology). Individual clones were grown and stained for dCas9^{KRAB} with a Cas9 antibody (Mouse mAb IgG1 clone 7A9-3A3 Alexa Fluor 647 Conjugate, Cell Signaling Technologies, 48796) to assess protein expression. Briefly, 1×10⁶ cells were harvested and washed once with 1×FACS buffer (1% BSA in PBS). The cells were then fixed and permeabilized for 30 minutes at room temperature with 500 μL of fixation and permeabilization buffer (eBioscience Foxp3/TF/nuclear staining kit, ThermoFisher, 00-5523-00). Next, 1 mL of permeabilization buffer was added and cells were pelleted (600 RCF for 5 min) and washed again in 1 mL of permeabilization buffer. Cells were pelleted again and resuspended in 50 μL of permeabilization buffer with 2% mouse serum (Millipore Sigma, M5905) to block for 10 minutes at room temperature. Following blocking, 50 μL of permeabilization buffer with 2% mouse serum and 1 μL of Cas9 antibody was added and allowed to incubate for 30 minutes at room temperature. Following incubation, 1 mL of permeabilization buffer was added, cells were pelleted and washed once more with 1 mL of permeabilization buffer. Finally, cells were resuspended in 1×FACS buffer for analysis. Each clone was analyzed using an Accuri C6 flow cytometer (BD Biosciences). A clone was selected based on high and uniform expression of dCas9^{KRAB} and expanded for further use.

For the secondary sub-library screens, polyclonal K562 and OCI-AML2 cell lines that express the dCas9^{KRAB} repressor were used. Polyclonal lines were used to account for possible hits in the first screen that could be specific to the clonal line used. K562 and OCI-AML2 cells were transduced with dCas9-KRAB-P2A-HygroR lentivirus with polybrene at a concentration of 8 μg/mL. At two days post-transduction, cells were selected for 10 days in Hygromycin B (600 μg/mL). Following selection, polyclonal cells were stained to detect expression of dCas9^{KRAB} protein as described above.

gRNA Library Design. DNase I hypersensitive sites (DHSs) for the K562 cell line were downloaded from encodeproject.org (ENCFF001UWQ) and used to extract genomic sequences as input for gRNA identification. The gt-scan algorithm was used to identify gRNA protospacers within each DHS region and identify possible alignments to other regions of the genome (O'Brien et al., Bioinformatics. 2014, 30, 2673-2675, which is incorporated herein by reference in its entirety). The result was a database containing all possible gRNAs targeting all targetable DHSs in K562 cells and each gRNA's possible off-target locations. gRNAs were selected based on minimizing the number of off-target alignments. For the initial genome-wide library, 1,092,706 gRNAs were selected (see, for example, TABLES S1-S4 and as in Klann et al. 2021. “Genome-wide annotation of gene regulatory elements linked to cell fitness” bioRxiv doi: 10.1101/2021.03.08.434470, which is incorporated herein by reference in its entirety), targeting 111,756 DHSs (269 DHSs contained no NGG SpCas9 PAM), limited to a maximum of 10 gRNAs per DHS (mean, 9.77 gRNAs per DHS).

For the second sub-library targeting distal non-promoter hits (>3 kb from TSS) identified in the first screen, 234,593 gRNAs were selected (see, for example, TABLES S6 to S13 and as in Klann et al. 2021. “Genome-wide annotation of gene regulatory elements linked to cell fitness” bioRxiv doi: 10.1101/2021.03.08.434470, which is incorporated herein by reference in its entirety), targeting 8,850 distal DHSs identified as significant (FDR-adjusted p-value <0.1) from the first screen. For each DHS, gRNAs were chosen to be spread evenly across the region by dividing each DHS into bins of 100 bp and selecting up to 7 gRNAs per bin. The gRNAs for each bin were selected in order by the fewest number of off-target alignments calculated by gt-scan. 15,407 non-targeting gRNAs were designed as previously described (Horlbeck et al., eLife. 2016, 5, doi:10.7554/elife.19760, which is incorporated herein by reference in its entirety). A larger number of gRNAs per DHS were designed in the second screen (˜24 per DHS) compared to the first screen (10 per DHS).

All libraries were synthesized by Twist Biosciences and the oligo pools were cloned into the lentiviral gRNA expression plasmid using Gibson assembly as previously described (Klann et al., Curr. Opin. Biotechnol. 2018, 52, 32-41, which is incorporated herein by reference in its entirety). Briefly, oligo pools were amplified across 16 PCRs (100 ng oligo per PCR) with the following primers for 10 cycles using Q5 2×master mix and the following primers:

Fwd:
(SEQ ID NO: 480)
5′-TAACTTGAAAGTATTTCGATTTCTTGGCTTTATATATCTTGTGGAA
AGGACGAAACACCG
Rev:
(SEQ ID NO: 481)
5′-GTTGATAACGGACTAGCCTTATTTAAACTTGCTATGCTGTTTCCAG
CATAGCTCTTAAAC

Pools were gel purified (Qiagen, 28704) and used to assemble plasmid pools with Gibson assembly (NEB, E2611L). Pools were assembled across 16 Gibson assembly reactions (˜900 ng backbone, 1:3 backbone to insert) for the first screen, and 4 reactions for the second sub-library screen.

Lentivirus Production. The lentivirus encoding gRNA libraries or dCas9^{KRAB} was produced by transfecting 5×10⁶ HEK293T cells with the lentiviral gRNA expression plasmid pool or dCas9^{KRAB} plasmid (20 μg), psPAX2 (Addgene, 12260, 15 μg), and pMD2.G (Addgene, 12259, 6 μg) using calcium phosphate precipitation (Salmon P, Trono D. Curr. Protoc. Neurosci. 2006 November; Chapter 4:Unit 4.21. doi: 10.1002/0471142301.ns0421s37. PMID: 18428637, which is incorporated herein by reference in its entirety). After 14-20 hours, the transfection media was exchanged with fresh media. Media containing lentivirus was collected 24 and 48 hours later. Lentiviral supernatant was filtered with a 0.45 μm CA filter (Corning, 430627). The dCas9^{KRAB} lentivirus was concentrated 20× the initial media volume using Lenti-X concentrator (Clontech, 631232), following manufacturer's instructions. The lentivirus encoding gRNA libraries was used unconcentrated.

The titer of the lentivirus containing either the genome-wide library or distal sub-library of gRNAs was determined by transducing 5×10⁵ cells with varying dilutions of lentivirus and measuring the percentage of GFP-positive cells 4 days later using the Accuri C6 flow cytometer (BD Biosciences).

To produce lentivirus for individual gRNA validations, 8×10⁵ cells were transfected with gRNA plasmid (2440 ng), psPAX2 (1830 ng), and pMD2.G (730 ng) using Lipofectamine 3000 following the manufacturer's instructions. After 14 to 20 hours, transfection media was exchanged with fresh media. Media containing produced lentivirus was harvested 24 and 48 hours later, centrifuged for 10 minutes at 800×g, and directly used to transduce cells.

Lentiviral gRNA Screens. For the first genome-wide screen, 1.7×10⁹ cells were transduced with the gRNA library during seeding in 3 L spinner flasks across 4 replicates for controls (K562 cells without dCas9^{KRAB}) and 4 replicates for dCas9^{KRAB}-expressing cells. For sub-library screens, 4.17×10⁸ cells were transduced during seeding in 500 mL spinner flasks across 4 replicates for both controls and dCas9^{KRAB}-expressing cells. Cells were transduced at a multiplicity of infection (MOI) of 0.4 to generate a cell population with >80% of cells harboring only 1 gRNA and 500-fold coverage of each gRNA library. After 2 days, cells were treated with puromycin (Millipore Sigma, P8833) at a concentration of 2 μg/mL. Cells (control and dCas9^{KRAB}-expressing) were selected for 7 days and allowed to grow for a total of 16 days (including 7 days of selection, or ˜14 doublings). Cells were passaged to ensure at least 500× fold coverage of the gRNA library to maintain representation. After culturing, for the genome-wide screen, 5.5×10⁸ K562 cells were harvested for genomic DNA isolation. For the sub-library distal screens in K562 cells or OCI-AML2 cells, 1.5×10⁸ cells were harvested. Genomic DNA was harvested from cells as described (Chen et al., Cell. 2015, 160, 1246-1260, which is incorporated herein by reference in its entirety).

Single-Cell RNA-seq Screen. For the single-cell RNA-seq screen, cells constitutively expressing dCas9^{KRAB} were transduced with a library of gRNAs cloned into the CROP-seq-opti vector (Addgene #106280) in order to capture gRNA information on the 10× platform. The library contains 3,201 total gRNAs consisting of the most significant gRNA for all 3,051 distal DHS hits identified in the second K562 distal sub-library screen, as well as the most significant gRNA for a subset of TSS DHSs as positive controls, and 150 non-targeting gRNAs as negative controls (see, for example, TABLES S16-S17 and as in Klann et al. 2021. “Genome-wide annotation of gene regulatory elements linked to cell fitness” bioRxiv doi: 10.1101/2021.03.08.434470, which is incorporated herein by reference in its entirety). Cells were transduced at an MOI of ˜7 to achieve multiple integrations of gRNAs per cell, as done previously (Gasperini et al., Cell. 2018, doi:10.1016/j.cell.2018.11.029, which is incorporated herein by reference in its entirety). Cells were grown for 5 days after transfection of the gRNA library and 56,882 cells were collected and barcoded with the 10×3′ v3 chemistry. gRNAs were amplified from barcoded cDNA as described in previously (Gasperini et al., Cell. 2018, doi:10.1016/j.cell.2018.11.029, which is incorporated herein by reference in its entirety). Total transcriptome libraries were sequenced on a NovaSeq S4 flow cell and gRNA-enriched libraries were sequenced on a NextSeq 550 flow cell.

Genomic DNA Sequencing. To amplify the genome-wide gRNA libraries from each sample, 5.25 mg of genomic DNA (gDNA) was used as template across 525×100 μL PCR reactions using Q5 2× Master Mix (NEB, M0492L). For the distal sub-library screens, 1.2 mg of gDNA was used as template across 120 PCR reactions using Q5 2× Master Mix. Amplification was carried out following the manufacturer's instructions using 25 cycles at an annealing temperature of 60° C. using the following primers:

Fwd
(SEQ ID NO: 482)
5′-AATGATACGGCGACCACCGAGATCTACACAATTTCTTGGGTAGTTT
GCAGTT
Rev
(SEQ ID NO: 483)
5′-CAAGCAGAAGACGGCATACGAGAT(6 bp index
sequence)GACTCGGTGCCACTTTTTCAA

Amplified libraries were purified using Agencourt AMPure XP beads (Beckman Coulter, A63881) using double size selection of 0.65× and then to 1× the original volume. Each sample was quantified after purification using the Qubit dsDNA High Sensitivity Assay Kit (ThermoFisher, Q32854). Samples were pooled and sequenced on a HiSeq 4000 or NovaSeq 6000 (IIlumina) at the Duke GCB sequencing core, with 21 bp single read sequencing using the following custom read and index primers:

Read1
(SEQ ID NO: 484)
5′-GATTTCTTGGCTTTATATATCTTGTGGAAAGGACGAAACACCG
Index
(SEQ ID NO: 485)
5′-GCTAGTCCGTTATCAACTTGAAAAAGTGGCACCGAGTC

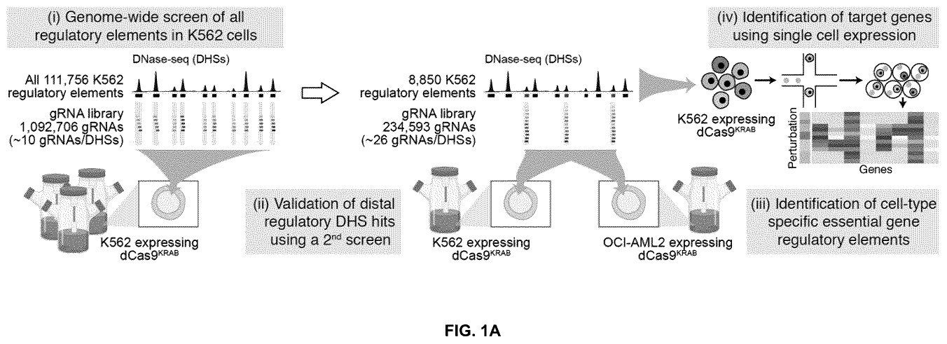

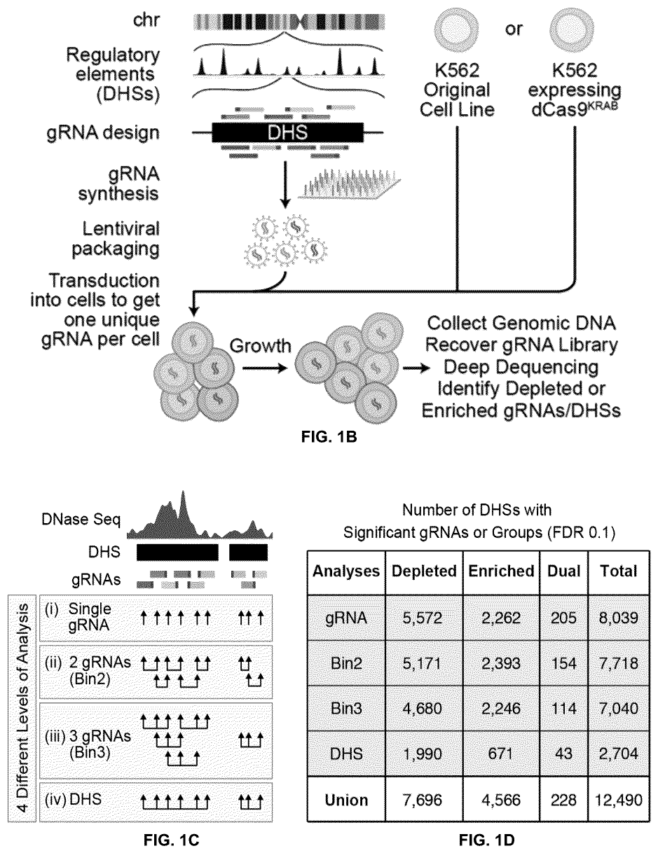

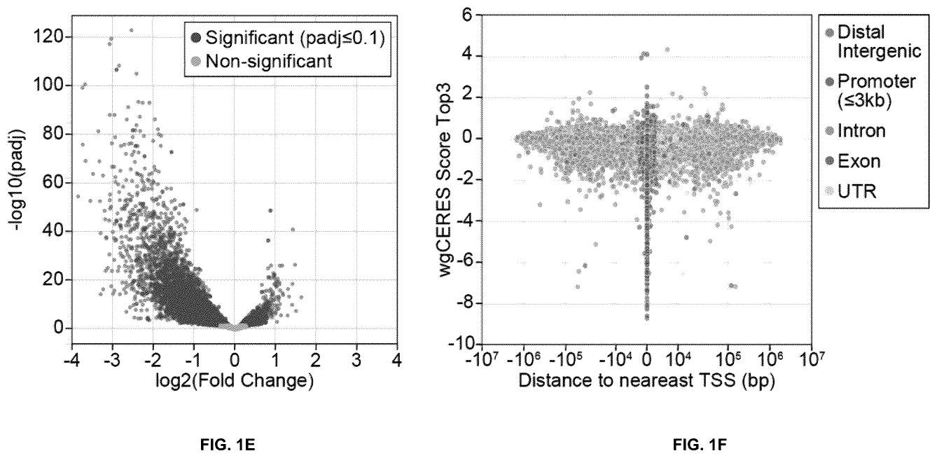

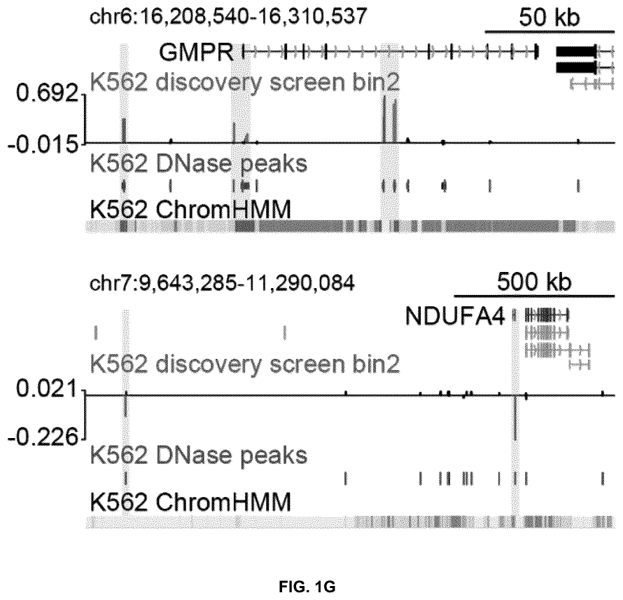

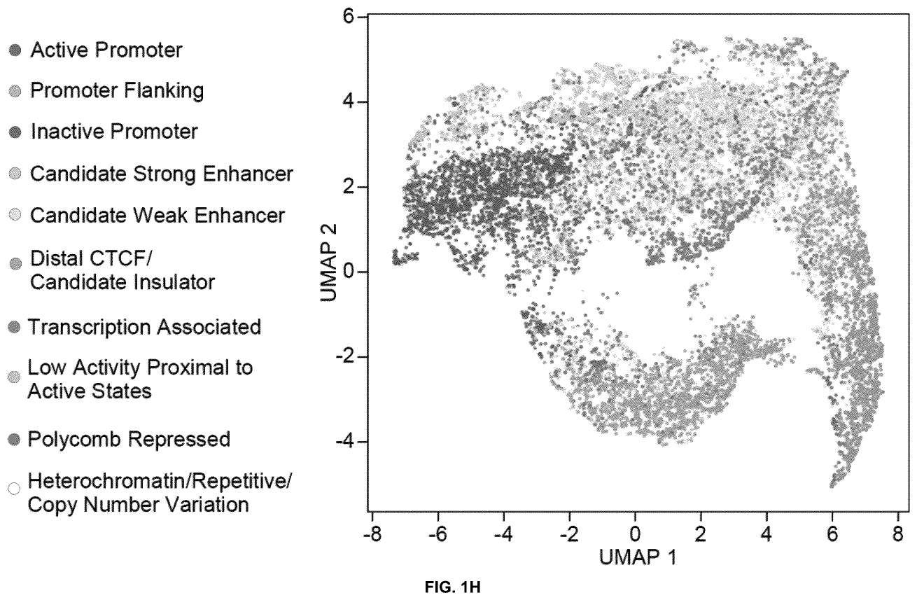

Data Processing and Differential Expression Analysis of gRNA libraries. To identify and quantify the effects of regulatory element perturbation on cell fitness, gRNA abundance was compared before and after cell growth. Since library size constraints limited the number of gRNAs per DHS and as the effect of any individual gRNA may be subtle, the effects of perturbing each DHS were characterized by four levels of gRNA analyses: 1) individual gRNAs, 2) a sliding window across each DHS in bins of two gRNAs, 3) a sliding window across each DHS in bins of three gRNAs, and 4) grouping all gRNAs in a DHS together (FIG. 1B).

FASTQ files were aligned to custom indexes (generated from the bowtie2-build function) using Bowtie2 (Langmead et al., Nat. Methods. 2012, 9, 357-359, which is incorporated herein by reference in its entirety) (options -p 24 --no-unal --end-to-end --trim3 6 -D 20 -R 3 -N 0 -L 20 -a). Counts for each gRNA were extracted and used for further analysis. All gRNA enrichment analysis was performed using R. For differential expression analysis, the DESeq2 package was used to compare between dCas9^{KRAB} and control (no dCas9^{KRAB}) conditions for each screen.

To summarize enrichment or depletion across a DHS in the first screen, composite scores (wgCERES-top3 score) were generated where the list of gRNAs, bins of 2 gRNAs, or bins of 3 gRNAs for each DHS were sorted by adjusted p-value (ascending order, calculated from DESeq2) and the average of the top three log 2(fold-change) values in each category was calculated. The log 2 (fold-change) averages (or single value for the DHS group) of each analysis category (gRNA/bin2/bin3/DHS) were then summed to calculate the wgCERES-top3 score. For the distal screens, the same procedure was performed except instead of the top 3 gRNAs/bins, the top 5 were averaged since gRNAs were more densely tiled for each DHS (wgCERES-top5 score).

Data Processing and Differential Expression Analysis of Single-Cell RNA-seq Screen. Sequencing data from transcriptome and gRNA libraries generally used distinct pre-processing pipelines, as detailed below. However, for both types of libraries, reads were first demultiplexed using the mkfastq command from 10× Genomics Cell Ranger 3.1.0 with the default configuration and BAM files with transcript counts were generated using the count command and the hg19 reference dataset included in Cell Ranger 3.1.0. At that point, the preprocessing of the transcriptomic data finished.

Custom processing of the gRNA library sequencing data. For the gRNA libraries, properly aligned reads were filtered out, since usable reads should not map against the hg19 transcriptome. BAM files containing unaligned reads were converted into FASTQ files using the bam2fq command in samtools. Next, the custom bowtie2 index from the wgCERES library described above was used to align the reads again using bowtie2. 23 and 48 bp were trimmed from the 5′ and 3′ ends respectively of the reads to remove scaffolding sequences. The full set of bowtie2 params were --trim5 23 --trim3 48 --no-unal --end-to-end -D 15 -R 2 -N 1 -L 18 -i S,1,0 --score-min G,0,0 --ignore-quals. Because of this extra step, we lost the corrected cell barcodes and UMI tags assigned by the cellranger software. Those were recovered by extracting into FASTQ files the optional fields CB for cell barcodes and UB for UMI barcodes, and reassigning these to the BAM files created with the custom bowtie index using the AnnotateBamVVithUmis function in the fgbio package (v.0.8.1). Finally, a custom script (scRNAseq.extract_umi_counts_from_grna_bam.py) was written to extract unique UMI counts per gRNA per cell. The resulting sparse matrix was saved in MarketMatrix format, compatible with existing single-cell RNA-seq software.

Differential expression analysis of single-cell CERES. Both the gRNA library and transcriptomic data were loaded in R using the Read10× function from Seurat v3.1.2 and merged the information in a single Seurat object. gRNAs were assigned to cells by requiring gRNAs to have ≥5 UMI counts and ≥0.5% of the total UMI counts in a cell (library size). Cells with >20% of mitochondrial UMI counts or <10,000 transcript UMIs were filtered out. Transcriptomic UMI counts were normalized using the NormalizeData function with default parameters. Cells with no gRNA assigned were discarded.

For each target gRNA, the FindMarkers function was used to test genes in the ±1 Mb window around the gRNA midpoint. MAST was the test used to recover significant differences in the expression of transcripts from cells containing the gRNA versus all other cells. The union set of all genes tested at least once was used to run the same analysis for non-targeting guides.

Finally, for each target gRNA-gene pair, an empirical p-value was calculated by counting the number of instances in which the observed p-value was larger than those in the non-targeting gRNA-gene pairs.

Permutation analysis. To test whether significant DHS hits clustered at distances that were closer than random chance, 1000 permutations of non-significant DHS sets were generated. Each permutation had the same number of non-significant DHSs as the significant DHS set. Then, the distance for each significant DHS to any non-significant DHS from each permuted set was measured.

Individual gRNA Validations using qRT-PCR, RNA-seq and competition assays. Validation of individual gRNAs in distal (non-promoter) putative regulatory elements were chosen from a list of 81 element-gene connections predicted by the ABC model (Fulco et al., Science. 2016, 354, 769-773, which is incorporated herein by reference in its entirety; Fulco et al., Nature Genetics. 2019, 51, 1664-1669, which is incorporated herein by reference in its entirety) (see, for example, TABLES S14 and S15 and as in Klann et al. 2021. “Genome-wide annotation of gene regulatory elements linked to cell fitness” bioRxiv doi: 10.1101/2021.03.08.434470, which is incorporated herein by reference in its entirety). These validations were focused on distal DHS hits that also had a corresponding promoter DHS hit of the predicted ABC target gene. From the list of ABC-predicted element-gene connections, several gRNAs corresponding to nearby DHSs that were significant in the wgCERES screen but did not have a predicted gene by ABC were also included.

TABLE S14
23 individual validation gRNAs used for qRT-PCR, RNA-seq, and competition
assays.

| | | | Validation Screen |
|---|---|---|---|
| Guide ID | Spacer | ABC Gene | Enriched or Depleted |
| chr11.1735.21 | TAACTGTTACATGAAGACAA | LMO2 | depleted |
| | (SEQ ID NO: 486) | | |
| chr11.1734.4 | TGATTAGGGACAGTTCCCCG | LMO2 | depleted |
| | (SEQ ID NO: 487) | | |
| chr11.1733.94 | CTTGATCCTGAAGGCAACGA | LMO2 | depleted |
| | (SEQ ID NO: 488) | | |
| chr19.727.22 | GTGAGGCAGGAGAGAGAGGG | CELF5 | depleted |
| | (SEQ ID NO: 489) | | |
| chr19.2258.29 | CAACGCTCTTGGGGACCCGC | C19orf53 | depleted |
| | (SEQ ID NO: 490) | | |
| chr4.1510.19 | CGGCCCCCTGGGATCCCCTG | COMMD8 | depleted |
| | (SEQ ID NO: 491) | | |
| chr16.579.2 | CGGGCGTTAGTCCGAGGAGG | C16orf59 | depleted |
| | (SEQ ID NO: 492) | | |
| chr12.662.25 | TGCCAAACTGGAGAGGCCGG | ATF7IP | depleted |
| | (SEQ ID NO: 493) | | |
| chr2.1528.50 | TGCAAGGGCCAGGCGAGGTC | GALM | depleted |
| | (SEQ ID NO: 494) | | |
| chrX.952.16 | GGGCAGATAAGGGAATCAGT | GATA1 | depleted |
| | (SEQ ID NO: 495) | | |
| chrX.2277.6 | CCGCCAGAGGGTGCACTAGC | CXorf48 | enriched |
| | (SEQ ID NO: 496) | | |
| chr6.847.37 | TCCATTTCAGTTTATACCAG | GMPR | enriched |
| | (SEQ ID NO: 497) | | |
| chr20.2352.1 | TACGTCATTCTCGGCTGAGC | CEBPB | enriched |
| | (SEQ ID NO: 498) | | |
| chr20.2325.7 | AGCCAGTGACCAATGAGACC | CEBPB | enriched |
| | (SEQ ID NO: 499) | | |
| chr17.2866.74 | CAGCTGGAAGGGTCAGAAGT | SLC4A1 | enriched |
| | (SEQ ID NO: 500) | | |
| chr17.2866.15 | CGGGACGCAGGCCTGGCGTA | SLC4A1 | enriched |
| | (SEQ ID NO: 501) | | |
| chr12.3340.2 | GAAACTGATTCCGAACCAGG | C12orf75 | enriched |
| | (SEQ ID NO: 502) | | |
| chr11.1723.3 | GAGGTTTATTGTGCCCAATG | LMO2 | enriched |
| | (SEQ ID NO: 503) | | |
| chr17.2862.59 | GGTGACTGAGGCCTACAGGC | SLC4A1 | enriched |
| | (SEQ ID NO: 504) | | |
| chr17.2868.27 | AGGAGGAAGACTAGCTAGCC | SLC4A1 | enriched |
| | (SEQ ID NO: 505) | | |
| chr6.852.11 | AGTTCAGGCTTGCTGGTGAG | GMPR | enriched |
| | (SEQ ID NO: 506) | | |
| chr6.853.33 | GAGGTCCCTGTGTTGGCTCT | GMPR | enriched |
| | (SEQ ID NO: 507) | | |
| chr6.854.58 | AATATGACAGGAGTGTGGTC | GMPR | enriched |
| | (SEQ ID NO: 508) | | |
| nontargeting.15405 | CCTCTTCCCGTCCAGCAGTA | NA | nontargeting |
| | (SEQ ID NO: 509) | | |

TABLE S15
Taqman probes for qRT-PCR validation.

| Supplier | Taqman Probe |
|---|---|
| Thermo Fisher | Catalog #: 4453320 |
| | Assay ID: Hs00153473_m1 |
| | Gene Symbol: LMO2 |
| Thermo Fisher | Catalog #: 4331182 |
| | Assay ID: Hs00254283_m1 Gene |
| | Symbol: CELF5 |
| Thermo Fisher | Catalog #: 4331182 |
| | Assay ID: Hs00958839_m1 |
| | Gene Symbol: C19orf53 |
| Thermo Fisher | Catalog #: 4448892 |
| | Assay ID: Hs01060714_m1 |
| | Gene Symbol: COMMD8 |
| Thermo Fisher | Catalog #: 4331182 |
| | Assay ID: Hs00228308_m1 |
| | Gene Symbol: C16orf59 |
| Thermo Fisher | Catalog #: 4331182 |
| | Assay ID: Hs00250569_s1 |
| | Gene Symbol: ATF7IP |
| Thermo Fisher | Catalog #: 4331182 |
| | Assay ID: Hs00373403_m1 |
| | Gene Symbol: GALM |
| Thermo Fisher | Catalog #: 4453320 |
| | Assay ID: Hs01085823_m1 |
| | Gene Symbol: GATA1 |
| Thermo Fisher | Catalog #: 4331182 |
| | Assay ID: Hs00250428_m1 |
| | Gene Symbol: CT55 |
| Thermo Fisher | Catalog #: 4331182 |
| | Assay ID: Hs00199328_m1 |
| | Gene Symbol: GMPR |
| Thermo Fisher | Catalog #: 4453320 |
| | Assay ID: Hs00942496_s1 |
| | Gene Symbol: CEBPB |
| Thermo Fisher | Catalog #: 4331182 |
| | Assay ID: Hs00978607_g1 |
| | Gene Symbol: SLC4A1 |
| Thermo Fisher | Catalog #: 4331182 |
| | Assay ID: Hs00329098_m1 |
| | Gene Symbol: C12orf75 |
| Thermo Fisher | Catalog #: 4448484 |
| | Assay ID: Hs00427620_m1 |
| | Gene Symbol: TBP |
| | Dye Label and Assay Concentration: VIC-MGB_PL |

The protospacers from the top enriched gRNAs found in each screen (see, for example, TABLES 51 to S13 and as in Klann et al. 2021. “Genome-wide annotation of gene regulatory elements linked to cell fitness” bioRxiv doi: 10.1101/2021.03.08.434470, which is incorporated herein by reference in its entirety) were ordered as oligonucleotides from IDT and cloned into a lentiviral gRNA expression vector as described earlier. The same modified cell lines used in the corresponding screen were used for the individual gRNA validations. The cells were transduced with individual gRNAs and after 2 days were selected with puromycin (2 μg/mL) for 7 days for the four distal gRNAs not connected with an ABC connection or 4 days for the gRNAs targeting DHSs connected to genes by ABC model predictions.

For all screen validations by qRT-PCR and RNA-seq, mRNA expression analysis was done in triplicate. Total mRNA was harvested from cells and cDNA was generated using the TaqMan Fast Advanced Cells-to-CT kit (ThermoFisher, A35377). qRT-PCR was performed using the TaqMan Fast Advanced Cells-to-CT kit with the FX96 Real-Time PCR Detection System (Bio-Rad) with the TaqMan probes listed in TABLE S15. The results are expressed as fold-increase mRNA expression of the gene of interest normalized to TBP expression by the 44Ct method.

RNA-seq analysis was performed as follows. Raw reads were trimmed to remove adapters and bases with average quality score (Q) (Phred33) of <20 using a 4 bp sliding window (SLIDINGWINDOW:4:20) with Trimmomatic v0.32 (Bolger et al., Bioinformatics. 2014, 30, 2114-2120, which is incorporated herein by reference in its entirety). Trimmed reads were subsequently aligned to the primary assembly of the GRCh37 human genome using STAR v2.4.1a (Dobin et al., Bioinformatics. 2013. 29, 15-21, which is incorporated herein by reference in its entirety). Aligned reads were assigned to genes in the GENCODE v19 comprehensive gene annotation (Harrow et al., Genome Res. 2012, 22, 1760-1774, which is incorporated herein by reference in its entirety) using the featureCounts command in the subread package v1.4.6-p4 with default settings (Liao et al., Nucleic Acids Res. 2013, 41, e108, which is incorporated herein by reference in its entirety). Differential expression analysis was performed using DESeq2 v1.22.0 (Love et al., Genome Biol. 2014, 15, 550, which is incorporated herein by reference in its entirety) in R (v3.5.1). Briefly, raw counts were imported and filtered to remove genes with low or no expression (i.e., keeping genes having counts per million (CPMs) in samples). Filtered counts were then normalized using the DESeq function, which internally uses estimated size factors accounting for library size, estimated gene and global dispersion. To find significantly differentially expressed genes, the nbinomWaldTest was used to test the coefficients in the fitted Negative Binomial GLM using the previously calculated size factors and dispersion estimates. Genes having a Benjamini-Hochberg false discovery rate (FDR) less than 0.05 were considered significant (unless otherwise indicated). Log₂ fold-change values were shrunk towards zero using the adaptive shrinkage estimator from the ‘ashr’ R package (Stephens, Biostatistics. 2017, 18, 275-294, which is incorporated herein by reference in its entirety). For estimating transcript abundance, transcripts per million (TPMs) were computed using the rsem-calculate-expression function in the RSEM v1.2.21 package (Li and Dewey, BMC Bioinformatics. 2011, 12, 323, which is incorporated herein by reference in its entirety).

For growth competition assays, 1×10⁶ cells were transduced with lentivirus encoding a single gRNA into polyclonal K562 dCas9^{KRAB} cells. Cells were transduced with either 1) an individual targeting gRNA and GFP or 2) a non-targeting gRNA and mCherry. After 2 days, cells were selected with puromycin (2 μg/mL) for 5 days. After selection, for each validation gRNA, 5×10⁴ GFP-positive cells were seeded with 5×10⁴ mCherry-positive cells expressing the non-targeting gRNA. The percent of GFP- and mCherry-positive cells in each well was assayed 1, 7, and 14 days later using a FACSCanto II flow cytometer (BD Biosciences).

### Example 2

### Genome-Wide Screen of all Regulatory Elements in K562 Cells

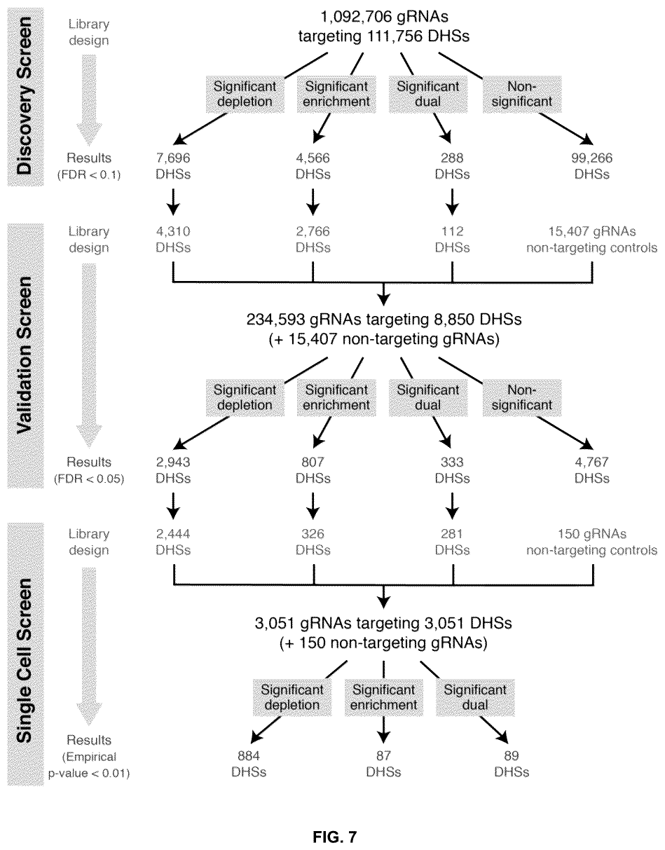

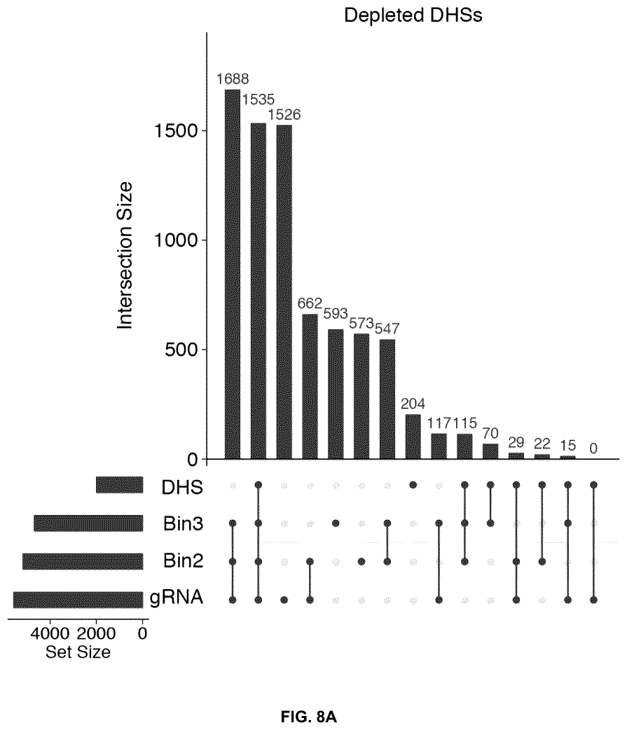

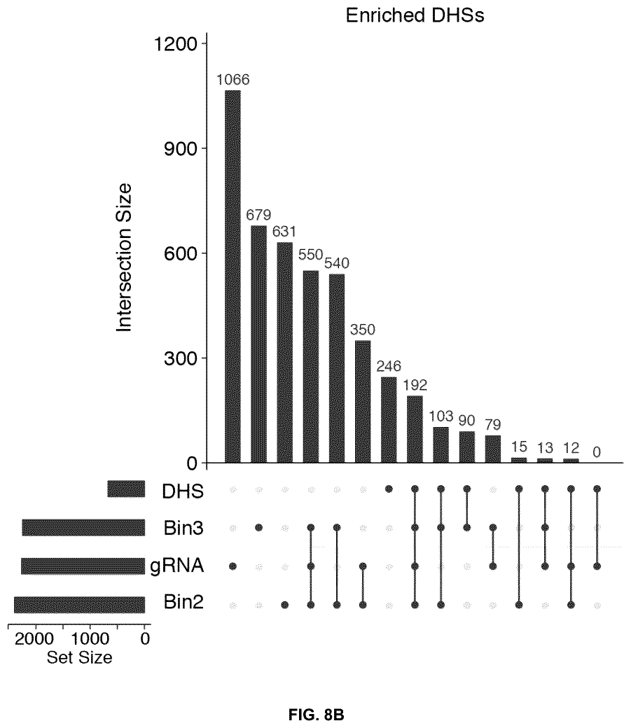

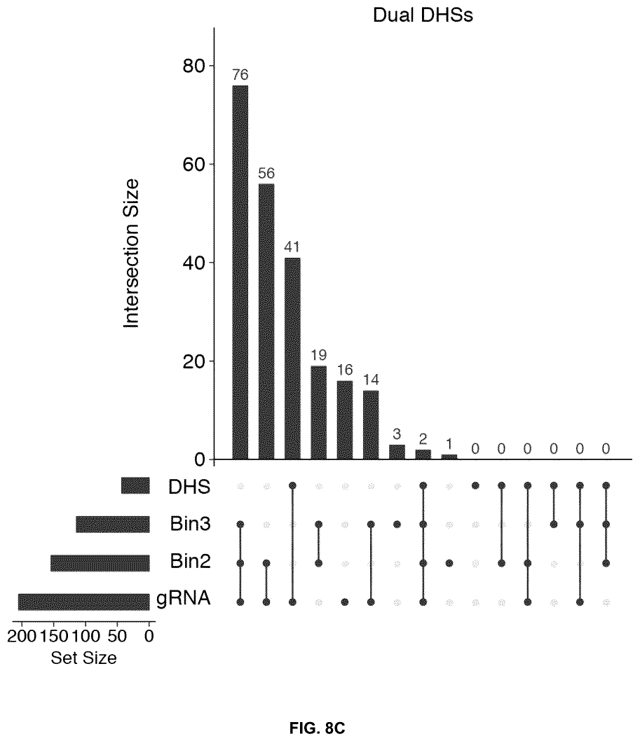

Whole-genome CERES (wgCERES) was used to measure the effect of epigenetically silencing 111,756 putative regulatory elements, defined by DNase-I hypersensitive sites (DHS), on cell fitness in K562 cells (FIGS. 1A-1B and FIG. 7) (ENCODE Project Consortium, Nature. 2012, 489, 57-74, which is incorporated herein by reference in its entirety). K562 cells were assayed because they are one of the most extensively characterized cell models in terms of chromatin accessibility, histone marks, transcription factor binding, and gene expression. The library herein contained 1,092,706 unique gRNAs averaging ˜10 gRNAs per DHS (see, for example, TABLES S1 to S4 and as in Klann et al. 2021. “Genome-wide annotation of gene regulatory elements linked to cell fitness” bioRxiv doi: 10.1101/2021.03.08.434470, which is incorporated herein by reference in its entirety), and this library was transduced into a clonal K562 cell line stably expressing the dCas9^{KRAB} transcriptional repressor (Gilbert et al., Cell. 2013, 154, 442-451, which is incorporated herein by reference in its entirety; Thakore et al., Nat. Methods. 2015, 12, 1143-1149, which is incorporated herein by reference in its entirety). After ˜14 population doublings, a significant depletion of gRNAs for 7,696 DHSs was identified, indicating that repressing those DHSs impaired cell viability or proliferation (FIGS. 1C-1D and FIG. 8A). 4,566 DHSs with gRNAs or combinations of gRNAs that were significantly enriched were also found, indicating that repressing these elements increased cell fitness (FIG. 1D, FIG. 8B)(see also, for example, TABLE S5 and as in Klann et al. 2021. “Genome-wide annotation of gene regulatory elements linked to cell fitness” bioRxiv doi: 10.1101/2021.03.08.434470, which is incorporated herein by reference in its entirety). A relatively small number of DHS hits (n=228) contained both enriched and depleted gRNAs (FIG. 1D and FIG. 8C).

### Example 3

### Attributes of gRNAs that Impact Cell Fitness

Effect sizes for gRNAs that reduced cell fitness were overall greater (average log 2 (fold-change)=−0.91) than those that increased cell fitness (average log 2 (fold-change)=0.48; FIG. 1E). That result is consistent with a model that it is easier to reduce fitness than increase fitness of the rapidly growing K562 cell line.

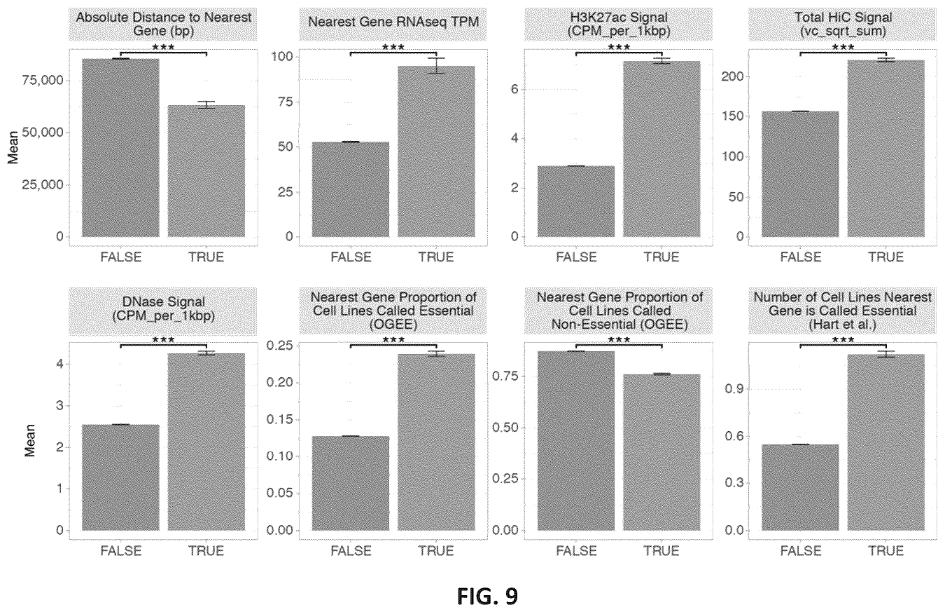

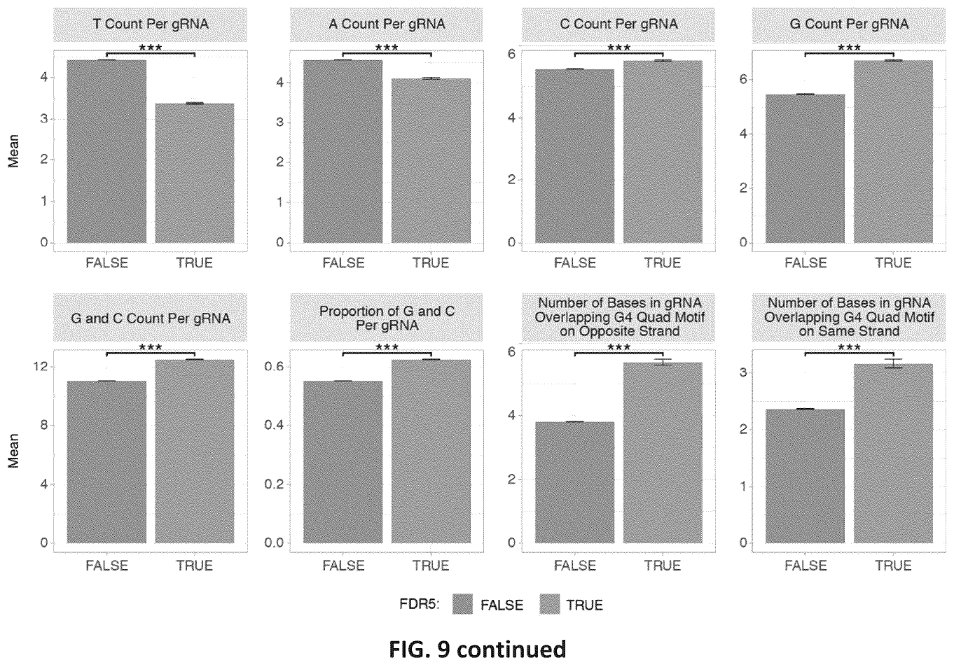

To better understand the characteristics that distinguish the significantly enriched or depleted gRNAs, each gRNA in the library was annotated with a selection of features (FIG. 9). The gRNAs with significantly changed abundance were enriched for GC content in the protospacer, G4 quadruplex motifs (Rhodes and Lipps, Nucleic Acids Res. 2015, 43, 8627-8637, which is incorporated herein by reference in its entirety; Gray et al., Nat. Chem. Biol. 2014, 10, 313-318, which is incorporated herein by reference in its entirety), nearby genes that were more highly expressed, higher accessibility, higher H3K27ac marks, and higher Hi-C contact frequency (FIG. 9). Additionally, genes nearest to significant gRNAs were enriched for genes previously identified as essential (Hart et al., Cell. 2015, 163, 1515-1526, which is incorporated herein by reference in its entirety; Wang et al., Cell. 2017, 168, 890-903.e15, which is incorporated herein by reference in its entirety) (FIG. 9). These features have been used previously to predict enhancer-gene interactions (Fulco et al., Science. 2016, 354, 769-773, which is incorporated herein by reference in its entirety; Fulco et al., Nature Genetics. 2019, 51, 1664-1669, which is incorporated herein by reference in its entirety), and support the power of this genome-wide screen to identify active regulatory elements associated with the selection criteria described herein.

### Example 4

### Attributes of Significant OHS Hits

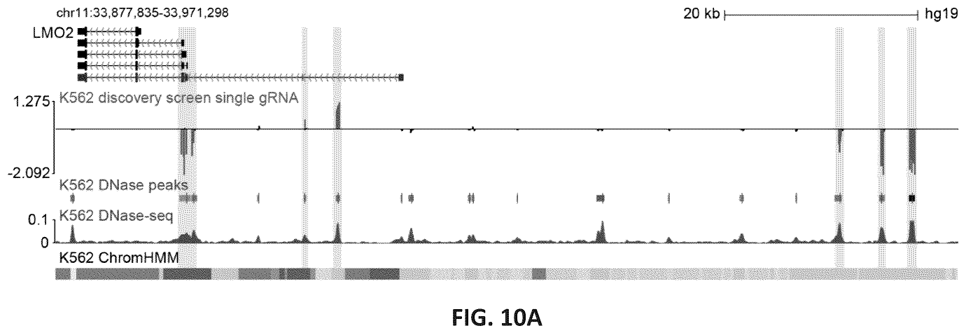

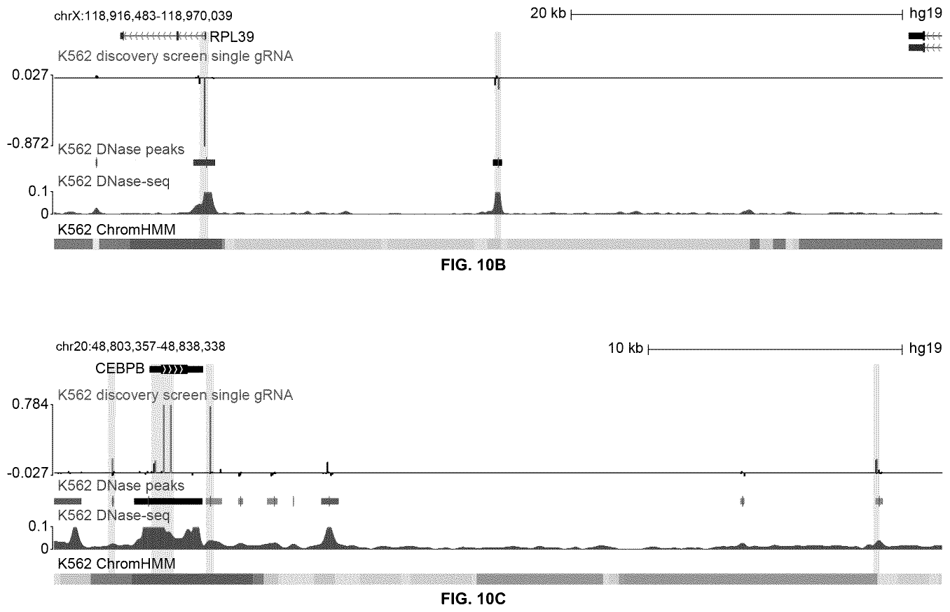

While significant DHS hits are called at a continuum of distances from the nearest gene, the strongest observed signals centered on DHSs that overlapped with transcriptional start sites (TSSs; FIG. 1F). This is consistent with previous studies showing that repressing promoters with dCas9^{KRAB} has a larger effect on gene expression than repressing distal regulatory elements. Although overall scores decrease away from TSSs, some distal DHSs have particularly strong signals, similar to TSS DHS hits. For example, several DHS hits 10 kb-1 Mb upstream of their putative target genes scored similarly to gRNAs that target the promoter of the same gene (FIG. 1G and FIGS. 10A-10C). Some of the distal elements were previously validated in mice to control genes such as the oncogene Lmo2 in erythroid cell lineages. Together, this indicates that wgCERES can identify regulatory elements distal from target genes, and quantify the relative impact of those regulatory elements on cell proliferation.

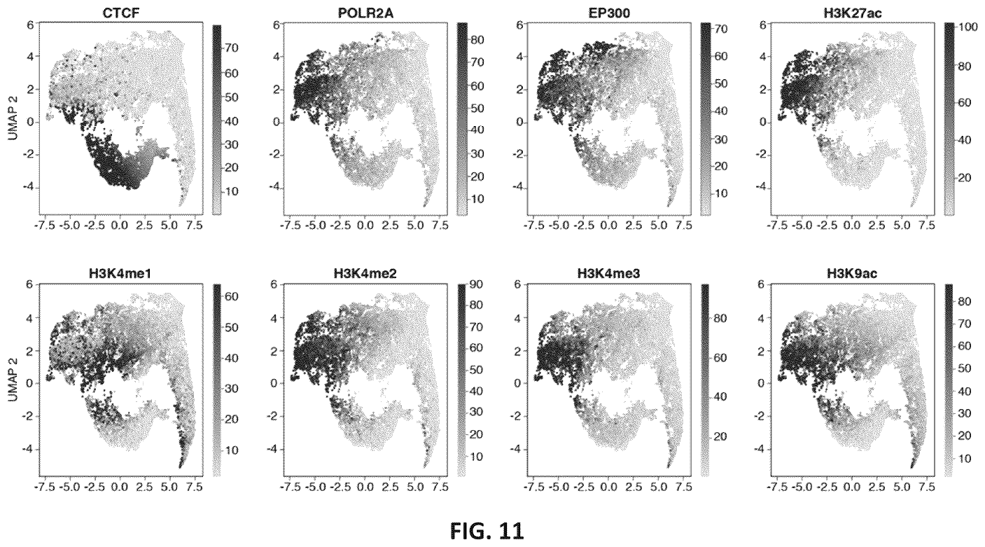

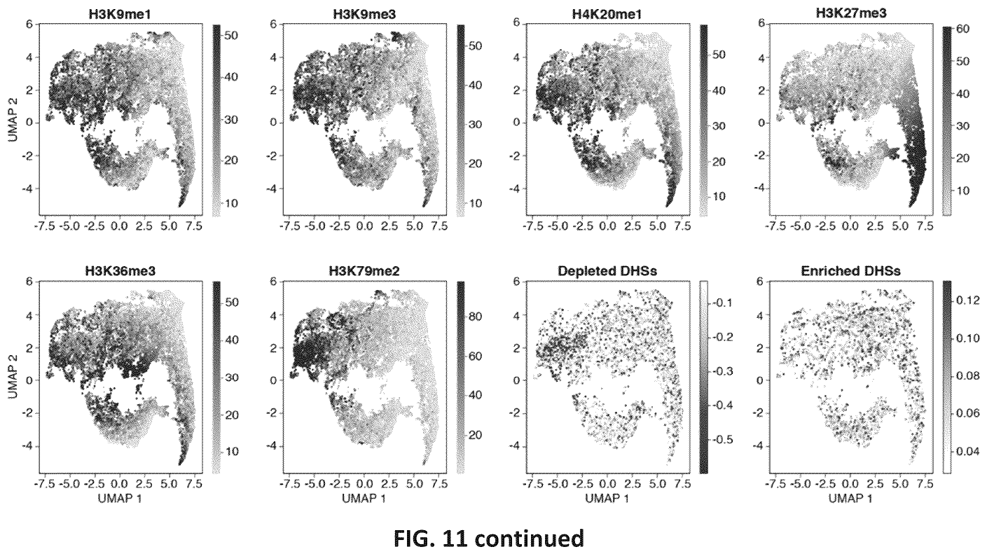

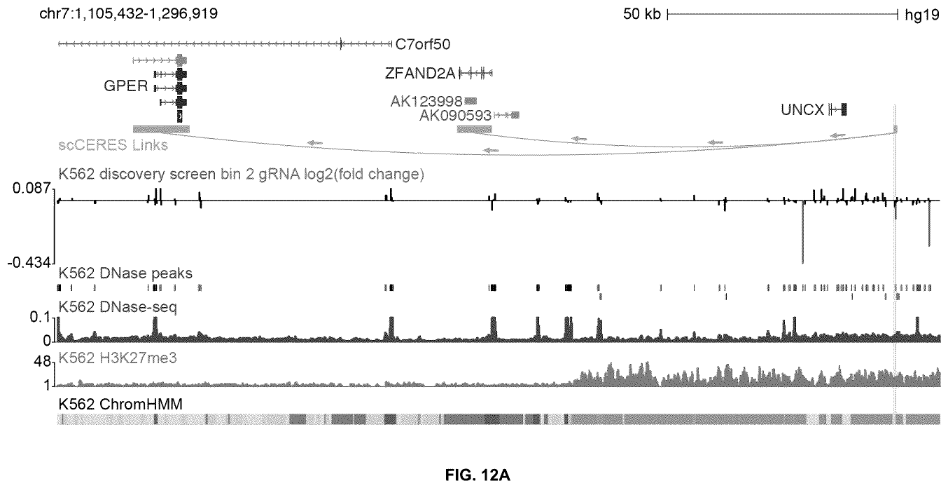

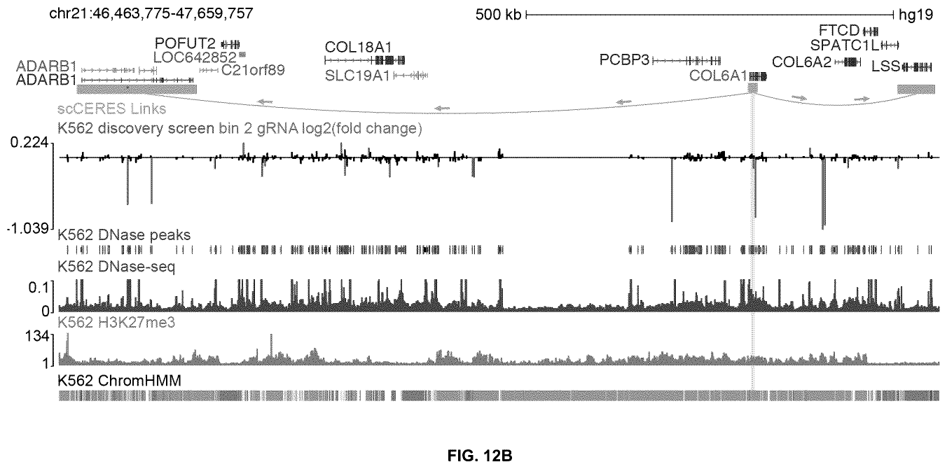

To identify epigenetic characteristics of DHS that control cell fitness, dimensionality reduction analysis was used, and DHS hits were compared to K562 ChIP-seq data for several histone modifications and epigenome-modifying proteins from the ENCODE project (FIG. 11) (ENCODE Project Consortium, Moore et al., Nature. 2020, 583, 699-710, which is incorporated herein by reference in its entirety). ChromHMM genome annotations (Ernst and Kellis, Nat. Methods. 2012, 9, 215-216, which is incorporated herein by reference in its entirety) was then used to identify classes of regulatory elements that were overrepresented in the enriched or depleted DHS hits (FIG. 1H). Hits in almost every class of annotation were observed, including regions classified as polycomb-repressed (FIGS. 12A-12B). Relative to all DHS sites, depleted DHS hits were overrepresented at active promoters and underrepresented at enhancers and CTCF sites (FIG. 1I). In contrast, enriched DHS hits have similar genomic location characteristics as all DHS (FIG. 1I). Together, these results indicate that promoters, enhancers, insulators, and polycomb-repressed regions can all contribute to cell fitness.

### Example 5

### Clustering of Significant DHS Hits

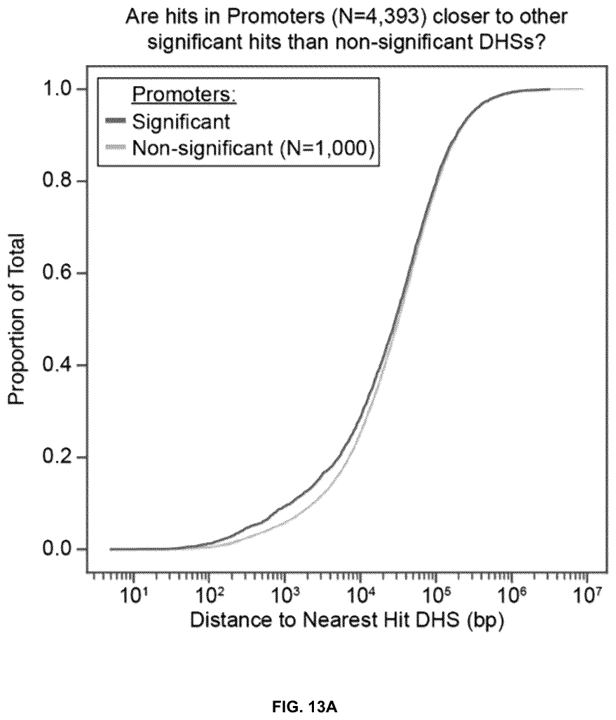

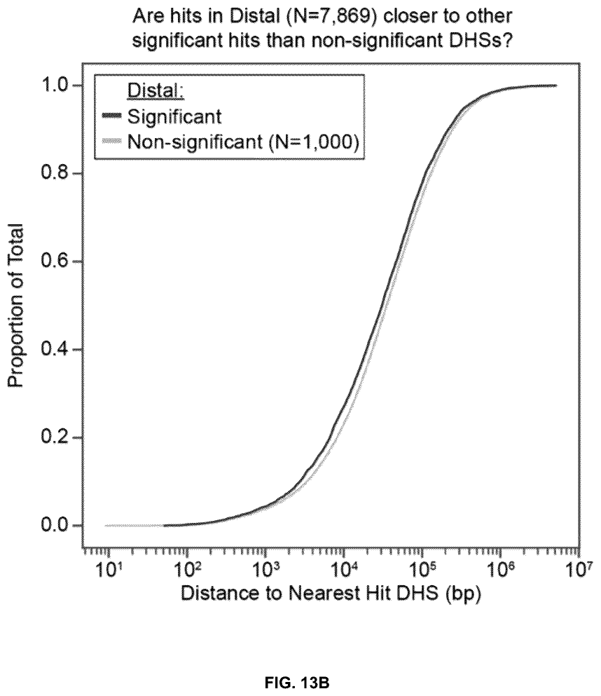

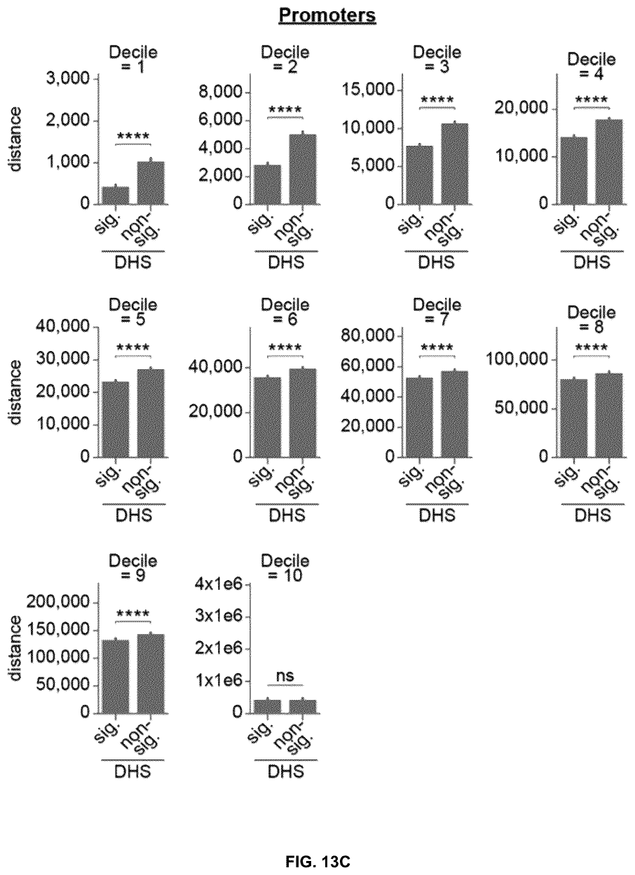

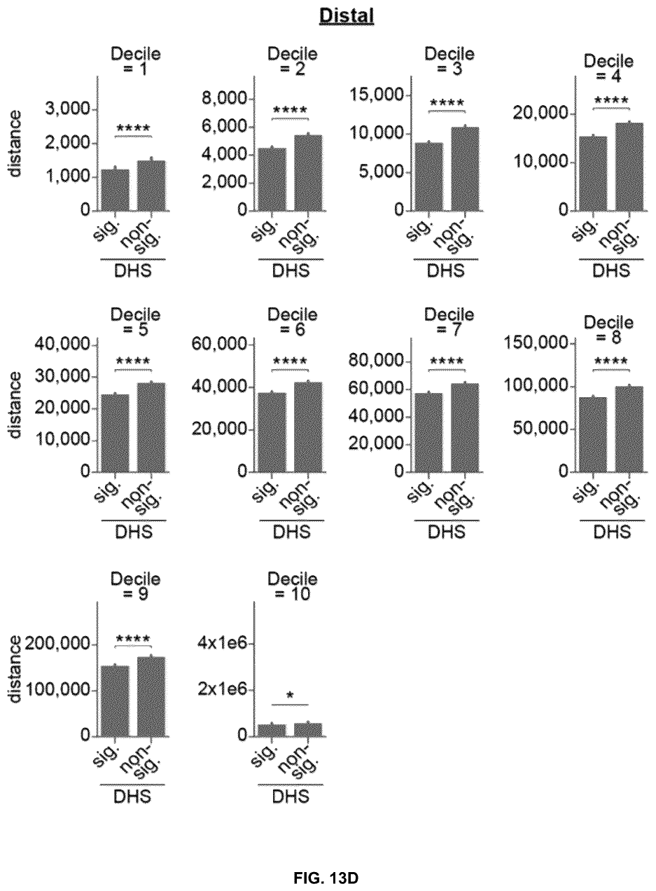

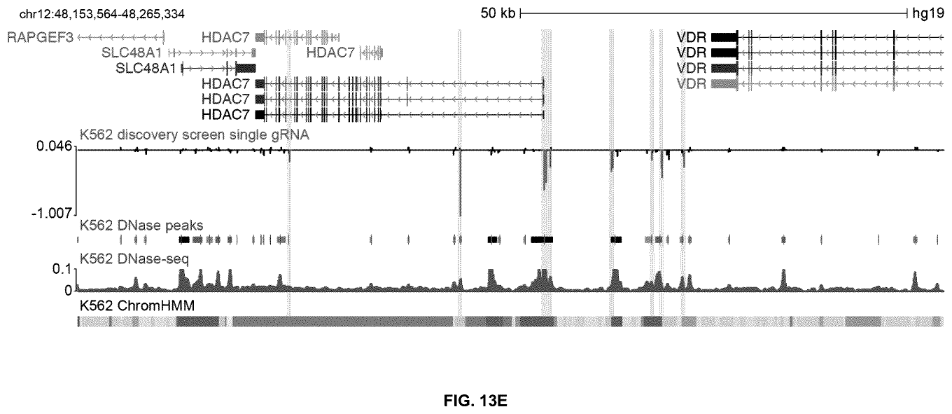

Clusters of individual regulatory elements can function together as larger ensembles to coordinate gene expression, as seen with the β-globin locus control region. To determine if DHS hits from this screen cluster together, the distances between adjacent DHS hits were compared. Distances for proximal DHS hits (TSSs; FIG. 13A) and distal DHS hits (>3 kb away from TSS; FIG. 13B) were separately measured. It was observed that DHS hits are significantly closer to each other than expected by chance using permutation analysis (FIG. 13A and FIG. 13B). Dividing the data into deciles (FIG. 13C and FIG. 13D), DHS hits were significantly closer to each other in all but the most distant decile (t-test, p<0.0001). Some clusters included up to 7 significant DHS hits, such as around the HDAC7/VDR locus that is known to be involved in cancer cell proliferation (FIG. 13E). Together, these results indicated that regulatory elements that influence cell fitness tend to cluster. That may be due to coordinated effects on key cell fitness genes and/or the presence of clustered genes that contribute to cell fitness.

### Example 6

### Validation of Distal Regulatory DNS Hits Using a Secondary Screen

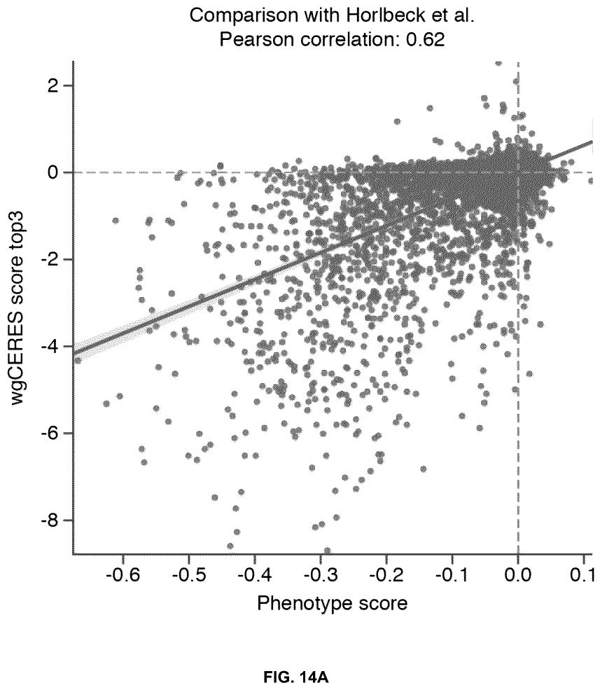

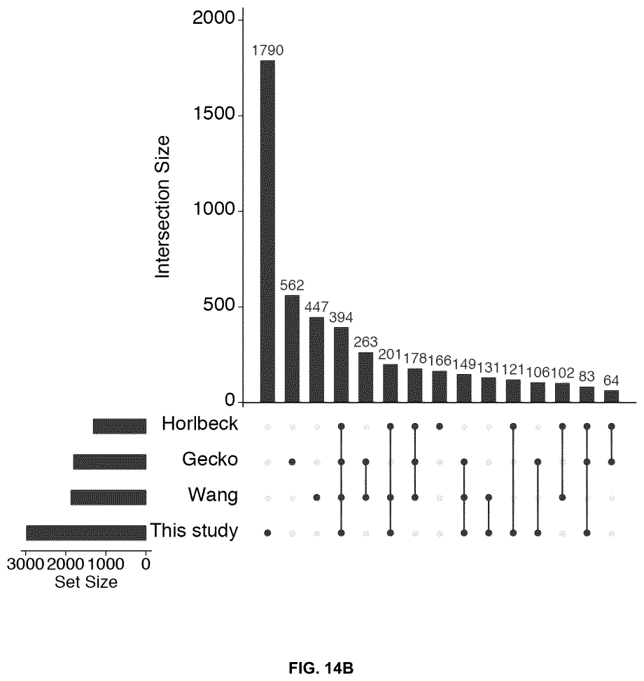

Primary screen hits were validated using both comparisons to previously identified essential genes and a secondary screen targeted to positive hits. For promoter hits, the results herein were compared to other studies of promoter inactivation (Horlbeck et al., eLife. 2016, 5, doi:10.7554/elife.19760, which is incorporated herein by reference in its entirety) or gene disruption (Lenoir et al., Nucleic Acids Res. 2018, 46, D776-D780, which is incorporated herein by reference in its entirety; Wang et al., Cell. 2017, 168, 890-903.e15, which is incorporated herein by reference in its entirety) in K562 cells. The observed promoter hits positively correlated (Pearson p=0.62, Spearman p=0.18) with the promoter CRISPRi screen (Horlbeck et al., eLife. 2016, 5, doi:10.7554/elife.19760, which is incorporated herein by reference in its entirety)(FIG. 14A)(see also, for example, TABLE S5 and as in Klann et al. 2021. “Genome-wide annotation of gene regulatory elements linked to cell fitness” bioRxiv doi: 10.1101/2021.03.08.434470, which is incorporated herein by reference in its entirety). Overall, ˜400 genes were hits in all four studies (FIG. 14B). That was the most common configuration for overlapping hits, indicating substantial concordance between gene- and promoter-based screens for effects on cell fitness.

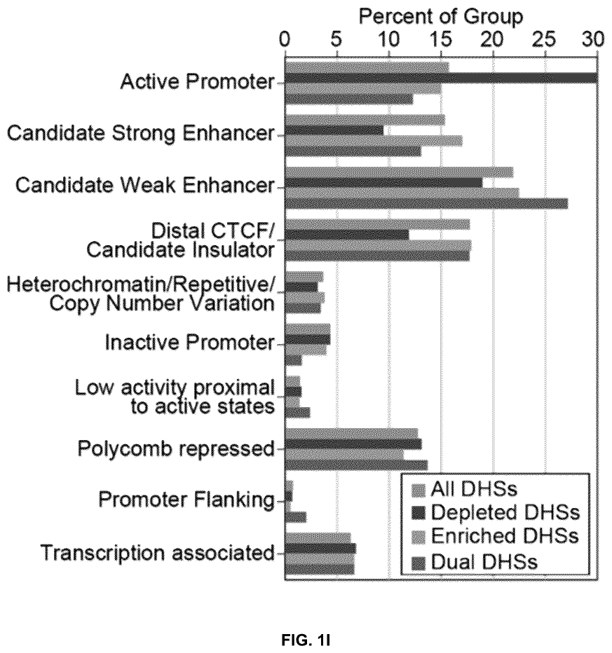

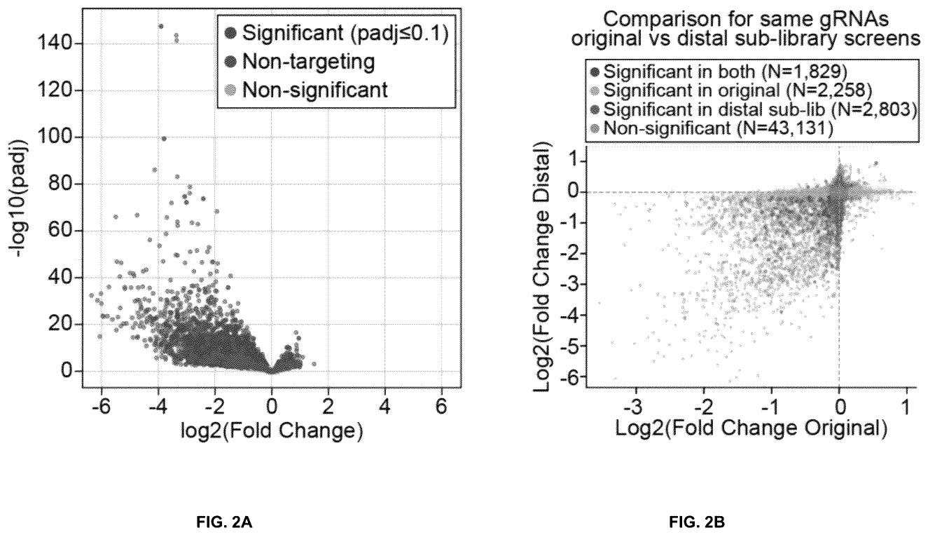

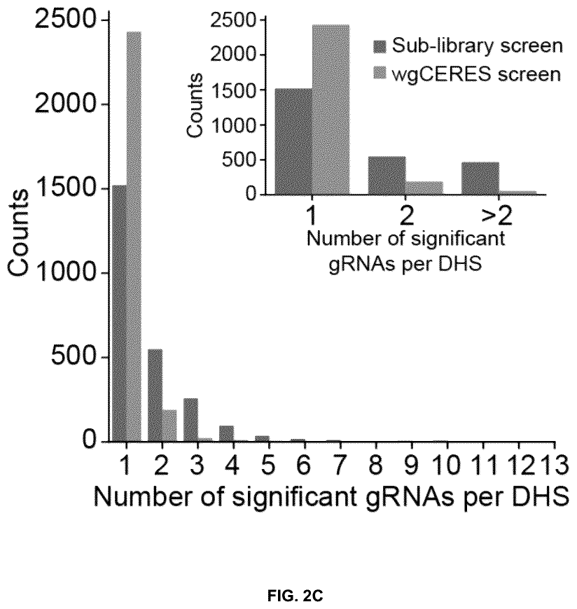

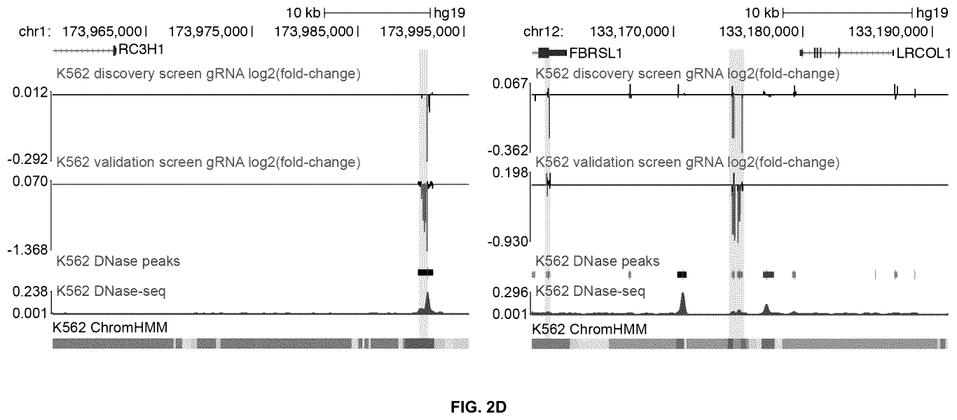

The screen herein was distinct from previous efforts in that most of the gRNAs described herein targeted putative distal regulatory elements. To validate and characterize the effects of individual gRNA and DHS hits, a validation screen of 234,593 gRNAs that collectively target 8,850 DHSs was completed, of which 7,188 were hits called at an FDR<0.1 in the initial discovery screen (FIG. 7)(see also, for example, TABLES S5 to S13 and as in Klann et al. 2021. “Genome-wide annotation of gene regulatory elements linked to cell fitness” bioRxiv doi: 10.1101/2021.03.08.434470, which is incorporated herein by reference in its entirety). Individual gRNA effects in the validation screen had a similar distribution as the original screen, both in terms of effects sizes and that most hits corresponded to a decrease in cell fitness (FIG. 2A)(see also, for example, TABLE S5 and as in Klann et al. 2021. “Genome-wide annotation of gene regulatory elements linked to cell fitness” bioRxiv doi: 10.1101/2021.03.08.434470, which is incorporated herein by reference in its entirety).

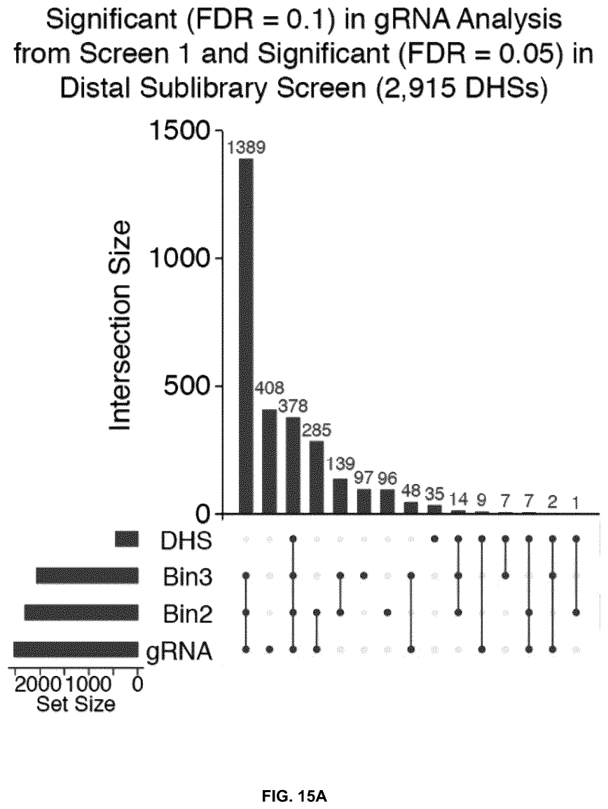

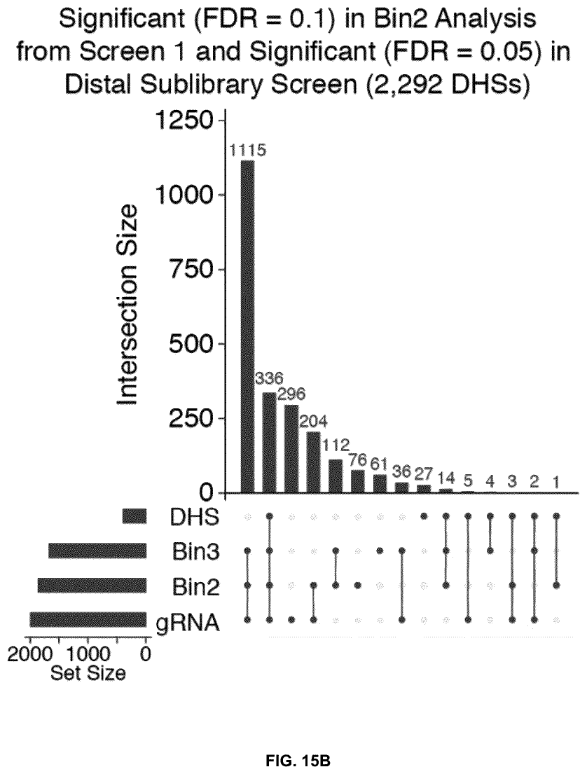

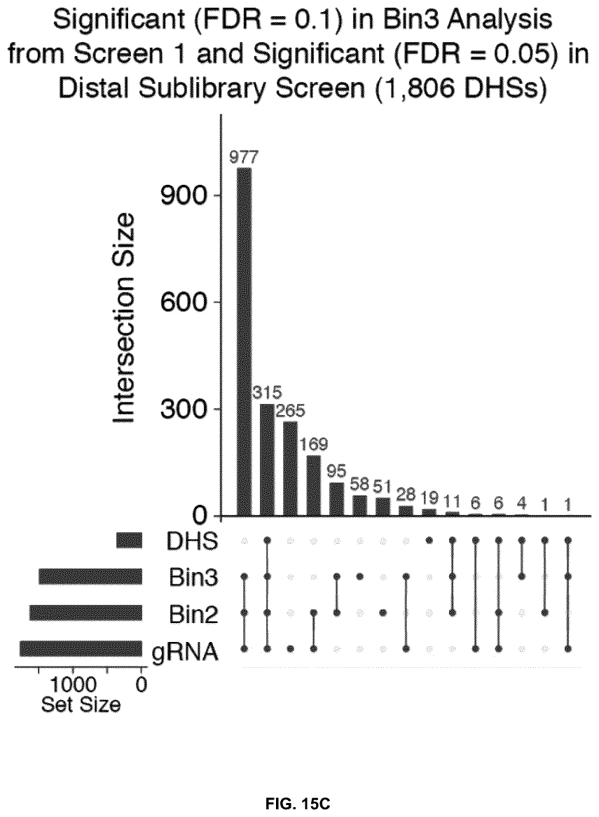

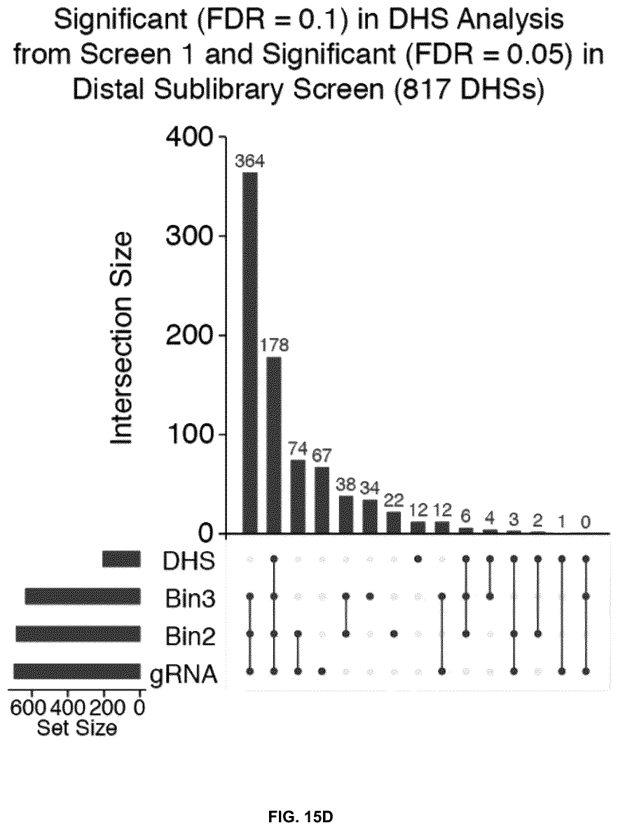

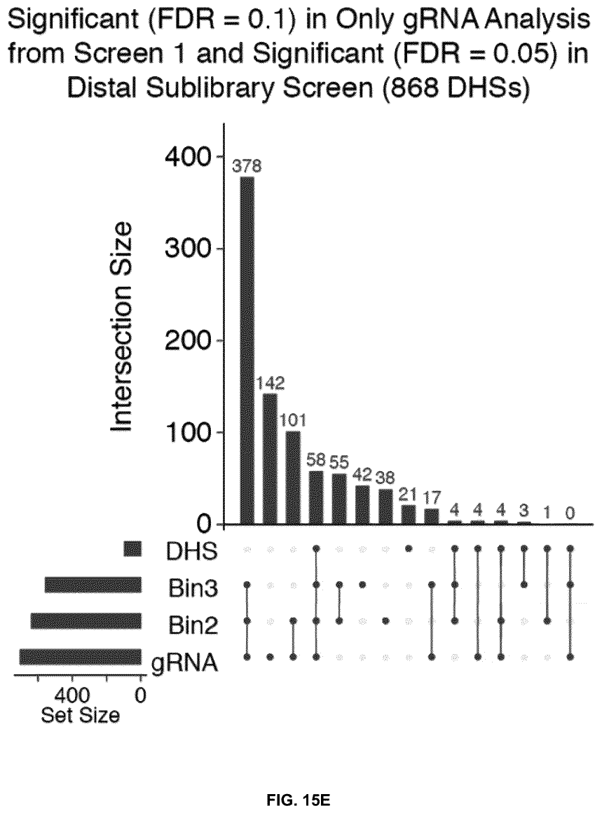

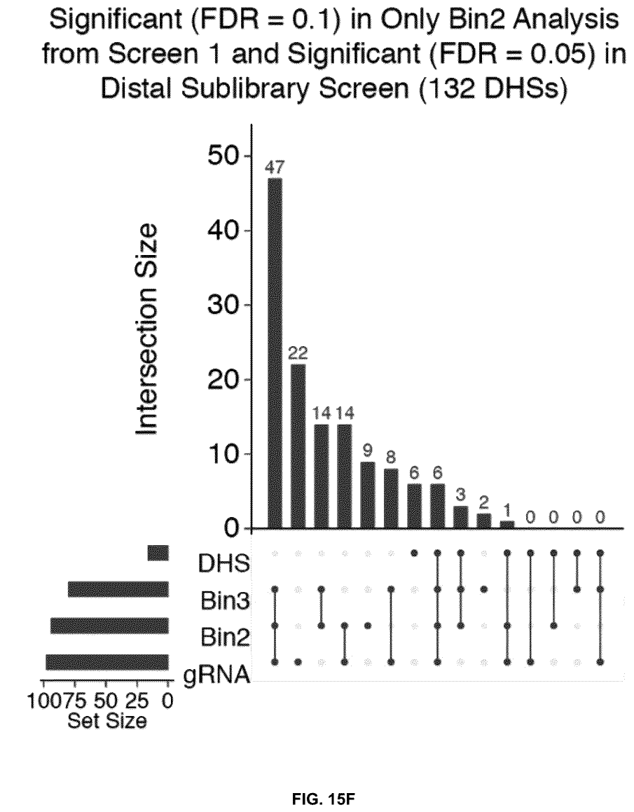

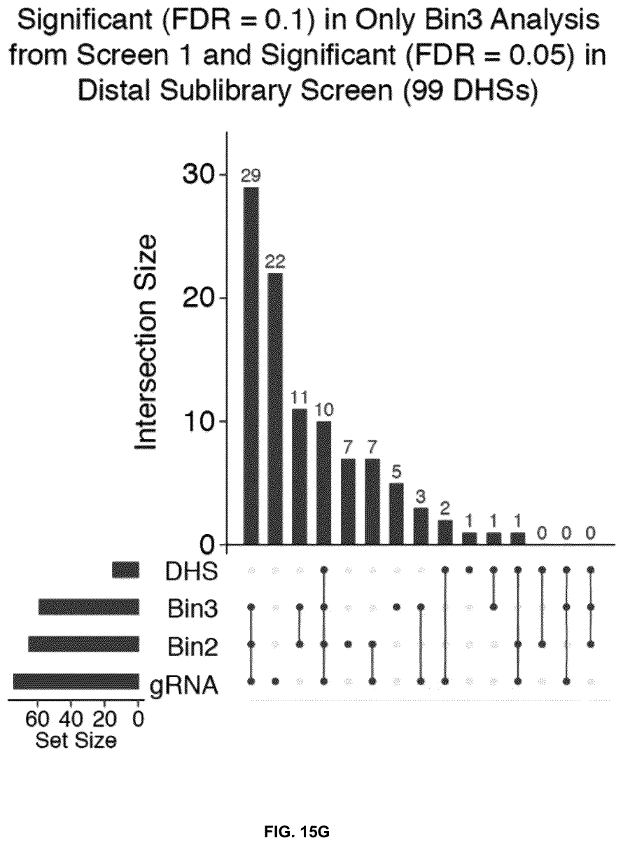

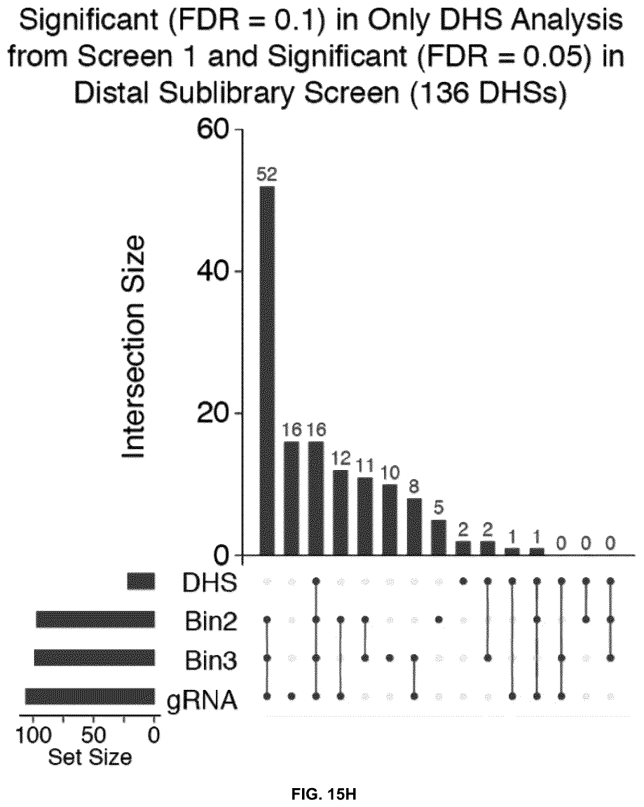

To evaluate performance for single gRNAs, 50,021 individual gRNAs assayed in both the discovery screen and the validation screen were characterized. Of those, 4,087 gRNAs were individually significant hits in the discovery screen at an FDR<0.1, and 1,829 were also significant in the validation screen at an FDR<0.1 (FIG. 2B and FIGS. 15A-15H). The remaining 45,934 gRNAs were included because they were in DHSs that had a significant effect, but the individual gRNA did not. Of the 45,934 gRNAs that were negative in the discovery screen, 43,131 gRNAs were also negative in the validation screen. Together, the validation screen indicated gRNA-level sensitivity of 40% with 45% precision and 95% specificity. Performance of the DHS-level analysis was also evaluated. Of the 8,850 DHS targeted, 7,188 had significant effects at FDR<0.1 in the discovery screen, and 3,532 (49%) positively validated at a more stringent FDR<0.05 in the validation screen.

The validation screen had more significant gRNA hits per DHS (FIGS. 2C-2D), suggesting that increasing the density of gRNAs tested per DHS from 10 to 26 improved detection of regulatory elements that impact cell fitness. That improved detection may be in part due to variation in the effects of gRNAs targeting the same DHS.

### Example 7

### Functional Validations of Target Genes for OHS Hits by Expression and Competition Assays

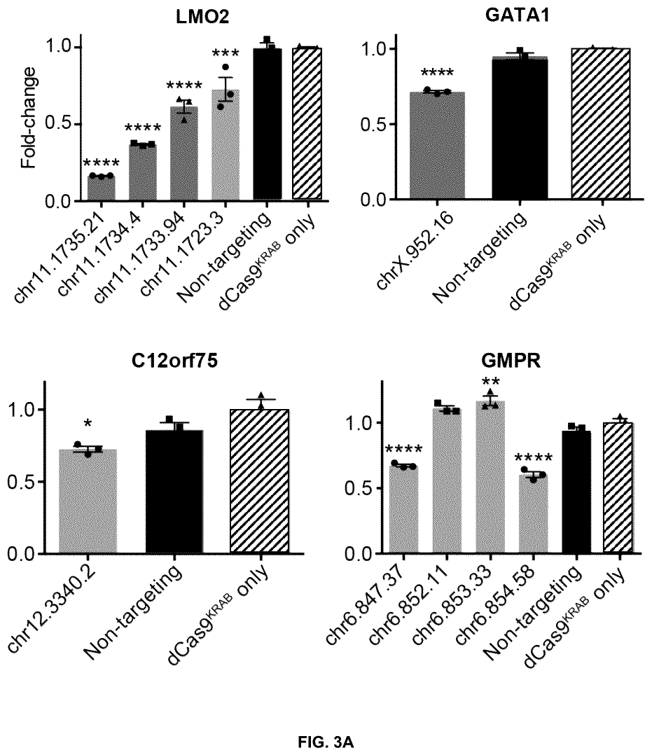

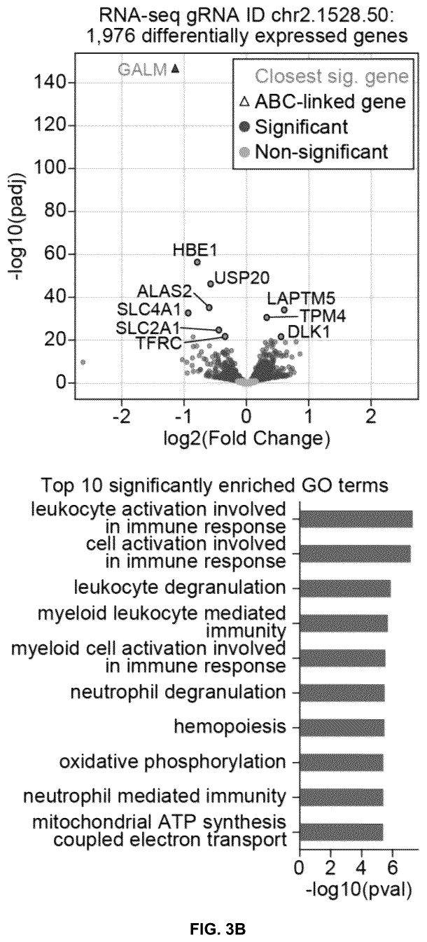

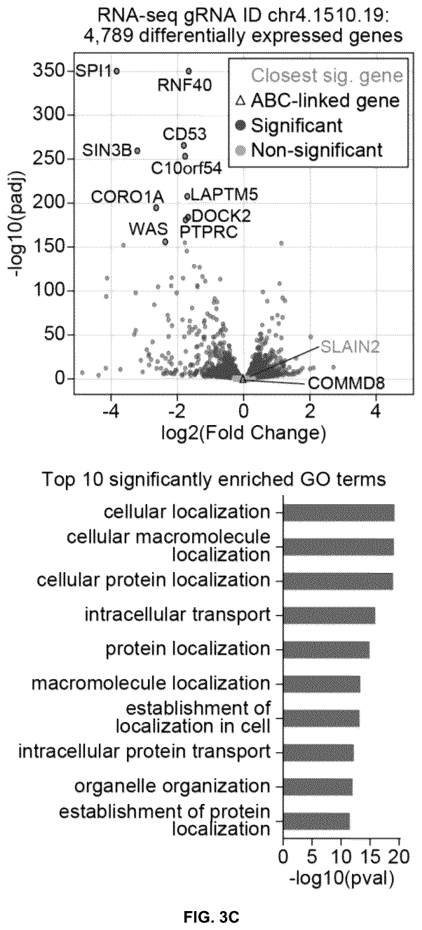

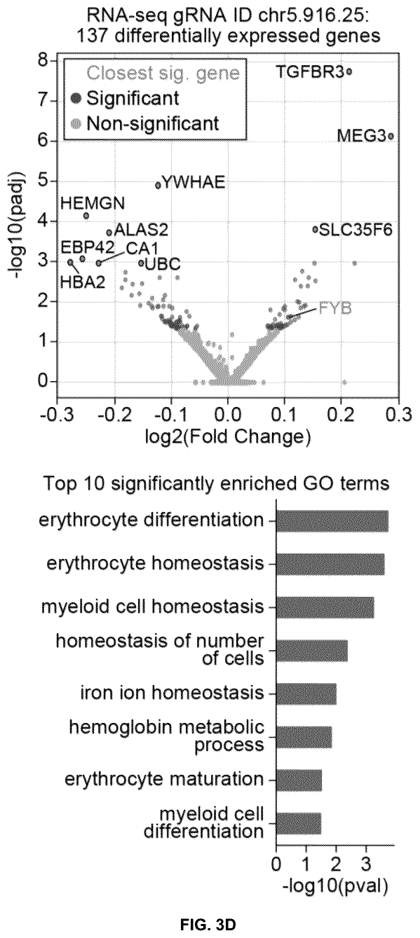

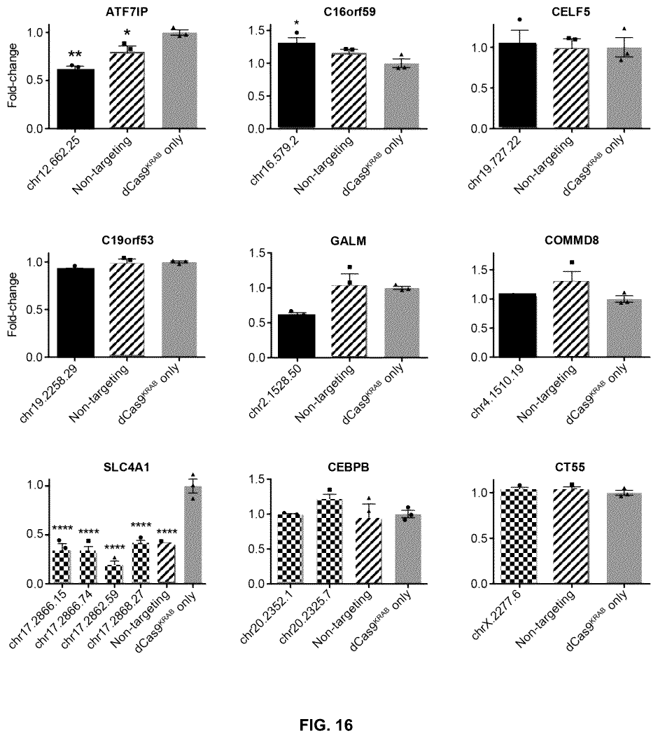

To test the effects of distal regulatory elements on target gene expression, the effects of individually targeting dCas9^{KRAB} via 23 gRNAs on the expression of 22 predicted target genes using qRT-PCR were measured (FIG. 3A, FIG. 16)(see also, for example, TABLES S14 and S15 and as in Klann et al. 2021. “Genome-wide annotation of gene regulatory elements linked to cell fitness” bioRxiv doi: 10.1101/2021.03.08.434470, which is incorporated herein by reference in its entirety). Predicted DHS-gene links were obtained using the Activity-by-Contact (ABC) model (Fulco et al., Nature Genetics. 2019, 51, 1664-1669, which is incorporated herein by reference in its entirety). There were significant changes in predicted target gene expression for 15 of the 23 gRNAs targeting 7 of 13 DHSs. The genes altered by targeting these DHSs include LMO2, GATA1, and GMPR. Previous studies have shown LMO2 and GATA1 are essential for K562 cell growth.

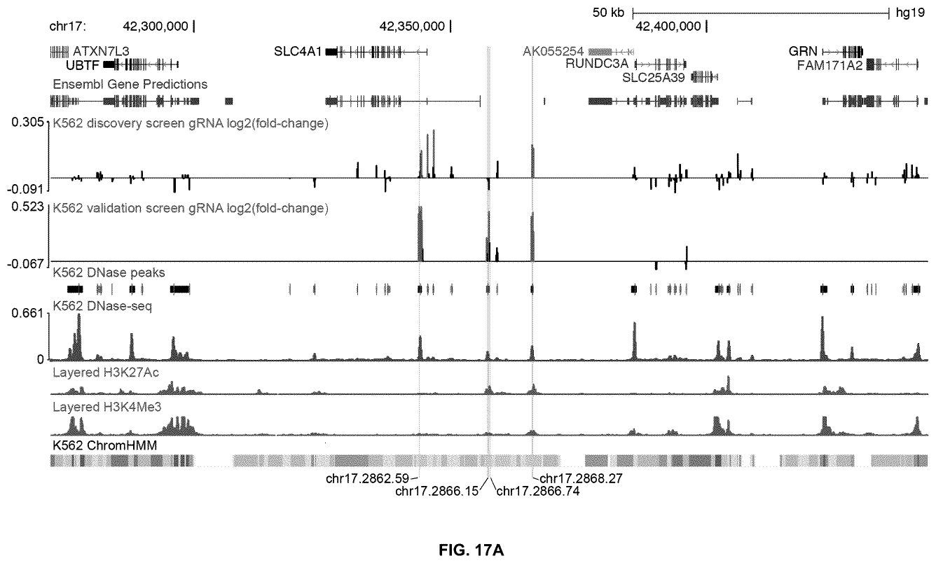

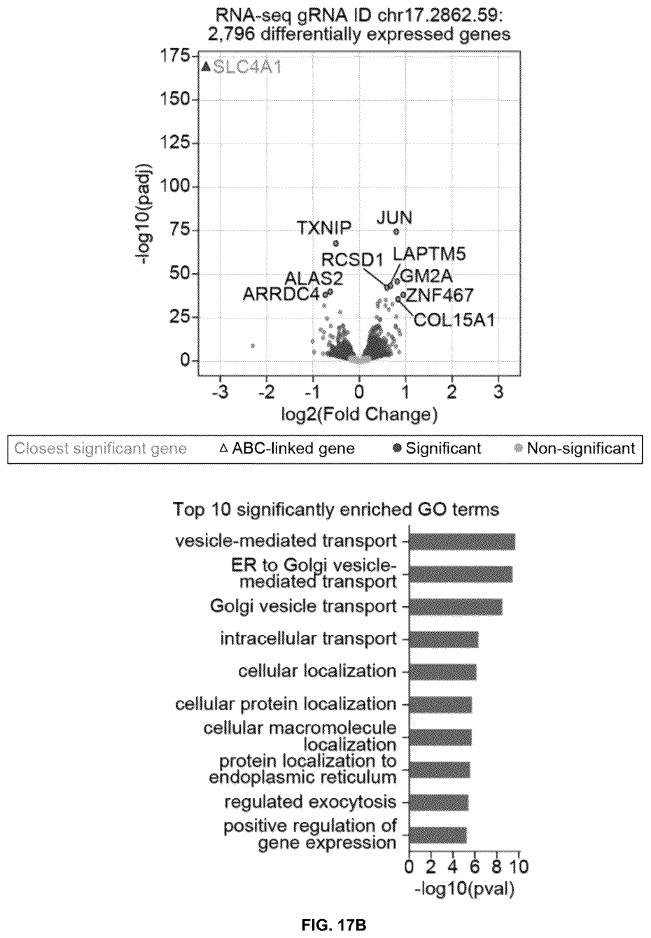

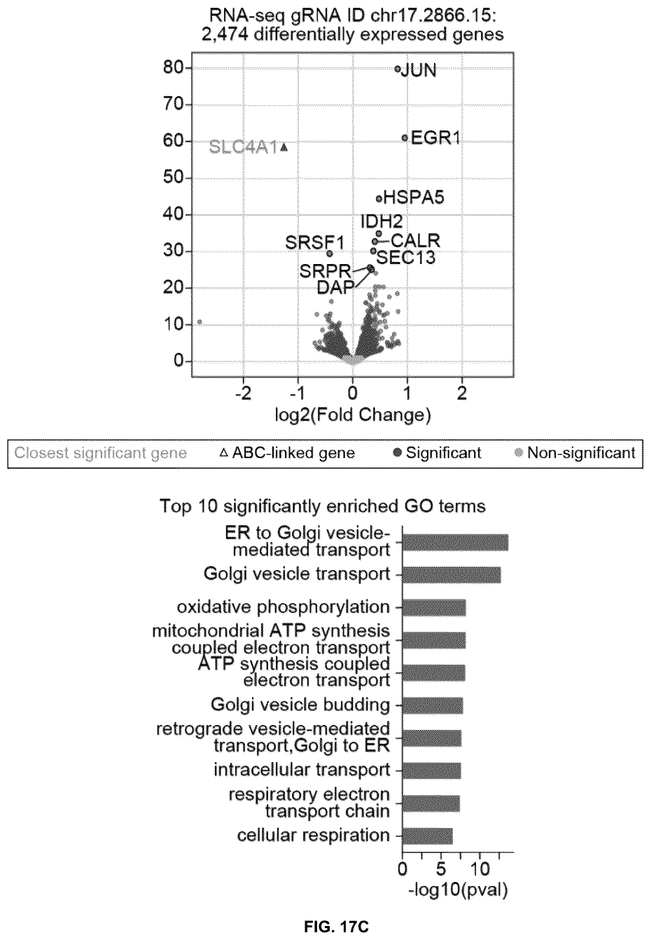

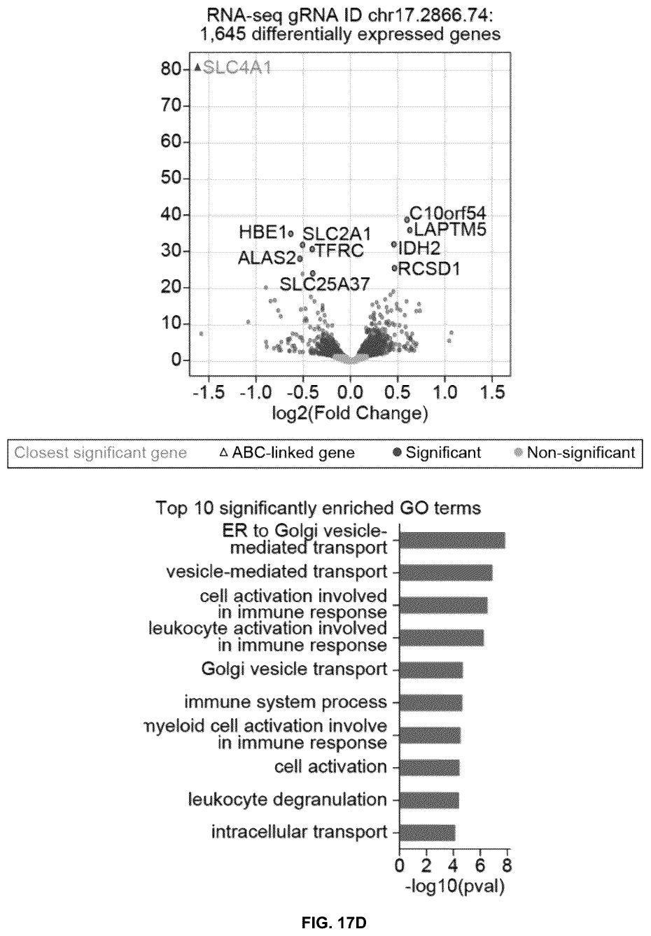

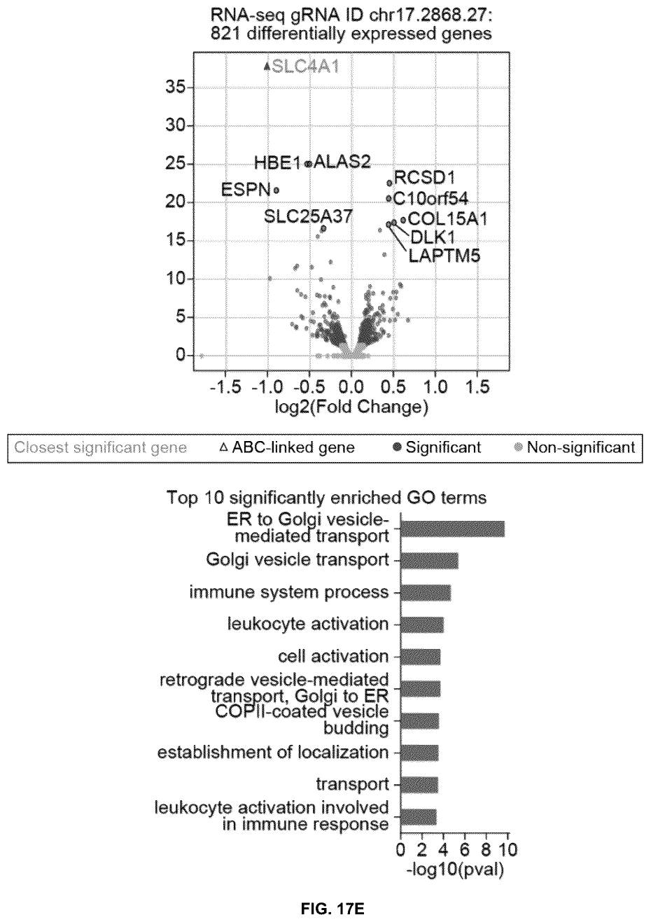

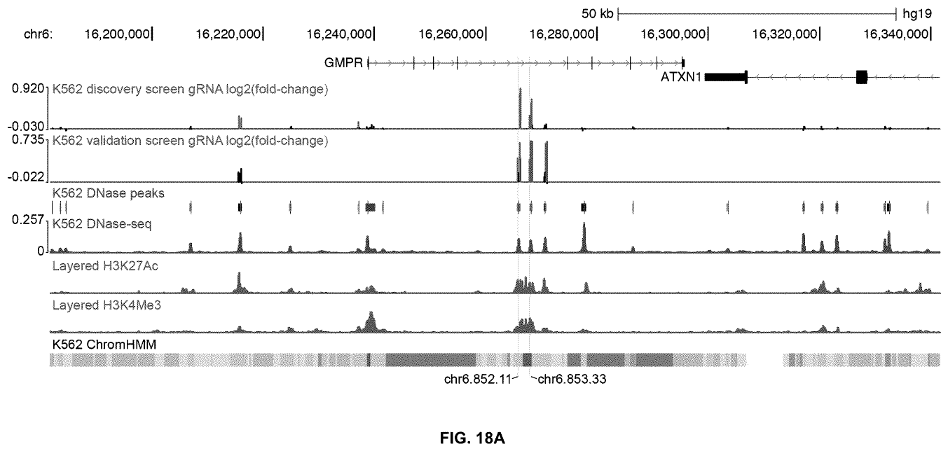

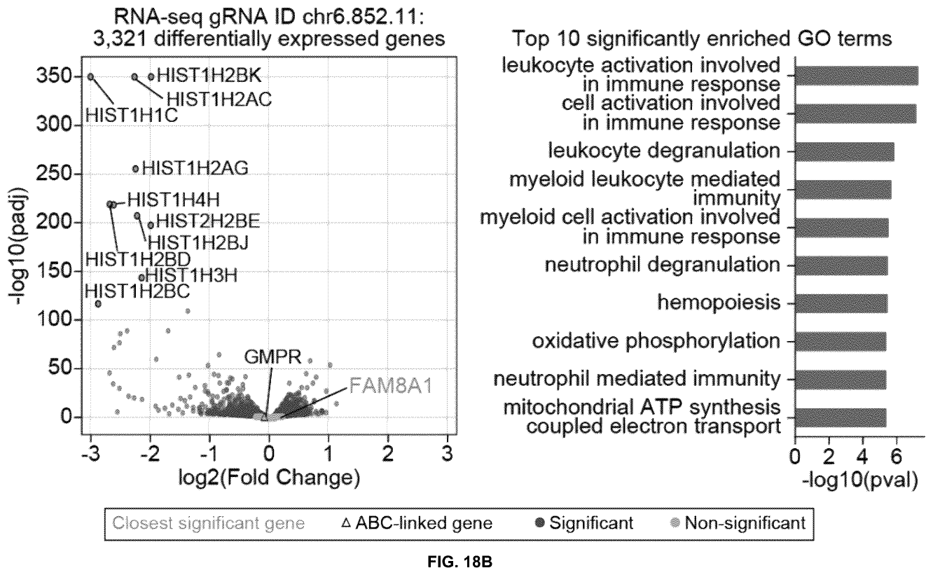

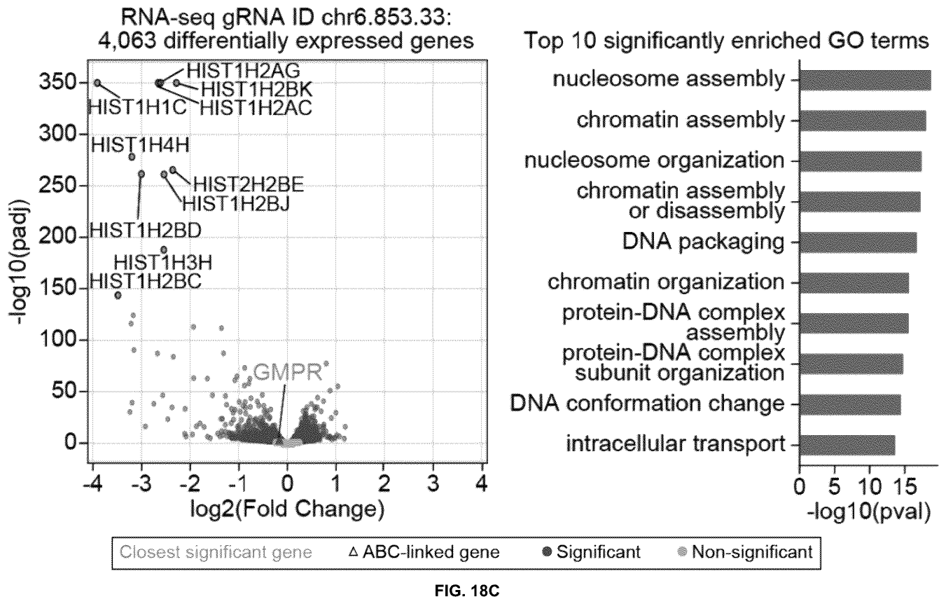

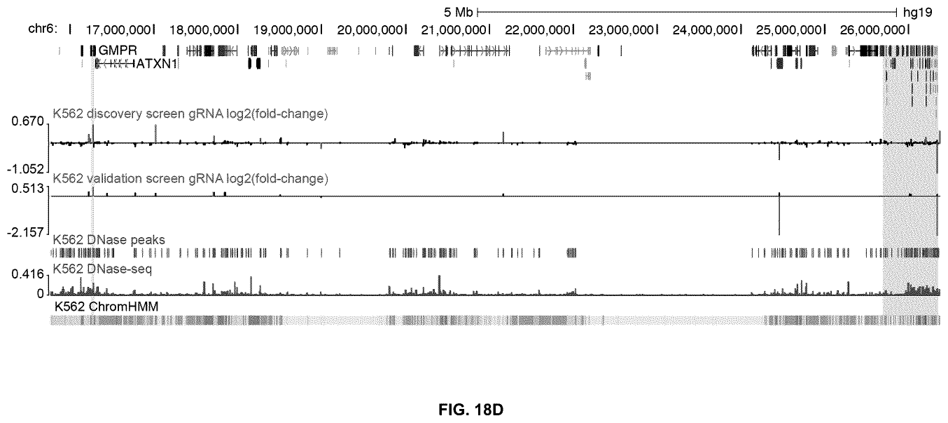

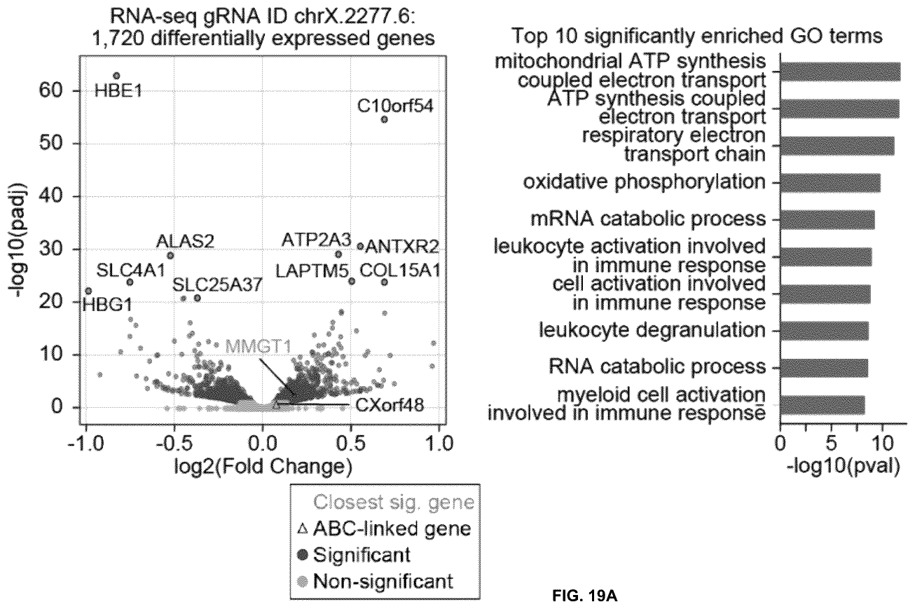

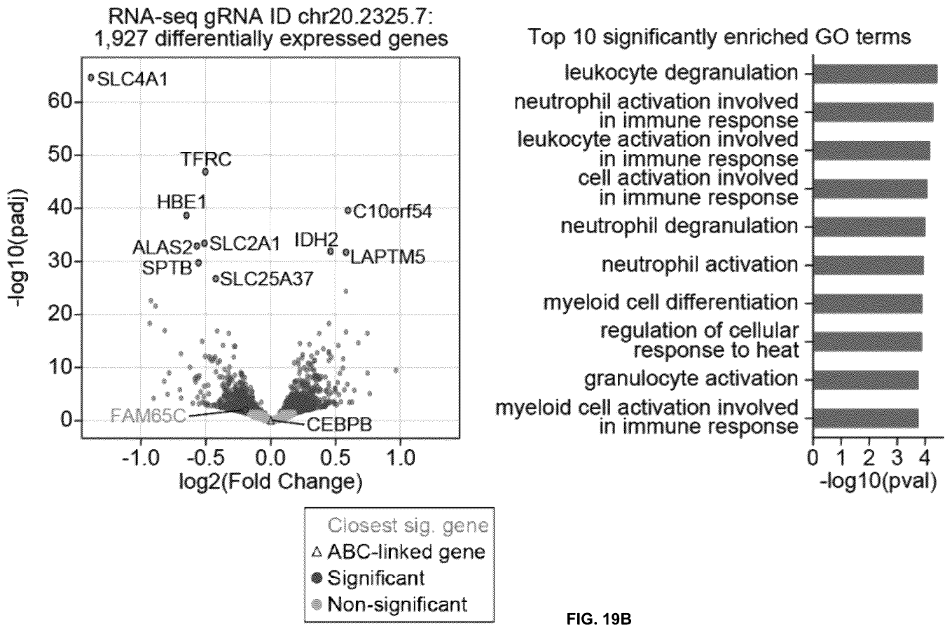

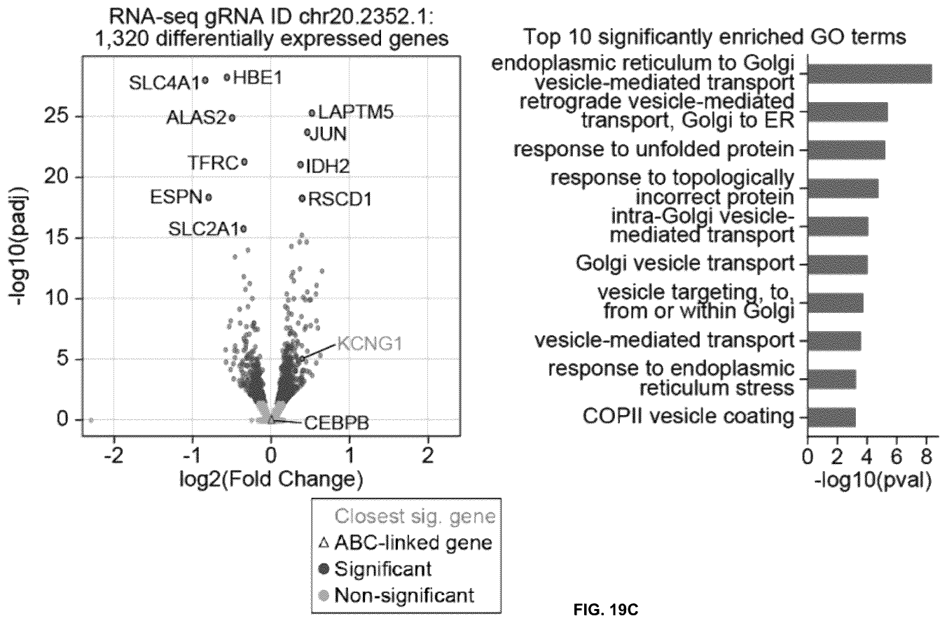

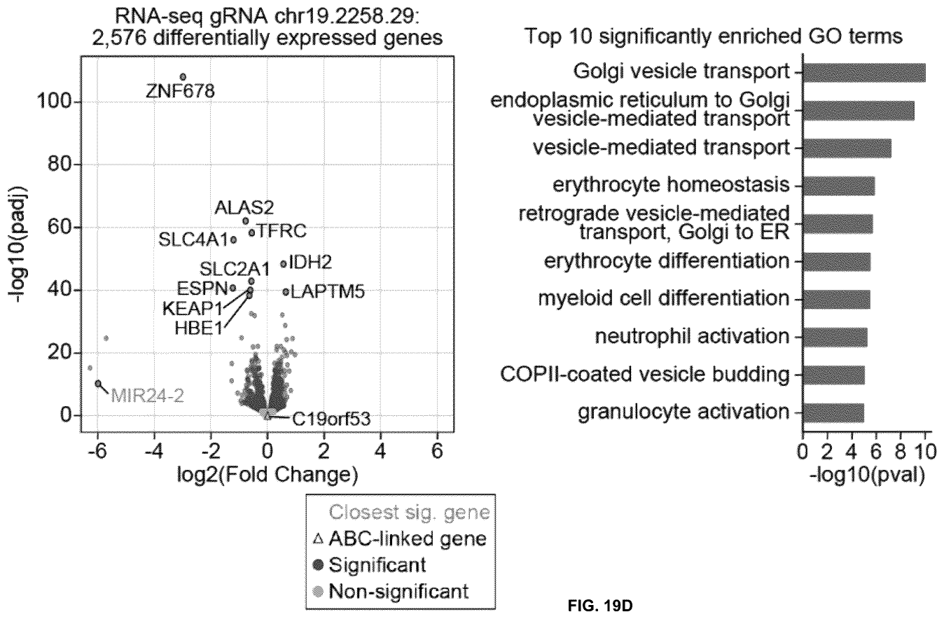

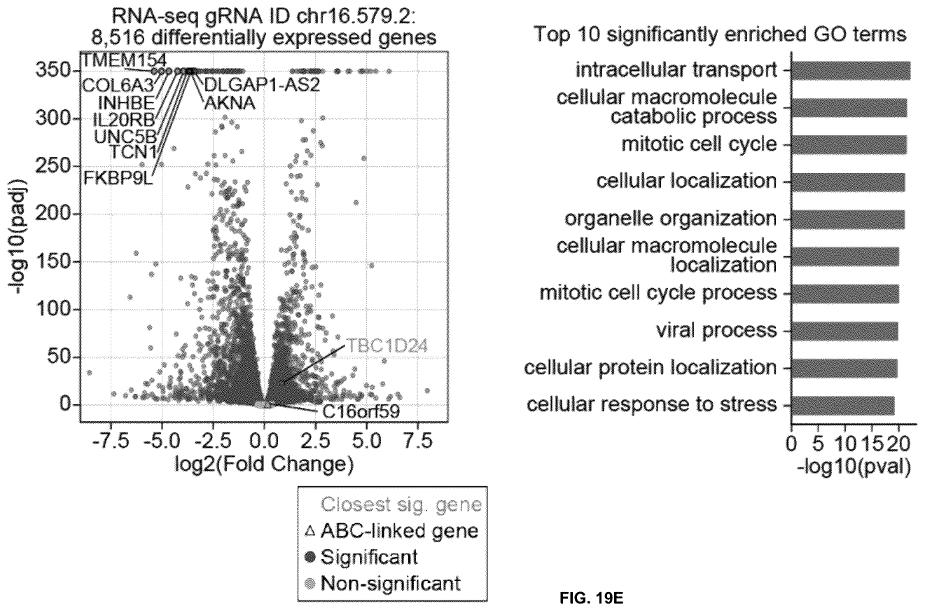

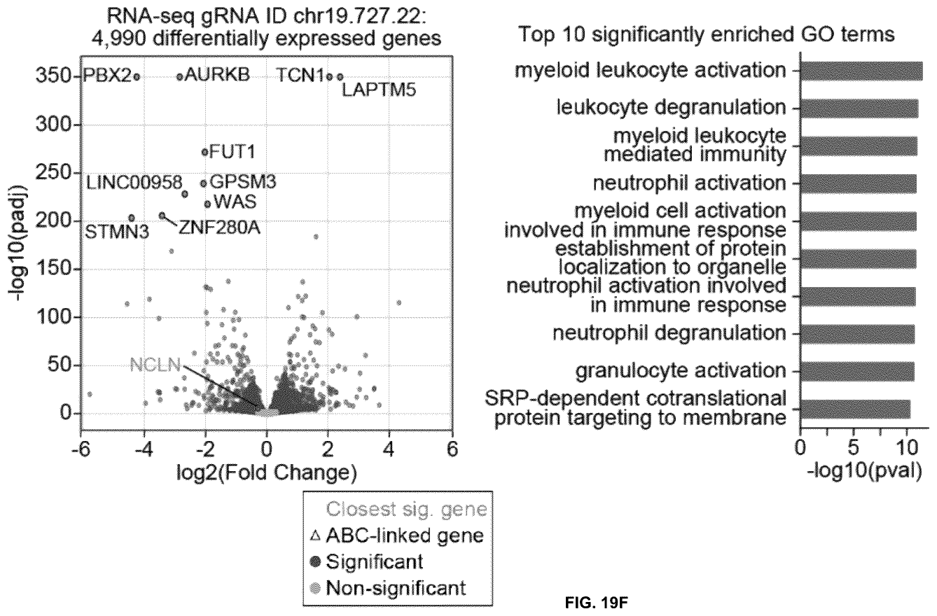

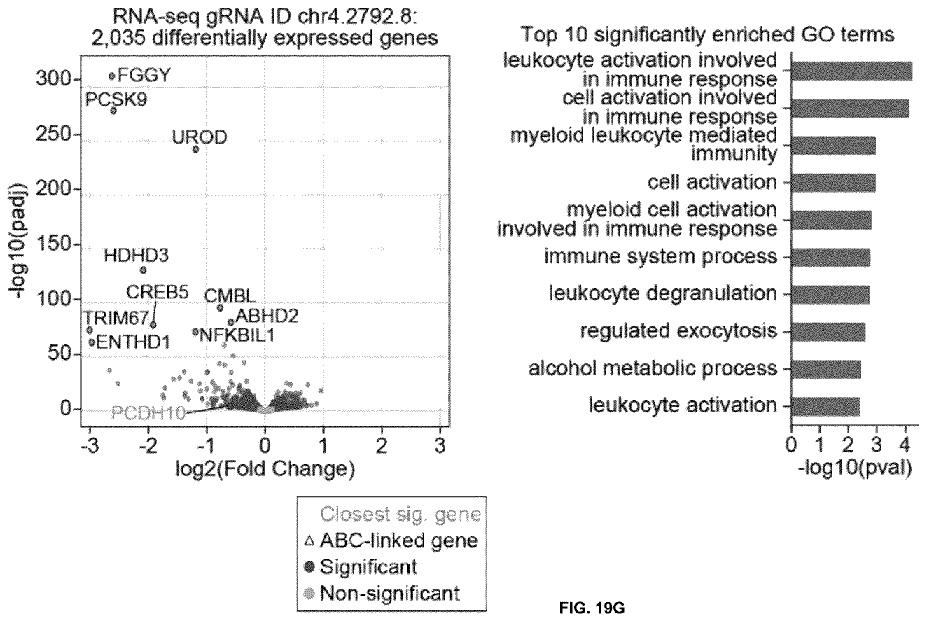

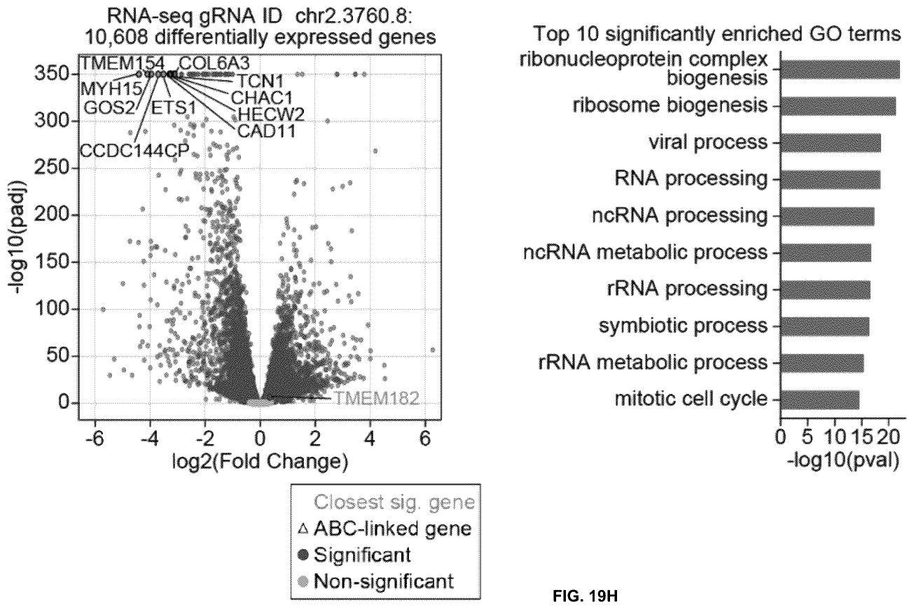

To measure transcriptome-wide effects of a subset of the above-described perturbations, RNA-seq was used. The analyses herein revealed that epigenetic perturbations of individual DHS resulted in many differentially expressed genes, and sometimes the predicted target gene was most affected (FIG. 3B and FIGS. 17A-17E). As one example, the effect of perturbing four different distal DHS hits around the SLC4A1 gene was evaluated, which is a gene involved in differentiation and when mutated causes hereditary spherocytosis and erythrocyte fragility. After perturbing each of these regions, expression of the SLC4A1 gene was the most significantly reduced, and there was also a high correspondence of gene ontology similarities for other significant differentially expressed genes (FIGS. 17A-17E). There were also instances that the ABC predicted target gene was not the most differentially expressed gene (FIGS. 3C-3D, FIGS. 18A-18D, and FIGS. 19A-19I). For example, targeting two DHS hits in an intron of the GMPR gene did not impact GMPR expression, but did impact sets of histone genes 8 Mb away, and overall displayed similar gene ontologies (FIGS. 18A-18D). Indeed, that result may explain why repressing those DHS impacted cell fitness even though GMPR has not been shown to be essential previously.

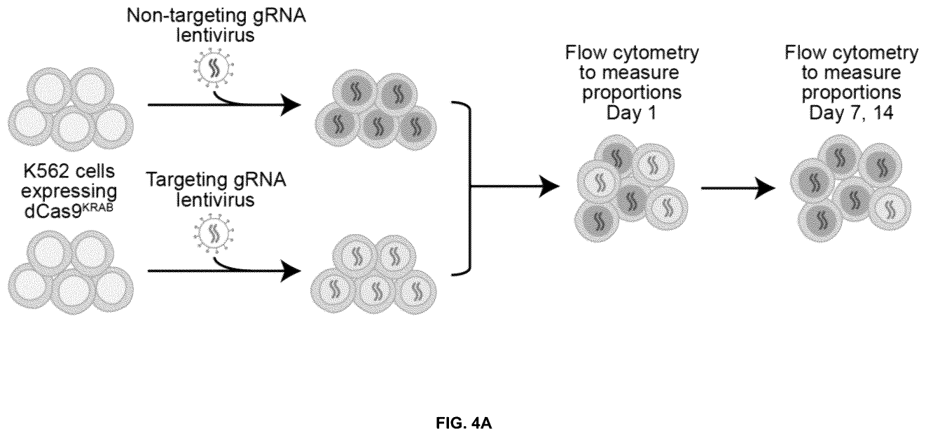

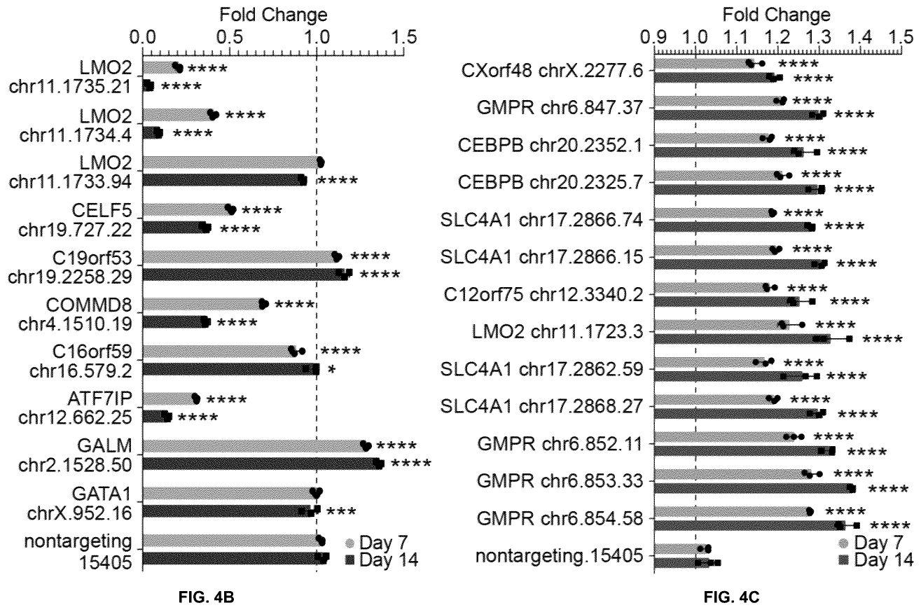

To further functionally characterize the targeted group of DHS hits, a cell growth competition assay was used to validate whether silencing each distal regulatory element reduces cellular fitness (FIG. 4A). Seven of 10 gRNAs that were depleted in the secondary screen also reduced cell fitness in the competition assay (FIG. 4B). Similarly, all 10 gRNAs that were enriched in the secondary screen also increased cell fitness in the competition assay (FIG. 4C). Therefore, the effect of the epigenetic perturbations on the selected phenotype was robust and reproducible, even if the target gene of the regulatory element was not immediately apparent.

### Example 8

### Identification of Cell-Type Specific Essential Gene Regulatory Elements

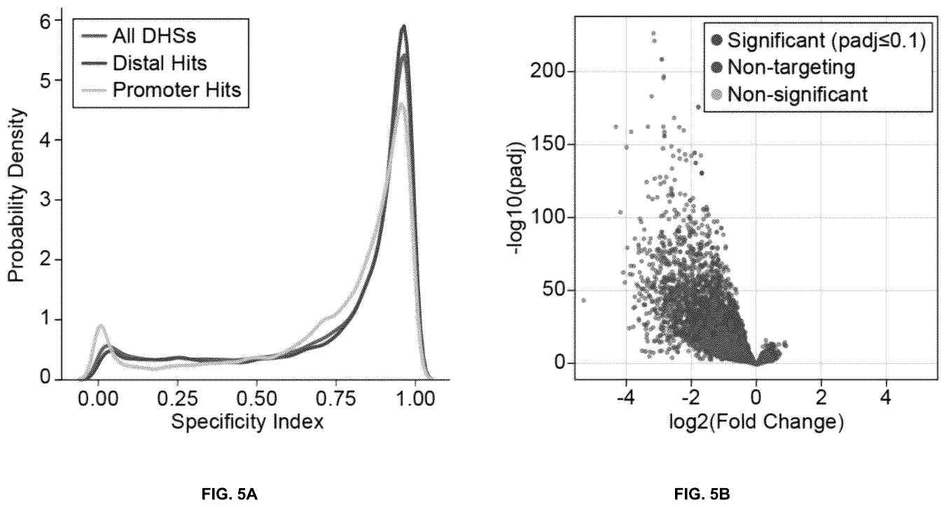

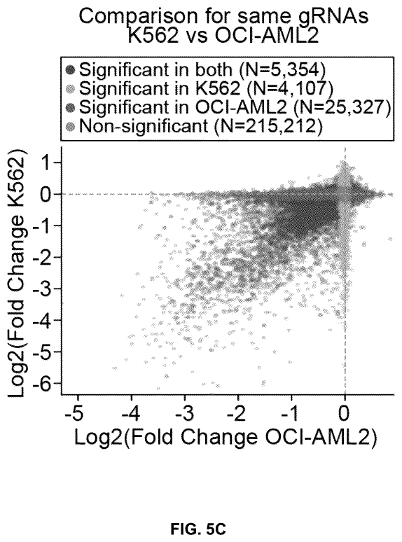

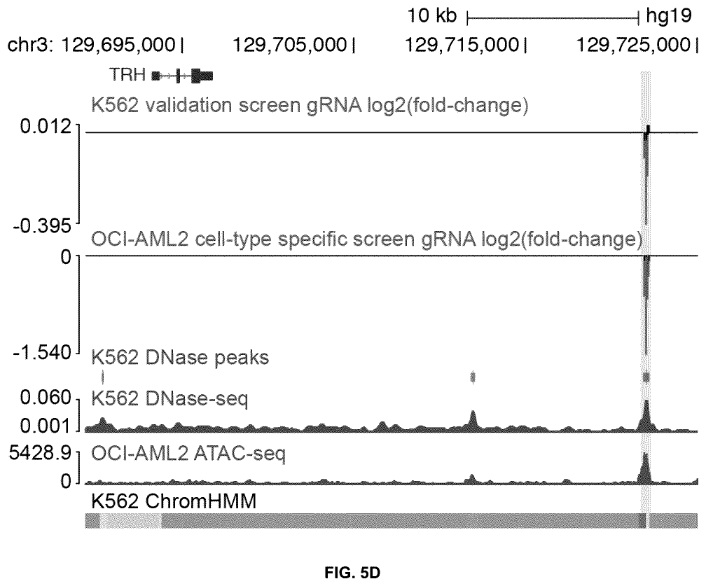

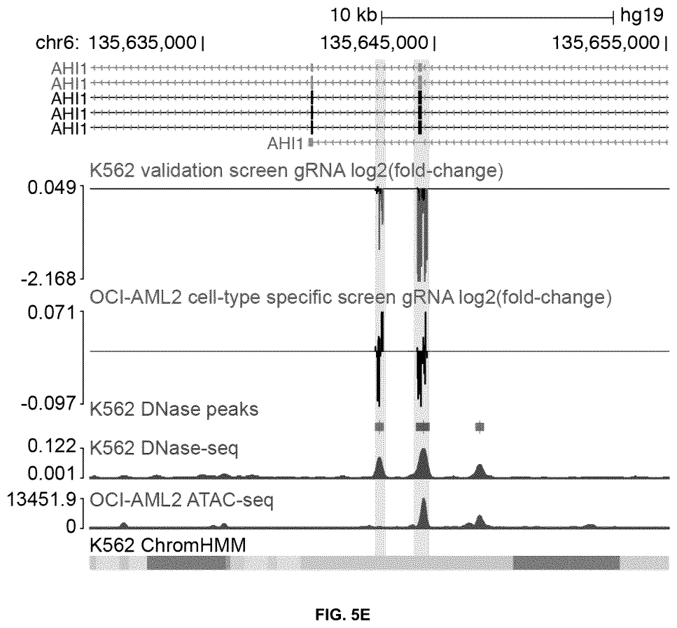

Chromatin accessibility data from 53 different cell types (personal.broadinstitute.org/meuleman/reg2map/) was used to characterize cell type specificity of DHS hits involved in cell fitness in K562 cells. For the significant DHS hits in our screen, most of the regions only overlapped open chromatin in K562 cells, while fewer regions overlapped open chromatin shared across many cell types (FIG. 5A). This suggests that many of the DHS hits identified herein affect fitness in a cell-type specific manner.

To functionally assess the generalizability of essential regulatory elements across cell types, the validation gRNA library used on the chronic myeloid leukemia (CML) K562 cell line was re-purposed (FIGS. 2A-2D) to perform an additional screen in the acute myeloid leukemia (AML) cell line OCI-AML2 (see, for example, TABLES S10-S13 and as in Klann et al. 2021. “Genome-wide annotation of gene regulatory elements linked to cell fitness” bioRxiv doi: 10.1101/2021.03.08.434470, which is incorporated herein by reference in its entirety). Similar to the results in K562 cells, depleted gRNAs with larger effect sizes in OCI-AM L2 cells were also detected (FIG. 5B). When comparing individual gRNAs between OCI-AML2 and K562 cells, 5,088 gRNAs that are significantly depleted in both cell types were detected, indicating these gRNAs lie in DHSs that are essential across different cancer cell lines (FIG. 5C). 1,855 gRNAs that are significantly depleted only in K562 cells and 15,670 gRNAs significantly depleted only in OCI-AML2 cells were also detected, indicating that OCI-AML2 cells may be more sensitive to regulatory element perturbation. Small numbers of non-targeting guides were also detected in shared and cell type specific hits, representing around 2-5% of the pool of significant gRNAs, which supports our estimates of FDR of 5% (see, for example, TABLES S6-S13 and as in Klann et al. 2021. “Genome-wide annotation of gene regulatory elements linked to cell fitness” bioRxiv doi: 10.1101/2021.03.08.434470, which is incorporated herein by reference in its entirety). A number of DHS hits overlap with chromatin accessibility that are shared between cell types (FIG. 5D) or are cell type-specific (FIG. 5E), indicating that chromatin accessibility may define essential regulatory element activity.

### Example 9

### Identification of Regulatory Element Target Genes Using Single Cell Expression

To empirically identify the target genes for the distal regulatory elements detected in these screens, a method that combines single cell RNA-seq readout with CRISPR screens (Gasperini et al., Cell. 2018, doi:10.1016/j.cell.2018.11.029, which is incorporated herein by reference in its entirety; Adamson et al., Cell. 2016, 167, 1867-1882.e21, which is incorporated herein by reference in its entirety; Dixit et al., Cell. 2016, 167, 1853-1866.e17, which is incorporated herein by reference in its entirety; Datlinger et al., Nat. Methods. 2017, 14, 297-301, which is incorporated herein by reference in its entirety; Xie et al., Mol. Cell. 2017, 66, 285-299.e5, which is incorporated herein by reference in its entirety) was adapted, which is referred to herein as single-cell CERES (scCERES). This allows the capture and quantification of all mRNA and gRNA identity on a per-cell basis, enabling the identification of genes that change in response to regulatory element perturbations. For this screen, polyclonal K562 cells constitutively expressing dCas9^{KRAB} were transduced with a library of 3,201 gRNAs (FIG. 7)(see also, for example, TABLES S16 and S17 and as in Klann et al. 2021. “Genome-wide annotation of gene regulatory elements linked to cell fitness” bioRxiv doi: 10.1101/2021.03.08.434470, which is incorporated herein by reference in its entirety) cloned into the opti-CROP-seq plasmid (Gasperini et al., Cell. 2018, doi:10.1016/j.cell.2018.11.029, which is incorporated herein by reference in its entirety; Datlinger et al., Nat. Methods. 2017, 14, 297-301, which is incorporated herein by reference in its entirety) in order to capture gRNA sequence identity on the 10× Genomics platform.

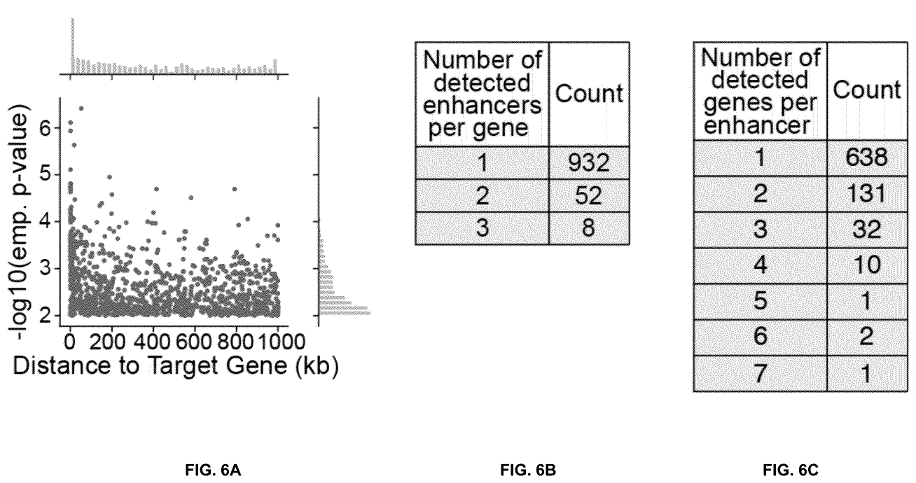

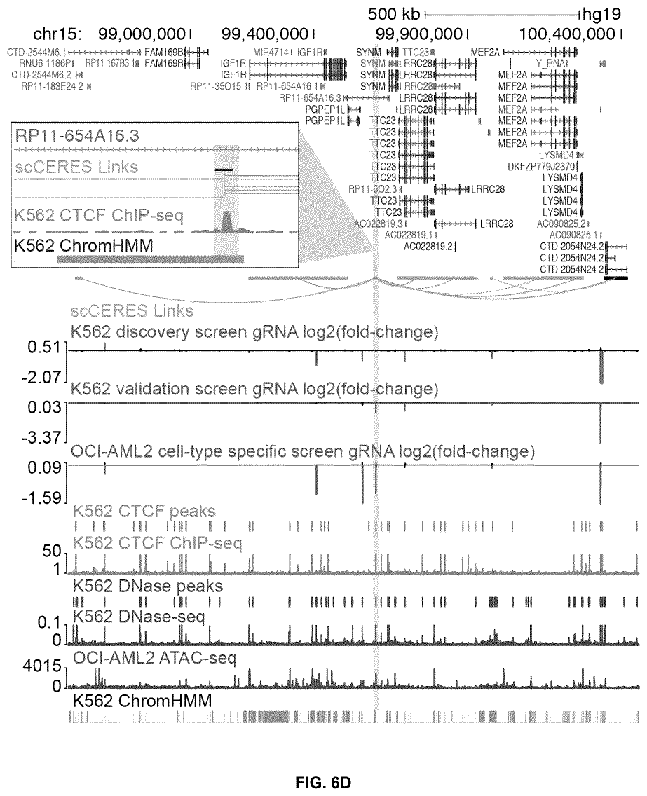

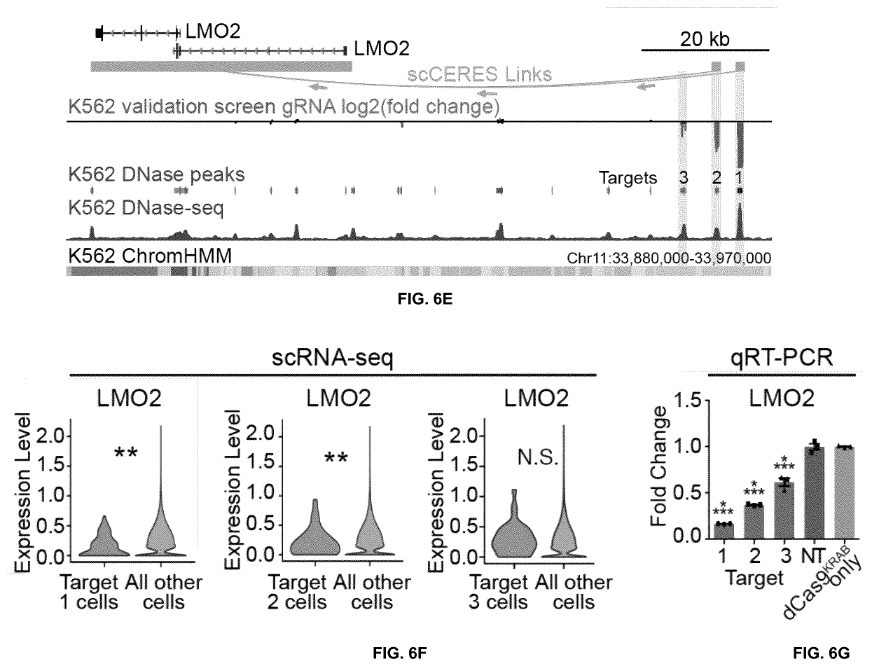

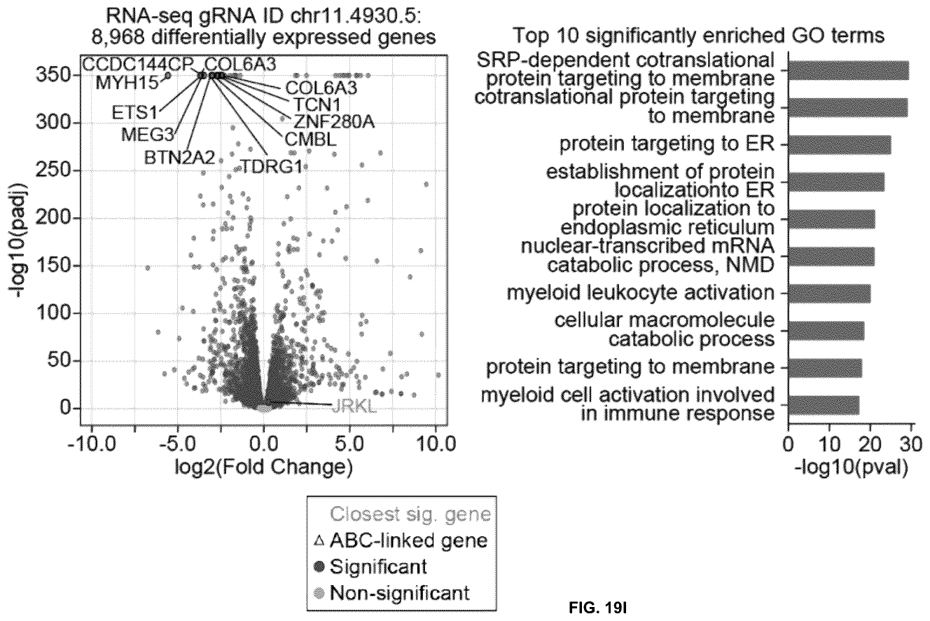

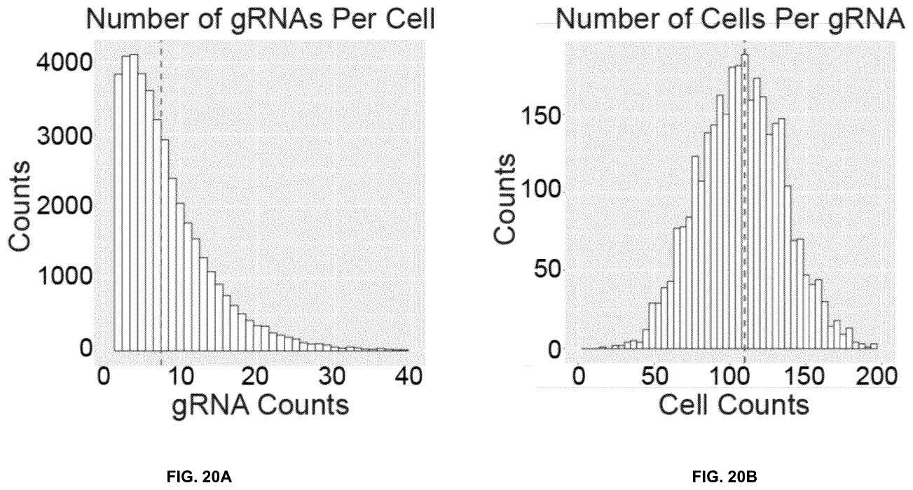

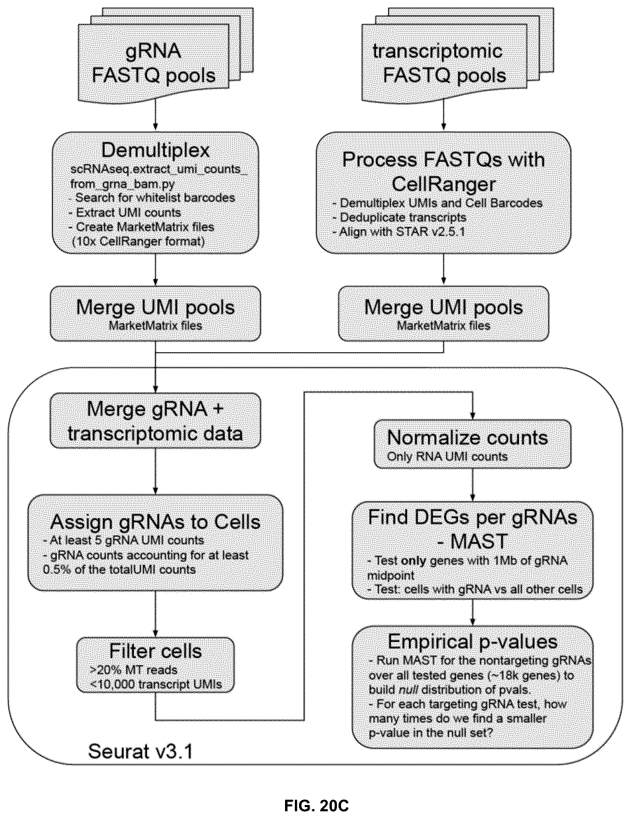

Cells were transduced at an MOI of ˜7 to increase overall library coverage, it was found that each cell contained an average of 8 gRNAs, and each gRNA was represented by an average of 111 cells (FIGS. 20A-20B). After library preparation and sequencing, differential expression analysis was performed by grouping cells that expressed the same gRNA (FIG. 20C). To increase statistical power to detect changes in gene expression, differential expression tests were limited to genes in a 2 megabase window centered on the DHS (FIG. 6A).

Collectively, 992 genes were identified that were affected by perturbing 815 unique regulatory elements. While most genes (N=932) had only a single link to a regulatory element, 52 genes were linked to 2 regulatory elements, and 8 genes were connected to 3 regulatory elements (FIG. 6B). The majority of the regulatory elements (N=638) only affected a single gene. However, perturbation of 177 regulatory elements altered expression of 2 or more genes within the 2 Mb window, including one element that affected 7 genes (FIGS. 6C-6D). Interestingly, this multi-gene affector overlaps a CTCF site, suggesting it may impact a TAD domain (FIG. 6D). DHS hits in polycomb regions that affect genes outside of the polycomb repressed region were also found (FIGS. 12A-12B).

Several gene-regulatory element links were corroborated by validating changes in gene expression by RT-qPCR following delivery of a single gRNA, including the ATF7IP (FIG. 16), GMPR (FIG. 3A), and LMO2 loci (FIGS. 6E-6G). For LMO2, gene-enhancer connections were identified for two regulatory elements ˜60 kb upstream of the LMO2 gene that were among the strongest hits from the initial wgCERES screen (Target 1 and 2; FIG. 6E). Repression of either element by dCas9^{KRAB} led to significantly reduced expression of the LMO2 gene (FIGS. 6E-6F). A third regulatory element (Target 3) in the same cluster did not show statistically significant changes in LMO2 expression by scCERES (FIGS. 6E-6F), but did show comparatively modest repression by RT-qPCR (FIG. 6G). This may represent the need for increased sequencing depth to achieve better sensitivity. Regardless, this single cell readout identifies a substantial number of regulatory elements that are simultaneously linked to both target genes and cell fitness.

### Example 10

### DISCUSSION

Cancer genetics and the discovery of oncogenic driver mutations has historically been limited to analysis of protein coding sequences because (i) whole-genome sequencing of primary tumors is costly, and (ii) our functional understanding of noncoding genetic variation is still in its infancy. This study is a significant step towards addressing these limitations and realizing the potential of whole genome sequencing for cancer biology. Herein is described a systematic genome-wide screen of all putative regulatory elements in a commonly used cancer cell line and describe their role in cell fitness. Greater than 12,000 regulatory elements were identified herein that have negative or positive impacts on cellular viability and/or proliferation, and ˜1,000 element-gene links that drive this phenotype were reported herein. The data herein provide a rich resource of regulatory element function and connection to target genes that will be broadly useful for understanding gene network regulation and the mechanisms of non-coding element control on gene expression. These characterizations that relate the non-coding genome to cell fitness will identify functional noncoding sequence variants that contribute to cancer phenotypes. These functional annotations also complement the growing body of chromatin conformation maps that provide structural relationships between regulatory elements and genes. Moreover, this work provides a blueprint for executing similar studies in other cell types, genetic backgrounds, environmental conditions, or pharmacologic treatments. In the future, this approach may facilitate the development of methods to predict element-gene relationships and inform efforts to learn the quantitative rules of gene regulation.

Another challenge to implementing genome-wide screens of the non-coding genome is the sheer scale of the experiment, which is dictated by the number of putative elements in any cell type and the required numbers of gRNAs per element and cells per gRNA. As the field of CRISPR-based screens is still in its relative infancy, an important area of future focus is the design of more efficient and sensitive screening methods. For example, the dataset herein may be used to define the properties for effective gRNA design in distal regulatory elements, similar to what has been done for designing optimal gRNA libraries for genes and promoters (Horlbeck et al., eLife. 2016, 5, doi:10.7554/elife.19760, which is incorporated herein by reference in its entirety; Gilbert et al., Cell. 2014, 159, 647-661, which is incorporated herein by reference in its entirety; Konermann et al., Nature. 2015, 517, 583-588, which is incorporated herein by reference in its entirety). The work herein depended on extensive characterization of gRNA libraries targeting these classes of elements. In contrast, relatively little is known about which key gRNA attributes contribute to effective perturbation of distal regulatory elements. The knowledge gained from thousands of gRNAs that impact cellular growth from distal regulatory elements as described herein may facilitate the design of more compact and robust libraries, and enable similar genome-wide screens in cell lines or primary cells that are more difficult to culture at scale.

Many epigenetic modifying drugs used as potential cancer treatments cause widespread changes throughout the genome. However, it is currently unclear what subset of gene regulatory elements drive drug response. Using maps of essential regulatory elements in conjunction with the epigenetic profiles of cells after drug treatment could help identify modifications to specific gene regulatory elements necessary and sufficient for drug response. This may ultimately inform the development of safer and more specific cancer therapies.

One of the loci with the strongest effect on cellular proliferation was the LMO2 locus. This locus is also the location of retroviral insertions in gene therapy patients which lead to increased expression of LMO2 via viral enhancer elements and ultimately led to leukemia. Better understanding the regulatory landscape of these and other types of regions will help elucidate mechanisms of aberrant gene expression and tumorigenesis that will ultimately also inform design, safety monitoring, and regulation of emerging classes of genetic medicines such as gene therapy and genome editing. Therefore, the approach described herein will be a valuable resource to diverse fields of the biomedical research community.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A composition for treating leukemia, the composition comprising: a Cas9 protein or a fusion protein, wherein the fusion protein comprises two heterologous polypeptide domains, wherein the first polypeptide domain comprises a Cas9 protein and the second polypeptide domain has an activity selected from the group consisting of transcription activation activity, transcription repression activity, transcription release factor activity, histone modification activity, nuclease activity, nucleic acid association activity, methylase activity, and demethylase activity; and at least one guide RNA (gRNA) that targets the Cas9 protein to a regulatory element of a target gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GM PR.

Clause 2. The composition of clause 1, wherein the gRNA targets the Cas9 protein to a polynucleotide sequence comprising a sequence selected from SEQ ID NOs: 339-479.

Clause 3. The composition of clause 1 or 2, wherein the gRNA is encoded by a polynucleotide sequence comprising a sequence selected from SEQ ID NOs: 57-197 or comprises a polynucleotide sequence comprising a sequence selected from SEQ ID NOs: 198-338.

Clause 4. The composition of any one of clauses 1-3, wherein the composition inhibits cell viability.

Clause 5. The composition of clause 4, wherein the target gene is selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1.

Clause 6. The composition of clause 4 or 5, wherein the gRNA targets the Cas9 protein to a polynucleotide sequence comprising a sequence selected from SEQ ID NOs: 339-473.

Clause 7. The composition of any one of clauses 4-6, wherein the gRNA is encoded by a polynucleotide sequence comprising a sequence selected from SEQ ID NOs: 57-191 or comprises a polynucleotide sequence comprising a sequence selected from SEQ ID NOs: 198-332.

Clause 8. The composition of any one of clauses 1-3, wherein the composition increases cell viability.

Clause 9. The composition of clause 8, wherein the target gene is selected from FADS3, RPAP1, SLC25A39, RP13-20L14.6, FOXA2, and GMPR.

Clause 10. The composition of clause 8 or 9, wherein the gRNA targets the Cas9 protein to a polynucleotide sequence comprising a sequence selected from SEQ ID NOs: 474-479.

Clause 11. The composition of any one of clauses 8-10, wherein the gRNA is encoded by a polynucleotide sequence comprising a sequence selected from SEQ ID NOs: 192-197 or comprises a polynucleotide sequence comprising a sequence selected from SEQ ID NOs: 333-338.

Clause 12. The composition of any one of clauses 1-11, wherein the Cas protein comprises a Streptococcus pyogenes Cas9 protein, a Staphylococcus aureus Cas9 protein, or any fragment thereof.

Clause 13. The composition of any one of clauses 1-12, wherein the Cas9 protein comprises an amino acid sequence having at least 90% or greater identity to a sequence selected from SEQ ID NOs: 20-23, or any fragment thereof, or is encoded by a polynucleotide comprising a sequence having at least 90% or greater identity to a sequence selected from SEQ ID NOs: 24-26, or any fragment thereof.

Clause 14. The composition of clause 13, wherein the Cas9 protein comprises an amino acid sequence having one, two, three, four, five or more changes selected from amino acid substitutions, insertions, or deletions, relative to a sequence selected from SEQ ID NOs: 20-23, or any fragment thereof, or is encoded by a polynucleotide comprising a sequence having one, two, three, four, five or more changes selected from nucleotide substitutions, insertions, or deletions, relative to a sequence selected from SEQ ID NOs: 24-26, or any fragment thereof.

Clause 15. The composition of clause 13, wherein the Cas9 protein comprises the amino acid sequence of SEQ ID NO: 20 or 21 or 22 or 23, or any fragment thereof, or is encoded by a polynucleotide comprising a sequence selected from SEQ ID NOs: 24 or 25 or 26.

Clause 16. The composition of any one of clauses 1-15, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

Clause 17. The composition of any one of clauses 1-15, wherein the second polypeptide domain has transcription repression activity.

Clause 18. The composition of clause 17, wherein the second polypeptide domain comprises KRAB.

Clause 19. The composition of clause 18, wherein KRAB comprises an amino acid sequence having at least 90% or greater identity to SEQ ID NO: 55, or any fragment thereof.

Clause 20. The composition of clause 19, wherein KRAB comprises an amino acid sequence having one, two, three, four, five or more changes selected from amino acid substitutions, insertions, or deletions, relative to SEQ ID NO: 55, or any fragment thereof.

Clause 21. The composition of clause 19, wherein KRAB comprises the amino acid sequence of SEQ ID NO: 55, or any fragment thereof.

Clause 22. The composition of any one of clauses 1-21, wherein fusion protein comprises an amino acid sequence having at least 90% or greater identity to SEQ ID NO: 40 or 42, or any fragment thereof.

Clause 23. The composition of clause 22, wherein fusion protein comprises an amino acid sequence having one, two, three, four, five or more changes selected from amino acid substitutions, insertions, or deletions, relative to SEQ ID NO: 40 or 42, or any fragment thereof.

Clause 24. The composition of clause 22, wherein fusion protein comprises the amino acid sequence of SEQ ID NO: 40 or 42, or any fragment thereof.

Clause 25. An isolated polynucleotide sequence comprising a sequence selected from SEQ ID NOs: 57-338.

Clause 26. An isolated polynucleotide sequence encoding the composition of any one of clauses 1-24.

Clause 27. A vector comprising the isolated polynucleotide sequence of clause 25 or 26.

Clause 28. A vector encoding the composition of any one of clauses 1-24.

Clause 29. A cell comprising the composition of any one of clauses 1-24, the isolated polynucleotide sequence of clause 25 or 26, or the vector of clause 27 or 28, or a combination thereof.

Clause 30. A pharmaceutical composition comprising the composition of any one of clauses 1-24, the isolated polynucleotide sequence of clause 25 or 26, the vector of clause 27 or 28, or the cell of clause 29, or a combination thereof.

Clause 31. A method of treating leukemia in a subject, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the subject.

Clause 32. The method of clause 31, wherein modifying the expression of the gene comprises reducing expression of the gene.

Clause 33. The method of clause 31 or 32, wherein the method comprises administering to the subject the composition of any one of clauses 1-24, the isolated polynucleotide sequence of clause 25 or 26, the vector of clause 27 or 28, the cell of clause 29, or the pharmaceutical composition of clause 30, or a combination thereof.

Clause 34. A method of modifying growth of a cell, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR in the cell.

Clause 35. The method of clause 34, wherein the method comprises administering to the cell the composition of any one of clauses 1-24, the isolated polynucleotide sequence of clause 25 or 26, or the vector of clause 27 or 28, or a combination thereof.

Clause 36. A method of decreasing cell fitness, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-536I6.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the cell.

Clause 37. The method of clause 36, wherein the targeting comprises administering to a cell the composition of any one of clauses 1-24, the isolated polynucleotide sequence of clause 25 or 26, or the vector of clause 27 or 28, or a combination thereof.

Clause 38. The method of clause 36 or 37, wherein decreasing cell fitness comprises decreasing cell growth rate, decreasing cell growth duration, decreasing cell size, increasing cell death, or a combination thereof.

Clause 39. A method of increasing cell fitness, the method comprising targeting a regulatory element of, or modifying the expression of, a gene selected from FADS3, RPAP1, SLC25A39, RP13-20L14.6, FOXA2, and GMPR in the cell.

Clause 40. The method of clause 39, wherein the targeting comprises administering to a cell the composition of any one of clauses 1-24, the isolated polynucleotide sequence of clause 25 or 26, or the vector of clause 27 or 28, or a combination thereof.

Clause 41. The method of clause 39 or 40, wherein increasing cell fitness comprises increasing cell growth rate, increasing cell growth duration, increasing cell size, or a combination thereof.

Legends for TABLES S1-S13 and S17, as in Klann et al. 2021, “Genome-wide annotation of gene regulatory elements linked to cell fitness” bioRxiv doi: 10.1101/2021.03.08.434470, which is incorporated herein by reference in its entirety:

Legend for TABLE S1:

| Column | Description |
|---|---|
| chrom | Chromosome |
| chromStart | gRNA start coordinate (hg19) |
| chromEnd | gRNA end coordinate (hg19) |
| strand | Orientation of the gRNA (positive/forward or negative/reverse) |
| gRNAid | gRNA identifier in this study. It is constructed as the |
| | {DHS}.{NUM_GUIDE_IN_DHS} |
| DHS | DHS identifier. It refers to the unique Dnaseq-seq peak and can be |
| | found in https://www.encodeproject.org/files/ENCFF001UWQ/ |
| protospacer | protospacer DNA sequence targeted by the gRNA |
| baseMean | Measure of gRNA expression across all conditions [DESeq2] |
| log2FoldChange | Relative enrichment in gRNA expression for treatments over controls |
| | (in log2) [DESeq2] |
| lfcSE | Standard error in the relative enrichment in gRNA expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg |
| | (BH) to the weighted p-value assigned by Independent Hypothesis |
| | Weighting (IHW) [DESeq2] |
| weight | P-value weight assigned by Independent Hypothesis Weighting (IHW) |
| | [DESeq2] |
| ctrl1 | gRNA raw counts for control biological replicate 1 |
| ctrl2 | gRNA raw counts for control biological replicate 2 |
| ctrl3 | gRNA raw counts for control biological replicate 3 |
| ctrl4 | gRNA raw counts for control biological replicate 4 |
| rep1 | gRNA raw counts for treatment biological replicate 1 |
| rep2 | gRNA raw counts for treatment biological replicate 2 |
| rep3 | gRNA raw counts for treatment biological replicate 3 |
| rep4 | gRNA raw counts for treatment biological replicate 4 |

Legend for TABLE S2:

| Column | Description |
|---|---|
| chrom | Chromosome |
| chromStart | gRNA-bin2 start coordinate (hg19) |
| chromEnd | gRNA-bin2 end coordinate (hg19) |
| strand | Orientation of the gRNA-bin2 (positive/forward or negative/reverse) |
| binID | gRNA-bin2 identifier in this study. It is constructed as the |
| | {DHS}.bin2_{NUM_BIN2_IN_DHS} |
| DHS | DHS identifier. It refers to the unique Dnaseq-seq peak and can be |
| | found in https://www.encodeproject.org/files/ENCFF001UWQ/ |
| baseMean | Measure of gRNA-bin2 expression across all conditions [DESeq2] |
| log2FoldChange | Relative enrichment in gRNA-bin2 expression for treatments over |
| | controls (in log2) [DESeq2] |
| lfcSE | Standard error in the relative enrichment in gRNA-bin2 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg |
| | (BH) to the weighted p-value assigned by Independent Hypothesis |
| | Weighting (IHW) [DESeq2] |
| weight | P-value weight assigned by Independent Hypothesis Weighting (IHW) |
| | [DESeq2] |
| ctrl1 | gRNA-bin2 raw counts for control biological replicate 1 |
| ctrl2 | gRNA-bin2 raw counts for control biological replicate 2 |
| ctrl3 | gRNA-bin2 raw counts for control biological replicate 3 |
| ctrl4 | gRNA-bin2 raw counts for control biological replicate 4 |
| rep1 | gRNA-bin2 raw counts for treatment biological replicate 1 |
| rep2 | gRNA-bin2 raw counts for treatment biological replicate 2 |
| rep3 | gRNA-bin2 raw counts for treatment biological replicate 3 |
| rep4 | gRNA-bin2 raw counts for treatment biological replicate 4 |

Legend for TABLE S3:

| Column | Description |
|---|---|
| chrom | Chromosome |
| chromStart | gRNA-bin3 start coordinate (hg19) |
| chromEnd | gRNA-bin3 end coordinate (hg19) |
| strand | Orientation of the gRNA-bin3 (positive/forward or negative/reverse) |
| binID | gRNA-bin3 identifier in this study. It is constructed as the |
| | {DHS}.bin3_{NUM_bin3_IN_DHS} |
| DHS | DHS identifier. It refers to the unique Dnaseq-seq peak and can be found |
| | in https://www.encodeproject.org/files/ENCFF001UWQ/ |
| baseMean | Measure of gRNA-bin3 expression across all conditions [DESeq2] |
| log2FoldChange | Relative enrichment in gRNA-bin3 expression for treatments over controls |
| | (in log2) [DESeq2] |
| lfcSE | Standard error in the relative enrichment in gRNA-bin3 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting |
| | (IHW) [DESeq2] |
| weight | P-value weight assigned by Independent Hypothesis Weighting (IHW) |
| | [DESeq2] |
| ctrl1 | gRNA-bin3 raw counts for control biological replicate 1 |
| ctrl2 | gRNA-bin3 raw counts for control biological replicate 2 |
| ctrl3 | gRNA-bin3 raw counts for control biological replicate 3 |
| ctrl4 | gRNA-bin3 raw counts for control biological replicate 4 |
| rep1 | gRNA-bin3 raw counts for treatment biological replicate 1 |
| rep2 | gRNA-bin3 raw counts for treatment biological replicate 2 |
| rep3 | gRNA-bin3 raw counts for treatment biological replicate 3 |
| rep4 | gRNA-bin3 raw counts for treatment biological replicate 4 |

Legend for TABLE S4:

| Column | Description |
|---|---|
| DHS | DHS identifier. It refers to the unique Dnaseq-seq peak and can be found in |
| | https://www.encodeproject.org/files/ENCFF001UWQ/ |
| baseMean | Measure of DHS expression across all conditions [DESeq2] |
| log2FoldChange | Relative enrichment in DHS expression for treatments over controls (in log2) |
| | [DESeq2] |
| lfcSE | Standard error in the relative enrichment in DHS expression for treatments |
| | over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | DHS raw counts for control biological replicate 1 |
| ctrl2 | DHS raw counts for control biological replicate 2 |
| ctrl3 | DHS raw counts for control biological replicate 3 |
| ctrl4 | DHS raw counts for control biological replicate 4 |
| rep1 | DHS raw counts for treatment biological replicate 1 |
| rep2 | DHS raw counts for treatment biological replicate 2 |
| rep3 | DHS raw counts for treatment biological replicate 3 |
| rep4 | DHS raw counts for treatment biological replicate 4 |

Legend for TABLE S5:

| Column | Description |
|---|---|
| chrom | chromosome number for DHS |
| chromStart | chromosome start position for DHS |
| chromEnd | chromosome end position for DHS |
| name | DHS name |
| score | Indicates how dark the peak will be displayed in the |
| | browser (0-1000). If all scores were ‘“0”’ when the data |
| | were submitted to the DCC, the DCC assigned scores 1- |
| | 1000 based on signal value. Ideally the average |
| | signalValue per base spread is between 100-1000. |
| strand | +/− to denote strand or orientation (whenever applicable). |
| | Use “.” if no orientation is assigned. |
| signalValue | Measurement of overall (usually, average) enrichment for |
| | the region. |
| pValue | Measurement of statistical significance (−log10). Use −1 if |
| | no pValue is assigned. |
| qValue | Measurement of statistical significance using false |
| | discovery rate (−log10). Use −1 if no qValue is assigned. |
| peak | Point-source called for this peak; 0-based offset from |
| | chromStart. Use −1 if no point-source called. |
| gRNA_0_1_wg | Number of significant gRNAs in the DHS (FDR < 0.1) |
| bin2_0_1_wg | Number of significant bins (2 gRNAs per bin) in the DHS |
| | (FDR < 0.1) All DHSs, whole-genome discovery screen |
| bin3_0_1_wg | Number of significant bins (3 gRNAs per bin) in the DHS |
| | (FDR < 0.1) All DHSs, whole-genome discovery screen |
| dhs_0_1_wg | Was DHS (all gRNAs grouped) significant? (1 yes, 0 no) |
| | (FDR < 0.1) All DHSs, whole-genome discovery screen |
| gRNA_dir_wg | Direction of fold change between control (no dCas9- |
| | KRAB) and treated (dCas9-KRAB) for gRNA analysis. 0 = |
| | no change, 1 = negative change (depletion), 2 = positive |
| | change (enrichment), 3 = mixed (significant gRNAs or |
| | bins that changed in both directions). FDR < 0.1 All |
| | DHSs, whole-genome discovery screen |
| bin2_dir_wg | Direction of fold change between control (no dCas9- |
| | KRAB) and treated (dCas9-KRAB) for bin2 analysis. 0 = |
| | no change, 1 = negative change (depletion), 2 = positive |
| | change (enrichment), 3 = mixed (significant gRNAs or |
| | bins that changed in both directions). FDR < 0.1 All |
| | DHSs, whole-genome discovery screen |
| bin3_dir_wg | Direction of fold change between control (no dCas9- |
| | KRAB) and treated (dCas9-KRAB) for bin3 analysis. 0 = |
| | no change, 1 = negative change (depletion), 2 = positive |
| | change (enrichment), 3 = mixed (significant gRNAs or |
| | bins that changed in both directions). FDR < 0.1 All |
| | DHSs, whole-genome discovery screen |
| dhs_dir_wg | Direction of fold change between control (no dCas9- |
| | KRAB) and treated (dCas9-KRAB) for DHS analysis. 0 = |
| | no change, 1 = negative change (depletion), 2 = positive |
| | change (enrichment), 3 = mixed (significant gRNAs or |
| | bins that changed in both directions). FDR < 0.1 All |
| | DHSs, whole-genome discovery screen |
| summary_direction_discovery_K562 | Mode of the directions across the 4 analyses (gRNA, |
| | bin2, bin3 and DHS), discovery screen in K562. Possible |
| | values are: depleted, enriched, non-significant (non-sig) |
| | and both. |
| annotation_wg | genomic location of DHS |
| gRNA_score_top3_wg | mean of log2 fold changes for the “top” 3 gRNAs in DHS |
| | (ranked by adjusted p-value) All DHSs, whole-genome |
| | discovery screen |
| bin2_score_top3_wg | mean of log2 fold changes for the “top” 3 bin2s in DHS |
| | (ranked by adjusted p-value) All DHSs, whole-genome |
| | discovery screen |
| bin3_score_top3_wg | mean of log2 fold changes for the “top” 3 bin3s in DHS |
| | (ranked by adjusted p-value) All DHSs, whole-genome |
| | discovery screen |
| dhs_score_top3_wg | log2 fold change of DHS (weither significant or not) All |
| | DHSs, whole-genome discovery screen |
| wgCERES_score_top3_wg | sum of each analysis top3 score (gRNA_score_top3 + |
| | bin2_score_top3 + bin3_score_top3 + dhs_score_top3) |
| | All DHSs, whole-genome discovery screen |
| geneChr | chromosome of nearest gene |
| geneStart | start coordinate of nearest gene |
| geneEnd | end coordinate of nearest gene |
| geneLength | length of nearest gene |
| geneStrand | strand of nearest gene |
| geneId | UCSC id of nearest gene |
| transcriptId | Entrez GeneID of nearest gene |
| distanceToTSS | distance to nearest gene TSS |
| geneSymbol | nearest gene symbol |
| DHS_length | Length of DHS |
| DHS_sequence | Sequence of DHS (hg19) |
| DHS_prop_repeat | Proportion of repetitive sequence in DHS (lower case |
| | DNA sequence) |
| DHS_prop_GC | Proportion of GCs in the DHS |
| ploidyZhou | Large scale ploidy of the region according to Zhou et al. |
| | 2019 (NA value if ploidy of the region was not reported or |
| | if gRNA overlap two regions with different ploidy) |
| LossHetZhou | True if region lost heterozygocity according to Zhou et al. |
| | 2019, False otherwise |
| SV_Zhou | True if structural variant overlap DHS according to Zhou |
| | et al. 2019, False otherwise |
| n_SNV_Zhou | Number of single nucleotide variants that overlap the |
| | DHS according to Zhou et al. 2019 |
| SNV_Zhou | True if single nucleotide variant overlap DHS according to |
| | Zhou et al. 2019, False otherwise |
| n_SNV_Zhou_per_bp | Number of single nucleotide variants that overlap the |
| | DHS according to Zhou et al. 2019 (normalized for size of |
| | DHS) |
| probIntolerantLoF | Probability that the closest gene is intolerant to loss of |
| | function (from Exac, Lek et al. Nature 2016) |
| probIntolerantLoF_gt_0.9 | True if probability that the closest gene is intolerant to |
| | loss of function is higher than 0.9 |
| numTKOHits_Hart | number of cell lines in which the gene is essential (from |
| | Hart et al., Cell 2018) |
| anyTKOHits_Hart | True if number of cell lines in which the gene is essential |
| | (from Hart et al., Cell 2018) is greater than 0 |
| HartEssential | True if genes is essential in more than 2 of the Hart et al. |
| | cell lines (their definition of essential genes, genes with 1 |
| | or 2 could be defined as conditionally essential) |
| OGEE_n_Essential | number of cell lines in which the gene is essential |
| | according to the OGEE database |
| | (http://ogee.medgenius.info) |
| OGEE_n_NonEssential | number of cell lines in which the gene is non-essential |
| | according to the OGEE database |
| | (http://ogee.medgenius.info) |
| OGEE_n | number of cell lines in which the gene was tested for |
| | essentiality according to the OGEE database |
| | (http://ogee.medgenius.info) |
| OGEE_prop_Essential | proportion of cell lines in which the gene is essential |
| | according to the OGEE database |
| | (http://ogee.medgenius.info) |
| OGEE_prop_NonEssential | proportion of cell lines in which the gene is non-essential |
| | according to the OGEE database |
| | (http://ogee.medgenius.info) |
| gene_id | Ensembl gene id |
| medianRNAseqTPM | Median TPM of the mean TPM of 4 ENCODE K562 RNA- |
| | seq experiments (i.e. 4 experiments |
| | (“ENCSR000AEM”, “ENCSR000AEO”, “ENCSR000CPH”, |
| | “ENCSR545DKY”) were done in technical replicates, I |
| | took the mean TPM across replicates for each |
| | experiment, then took the median of the 4 experiments for |
| | genes measured in all four experiments) |
| cancer_census_tier | cancer tier (https://cancer.sanger.ac.uk/). Tier1: To be |
| | classified into Tier 1, a gene must possess a documented |
| | activity relevant to cancer, Tier2: genes with strong |
| | indications of a role in cancer but with less extensive |
| | available evidence. Value set to 0 for non-cancer genes. |
| cancer_census_tissue_type | L −> leukaemia/lymphoma, E −> epithelial, M −> |
| | mesenchymal, O −> other, etc.. |
| cancer_census_role | function of gene in cancer (TSG: tumor supressor gene) |
| vc_sqrt_sum | sum of vc_sqrt (normalised Hi-C) for each extend DHS |
| | (from ABC file). Values are only shown for gRNA entirely |
| | within the extend DHS. If a gRNA entirely overlaps two |
| | extended DHS, the mean is shown. |
| DNase_CPM_per_1 kbp | DNAse CPM per 1 kbp for each extend DHS (from ABC |
| | file). Values are only shown for gRNA entirely within the |
| | extend DHS. If a gRNA entirely overlaps two (or more) |
| | extended DHS, the mean is shown. |
| H3K27ac_CPM_per_1 kbp | H3K27ac CPM per 1 kbp for each extend DHS (from ABC |
| | file). Values are only shown for gRNA entirely within the |
| | extend DHS. If a gRNA entirely overlaps two (or more) |
| | extended DHS, the mean is shown. |
| n_conserved_LindbladToh | Number of base pairs in the DHS that are highly |
| | conserved accross mammals (Lindblad-Toh et al., Nature |
| | 2011) |
| n_conserved_LindbladToh_per_bp | Number of base pairs in the DHS that are highly |
| | conserved accross mammals (Lindblad-Toh et al., Nature |
| | 2011) (normalized for size of DHS) |
| gRNA_0_05_validation_K562 | Number of significant gRNAs in the DHS (FDR < 0.05) |
| | sublibrary followup screen in K562s |
| bin2_0_05_validation_K562 | Number of significant bins (2 gRNAs per bin) in the DHS |
| | (FDR < 0.05) sublibrary followup screen in K562s |
| bin3_0_05_validation_K562 | Number of significant bins (3 gRNAs per bin) in the DHS |
| | (FDR < 0.05) sublibrary followup screen in K562s |
| dhs_0_05_validation_K562 | Was DHS (all gRNAs grouped) significant? (1 yes, 0 no) |
| | (FDR < 0.05) sublibrary followup screen in K562s |
| gRNA_dir_validation_K562 | Direction of fold change between control (no dCas9- |
| | KRAB) and treated (dCas9-KRAB) for gRNA analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in K562 |
| bin2_dir_validation_K562 | Direction of fold change between control (no dCas9- |
| | KRAB) and treated (dCas9-KRAB) for bin2 analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in K562 |
| bin3_dir_validation_K562 | Direction of fold change between control (no dCas9- |
| | KRAB) and treated (dCas9-KRAB) for bin3 analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in K562 |
| dhs_dir_validation_K562 | Direction of fold change between control (no dCas9- |
| | KRAB) and treated (dCas9-KRAB) for DHS analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in K562 |
| gRNA_score_top5_validation_K562 | mean of log2 fold changes for the “top” 5 gRNAs in DHS |
| | (ranked by adjusted p-value) All DHSs, validation screen |
| | in K562 |
| bin2_score_top5_validation_K562 | mean of log2 fold changes for the “top” 5 bin2s in DHS |
| | (ranked by adjusted p-value) All DHSs, validation screen |
| | in K562 |
| bin3_score_top5_validation_K562 | mean of log2 fold changes for the “top” 5 bin3s in DHS |
| | (ranked by adjusted p-value) All DHSs, validation screen |
| | in K562 |
| dhs_score_top5_validation_K562 | log2 fold change of DHS (weither significant or not) All |
| | DHSs, validation screen in K562 |
| wgCERES_score_top5_validation_K562 | sum of each analysis top5 score (gRNA_score_top5 + |
| | bin2_score_top5 + bin3_score_top5 + dhs_score_top5) |
| | validation screen in K562s |
| summary_direction_validation_K562 | Mode of the directions across the 4 analyses (gRNA, |
| | bin2, bin3 and DHS), validation screen in K562 |
| chromHMM_cat_longest | chromHMM automated annotation |
| segway_cat_longest | segway automated annotation |
| gRNA_0_05_validation_OCIAML2 | Number of significant gRNAs in the DHS (FDR < 0.05) |
| | sublibrary followup screen in OCI-AML2s |
| bin2_0_05_validation_OCIAML2 | Number of significant bins (2 gRNAs per bin) in the DHS |
| | (FDR < 0.05) sublibrary followup screen in OCI-AML2s |
| bin3_0_05_validation_OCIAML2 | Number of significant bins (3 gRNAs per bin) in the DHS |
| | (FDR < 0.05) sublibrary followup screen in OCI-AML2s |
| dhs_0_05_validation_OCIAML2 | Was DHS (all gRNAs grouped) significant? (1 yes, 0 no) |
| | (FDR < 0.05) sublibrary followup screen in OCI-AML2s |
| gRNA_dir_validation_OCIAML2 | Direction of fold change between control (no dCas9- |
| | KRAB) and treated (dCas9-KRAB) for gRNA analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in OCI-AML2 |
| bin2_dir_validation_OCIAML2 | Direction of fold change between control (no dCas9- |
| | KRAB) and treated (dCas9-KRAB) for bin2 analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in OCI-AML2 |
| bin3_dir_validation_OCIAML2 | Direction of fold change between control (no dCas9- |
| | KRAB) and treated (dCas9-KRAB) for bin3 analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in OCI-AML2 |
| dhs_dir_validation_OCIAML2 | Direction of fold change between control (no dCas9- |
| | KRAB) and treated (dCas9-KRAB) for DHS analysis. 0 = |
| | no change, 1 = negative change (depletion), 2 = positive |
| | change (enrichment), 3 = mixed (significant gRNAs or |
| | bins that changed in both directions). FDR < 0.05 All |
| | DHSs, validation screen in OCI-AML2 |
| gRNA_score_top5_validation_OCIAML2 | mean of log2 fold changes for the “top” 5 gRNAs in DHS |
| | (ranked by adjusted p-value) All DHSs, validation screen |
| | in OCI-AML2 |
| bin2_score_top5_validation_OCIAML2 | mean of log2 fold changes for the “top” 5 bin2s in DHS |
| | (ranked by adjusted p-value) All DHSs, validation screen |
| | in OCI-AML2 |
| bin3_score_top5_validation_OCIAML2 | mean of log2 fold changes for the “top” 5 bin3s in DHS |
| | (ranked by adjusted p-value) All DHSs, validation screen |
| | in OCI-AML2 |
| dhs_score_top5_validation_OCIAML2 | log2 fold change of DHS (weither significant or not) All |
| | DHSs, validation screen in OCI-AML2 |
| wgCERES_score_top5_validation_OCIAML2 | sum of each analysis top5 score (gRNA_score_top5 + |
| | bin2_score_top5 + bin3_score_top5 + dhs_score_top5) |
| | validation screen in OCI-AML2s |
| summary_direction_validation_OCIAML2 | Mode of the directions across the 4 analyses (gRNA, |
| | bin2, bin3 and DHS), validation screen in OCI-AML2 |

Legend for TABLE S6:

| Column | Description |
|---|---|
| chrom | Chromosome |
| chromStart | gRNA start coordinate (hg19) |
| chromEnd | gRNA end coordinate (hg19) |
| strand | Orientation of the gRNA (positive/forward or negative/reverse) |
| gRNAid | gRNA identifier in this study. It is constructed as the |
| | {DHS}.{NUM_GUIDE_IN_DHS} |
| DHS | DHS identifier. It refers to the unique Dnaseq-seq peak and can be found |
| | in https://www.encodeproject.org/files/ENCFF001UWQ/ |
| protospacer | protspacer DNA sequence targeted by the gRNA |
| baseMean | Measure of gRNA expression across all conditions [DESeq2] |
| log2FoldChange | Relative enrichment in gRNA expression for treatments over controls (in |
| | log2) [DESeq2] |
| lfcSE | Standard error in the relative enrichment in gRNA expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting |
| | (IHW) [DESeq2] |
| weight | P-value weight assigned by Independent Hypothesis Weighting (IHW) |
| | [DESeq2] |
| ctrl1 | gRNA raw counts for control biological replicate 1 |
| ctrl2 | gRNA raw counts for control biological replicate 2 |
| ctrl3 | gRNA raw counts for control biological replicate 3 |
| ctrl4 | gRNA raw counts for control biological replicate 4 |
| rep1 | gRNA raw counts for treatment biological replicate 1 |
| rep2 | gRNA raw counts for treatment biological replicate 2 |
| rep3 | gRNA raw counts for treatment biological replicate 3 |
| rep4 | gRNA raw counts for treatment biological replicate 4 |

Legend for TABLE S7:

| Column | Description |
|---|---|
| chrom | Chromosome |
| chromStart | gRNA-bin2 start coordinate (hg19) |
| chromEnd | gRNA-bin2 end coordinate (hg19) |
| strand | Orientation of the gRNA-bin2 (positive/forward or negative/reverse) |
| binID | gRNA-bin2 identifier in this study. It is constructed as the |
| | {DHS}.bin2_{NUM_BIN2_IN_DHS} |
| DHS | DHS identifier. It refers to the unique Dnaseq-seq peak and can be found in |
| | https://www.encodeproject.org/files/ENCFF001UWQ/ |
| baseMean | Measure of gRNA-bin2 expression across all conditions [DESeq2] |
| log2FoldChange | Relative enrichment in gRNA-bin2 expression for treatments over controls |
| | (in log2) [DESeq2] |
| lfcSE | Standard error in the relative enrichment in gRNA-bin2 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | gRNA-bin2 raw counts for control biological replicate 1 |
| ctrl2 | gRNA-bin2 raw counts for control biological replicate 2 |
| ctrl3 | gRNA-bin2 raw counts for control biological replicate 3 |
| ctrl4 | gRNA-bin2 raw counts for control biological replicate 4 |
| rep1 | gRNA-bin2 raw counts for treatment biological replicate 1 |
| rep2 | gRNA-bin2 raw counts for treatment biological replicate 2 |
| rep3 | gRNA-bin2 raw counts for treatment biological replicate 3 |
| rep4 | gRNA-bin2 raw counts for treatment biological replicate 4 |

Legend for TABLE S8:

| Column | Description |
|---|---|
| chrom | Chromosome |
| chromStart | gRNA-bin3 start coordinate (hg19) |
| chromEnd | gRNA-bin3 end coordinate (hg19) |
| strand | Orientation of the gRNA-bin3 (positive/forward or negative/reverse) |
| binID | gRNA-bin3 identifier in this study. It is constructed as the |
| | {DHS}.bin3_{NUM_bin3_IN_DHS} |
| DHS | DHS identifier. It refers to the unique Dnaseq-seq peak and can be found in |
| | https://www.encodeproject.org/files/ENCFF001UWQ/ |
| baseMean | Measure of gRNA-bin3 expression across all conditions [DESeq2] |
| log2FoldChange | Relative enrichment in gRNA-bin3 expression for treatments over controls |
| | (in log2) [DESeq2] |
| lfcSE | Standard error in the relative enrichment in gRNA-bin3 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | gRNA-bin3 raw counts for control biological replicate 1 |
| ctrl2 | gRNA-bin3 raw counts for control biological replicate 2 |
| ctrl3 | gRNA-bin3 raw counts for control biological replicate 3 |
| ctrl4 | gRNA-bin3 raw counts for control biological replicate 4 |
| rep1 | gRNA-bin3 raw counts for treatment biological replicate 1 |
| rep2 | gRNA-bin3 raw counts for treatment biological replicate 2 |
| rep3 | gRNA-bin3 raw counts for treatment biological replicate 3 |
| rep4 | gRNA-bin3 raw counts for treatment biological replicate 4 |

Legend for TABLE S9:

| Column | Description |
|---|---|
| DHS | DHS identifier. It refers to the unique Dnaseq-seq peak and can be found in |
| | https://www.encodeproject.org/files/ENCFF001UWQ/ |
| baseMean | Measure of DHS expression across all conditions [DESeq2] |
| log2FoldChange | Relative enrichment in DHS expression for treatments over controls (in log2) |
| | [DESeq2] |
| lfcSE | Standard error in the relative enrichment in DHS expression for treatments |
| | over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | DHS raw counts for control biological replicate 1 |
| ctrl2 | DHS raw counts for control biological replicate 2 |
| ctrl3 | DHS raw counts for control biological replicate 3 |
| ctrl4 | DHS raw counts for control biological replicate 4 |
| rep1 | DHS raw counts for treatment biological replicate 1 |
| rep2 | DHS raw counts for treatment biological replicate 2 |
| rep3 | DHS raw counts for treatment biological replicate 3 |
| rep4 | DHS raw counts for treatment biological replicate 4 |

Legend for TABLE S10:

| Column | Description |
|---|---|
| chrom | Chromosome |
| chromStart | gRNA start coordinate (hg19) |
| chromEnd | gRNA end coordinate (hg19) |
| strand | Orientation of the gRNA (positive/forward or negative/reverse) |
| gRNAid | gRNA identifier in this study. It is constructed as the |
| | {DHS}.{NUM_GUIDE_IN_DHS} |
| DHS | DHS identifier. It refers to the unique Dnaseq-seq peak and can be found in |
| | https://www.encodeproject.org/files/ENCFF001UWQ/ |
| protospacer | protspacer DNA sequence targeted by the gRNA |
| baseMean | Measure of gRNA expression across all conditions [DESeq2] |
| log2FoldChange | Relative enrichment in gRNA expression for treatments over controls (in |
| | log2) [DESeq2] |
| lfcSE | Standard error in the relative enrichment in gRNA expression for treatments |
| | over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | gRNA raw counts for control biological replicate 1 |
| ctrl2 | gRNA raw counts for control biological replicate 2 |
| ctrl3 | gRNA raw counts for control biological replicate 3 |
| ctrl4 | gRNA raw counts for control biological replicate 4 |
| rep1 | gRNA raw counts for treatment biological replicate 1 |
| rep2 | gRNA raw counts for treatment biological replicate 2 |
| rep3 | gRNA raw counts for treatment biological replicate 3 |
| rep4 | gRNA raw counts for treatment biological replicate 4 |

Legend for TABLE S11:

| Column | Description |
|---|---|
| chrom | Chromosome |
| chromStart | gRNA-bin2 start coordinate (hg19) |
| chromEnd | gRNA-bin2 end coordinate (hg19) |
| strand | Orientation of the gRNA-bin2 (positive/forward or negative/reverse) |
| binID | gRNA-bin2 identifier in this study. It is constructed as the |
| | {DHS}.bin2_{NUM_BIN2_IN_DHS} |
| DHS | DHS identifier. It refers to the unique Dnaseq-seq peak and can be found in |
| | https://www.encodeproject.org/files/ENCFF001UWQ/ |
| baseMean | Measure of gRNA-bin2 expression across all conditions [DESeq2] |
| log2FoldChange | Relative enrichment in gRNA-bin2 expression for treatments over controls |
| | (in log2) [DESeq2] |
| lfcSE | Standard error in the relative enrichment in gRNA-bin2 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | gRNA-bin2 raw counts for control biological replicate 1 |
| ctrl2 | gRNA-bin2 raw counts for control biological replicate 2 |
| ctrl3 | gRNA-bin2 raw counts for control biological replicate 3 |
| ctrl4 | gRNA-bin2 raw counts for control biological replicate 4 |
| rep1 | gRNA-bin2 raw counts for treatment biological replicate 1 |
| rep2 | gRNA-bin2 raw counts for treatment biological replicate 2 |
| rep3 | gRNA-bin2 raw counts for treatment biological replicate 3 |
| rep4 | gRNA-bin2 raw counts for treatment biological replicate 4 |

Legend for TABLE S12:

| Column | Description |
|---|---|
| chrom | Chromosome |
| chromStart | gRNA-bin3 start coordinate (hg19) |
| chromEnd | gRNA-bin3 end coordinate (hg19) |
| strand | Orientation of the gRNA-bin3 (positive/forward or negative/reverse) |
| binID | gRNA-bin3 identifier in this study. It is constructed as the |
| | {DHS}.bin3_{NUM_bin3_IN_DHS} |
| DHS | DHS identifier. It refers to the unique Dnaseq-seq peak and can be found in |
| | https://www.encodeproject.org/files/ENCFF001UWQ/ |
| baseMean | Measure of gRNA-bin3 expression across all conditions [DESeq2] |
| log2FoldChange | Relative enrichment in gRNA-bin3 expression for treatments over controls |
| | (in log2) [DESeq2] |
| lfcSE | Standard error in the relative enrichment in gRNA-bin3 expression for |
| | treatments over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | gRNA-bin3 raw counts for control biological replicate 1 |
| ctrl2 | gRNA-bin3 raw counts for control biological replicate 2 |
| ctrl3 | gRNA-bin3 raw counts for control biological replicate 3 |
| ctrl4 | gRNA-bin3 raw counts for control biological replicate 4 |
| rep1 | gRNA-bin3 raw counts for treatment biological replicate 1 |
| rep2 | gRNA-bin3 raw counts for treatment biological replicate 2 |
| rep3 | gRNA-bin3 raw counts for treatment biological replicate 3 |
| rep4 | gRNA-bin3 raw counts for treatment biological replicate 4 |

Legend for TABLE S13:

| Column | Description |
|---|---|
| DHS | DHS identifier. It refers to the unique Dnaseq-seq peak and can be found in |
| | https://www.encodeproject.org/files/ENCFF001UWQ/ |
| baseMean | Measure of DHS expression across all conditions [DESeq2] |
| log2FoldChange | Relative enrichment in DHS expression for treatments over controls (in log2) |
| | [DESeq2] |
| lfcSE | Standard error in the relative enrichment in DHS expression for treatments |
| | over controls (in log2) [DESeq2] |
| stat | Wald test statistic (log2FoldChange/lfcSE) [DESeq2] |
| pvalue | Two tailed p-value generated by comparing Wald statistics to a Normal |
| | distribution, |
| padj | Adjusted p-value by applying the procedure of Benjamini Hochberg (BH) to |
| | the weighted p-value assigned by Independent Hypothesis Weighting (IHW) |
| | [DESeq2] |
| weight | P-value weight assigned by Independent Hypothesis Weighting (IHW) |
| | [DESeq2] |
| ctrl1 | DHS raw counts for control biological replicate 1 |
| ctrl2 | DHS raw counts for control biological replicate 2 |
| ctrl3 | DHS raw counts for control biological replicate 3 |
| ctrl4 | DHS raw counts for control biological replicate 4 |
| rep1 | DHS raw counts for treatment biological replicate 1 |
| rep2 | DHS raw counts for treatment biological replicate 2 |
| rep3 | DHS raw counts for treatment biological replicate 3 |
| rep4 | DHS raw counts for treatment biological replicate 4 |

Legend for TABLE S17:

| Column | Description |
|---|---|
| p_val | P-value associated with the enrichement of a given gene, |
| | estimated by MAST (PMID: 26653891) |
| avg_logFC | log fold-chage of the average expression between the two |
| | groups. Positive values indicate that the gene is more highly |
| | expressed in the first group |
| pct.1 | The percentage of cells where the gene is detected in the first |
| | group of cells where the gRNA is estimated to be present |
| pct.2 | The percentage of cells where the gene is detected in the |
| | second group of cells where the gRNA is estimated to not be |
| | present |
| p_val_adj | Bonferroni corrected p-value, using the number of genes |
| | overlapping the +/−1 Mb window around the gRNA |
| pval_fdr_corrected | Adjusted p-value by applying the procedure of Benjamini |
| | Hochberg (BH) |
| grna | gRNA ID followed by the protospacer sequence |
| gene_symbol | Gene symbol |
| pval_empirical | Empirical p-value using the p-values from all nontargeting |
| | gRNAs and the union set of genes in all +/−1 Mb windows |
| | around all gRNAs tested. |
| chrom | Chromosome |
| start | gRNA start coordinate (hg19) |
| end | gRNA end coordinate (hg19) |
| strand | Orientation of the gRNA-bin2 (positive/forward or |
| | negative/reverse) |
| dhs_id | DHS identifier. It refers to the unique Dnaseq-seq peak and can |
| | be found in |
| | https://www.encodeproject.org/files/ENCFF001UWQ/ |
| dhs_chrom | DHS raw counts for treatment biological replicate 4 |
| dhs_start | DHS start coordinate (hg19) |
| dhs_end | DHS end coordinate (hg19) |
| distance_to_tss_of_linked_gene | Distance from the midpoint of the gRNA to the TSS of the target |
| | gene |

SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T; V = A or
C or G)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
DNA sequence of the gRNA constant region
gtttaagagctatgctggaaacagcatagcaagtttaaataaggctagtccgttatcaacttgaaaaagt
ggcaccgagtcggtgc
SEQ ID NO: 19
RNA sequence of the gRNA constant region
guuuaagagcuaugcuggaaacagcauagcaaguuuaaauaaggcuaguccguuaucaacuugaaaaagu
ggcaccgagucggugc
SEQ ID NO: 20
Streptococcus pyogenes Cas9
MDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESELVEEDKKHERHPIFGNIVDEVAYHEKYPTIYHLRK
KLVDSTDKADLRLIYLALAHMIKERGHFLIEGDLNPDNSDVDKLFIQLVQTYNQLFEENPINASGVDAKA
ILSARLSKSRRLENLIAQLPGEKKNGLFGNLIALSLGLTPNEKSNEDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTEDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLEKTNRKVT
VKQLKEDYFKKIECFDSVEISGVEDRFNASLGTYHDLLKIIKDKDELDNEENEDILEDIVLTLTLFEDRE
MIEERLKTYAHLEDDKVMKQLKRRRYTGWGRLSRKLINGIRDKQSGKTILDELKSDGFANRNEMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKEDNLTKAERGGLSE
LDKAGFIKRQLVETRQITKHVAQILDSRMNTKYDENDKLIREVKVITLKSKLVSDERKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLTNLGAPAAFKYEDTT
IDRKRYTSTKEVLDATLIHQSITGLYETRIDLSQLGGD
SEQ ID NO: 21
Staphylococcus aureus Cas9
MKRNYILGLDIGITSVGYGIIDYETRDVIDAGVRLFKEANVENNEGRRSKRGARRLKRRRRHRIQRVKKL
LEDYNLLTDHSELSGINPYEARVKGLSQKLSEEEFSAALLHLAKRRGVHNVNEVEEDTGNELSTKEQISR
NSKALEEKYVAELQLERLKKDGEVRGSINRFKTSDYVKEAKQLLKVQKAYHQLDQSFIDTYIDLLETRRT
YYEGPGEGSPFGWKDIKEWYEMLMGHCTYFPEELRSVKYAYNADLYNALNDLNNLVITRDENEKLEYYEK
FQIIENVFKQKKKPTLKQIAKEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAINLILDELWHTNDNQIAIENR
LKLVPKKVDLSQQKEIPTTLVDDFILSPVVKRSFIQSIKVINAIIKKYGLPNDIIIELAREKNSKDAQKM
INEMQKRNRQTNERIEEIIRTTGKENAKYLIEKIKLHDMQEGKCLYSLEAIPLEDLLNNPENYEVDHIIP
RSVSFDNSFNNKVLVKQEENSKKGNRTPFQYLSSSDSKISYETFKKHILNLAKGKGRISKTKKEYLLEER
DINRFSVQKDFINRNLVDTRYATRGLMNLLRSYFRVNNLDVKVKSINGGFTSELRRKWKFKKERNKGYKH
HAEDALIIANADFIFKEWKKLDKAKKVMENQMFEEKQAESMPEIETEQEYKEIFITPHQIKHIKDEKDYK
YSHRVDKKPNRELINDTLYSTRKDDKGNTLIVNNLNGLYDKDNDKLKKLINKSPEKLLMYHHDPQTYQKL
KLIMEQYGDEKNPLYKYYEETGNYLTKYSKKDNGPVIKKIKYYGNKLNAHLDITDDYPNSRNKVVKLSLK
PYRFDVYLDNGVYKFVTVKNLDVIKKENYYEVNSKCYEEAKKLKKISNQAEFIASFYNNDLIKINGELYR
VIGVNNDLLNRIEVNMIDITYREYLENMNDKRPPRIIKTIASKTQSIKKYSTDILGNLYEVKSKKHPQII
KKG
SEQ ID NO: 22
Streptococcus pyogenes Cas9 (with D10A)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESELVEEDKKHERHPIFGNIVDEVAYHEKYPTIYHLRK
KLVDSTDKADLRLIYLALAHMIKERGHFLIEGDLNPDNSDVDKLFIQLVQTYNQLFEENPINASGVDAKA
ILSARLSKSRRLENLIAQLPGEKKNGLFGNLIALSLGLTPNEKSNEDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTEDNGSIPHQIHLGELH
AILRRQEDFYPELKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLEKTNRKVT
VKQLKEDYFKKIECFDSVEISGVEDRFNASLGTYHDLLKIIKDKDELDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKEDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYEDTT
IDRKRYTSTKEVLDATLIHQSITGLYETRIDLSQLGGD
SEQ ID NO: 23
Streptococcus pyogenes Cas9 (with D10A, H849A)
MDKKYSIGLAIGTNSVGWAVITDEYKVPSKKFKVLGNTDRHSIKKNLIGALLEDSGETAEATRLKRTARR
RYTRRKNRICYLQEIFSNEMAKVDDSFFHRLEESELVEEDKKHERHPIFGNIVDEVAYHEKYPTIYHLRK
KLVDSTDKADLRLIYLALAHMIKERGHFLIEGDLNPDNSDVDKLFIQLVQTYNQLFEENPINASGVDAKA
ILSARLSKSRRLENLIAQLPGEKKNGLFGNLIALSLGLTPNEKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTEDNGSIPHQIHLGELH
AILRRQEDFYPELKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLEKTNRKVT
VKQLKEDYFKKIECFDSVEISGVEDRFNASLGTYHDLLKIIKDKDELDNEENEDILEDIVLTLTLFEDRE
MIEERLKTYAHLEDDKVMKQLKRRRYTGWGRLSRKLINGIRDKQSGKTILDELKSDGFANRNEMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRKEDNLTKAERGGLSE
LDKAGFIKRQLVETRQITKHVAQILDSRMNTKYDENDKLIREVKVITLKSKLVSDERKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLEVE
QHKHYLDEIIEQISEFSKRVILADANLDKVLSAYNKHRDKPIREQAENIIHLFTLTNLGAPAAFKYEDTT
IDRKRYTSTKEVLDATLIHQSITGLYETRIDLSQLGGD
SEQ ID NO: 24
Polynucleotide sequence of D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag ctctggaaga gaagtatgtc cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc ccaatctgaa agtgtatcac gatattaagg gggtgacaag cactggaaaa
ccagagttca acgccgaact gctggatcag attgctaaga acatcacagc acggaaagaa
atcattgaga tccaggaaga gctgactaac ctgaacagcg tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 25
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 26
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tottgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 27
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 28
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 29
codon optimized nucleic acid sequence encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 30
codon optimized nucleic acid sequence encoding S. aureus Cas9
atggccccaaagaagaagcggaaggtcggtatccacggagtcccagcagccaagcggaactacatcctgg
gcctggacatcggcatcaccagcgtgggctacggcatcatcgactacgagacacgggacgtgatcgatgc
cggcgtgcggctgttcaaagaggccaacgtggaaaacaacgagggcaggcggagcaagagaggcgccaga
aggctgaagcggcggaggcggcatagaatccagagagtgaagaagctgctgttcgactacaacctgctga
ccgaccacagcgagctgagcggcatcaacccctacgaggccagagtgaagggcctgagccagaagctgag
cgaggaagagttctctgccgccctgctgcacctggccaagagaagaggcgtgcacaacgtgaacgaggtg
gaagaggacaccggcaacgagctgtccaccagagagcagatcagccggaacagcaaggccctggaagaga
aatacgtggccgaactgcagctggaacggctgaagaaagacggcgaagtgcggggcagcatcaacagatt
caagaccagcgactacgtgaaagaagccaaacagctgctgaaggtgcagaaggcctaccaccagctggac
cagagcttcatcgacacctacatcgacctgctggaaacccggcggacctactatgagggacctggcgagg
gcagccccttcggctggaaggacatcaaagaatggtacgagatgctgatgggccactgcacctacttccc
cgaggaactgcggagcgtgaagtacgcctacaacgccgacctgtacaacgccctgaacgacctgaacaat
ctcgtgatcaccagggacgagaacgagaagctggaatattacgagaagttccagatcatcgagaacgtgt
tcaagcagaagaagaagcccaccctgaagcagatcgccaaagaaatcctcgtgaacgaagaggatattaa
gggctacagagtgaccagcaccggcaagcccgagttcaccaacctgaaggtgtaccacgacatcaaggac
attaccgcccggaaagagattattgagaacgccgagctgctggatcagattgccaagatcctgaccatct
accagagcagcgaggacatccaggaagaactgaccaatctgaactccgagctgacccaggaagagatcga
gcagatctctaatctgaagggctataccggcacccacaacctgagcctgaaggccatcaacctgatcctg
gacgagctgtggcacaccaacgacaaccagatcgctatcttcaaccggctgaagctggtgcccaagaagg
tggacctgtcccagcagaaagagatccccaccaccctggtggacgacttcatcctgagccccgtcgtgaa
gagaagcttcatccagagcatcaaagtgatcaacgccatcatcaagaagtacggcctgcccaacgacatc
attatcgagctggcccgcgagaagaactccaaggacgcccagaaaatgatcaacgagatgcagaagcgga
accggcagaccaacgagcggatcgaggaaatcatccggaccaccggcaaagagaacgccaagtacctgat
cgagaagatcaagctgcacgacatgcaggaaggcaagtgcctgtacagcctggaagccatccctctggaa
gatctgctgaacaaccccttcaactatgaggtggaccacatcatccccagaagcgtgtccttcgacaaca
gcttcaacaacaaggtgctcgtgaagcaggaagaaaacagcaagaagggcaaccggaccccattccagta
cctgagcagcagcgacagcaagatcagctacgaaaccttcaagaagcacatcctgaatctggccaagggc
aagggcagaatcagcaagaccaagaaagagtatctgctggaagaacgggacatcaacaggttctccgtgc
agaaagacttcatcaaccggaacctggtggataccagatacgccaccagaggcctgatgaacctgctgcg
gagctacttcagagtgaacaacctggacgtgaaagtgaagtccatcaatggcggcttcaccagctttctg
cggcggaagtggaagtttaagaaagagcggaacaaggggtacaagcaccacgccgaggacgccctgatca
ttgccaacgccgatttcatcttcaaagagtggaagaaactggacaaggccaaaaaagtgatggaaaacca
gatgttcgaggaaaggcaggccgagagcatgcccgagatcgaaaccgagcaggagtacaaagagatcttc
atcaccccccaccagatcaagcacattaaggacttcaaggactacaagtacagccaccgggtggacaaga
agcctaatagagagctgattaacgacaccctgtactccacccggaaggacgacaagggcaacaccctgat
cgtgaacaatctgaacggcctgtacgacaaggacaatgacaagctgaaaaagctgatcaacaagagcccc
gaaaagctgctgatgtaccaccacgacccccagacctaccagaaactgaagctgattatggaacagtacg
gcgacgagaagaatcccctgtacaagtactacgaggaaaccgggaactacctgaccaagtactccaaaaa
ggacaacggccccgtgatcaagaagattaagtattacggcaacaaactgaacgcccatctggacatcacc
gacgactaccccaacagcagaaacaaggtcgtgaagctgtccctgaagccctacagattcgacgtgtacc
tggacaatggcgtgtacaagttcgtgaccgtgaagaatctggatgtgatcaaaaaagaaaactactacga
agtgaatagcaagtgctatgaggaagctaagaagctgaagaagatcagcaaccaggccgagtttatcgcc
tccttctacaacaacgatctgatcaagatcaacggcgagctgtatagagtgatcggcgtgaacaacgacc
tgctgaaccggatcgaagtgaacatgatcgacatcacctaccgcgagtacctggaaaacatgaacgacaa
gaggccccccaggatcattaagacaatcgcctccaagacccagagcattaagaagtacagcacagacatt
ctgggcaacctgtatgaagtgaaatctaagaagcaccctcagatcatcaaaaagggcaaaaggccggcgg
ccacgaaaaaggccggccaggcaaaaaagaaaaag
SEQ ID NO: 31
codon optimized nucleic acid sequence encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 32
codon optimized nucleic acid sequences encoding S. aureus Cas9
atggccccaaagaagaagcggaaggtcggtatccacggagtcccagcagccaagcggaactacatcctgg
gcctggacatcggcatcaccagcgtgggctacggcatcatcgactacgagacacgggacgtgatcgatgc
cggcgtgcggctgttcaaagaggccaacgtggaaaacaacgagggcaggcggagcaagagaggcgccaga
aggctgaagcggcggaggcggcatagaatccagagagtgaagaagctgctgttcgactacaacctgctga
ccgaccacagcgagctgagcggcatcaacccctacgaggccagagtgaagggcctgagccagaagctgag
cgaggaagagttctctgccgccctgctgcacctggccaagagaagaggcgtgcacaacgtgaacgaggtg
gaagaggacaccggcaacgagctgtccaccaaagagcagatcagccggaacagcaaggccctggaagaga
aatacgtggccgaactgcagctggaacggctgaagaaagacggcgaagtgcggggcagcatcaacagatt
caagaccagcgactacgtgaaagaagccaaacagctgctgaaggtgcagaaggcctaccaccagctggac
cagagcttcatcgacacctacatcgacctgctggaaacccggcggacctactatgagggacctggcgagg
gcagccccttcggctggaaggacatcaaagaatggtacgagatgctgatgggccactgcacctacttccc
cgaggaactgcggagcgtgaagtacgcctacaacgccgacctgtacaacgccctgaacgacctgaacaat
ctcgtgatcaccagggacgagaacgagaagctggaatattacgagaagttccagatcatcgagaacgtgt
tcaagcagaagaagaagcccaccctgaagcagatcgccaaagaaatcctcgtgaacgaagaggatattaa
gggctacagagtgaccagcaccggcaagcccgagttcaccaacctgaaggtgtaccacgacatcaaggac
attaccgcccggaaagagattattgagaacgccgagctgctggatcagattgccaagatcctgaccatct
accagagcagcgaggacatccaggaagaactgaccaatctgaactccgagctgacccaggaagagatcga
gcagatctctaatctgaagggctataccggcacccacaacctgagcctgaaggccatcaacctgatcctg
gacgagctgtggcacaccaacgacaaccagatcgctatcttcaaccggctgaagctggtgcccaagaagg
tggacctgtcccagcagaaagagatccccaccaccctggtggacgacttcatcctgagccccgtcgtgaa
gagaagcttcatccagagcatcaaagtgatcaacgccatcatcaagaagtacggcctgcccaacgacatc
attatcgagctggcccgcgagaagaactccaaggacgcccagaaaatgatcaacgagatgcagaagcgga
accggcagaccaacgagcggatcgaggaaatcatccggaccaccggcaaagagaacgccaagtacctgat
cgagaagatcaagctgcacgacatgcaggaaggcaagtgcctgtacagcctggaagccatccctctggaa
gatctgctgaacaaccccttcaactatgaggtggaccacatcatccccagaagcgtgtccttcgacaaca
gcttcaacaacaaggtgctcgtgaagcaggaagaaaacagcaagaagggcaaccggaccccattccagta
cctgagcagcagcgacagcaagatcagctacgaaaccttcaagaagcacatcctgaatctggccaagggc
aagggcagaatcagcaagaccaagaaagagtatctgctggaagaacgggacatcaacaggttctccgtgc
agaaagacttcatcaaccggaacctggtggataccagatacgccaccagaggcctgatgaacctgctgcg
gagctacttcagagtgaacaacctggacgtgaaagtgaagtccatcaatggcggcttcaccagctttctg
cggcggaagtggaagtttaagaaagagcggaacaaggggtacaagcaccacgccgaggacgccctgatca
ttgccaacgccgatttcatcttcaaagagtggaagaaactggacaaggccaaaaaagtgatggaaaacca
gatgttcgaggaaaagcaggccgagagcatgcccgagatcgaaaccgagcaggagtacaaagagatcttc
atcaccccccaccagatcaagcacattaaggacttcaaggactacaagtacagccaccgggtggacaaga
agcctaatagagagctgattaacgacaccctgtactccacccggaaggacgacaagggcaacaccctgat
cgtgaacaatctgaacggcctgtacgacaaggacaatgacaagctgaaaaagctgatcaacaagagcccc
gaaaagctgctgatgtaccaccacgacccccagacctaccagaaactgaagctgattatggaacagtacg
gcgacgagaagaatcccctgtacaagtactacgaggaaaccgggaactacctgaccaagtactccaaaaa
ggacaacggccccgtgatcaagaagattaagtattacggcaacaaactgaacgcccatctggacatcacc
gacgactaccccaacagcagaaacaaggtcgtgaagctgtccctgaagccctacagattcgacgtgtacc
tggacaatggcgtgtacaagttcgtgaccgtgaagaatctggatgtgatcaaaaaagaaaactactacga
agtgaatagcaagtgctatgaggaagctaagaagctgaagaagatcagcaaccaggccgagtttatcgcc
tccttctacaacaacgatctgatcaagatcaacggcgagctgtatagagtgatcggcgtgaacaacgacc
tgctgaaccggatcgaagtgaacatgatcgacatcacctaccgcgagtacctggaaaacatgaacgacaa
gaggccccccaggatcattaagacaatcgcctccaagacccagagcattaagaagtacagcacagacatt
ctgggcaacctgtatgaagtgaaatctaagaagcaccctcagatcatcaaaaagggcaaaaggccggcgg
ccacgaaaaaggccggccaggcaaaaaagaaaaag
SEQ ID NO: 33
codon optimized nucleic acid sequences encoding S. aureus Cas9
aagcggaactacatcctgggcctggacatcggcatcaccagcgtgggctacggcatcatcgactacgaga
cacgggacgtgatcgatgccggcgtgcggctgttcaaagaggccaacgtggaaaacaacgagggcaggcg
gagcaagagaggcgccagaaggctgaagcggcggaggcggcatagaatccagagagtgaagaagctgctg
ttcgactacaacctgctgaccgaccacagcgagctgagcggcatcaacccctacgaggccagagtgaagg
gcctgagccagaagctgagcgaggaagagttctctgccgccctgctgcacctggccaagagaagaggcgt
gcacaacgtgaacgaggtggaagaggacaccggcaacgagctgtccaccaaagagcagatcagccggaac
agcaaggccctggaagagaaatacgtggccgaactgcagctggaacggctgaagaaagacggcgaagtgc
ggggcagcatcaacagattcaagaccagcgactacgtgaaagaagccaaacagctgctgaaggtgcagaa
ggcctaccaccagctggaccagagcttcatcgacacctacatcgacctgctggaaacccggcggacctac
tatgagggacctggcgagggcagccccttcggctggaaggacatcaaagaatggtacgagatgctgatgg
gccactgcacctacttccccgaggaactgcggagcgtgaagtacgcctacaacgccgacctgtacaacgc
cctgaacgacctgaacaatctcgtgatcaccagggacgagaacgagaagctggaatattacgagaagttc
cagatcatcgagaacgtgttcaagcagaagaagaagcccaccctgaagcagatcgccaaagaaatcctcg
tgaacgaagaggatattaagggctacagagtgaccagcaccggcaagcccgagttcaccaacctgaaggt
gtaccacgacatcaaggacattaccgcccggaaagagattattgagaacgccgagctgctggatcagatt
gccaagatcctgaccatctaccagagcagcgaggacatccaggaagaactgaccaatctgaactccgagc
tgacccaggaagagatcgagcagatctctaatctgaagggctataccggcacccacaacctgagcctgaa
ggccatcaacctgatcctggacgagctgtggcacaccaacgacaaccagatcgctatcttcaaccggctg
aagctggtgcccaagaaggtggacctgtcccagcagaaagagatccccaccaccctggtggacgacttca
tcctgagccccgtcgtgaagagaagcttcatccagagcatcaaagtgatcaacgccatcatcaagaagta
cggcctgcccaacgacatcattatcgagctggcccgcgagaagaactccaaggacgcccagaaaatgatc
aacgagatgcagaagcggaaccggcagaccaacgagcggatcgaggaaatcatccggaccaccggcaaag
agaacgccaagtacctgatcgagaagatcaagctgcacgacatgcaggaaggcaagtgcctgtacagcct
ggaagccatccctctggaagatctgctgaacaaccccttcaactatgaggtggaccacatcatccccaga
agcgtgtccttcgacaacagcttcaacaacaaggtgctcgtgaagcaggaagaaaacagcaagaagggca
accggaccccattccagtacctgagcagcagcgacagcaagatcagctacgaaaccttcaagaagcacat
cctgaatctggccaagggcaagggcagaatcagcaagaccaagaaagagtatctgctggaagaacgggac
atcaacaggttctccgtgcagaaagacttcatcaaccggaacctggtggataccagatacgccaccagag
gcctgatgaacctgctgcggagctacttcagagtgaacaacctggacgtgaaagtgaagtccatcaatgg
cggcttcaccagctttctgcggcggaagtggaagtttaagaaagagcggaacaaggggtacaagcaccac
gccgaggacgccctgatcattgccaacgccgatttcatcttcaaagagtggaagaaactggacaaggcca
aaaaagtgatggaaaaccagatgttcgaggaaaagcaggccgagagcatgcccgagatcgaaaccgagca
ggagtacaaagagatcttcatcaccccccaccagatcaagcacattaaggacttcaaggactacaagtac
agccaccgggtggacaagaagcctaatagagagctgattaacgacaccctgtactccacccggaaggacg
acaagggcaacaccctgatcgtgaacaatctgaacggcctgtacgacaaggacaatgacaagctgaaaaa
gctgatcaacaagagccccgaaaagctgctgatgtaccaccacgacccccagacctaccagaaactgaag
ctgattatggaacagtacggcgacgagaagaatcccctgtacaagtactacgaggaaaccgggaactacc
tgaccaagtactccaaaaaggacaacggccccgtgatcaagaagattaagtattacggcaacaaactgaa
cgcccatctggacatcaccgacgactaccccaacagcagaaacaaggtcgtgaagctgtccctgaagccc
tacagattcgacgtgtacctggacaatggcgtgtacaagttcgtgaccgtgaagaatctggatgtgatca
aaaaagaaaactactacgaagtgaatagcaagtgctatgaggaagctaagaagctgaagaagatcagcaa
ccaggccgagtttatcgcctccttctacaacaacgatctgatcaagatcaacggcgagctgtatagagtg
atcggcgtgaacaacgacctgctgaaccggatcgaagtgaacatgatcgacatcacctaccgcgagtacc
tggaaaacatgaacgacaagaggccccccaggatcattaagacaatcgcctccaagacccagagcattaa
gaagtacagcacagacattctgggcaacctgtatgaagtgaaatctaagaagcaccctcagatcatcaaa
aagggc
SEQ ID NO: 34
Vector (pDO242) encoding codon optimized nucleic acid sequence encoding S. aureus Cas9
ctaaattgtaagcgttaatattttttaaaattcgcgttaaatttttgttaaatcagctcattttttaac
caataggccgaaatcggcaaaatcccttataaatcaaaagaatagaccgagatagggttgagtgttgttc
cagtttggaacaagagtccactattaaagaacgtggactccaacgtcaaagggcgaaaaaccgtctatca
gggcgatggcccactacgtgaaccatcaccctaatcaagttttttggggtcgaggtgccgtaaagcacta
aatcggaaccctaaagggagcccccgatttagagcttgacggggaaagccggcgaacgtggcgagaaagg
aagggaagaaagcgaaaggagcgggcgctagggcgctggcaagtgtagcggtcacgctgcgcgtaaccac
cacacccgccgcgcttaatgcgccgctacagggcgcgtcccattcgccattcaggctgcgcaactgttgg
gaagggcgatcggtgcgggcctcttcgctattacgccagctggcgaaagggggatgtgctgcaaggcgat
taagttgggtaacgccagggttttcccagtcacgacgttgtaaaacgacggccagtgagcgcgcgtaata
cgactcactatagggcgaattgggtacCtttaattctagtactatgcaTgcgttgacattgattattgac
tagttattaatagtaatcaattacggggtcattagttcatagcccatatatggagttccgcgttacataa
cttacggtaaatggcccgcctggctgaccgcccaacgacccccgcccattgacgtcaataatgacgtatg
ttcccatagtaacgccaatagggactttccattgacgtcaatgggtggagtatttacggtaaactgccca
cttggcagtacatcaagtgtatcatatgccaagtacgccccctattgacgtcaatgacggtaaatggccc
gcctggcattatgcccagtacatgaccttatgggactttcctacttggcagtacatctacgtattagtca
tcgctattaccatggtgatgcggttttggcagtacatcaatgggcgtggatagcggtttgactcacgggg
atttccaagtctccaccccattgacgtcaatgggagtttgttttggcaccaaaatcaacgggactttcca
aaatgtcgtaacaactccgccccattgacgcaaatgggcggtaggcgtgtacggtgggaggtctatataa
gcagagctctctggctaactaccggtgccaccATGAAAAGGAACTACATTCTGGGGCTGGACATCGGGAT
TACAAGCGTGGGGTATGGGATTATTGACTATGAAACAAGGGACGTGATCGACGCAGGCGTCAGACTGTTC
AAGGAGGCCAACGTGGAAAACAATGAGGGACGGAGAAGCAAGAGGGGAGCCAGGCGCCTGAAACGACGGA
GAAGGCACAGAATCCAGAGGGTGAAGAAACTGCTGTTCGATTACAACCTGCTGACCGACCATTCTGAGCT
GAGTGGAATTAATCCTTATGAAGCCAGGGTGAAAGGCCTGAGTCAGAAGCTGTCAGAGGAAGAGTTTTCC
GCAGCTCTGCTGCACCTGGCTAAGCGCCGAGGAGTGCATAACGTCAATGAGGTGGAAGAGGACACCGGCA
ACGAGCTGTCTACAAAGGAACAGATCTCACGCAATAGCAAAGCTCTGGAAGAGAAGTATGTCGCAGAGCT
GCAGCTGGAACGGCTGAAGAAAGATGGCGAGGTGAGAGGGTCAATTAATAGGTTCAAGACAAGCGACTAC
GTCAAAGAAGCCAAGCAGCTGCTGAAAGTGCAGAAGGCTTACCACCAGCTGGATCAGAGCTTCATCGATA
CTTATATCGACCTGCTGGAGACTCGGAGAACCTACTATGAGGGACCAGGAGAAGGGAGCCCCTTCGGATG
GAAAGACATCAAGGAATGGTACGAGATGCTGATGGGACATTGCACCTATTTTCCAGAAGAGCTGAGAAGC
GTCAAGTACGCTTATAACGCAGATCTGTACAACGCCCTGAATGACCTGAACAACCTGGTCATCACCAGGG
ATGAAAACGAGAAACTGGAATACTATGAGAAGTTCCAGATCATCGAAAACGTGTTTAAGCAGAAGAAAAA
GCCTACACTGAAACAGATTGCTAAGGAGATCCTGGTCAACGAAGAGGACATCAAGGGCTACCGGGTGACA
AGCACTGGAAAACCAGAGTTCACCAATCTGAAAGTGTATCACGATATTAAGGACATCACAGCACGGAAAG
AAATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATCTG
AAGGGGTACACCGGAACACACAACCTGTCCCTGAAAGCTATCAATCTGATTCTGGATGAGCTGTGGCATA
CAAACGACAATCAGATTGCAATCTTTAACCGGCTGAAGCTGGTCCCAAAAAAGGTGGACCTGAGTCAGCA
GAAAGAGATCCCAACCACACTGGTGGACGATTTCATTCTGTCACCCGTGGTCAAGCGGAGCTTCATCCAG
AGCATCAAAGTGATCAACGCCATCATCAAGAAGTACGGCCTGCCCAATGATATCATTATCGAGCTGGCTA
GGGAGAAGAACAGCAAGGACGCACAGAAGATGATCAATGAGATGCAGAAACGAAACCGGCAGACCAATGA
ACGCATTGAAGAGATTATCCGAACTACCGGGAAAGAGAACGCAAAGTACCTGATTGAAAAAATCAAGCTG
CACGATATGCAGGAGGGAAAGTGTCTGTATTCTCTGGAGGCCATCCCCCTGGAGGACCTGCTGAACAATC
CATTCAACTACGAGGTCGATCATATTATCCCCAGAAGCGTGTCCTTCGACAATTCCTTTAACAACAAGGT
GCTGGTCAAGCAGGAAGAGAACTCTAAAAAGGGCAATAGGACTCCTTTCCAGTACCTGTCTAGTTCAGAT
TCCAAGATCTCTTACGAAACCTTTAAAAAGCACATTCTGAATCTGGCCAAAGGAAAGGGCCGCATCAGCA
AGACCAAAAAGGAGTACCTGCTGGAAGAGCGGGACATCAACAGATTCTCCGTCCAGAAGGATTTTATTAA
CCGGAATCTGGTGGACACAAGATACGCTACTCGCGGCCTGATGAATCTGCTGCGATCCTATTTCCGGGTG
AACAATCTGGATGTGAAAGTCAAGTCCATCAACGGCGGGTTCACATCTTTTCTGAGGCGCAAATGGAAGT
TTAAAAAGGAGCGCAACAAAGGGTACAAGCACCATGCCGAAGATGCTCTGATTATCGCAAATGCCGACTT
CATCTTTAAGGAGTGGAAAAAGCTGGACAAAGCCAAGAAAGTGATGGAGAACCAGATGTTCGAAGAGAAG
CAGGCCGAATCTATGCCCGAAATCGAGACAGAACAGGAGTACAAGGAGATTTTCATCACTCCTCACCAGA
TCAAGCATATCAAGGATTTCAAGGACTACAAGTACTCTCACCGGGTGGATAAAAAGCCCAACAGAGAGCT
GATCAATGACACCCTGTATAGTACAAGAAAAGACGATAAGGGGAATACCCTGATTGTGAACAATCTGAAC
GGACTGTACGACAAAGATAATGACAAGCTGAAAAAGCTGATCAACAAAAGTCCCGAGAAGCTGCTGATGT
ACCACCATGATCCTCAGACATATCAGAAACTGAAGCTGATTATGGAGCAGTACGGCGACGAGAAGAACCC
ACTGTATAAGTACTATGAAGAGACTGGGAACTACCTGACCAAGTATAGCAAAAAGGATAATGGCCCCGTG
ATCAAGAAGATCAAGTACTATGGGAACAAGCTGAATGCCCATCTGGACATCACAGACGATTACCCTAACA
GTCGCAACAAGGTGGTCAAGCTGTCACTGAAGCCATACAGATTCGATGTCTATCTGGACAACGGCGTGTA
TAAATTTGTGACTGTCAAGAATCTGGATGTCATCAAAAAGGAGAACTACTATGAAGTGAATAGCAAGTGC
TACGAAGAGGCTAAAAAGCTGAAAAAGATTAGCAACCAGGCAGAGTTCATCGCCTCCTTTTACAACAACG
ACCTGATTAAGATCAATGGCGAACTGTATAGGGTCATCGGGGTGAACAATGATCTGCTGAACCGCATTGA
AGTGAATATGATTGACATCACTTACCGAGAGTATCTGGAAAACATGAATGATAAGCGCCCCCCTCGAATT
ATCAAAACAATTGCCTCTAAGACTCAGAGTATCAAAAAGTACTCAACCGACATTCTGGGAAACCTGTATG
AGGTGAAGAGCAAAAAGCACCCTCAGATTATCAAAAAGGGCagcggaggcaagcgtcctgctgctactaa
gaaagctggtcaagctaagaaaaagaaaggatcctacccatacgatgttccagattacgcttaagaattc
ctagagctcgctgatcagcctcgactgtgccttctagttgccagccatctgttgtttgcccctcccccgt
gccttccttgaccctggaaggtgccactcccactgtcctttcctaataaaatgaggaaattgcatcgcat
tgtctgagtaggtgtcattctattctggggggtggggggggcaggacagcaagggggaggattgggaag
agaatagcaggcatgctggggaggtagcggccgcCCgcggtggagctccagcttttgttccctttagtga
gggttaattgcgcgcttggcgtaatcatggtcatagctgtttcctgtgtgaaattgttatccgctcacaa
ttccacacaacatacgagccggaagcataaagtgtaaagcctggggtgcctaatgagtgagctaactcac
attaattgcgttgcgctcactgcccgctttccagtcgggaaacctgtcgtgccagctgcattaatgaatc
ggccaacgcgcggggagaggcggtttgcgtattgggcgctcttccgcttcctcgctcactgactcgctgc
gctcggtcgttcggctgcggcgagcggtatcagctcactcaaaggcggtaatacggttatccacagaatc
aggggataacgcaggaaagaacatgtgagcaaaaggccagcaaaaggccaggaaccgtaaaaaggccgcg
ttgctggcgtttttccataggctccgcccccctgacgagcatcacaaaaatcgacgctcaagtcagaggt
ggcgaaacccgacaggactataaagataccaggcgtttccccctggaagctccctcgtgcgctctcctgt
tccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccg
ttcagcccgaccgctgcgccttatccggtaactatcgtcttgagtccaacccggtaagacacgacttatc
gccactggcagcagccactggtaacaggattagcagagcgaggtatgtaggcggtgctacagagttcttg
aagtggtggcctaactacggctacactagaaggacagtatttggtatctgcgctctgctgaagccagtta
ccttcggaaaaagagttggtagctcttgatccggcaaacaaaccaccgctggtagcggtggtttttttgt
ttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcacct
agatccttttaaattaaaaatgaagttttaaatcaatctaaagtatatatgagtaaacttggtctgacag
ttaccaatgcttaatcagtgaggcacctatctcagcgatctgtctatttcgttcatccatagttgcctga
ctccccgtcgtgtagataactacgatacgggagggcttaccatctggccccagtgctgcaatgataccgc
gagacccacgctcaccggctccagatttatcagcaataaaccagccagccggaagggccgagcgcagaag
tggtcctgcaactttatccgcctccatccagtctattaattgttgccgggaagctagagtaagtagttcg
ccagttaatagtttgcgcaacgttgttgccattgctacaggcatcgtggtgtcacgctcgtcgtttggta
tggcttcattcagctccggttcccaacgatcaaggcgagttacatgatcccccatgttgtgcaaaaaagc
ggttagctccttcggtcctccgatcgttgtcagaagtaagttggccgcagtgttatcactcatggttatg
gcagcactgcataattctcttactgtcatgccatccgtaagatgcttttctgtgactggtgagtactcaa
ccaagtcattctgagaatagtgtatgcggcgaccgagttgctcttgcccggcgtcaatacgggataatac
cgcgccacatagcagaactttaaaagtgctcatcattggaaaacgttcttcggggcgaaaactctcaagg
atcttaccgctgttgagatccagttcgatgtaacccactcgtgcacccaactgatcttcagcatctttta
ctttcaccagcgtttctgggtgagcaaaaacaggaaggcaaaatgccgcaaaaaagggaataagggcgac
acggaaatgttgaatactcatactcttcctttttcaatattattgaagcatttatcagggttattgtctc
atgagcggatacatatttgaatgtatttagaaaaataaacaaataggggttccgcgcacatttccccgaa
aagtgccac
SEQ ID NO: 35
Human p300 (with L553M mutation) protein
MAENVVEPGPPSAKRPKLSSPALSASASDGTDFGSLEDLEHDLPDELINSTELGLINGGDINQLQTSLGM
VQDAASKHKQLSELLRSGSSPNLNMGVGGPGQVMASQAQQSSPGLGLINSMVKSPMTQAGLTSPNMGMGT
SGPNQGPTQSTGMMNSPVNQPAMGMNTGMNAGMNPGMLAAGNGQGIMPNQVMNGSIGAGRGRQNMQYPNP
GMGSAGNLLTEPLQQGSPQMGGQTGLRGPQPLKMGMMNNPNPYGSPYTQNPGQQIGASGLGLQIQTKTVL
SNNLSPFAMDKKAVPGGGMPNMGQQPAPQVQQPGLVTPVAQGMGSGAHTADPEKRKLIQQQLVLLLHAHK
CQRREQANGEVRQCNLPHCRTMKNVLNHMTHCQSGKSCQVAHCASSRQIISHWKNCTRHDCPVCLPLKNA
GDKRNQQPILTGAPVGLGNPSSLGVGQQSAPNLSTVSQIDPSSIERAYAALGLPYQVNQMPTQPQVQAKN
QQNQQPGQSPQGMRPMSNMSASPMGVNGGVGVQTPSLLSDSMLHSAINSQNPMMSENASVPSMGPMPTAA
QPSTTGIRKQWHEDITQDLRNHLVHKLVQAIFPTPDPAALKDRRMENLVAYARKVEGDMYESANNRAEYY
HLLAEKIYKIQKELEEKRRTRLQKQNMLPNAAGMVPVSMNPGPNMGQPQPGMTSNGPLPDPSMIRGSVPN
QMMPRITPQSGLNQFGQMSMAQPPIVPRQTPPLQHHGQLAQPGALNPPMGYGPRMQQPSNQGQFLPQTQF
PSQGMNVTNIPLAPSSGQAPVSQAQMSSSSCPVNSPIMPPGSQGSHIHCPQLPQPALHQNSPSPVPSRTP
TPHHTPPSIGAQQPPATTIPAPVPTPPAMPPGPQSQALHPPPRQTPTPPTTQLPQQVQPSLPAAPSADQP
QQQPRSQQSTAASVPTPTAPLLPPQPATPLSQPAVSIEGQVSNPPSTSSTEVNSQAIAEKQPSQEVKMEA
KMEVDQPEPADTQPEDISESKVEDCKMESTETEERSTELKTEIKEEEDQPSTSATQSSPAPGQSKKKIFK
PEELRQALMPTLEALYRQDPESLPFRQPVDPQLLGIPDYFDIVKSPMDLSTIKRKLDTGQYQEPWQYVDD
IWLMENNAWLYNRKTSRVYKYCSKLSEVFEQEIDPVMQSLGYCCGRKLEFSPQTLCCYGKQLCTIPRDAT
YYSYQNRYHFCEKCFNEIQGESVSLGDDPSQPQTTINKEQFSKRKNDTLDPELFVECTECGRKMHQICVL
HHEIIWPAGFVCDGCLKKSARTRKENKFSAKRLPSTRLGTFLENRVNDELRRQNHPESGEVTVRVVHASD
KTVEVKPGMKARFVDSGEMAESFPYRTKALFAFEEIDGVDLCFFGMHVQEYGSDCPPPNQRRVYISYLDS
VHFFRPKCLRTAVYHEILIGYLEYVKKLGYTTGHIWACPPSEGDDYIFHCHPPDQKIPKPKRLQEWYKKM
LDKAVSERIVHDYKDIFKQATEDRLTSAKELPYFEGDFWPNVLEESIKELEQEEEERKREENTSNESTDV
TKGDSKNAKKKNNKKTSKNKSSLSRGNKKKPGMPNVSNDLSQKLYATMEKHKEVFFVIRLIAGPAANSLP
PIVDPDPLIPCDLMDGRDAFLTLARDKHLEFSSLRRAQWSTMCMLVELHTQSQDRFVYTCNECKHHVETR
WHCTVCEDYDLCITCYNTKNHDHKMEKLGLGLDDESNNQQAAATQSPGDSRRLSIQRCIQSLVHACQCRN
ANCSLPSCQKMKRVVQHTKGCKRKTNGGCPICKQLIALCCYHAKHCQENKCPVPFCLNIKQKLRQQQLQH
RLQQAQMLRRRMASMQRTGVVGQQQGLPSPTPATPTTPTGQQPTTPQTPQPTSQPQPTPPNSMPPYLPRT
QAAGPVSQGKAAGQVTPPTPPQTAQPPLPGPPPAAVEMAMQIQRAAETQRQMAHVQIFQRPIQHQMPPMT
PMAPMGMNPPPMTRGPSGHLEPGMGPTGMQQQPPWSQGGLPQPQQLQSGMPRPAMMSVAQHGQPLNMAPQ
PGLGQVGISPLKPGTVSQQALQNLLRTLRSPSSPLQQQQVLSILHANPQLLAAFIKQRAAKYANSNPQPI
PGQPGMPQGQPGLQPPTMPGQQGVHSNPAMQNMNPMQAGVQRAGLPQQQPQQQLQPPMGGMSPQAQQMNM
NHNTMPSQFRDILRRQQMMQQQQQQGAGPGIGPGMANHNQFQQPQGVGYPPQQQQRMQHHMQQMQQGNMG
QIGQLPQALGAEAGASLQAYQQRLLQQQMGSPVQPNPMSPQQHMLPNQAQSPHLQGQQIPNSLSNQVRSP
QPVPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 36
Human p300 Core Effector protein (aa 1048-1664 of SEQ ID NO: 35)
IFKPEELRQALMPTLEALYRQDPESLPFRQPVDPQLLGIPDYFDIVKSPMDLSTIKRKLDTGQYQEPWQY
VDDIWLMENNAWLYNRKTSRVYKYCSKLSEVFEQEIDPVMQSLGYCCGRKLEFSPQTLCCYGKQLCTIPR
DATYYSYQNRYHFCEKCFNEIQGESVSLGDDPSQPQTTINKEQFSKRKNDTLDPELFVECTECGRKMHQI
CVLHHEIIWPAGFVCDGCLKKSARTRKENKFSAKRLPSTRLGTFLENRVNDELRRQNHPESGEVTVRVVH
ASDKTVEVKPGMKARFVDSGEMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYIFHCHPPDQKIPKPKRLQEWY
KKMLDKAVSERIVHDYKDIFKQATEDRLTSAKELPYFEGDEWPNVLEESIKELEQEEEERKREENTSNES
TDVTKGDSKNAKKKNNKKTSKNKSSLSRGNKKKPGMPNVSNDLSQKLYATMEKHKEVFFVIRLIAGPAAN
SLPPIVDPDPLIPCDLMDGRDAFLTLARDKHLEFSSLRRAQWSTMCMLVELHTQSQD
SEQ ID NO: 37
VP64-dCas9-VP64 protein
RADALDDEDLDMLGSDALDDEDLDMLGSDALDDEDLDMLGSDALDDEDLDMVNPKKKRKVGRGMDKKYSI
GLAIGTNSVGWAVITDEYKVPSKKFKVLGNTDRHSIKKNLIGALLFDSGETAEATRLKRTARRRYTRRKN
RICYLQEIFSNEMAKVDDSFFHRLEESELVEEDKKHERHPIFGNIVDEVAYHEKYPTIYHLRKKLVDSTD
KADLRLIYLALAHMIKERGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTEDNGSIPHQIHLGELHAILRRQE
DFYPELKDNREKIEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFKTNRKVTVKQLKED
YFKKIECFDSVEISGVEDRENASLGTYHDLLKIIKDKDELDNEENEDILEDIVLTLTLFEDREMIEERLK
TYAHLFDDKVMKQLKRRRYTGWGRLSRKLINGIRDKQSGKTILDELKSDGFANRNFMQLIHDDSLTFKED
IQKAQVSGQGDSLHEHIANLAGSPAIKKGILQTVKVVDELVKVMGRHKPENIVIEMARENQTTQKGQKNS
RERMKRIEEGIKELGSQILKEHPVENTQLQNEKLYLYYLQNGRDMYVDQELDINRLSDYDVDAIVPQSFL
KDDSIDNKVLTRSDKNRGKSDNVPSEEVVKKMKNYWRQLLNAKLITQRKEDNLTKAERGGLSELDKAGFI
KRQLVETRQITKHVAQILDSRMNTKYDENDKLIREVKVITLKSKLVSDERKDFQFYKVREINNYHHAHDA
YLNAVVGTALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLETLTNLGAPAAFKYEDTTIDRKRYT
STKEVLDATLIHQSITGLYETRIDLSQLGGDSRADPKKKRKVASRADALDDFDLDMLGSDALDDEDLDML
GSDALDDFDLDMLGSDALDDEDLDMLI
SEQ ID NO: 38
VP64-dCas9-VP64 DNA
cgggctgacgcattggacgattttgatctggatatgctgggaagtgacgccctcgatgattttgaccttg
acatgcttggttcggatgcccttgatgactttgacctcgacatgctcggcagtgacgcccttgatgattt
cgacctggacatggttaaccccaagaagaagaggaaggtgggccgcggaatggacaagaagtactccatt
gggctcgccatcggcacaaacagcgtcggctgggccgtcattacggacgagtacaaggtgccgagcaaaa
aattcaaagttctgggcaataccgatcgccacagcataaagaagaacctcattggcgccctcctgttcga
ctccggggaaaccgccgaagccacgcggctcaaaagaacagcacggcgcagatatacccgcagaaagaat
cggatctgctacctgcaggagatctttagtaatgagatggctaaggtggatgactctttcttccataggc
tggaggagtcctttttggtggaggaggataaaaagcacgagcgccacccaatctttggcaatatcgtgga
cgaggtggcgtaccatgaaaagtacccaaccatatatcatctgaggaagaagcttgtagacagtactgat
aaggctgacttgcggttgatctatctcgcgctggcgcatatgatcaaatttcggggacacttcctcatcg
agggggacctgaacccagacaacagcgatgtcgacaaactctttatccaactggttcagacttacaatca
gcttttcgaagagaacccgatcaacgcatccggagttgacgccaaagcaatcctgagcgctaggctgtcc
aaatcccggcggctcgaaaacctcatcgcacagctccctggggagaagaagaacggcctgtttggtaatc
ttatcgccctgtcactcgggctgacccccaactttaaatctaacttcgacctggccgaagatgccaagct
tcaactgagcaaagacacctacgatgatgatctcgacaatctgctggcccagatcggcgaccagtacgca
gacctttttttggcggcaaagaacctgtcagacgccattctgctgagtgatattctgcgagtgaacacgg
agatcaccaaagctccgctgagcgctagtatgatcaagcgctatgatgagcaccaccaagacttgacttt
gctgaaggcccttgtcagacagcaactgcctgagaagtacaaggaaattttcttcgatcagtctaaaaat
ggctacgccggatacattgacggcggagcaagccaggaggaattttacaaatttattaagcccatcttgg
aaaaaatggacggcaccgaggagctgctggtaaagcttaacagagaagatctgttgcgcaaacagcgcac
tttcgacaatggaagcatcccccaccagattcacctgggcgaactgcacgctatcctcaggcggcaagag
gatttctacccctttttgaaagataacagggaaaagattgagaaaatcctcacatttcggataccctact
atgtaggccccctcgcccggggaaattccagattcgcgtggatgactcgcaaatcagaagagaccatcac
tccctggaacttcgaggaagtcgtggataagggggcctctgcccagtccttcatcgaaaggatgactaac
tttgataaaaatctgcctaacgaaaaggtgcttcctaaacactctctgctgtacgagtacttcacagttt
ataacgagctcaccaaggtcaaatacgtcacagaagggatgagaaagccagcattcctgtctggagagca
gaagaaagctatcgtggacctcctcttcaagacgaaccggaaagttaccgtgaaacagctcaaagaagac
tatttcaaaaagattgaatgtttcgactctgttgaaatcagcggagtggaggatcgcttcaacgcatccc
tgggaacgtatcacgatctcctgaaaatcattaaagacaaggacttcctggacaatgaggagaacgagga
cattcttgaggacattgtcctcacccttacgttgtttgaagatagggagatgattgaagaacgcttgaaa
acttacgctcatctcttcgacgacaaagtcatgaaacagctcaagaggcgccgatatacaggatgggggc
ggctgtcaagaaaactgatcaatgggatccgagacaagcagagtggaaagacaatcctggattttcttaa
gtccgatggatttgccaaccggaacttcatgcagttgatccatgatgactctctcacctttaaggaggac
atccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaaggca
taagcccgagaatatcgttatcgagatggcccgagagaaccaaactacccagaagggacagaagaacagt
agggaaaggatgaagaggattgaagagggtataaaagaactggggtcccaaatccttaaggaacacccag
ttgaaaacacccagcttcagaatgagaagctctacctgtactacctgcagaacggcagggacatgtacgt
ggatcaggaactggacatcaatcggctctccgactacgacgtggatgccatcgtgccccagtcttttctc
aaagatgattctattgataataaagtgttgacaagatccgataaaaatagagggaagagtgataacgtcc
cctcagaagaagttgtcaagaaaatgaaaaattattggcggcagctgctgaacgccaaactgatcacaca
acggaagttcgataatctgactaaggctgaacgaggtggcctgtctgagttggataaagccggcttcatc
aaaaggcagcttgttgagacacgccagatcaccaagcacgtggcccaaattctcgattcacgcatgaaca
ccaagtacgatgaaaatgacaaactgattcgagaggtgaaagttattactctgaagtctaagctggtctc
agatttcagaaaggactttcagttttataaggtgagagagatcaacaattaccaccatgcgcatgatgcc
tacctgaatgcagtggtaggcactgcacttatcaaaaaatatcccaagcttgaatctgaatttgtttacg
gagactataaagtgtacgatgttaggaaaatgatcgcaaagtctgagcaggaaataggcaaggccaccgc
taagtacttcttttacagcaatattatgaattttttcaagaccgagattacactggccaatggagagatt
cggaagcgaccacttatcgaaacaaacggagaaacaggagaaatcgtgtgggacaagggtagggatttcg
cgacagtccggaaggtcctgtccatgccgcaggtgaacatcgttaaaaagaccgaagtacagaccggagg
cttctccaaggaaagtatcctcccgaaaaggaacagcgacaagctgatcgcacgcaaaaaagattgggac
cccaagaaatacggcggattcgattctcctacagtcgcttacagtgtactggttgtggccaaagtggaga
aagggaagtctaaaaaactcaaaagcgtcaaggaactgctgggcatcacaatcatggagcgatcaagctt
cgaaaaaaaccccatcgactttctcgaggcgaaaggatataaagaggtcaaaaaagacctcatcattaag
cttcccaagtactctctctttgagcttgaaaacggccggaaacgaatgctcgctagtgcgggcgagctgc
agaaaggtaacgagctggcactgccctctaaatacgttaatttcttgtatctggccagccactatgaaaa
gctcaaagggtctcccgaagataatgagcagaagcagctgttcgtggaacaacacaaacactaccttgat
gagatcatcgagcaaataagcgaattctccaaaagagtgatcctcgccgacgctaacctcgataaggtgc
tttctgcttacaataagcacagggataagcccatcagggagcaggcagaaaacattatccacttgtttac
tctgaccaacttgggcgcgcctgcagccttcaagtacttcgacaccaccatagacagaaagcggtacacc
tctacaaaggaggtcctggacgccacactgattcatcagtcaattacggggctctatgaaacaagaatcg
acctctctcagctcggtggagacagcagggctgaccccaagaagaagaggaaggtggctagccgcgccga
cgcgctggacgatttcgatctcgacatgctgggttctgatgccctcgatgactttgacctggatatgttg
ggaagcgacgcattggatgactttgatctggacatgctcggctccgatgctctggacgatttcgatctcg
atatgttaatc
SEQ ID NO: 39
Polynucleotide sequence encoding Streptococcus pyogenes dCas9-KRAB
atggactacaaagaccatgacggtgattataaagatcatgacatcgattacaaggatgacgatgacaaga
tggcccccaagaagaagaggaaggtgggccgcggaatggacaagaagtactccattgggctcgccatcgg
cacaaacagcgtcggctgggccgtcattacggacgagtacaaggtgccgagcaaaaaattcaaagttctg
ggcaataccgatcgccacagcataaagaagaacctcattggcgccctcctgttcgactccggggaaaccg
ccgaagccacgcggctcaaaagaacagcacggcgcagatatacccgcagaaagaatcggatctgctacct
gcaggagatctttagtaatgagatggctaaggtggatgactctttcttccataggctggaggagtccttt
ttggtggaggaggataaaaagcacgagcgccacccaatctttggcaatatcgtggacgaggtggcgtacc
atgaaaagtacccaaccatatatcatctgaggaagaagcttgtagacagtactgataaggctgacttgcg
gttgatctatctcgcgctggcgcatatgatcaaatttcggggacacttcctcatcgagggggacctgaac
ccagacaacagcgatgtcgacaaactctttatccaactggttcagacttacaatcagcttttcgaagaga
acccgatcaacgcatccggagttgacgccaaagcaatcctgagcgctaggctgtccaaatcccggcggct
cgaaaacctcatcgcacagctccctggggagaagaagaacggcctgtttggtaatcttatcgccctgtca
ctcgggctgacccccaactttaaatctaacttcgacctggccgaagatgccaagcttcaactgagcaaag
acacctacgatgatgatctcgacaatctgctggcccagatcggcgaccagtacgcagacctttttttggc
ggcaaagaacctgtcagacgccattctgctgagtgatattctgcgagtgaacacggagatcaccaaagct
ccgctgagcgctagtatgatcaagcgctatgatgagcaccaccaagacttgactttgctgaaggcccttg
tcagacagcaactgcctgagaagtacaaggaaattttcttcgatcagtctaaaaatggctacgccggata
cattgacggcggagcaagccaggaggaattttacaaatttattaagcccatcttggaaaaaatggacggc
accgaggagctgctggtaaagcttaacagagaagatctgttgcgcaaacagcgcactttcgacaatggaa
gcatcccccaccagattcacctgggcgaactgcacgctatcctcaggcggcaagaggatttctacccctt
tttgaaagataacagggaaaagattgagaaaatcctcacatttcggataccctactatgtaggccccctc
gcccggggaaattccagattcgcgtggatgactcgcaaatcagaagagaccatcactccctggaacttcg
aggaagtcgtggataagggggcctctgcccagtccttcatcgaaaggatgactaactttgataaaaatct
gcctaacgaaaaggtgcttcctaaacactctctgctgtacgagtacttcacagtttataacgagctcacc
aaggtcaaatacgtcacagaagggatgagaaagccagcattcctgtctggagagcagaagaaagctatcg
tggacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatcac
gatctcctgaaaatcattaaagacaaggacttcctggacaatgaggagaacgaggacattcttgaggaca
ttgtcctcacccttacgttgtttgaagatagggagatgattgaagaacgcttgaaaacttacgctcatct
cttcgacgacaaagtcatgaaacagctcaagaggcgccgatatacaggatgggggcggctgtcaagaaaa
ctgatcaatgggatccgagacaagcagagtggaaagacaatcctggattttcttaagtccgatggatttg
ccaaccggaacttcatgcagttgatccatgatgactctctcacctttaaggaggacatccagaaagcaca
agtttctggccagggggacagtcttcacgagcacatcgctaatcttgcaggtagcccagctatcaaaaag
ggaatactgcagaccgttaaggtcgtggatgaactcgtcaaagtaatgggaaggcataagcccgagaata
tcgttatcgagatggcccgagagaaccaaactacccagaagggacagaagaacagtagggaaaggatgaa
gaggattgaagagggtataaaagaactggggtcccaaatccttaaggaacacccagttgaaaacacccag
cttcagaatgagaagctctacctgtactacctgcagaacggcagggacatgtacgtggatcaggaactgg
acatcaatcggctctccgactacgacgtggatgccatcgtgccccagtcttttctcaaagatgattctat
tgataataaagtgttgacaagatccgataaaaatagagggaagagtgataacgtcccctcagaagaagtt
gtcaagaaaatgaaaaattattggcggcagctgctgaacgccaaactgatcacacaacggaagttcgata
atctgactaaggctgaacgaggtggcctgtctgagttggataaagccggcttcatcaaaaggcagcttgt
tgagacacgccagatcaccaagcacgtggcccaaattctcgattcacgcatgaacaccaagtacgatgaa
aatgacaaactgattcgagaggtgaaagttattactctgaagtctaagctggtctcagatttcagaaagg
actttcagttttataaggtgagagagatcaacaattaccaccatgcgcatgatgcctacctgaatgcagt
ggtaggcactgcacttatcaaaaaatatcccaagcttgaatctgaatttgtttacggagactataaagtg
tacgatgttaggaaaatgatcgcaaagtctgagcaggaaataggcaaggccaccgctaagtacttctttt
acagcaatattatgaattttttcaagaccgagattacactggccaatggagagattcggaagcgaccact
tatcgaaacaaacggagaaacaggagaaatcgtgtgggacaagggtagggatttcgcgacagtccggaag
gtcctgtccatgccgcaggtgaacatcgttaaaaagaccgaagtacagaccggaggcttctccaaggaaa
gtatcctcccgaaaaggaacagcgacaagctgatcgcacgcaaaaaagattgggaccccaagaaatacgg
cggattcgattctcctacagtcgcttacagtgtactggttgtggccaaagtggagaaagggaagtctaaa
aaactcaaaagcgtcaaggaactgctgggcatcacaatcatggagcgatcaagcttcgaaaaaaacccca
tcgactttctcgaggcgaaaggatataaagaggtcaaaaaagacctcatcattaagcttcccaagtactc
tctctttgagcttgaaaacggccggaaacgaatgctcgctagtgcgggcgagctgcagaaaggtaacgag
ctggcactgccctctaaatacgttaatttcttgtatctggccagccactatgaaaagctcaaagggtctc
ccgaagataatgagcagaagcagctgttcgtggaacaacacaaacactaccttgatgagatcatcgagca
aataagcgaattctccaaaagagtgatcctcgccgacgctaacctcgataaggtgctttctgcttacaat
aagcacagggataagcccatcagggagcaggcagaaaacattatccacttgtttactctgaccaacttgg
gcgcgcctgcagccttcaagtacttcgacaccaccatagacagaaagcggtacacctctacaaaggaggt
cctggacgccacactgattcatcagtcaattacggggctctatgaaacaagaatcgacctctctcagctc
ggtggagacagcagggctgaccccaagaagaagaggaaggtggctagcgatgctaagtcactgactgcct
ggtcccggacactggtgaccttcaaggatgtgtttgtggacttcaccagggaggagtggaagctgctgga
cactgctcagcagatcctgtacagaaatgtgatgctggagaactataagaacctggtttccttgggttat
cagcttactaagccagatgtgatcctccggttggagaagggagaagagccctggctggtggagagagaaa
ttcaccaagagacccatcctgattcagagactgcatttgaaatcaaatcatcagttccgaaaaagaaacg
caaagtttga
SEQ ID NO: 40
Polypeptide sequence of Streptococcus pyogenes dCas9-KRAB protein
MDYKDHDGDYKDHDIDYKDDDDKMAPKKKRKVGRGMDKKYSIGLAIGTNSVGWAVITDEYKVPSKKEKVL
GNTDRHSIKKNLIGALLEDSGETAEATRLKRTARRRYTRRKNRICYLQEIFSNEMAKVDDSFFHRLEESE
LVEEDKKHERHPIFGNIVDEVAYHEKYPTIYHLRKKLVDSTDKADLRLIYLALAHMIKERGHFLIEGDLN
PDNSDVDKLFIQLVQTYNQLFEENPINASGVDAKAILSARLSKSRRLENLIAQLPGEKKNGLFGNLIALS
LGLTPNFKSNEDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKPILEKMDG
TEELLVKLNREDLLRKQRTEDNGSIPHQIHLGELHAILRRQEDFYPFLKDNREKIEKILTFRIPYYVGPL
ARGNSRFAWMTRKSEETITPWNFEEVVDKGASAQSFIERMTNEDKNLPNEKVLPKHSLLYEYFTVYNELT
KVKYVTEGMRKPAFLSGEQKKAIVDLLFKTNRKVTVKQLKEDYFKKIECFDSVEISGVEDRENASLGTYH
DLLKIIKDKDELDNEENEDILEDIVLTLTLFEDREMIEERLKTYAHLEDDKVMKQLKRRRYTGWGRLSRK
LINGIRDKQSGKTILDELKSDGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDAIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSAYN
KHRDKPIREQAENIIHLFTLTNLGAPAAFKYEDTTIDRKRYTSTKEVLDATLIHQSITGLYETRIDLSQL
GGDSRADPKKKRKVASDAKSLTAWSRTLVTFKDVFVDFTREEWKLLDTAQQILYRNVMLENYKNLVSLGY
QLTKPDVILRLEKGEEPWLVEREIHQETHPDSETAFEIKSSVPKKKRKV
SEQ ID NO: 41
Polynucleotide sequence of Staphylococcus aureus dCas9-KRAB protein
atggccccaaagaagaagcggaaggtcggtatccacggagtcccagcagccaagcggaactacatcctgg
gcctggccatcggcatcaccagcgtgggctacggcatcatcgactacgagacacgggacgtgatcgatgc
cggcgtgcggctgttcaaagaggccaacgtggaaaacaacgagggcaggcggagcaagagaggcgccaga
aggctgaagcggcggaggcggcatagaatccagagagtgaagaagctgctgttcgactacaacctgctga
ccgaccacagcgagctgagcggcatcaacccctacgaggccagagtgaagggcctgagccagaagctgag
cgaggaagagttctctgccgccctgctgcacctggccaagagaagaggcgtgcacaacgtgaacgaggtg
gaagaggacaccggcaacgagctgtccaccaaagagcagatcagccggaacagcaaggccctggaagaga
aatacgtggccgaactgcagctggaacggctgaagaaagacggcgaagtgcggggcagcatcaacagatt
caagaccagcgactacgtgaaagaagccaaacagctgctgaaggtgcagaaggcctaccaccagctggac
cagagcttcatcgacacctacatcgacctgctggaaacccggcggacctactatgagggacctggcgagg
gcagccccttcggctggaaggacatcaaagaatggtacgagatgctgatgggccactgcacctacttccc
cgaggaactgcggagcgtgaagtacgcctacaacgccgacctgtacaacgccctgaacgacctgaacaat
ctcgtgatcaccagggacgagaacgagaagctggaatattacgagaagttccagatcatcgagaacgtgt
tcaagcagaagaagaagcccaccctgaagcagatcgccaaagaaatcctcgtgaacgaagaggatattaa
gggctacagagtgaccagcaccggcaagcccgagttcaccaacctgaaggtgtaccacgacatcaaggac
attaccgcccggaaagagattattgagaacgccgagctgctggatcagattgccaagatcctgaccatct
accagagcagcgaggacatccaggaagaactgaccaatctgaactccgagctgacccaggaagagatcga
gcagatctctaatctgaagggctataccggcacccacaacctgagcctgaaggccatcaacctgatcctg
gacgagctgtggcacaccaacgacaaccagatcgctatcttcaaccggctgaagctggtgcccaagaagg
tggacctgtcccagcagaaagagatccccaccaccctggtggacgacttcatcctgagccccgtcgtgaa
gagaagcttcatccagagcatcaaagtgatcaacgccatcatcaagaagtacggcctgcccaacgacatc
attatcgagctggcccgcgagaagaactccaaggacgcccagaaaatgatcaacgagatgcagaagcgga
accggcagaccaacgagcggatcgaggaaatcatccggaccaccggcaaagagaacgccaagtacctgat
cgagaagatcaagctgcacgacatgcaggaaggcaagtgcctgtacagcctggaagccatccctctggaa
gatctgctgaacaaccccttcaactatgaggtggaccacatcatccccagaagcgtgtccttcgacaaca
gcttcaacaacaaggtgctcgtgaagcaggaagaagccagcaagaagggcaaccggaccccattccagta
cctgagcagcagcgacagcaagatcagctacgaaaccttcaagaagcacatcctgaatctggccaagggc
aagggcagaatcagcaagaccaagaaagagtatctgctggaagaacgggacatcaacaggttctccgtgc
agaaagacttcatcaaccggaacctggtggataccagatacgccaccagaggcctgatgaacctgctgcg
gagctacttcagagtgaacaacctggacgtgaaagtgaagtccatcaatggcggcttcaccagctttctg
cggcggaagtggaagtttaagaaagagcggaacaaggggtacaagcaccacgccgaggacgccctgatca
ttgccaacgccgatttcatcttcaaagagtggaagaaactggacaaggccaaaaaagtgatggaaaacca
gatgttcgaggaaaagcaggccgagagcatgcccgagatcgaaaccgagcaggagtacaaagagatcttc
atcaccccccaccagatcaagcacattaaggacttcaaggactacaagtacagccaccgggtggacaaga
agcctaatagagagctgattaacgacaccctgtactccacccggaaggacgacaagggcaacaccctgat
cgtgaacaatctgaacggcctgtacgacaaggacaatgacaagctgaaaaagctgatcaacaagagcccc
gaaaagctgctgatgtaccaccacgacccccagacctaccagaaactgaagctgattatggaacagtacg
gcgacgagaagaatcccctgtacaagtactacgaggaaaccgggaactacctgaccaagtactccaaaaa
ggacaacggccccgtgatcaagaagattaagtattacggcaacaaactgaacgcccatctggacatcacc
gacgactaccccaacagcagaaacaaggtcgtgaagctgtccctgaagccctacagattcgacgtgtacc
tggacaatggcgtgtacaagttcgtgaccgtgaagaatctggatgtgatcaaaaaagaaaactactacga
agtgaatagcaagtgctatgaggaagctaagaagctgaagaagatcagcaaccaggccgagtttatcgcc
tccttctacaacaacgatctgatcaagatcaacggcgagctgtatagagtgatcggcgtgaacaacgacc
tgctgaaccggatcgaagtgaacatgatcgacatcacctaccgcgagtacctggaaaacatgaacgacaa
gaggccccccaggatcattaagacaatcgcctccaagacccagagcattaagaagtacagcacagacatt
ctgggcaacctgtatgaagtgaaatctaagaagcaccctcagatcatcaaaaagggcaaaaggccggcgg
ccacgaaaaaggccggccaggcaaaaaagaaaaagggatccgatgctaagtcactgactgcctggtcccg
gacactggtgaccttcaaggatgtgtttgtggacttcaccagggaggagtggaagctgctggacactgct
cagcagatcctgtacagaaatgtgatgctggagaactataagaacctggtttccttgggttatcagctta
ctaagccagatgtgatcctccggttggagaagggagaagagccctggctggtggagagagaaattcacca
agagacccatcctgattcagagactgcatttgaaatcaaatcatcagttccgaaaaagaaacgcaaagtt
SEQ ID NO: 42
Polypeptide sequence of Staphylococcus aureus dCas9-KRAB protein
MAPKKKRKVGIHGVPAAKRNYILGLAIGITSVGYGIIDYETRDVIDAGVRLFKEANVENNEGRRSKRGAR
RLKRRRRHRIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKVQKAYHQLD
QSFIDTYIDLLETRRTYYEGPGEGSPFGWKDIKEWYEMLMGHCTYFPEELRSVKYAYNADLYNALNDLNN
LVITRDENEKLEYYEKFQIIENVFKQKKKPTLKQIAKEILVNEEDIKGYRVTSTGKPEFTNLKVYHDIKD
ITARKEIIENAELLDQIAKILTIYQSSEDIQEELTNLNSELTQEEIEQISNLKGYTGTHNLSLKAINLIL
DELWHTNDNQIAIFNRLKLVPKKVDLSQQKEIPTTLVDDFILSPVVKRSFIQSIKVINAIIKKYGLPNDI
IIELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEEASKKGNRTPFQYLSSSDSKISYETFKKHILNLAKG
KGRISKTKKEYLLEERDINRFSVQKDFINRNLVDTRYATRGLMNLLRSYFRVNNLDVKVKSINGGFTSEL
RRKWKFKKERNKGYKHHAEDALIIANADFIFKEWKKLDKAKKVMENQMFEEKQAESMPEIETEQEYKEIF
ITPHQIKHIKDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGNKLNAHLDIT
DDYPNSRNKVVKLSLKPYRFDVYLDNGVYKFVTVKNLDVIKKENYYEVNSKCYEEAKKLKKISNQAEFIA
SFYNNDLIKINGELYRVIGVNNDLLNRIEVNMIDITYREYLENMNDKRPPRIIKTIASKTQSIKKYSTDI
LGNLYEVKSKKHPQIIKKGKRPAATKKAGQAKKKKGSDAKSLTAWSRTLVTFKDVFVDFTREEWKLLDTA
QQILYRNVMLENYKNLVSLGYQLTKPDVILRLEKGEEPWLVEREIHQETHPDSETAFEIKSSVPKKKRKV
SEQ ID NO: 43
Polynucleotide sequence of Tet1CD
CTGCCCACCTGCAGCTGTCTTGATCGAGTTATACAAAAAGACAAAGGCCCATATTATACACACCTTGGGG
CAGGACCAAGTGTTGCTGCTGTCAGGGAAATCATGGAGAATAGGTATGGTCAAAAAGGAAACGCAATAAG
GATAGAAATAGTAGTGTACACCGGTAAAGAAGGGAAAAGCTCTCATGGGTGTCCAATTGCTAAGTGGGTT
TTAAGAAGAAGCAGTGATGAAGAAAAAGTTCTTTGTTTGGTCCGGCAGCGTACAGGCCACCACTGTCCAA
CTGCTGTGATGGTGGTGCTCATCATGGTGTGGGATGGCATCCCTCTTCCAATGGCCGACCGGCTATACAC
AGAGCTCACAGAGAATCTAAAGTCATACAATGGGCACCCTACCGACAGAAGATGCACCCTCAATGAAAAT
CGTACCTGTACATGTCAAGGAATTGATCCAGAGACTTGTGGAGCTTCATTCTCTTTTGGCTGTTCATGGA
GTATGTACTTTAATGGCTGTAAGTTTGGTAGAAGCCCAAGCCCCAGAAGATTTAGAATTGATCCAAGCTC
TCCCTTACATGAAAAAAACCTTGAAGATAACTTACAGAGTTTGGCTACACGATTAGCTCCAATTTATAAG
CAGTATGCTCCAGTAGCTTACCAAAATCAGGTGGAATATGAAAATGTTGCCCGAGAATGTCGGCTTGGCA
GCAAGGAAGGTCGACCCTTCTCTGGGGTCACTGCTTGCCTGGACTTCTGTGCTCATCCCCACAGGGACAT
TCACAACATGAATAATGGAAGCACTGTGGTTTGTACCTTAACTCGAGAAGATAACCGCTCTTTGGGTGTT
ATTCCTCAAGATGAGCAGCTCCATGTGCTACCTCTTTATAAGCTTTCAGACACAGATGAGTTTGGCTCCA
AGGAAGGAATGGAAGCCAAGATCAAATCTGGGGCCATCGAGGTCCTGGCACCCCGCCGCAAAAAAAGAAC
GTGTTTCACTCAGCCTGTTCCCCGTTCTGGAAAGAAGAGGGCTGCGATGATGACAGAGGTTCTTGCACAT
AAGATAAGGGCAGTGGAAAAGAAACCTATTCCCCGAATCAAGCGGAAGAATAACTCAACAACAACAAACA
ACAGTAAGCCTTCGTCACTGCCAACCTTAGGGAGTAACACTGAGACCGTGCAACCTGAAGTAAAAAGTGA
AACCGAACCCCATTTTATCTTAAAAAGTTCAGACAACACTAAAACTTATTCGCTGATGCCATCCGCTCCT
CACCCAGTGAAAGAGGCATCTCCAGGCTTCTCCTGGTCCCCGAAGACTGCTTCAGCCACACCAGCTCCAC
TGAAGAATGACGCAACAGCCTCATGCGGGTTTTCAGAAAGAAGCAGCACTCCCCACTGTACGATGCCTTC
GGGAAGACTCAGTGGTGCCAATGCTGCAGCTGCTGATGGCCCTGGCATTTCACAGCTTGGCGAAGTGGCT
CCTCTCCCCACCCTGTCTGCTCCTGTGATGGAGCCCCTCATTAATTCTGAGCCTTCCACTGGTGTGACTG
AGCCGCTAACGCCTCATCAGCCAAACCACCAGCCCTCCTTCCTCACCTCTCCTCAAGACCTTGCCTCTTC
TCCAATGGAAGAAGATGAGCAGCATTCTGAAGCAGATGAGCCTCCATCAGACGAACCCCTATCTGATGAC
CCCCTGTCACCTGCTGAGGAGAAATTGCCCCACATTGATGAGTATTGGTCAGACAGTGAGCACATCTTTT
TGGATGCAAATATTGGTGGGGTGGCCATCGCACCTGCTCACGGCTCGGTTTTGATTGAGTGTGCCCGGCG
AGAGCTGCACGCTACCACTCCTGTTGAGCACCCCAACCGTAATCATCCAACCCGCCTCTCCCTTGTCTTT
TACCAGCACAAAAACCTAAATAAGCCCCAACATGGTTTTGAACTAAACAAGATTAAGTTTGAGGCTAAAG
AAGCTAAGAATAAGAAAATGAAGGCCTCAGAGCAAAAAGACCAGGCAGCTAATGAAGGTCCAGAACAGTC
CTCTGAAGTAAATGAATTGAACCAAATTCCTTCTCATAAAGCATTAACATTAACCCATGACAATGTTGTC
ACCGTGTCCCCTTATGCTCTCACACACGTTGCGGGGCCCTATAACCATTGGGTC
SEQ ID NO: 44
Polypeptide sequence of Tet1CD
LPTCSCLDRVIQKDKGPYYTHLGAGPSVAAVREIMENRYGQKGNAIRIEIVVYTGKEGKSSHGCPIAKWV
LRRSSDEEKVLCLVRQRTGHHCPTAVMVVLIMVWDGIPLPMADRLYTELTENLKSYNGHPTDRRCTLNEN
RTCTCQGIDPETCGASESFGCSWSMYFNGCKFGRSPSPRRFRIDPSSPLHEKNLEDNLQSLATRLAPIYK
QYAPVAYQNQVEYENVARECRLGSKEGRPFSGVTACLDFCAHPHRDIHNMNNGSTVVCTLTREDNRSLGV
IPQDEQLHVLPLYKLSDTDEFGSKEGMEAKIKSGAIEVLAPRRKKRTCFTQPVPRSGKKRAAMMTEVLAH
KIRAVEKKPIPRIKRKNNSTTTNNSKPSSLPTLGSNTETVQPEVKSETEPHFILKSSDNTKTYSLMPSAP
HPVKEASPGFSWSPKTASATPAPLKNDATASCGFSERSSTPHCTMPSGRLSGANAAAADGPGISQLGEVA
PLPTLSAPVMEPLINSEPSTGVTEPLTPHQPNHQPSELTSPQDLASSPMEEDEQHSEADEPPSDEPLSDD
PLSPAEEKLPHIDEYWSDSEHIELDANIGGVAIAPAHGSVLIECARRELHATTPVEHPNRNHPTRLSLVE
YQHKNLNKPQHGFELNKIKFEAKEAKNKKMKASEQKDQAANEGPEQSSEVNELNQIPSHKALTLTHDNVV
TVSPYALTHVAGPYNHWV
SEQ ID NO: 45
Protein sequence for VPH
DALDDEDLDMLGSDALDDFDLDMLGSDALDDEDLDMLGSDALDDEDLDMLGSLPSASVEFEGSGGPSGQI
SNQALALAPSSAPVLAQTMVPSSAMVPLAQPPAPAPVLTPGPPQSLSAPVPKSTQAGEGTLSEALLHLQF
DADEDLGALLGNSTDPGVFTDLASVDNSEFQQLLNQGVSMSHSTAEPMLMEYPEAITRLVTGSQRPPDPA
PTPLGTSGLPNGLSGDEDFSSIADMDFSALLSQISSSGQGGGGSGFSVDTSALLDLESPSVTVPDMSLPD
LDSSLASIQELLSPQEPPRPPEAENSSPDSGKQLVHYTAQPLFLLDPGSVDTGSNDLPVLFELGEGSYES
EGDGFAEDPTISLLTGSEPPKAKDPTVS
SEQ ID NO: 46
DNA sequence for VPH
Gatgctttagacgattttgacttagatatgcttggttcagacgcgttagacgacttcgacctagacatgt
taggctcagatgcattggacgacttcgatttagatatgttgggctccgatgccctagatgactttgatct
agatatgctagggtcactacccagcgccagcgtcgagttcgaaggcagcggcgggccttcagggcagatc
agcaaccaggccctggctctggcccctagctccgctccagtgctggcccagactatggtgccctctagtg
ctatggtgcctctggcccagccacctgctccagcccctgtgctgaccccaggaccaccccagtcactgag
cgccccagtgcccaagtctacacaggccggcgaggggactctgagtgaagctctgctgcacctgcagttc
gacgctgatgaggacctgggagctctgctggggaacagcaccgatcccggagtgttcacagatctggcct
ccgtggacaactctgagtttcagcagctgctgaatcagggcgtgtccatgtctcatagtacagccgaacc
aatgctgatggagtaccccgaagccattacccggctggtgaccggcagccagcggccccccgaccccgct
ccaactcccctgggaaccagcggcctgcctaatgggctgtccggagatgaagacttctcaagcatcgctg
atatggactttagtgccctgctgtcacagatttcctctagtgggcagggaggaggtggaagcggcttcag
cgtggacaccagtgccctgctggacctgttcagcccctcggtgaccgtgcccgacatgagcctgcctgac
cttgacagcagcctggccagtatccaagagctcctgtctccccaggagccccccaggcctcccgaggcag
agaacagcagcccggattcagggaagcagctggtgcactacacagcgcagccgctgttcctgctggaccc
cggctccgtggacaccgggagcaacgacctgccggtgctgtttgagctgggagagggctcctacttctcc
gaaggggacggcttcgccgaggaccccaccatctccctgctgacaggctcggagcctcccaaagccaagg
accccactgtctcc
SEQ ID NO: 47
Protein sequence for VPR
DALDDEDLDMLGSDALDDEDLDMLGSDALDDEDLDMLGSDALDDEDLDMLGSPKKKRKVGSQYLPDTDDR
HRIEEKRKRTYETFKSIMKKSPFSGPTDPRPPPRRIAVPSRSSASVPKPAPQPYPFTSSLSTINYDEFPT
MVFPSGQISQASALAPAPPQVLPQAPAPAPAPAMVSALAQAPAPVPVLAPGPPQAVAPPAPKPTQAGEGT
LSEALLQLQFDDEDLGALLGNSTDPAVFTDLASVDNSEFQQLLNQGIPVAPHTTEPMLMEYPEAITRLVT
GAQRPPDPAPAPLGAPGLPNGLLSGDEDESSIADMDFSALLSQISSGSGSGSRDSREGMFLPKPEAGSAI
SDVFEGREVCQPKRIRPFHPPGSPWANRPLPASLAPTPTGPVHEPVGSLTPAPVPQPLDPAPAVTPEASH
LLEDPDEETSQAVKALREMADTVIPQKEEAAICGQMDLSHPPPRGHLDELTTTLESMTEDLNLDSPLTPE
LNEILDTFLNDECLLHAMHISTGLSIFDTSLF
SEQ ID NO: 48
DNA sequence for VPR
gatgctttagacgattttgacttagatatgcttggttcagacgcgttagacgacttcgacctagacatgt
taggctcagatgcattggacgacttcgatttagatatgttgggctccgatgccctagatgactttgatct
agatatgctaggtagtcccaaaaagaagaggaaagtgggatcccagtatctgcccgacacagatgataga
caccgaatcgaagagaaacgcaagcgaacgtatgaaaccttcaaatcgatcatgaagaaatcgcccttct
cgggtccgaccgatcccaggcccccaccgagaaggattgcggtcccgtcccgctcgtcggccagcgtgcc
gaagcctgcgccgcagccctaccccttcacgtcgagcctgagcacaatcaattatgacgagttcccgacg
atggtgttcccctcgggacaaatctcacaagcctcggcgctcgcaccagcgcctccccaagtccttccgc
aagcgcctgccccagcgcctgcaccggcaatggtgtccgccctcgcacaggcccctgcgcccgtccccgt
gctcgcgcctggaccgccccaggcggtcgctccaccggctccgaagccgacgcaggccggagagggaaca
ctctccgaagcacttcttcaactccagtttgatgacgaggatcttggagcactccttggaaactcgacag
accctgcggtgtttaccgacctcgcgtcagtagataactccgaatttcagcagcttttgaaccagggtat
cccggtcgcgccacatacaacggagcccatgttgatggaataccccgaagcaatcacgagacttgtgacg
ggagcgcagcggcctcccgatcccgcacccgcacctttgggggcacctggcctccctaacggacttttga
gcggcgacgaggatttctcctccatcgccgatatggatttctcagccttgctgtcacagatttccagcgg
ctctggcagcggcagccgggattccagggaagggatgtttttgccgaagcctgaggccggctccgctatt
agtgacgtgtttgagggccgcgaggtgtgccagccaaaacgaatccggccatttcatcctccaggaagtc
catgggccaaccgcccactccccgccagcctcgcaccaacaccaaccggtccagtacatgagccagtcgg
gtcactgaccccggcaccagtccctcagccactggatccagcgcccgcagtgactcccgaggccagtcac
ctgttggaggatcccgatgaagagacgagccaggctgtcaaagcccttcgggagatggccgatactgtga
ttccccagaaggaagaggctgcaatctgtggccaaatggacctttcccatccgcccccaaggggccatct
ggatgagctgacaaccacacttgagtccatgaccgaggatctgaacctggactcacccctgaccccggaa
ttgaacgagattctggataccttcctgaacgacgagtgcctcttgcatgccatgcatatcagcacaggac
tgtccatcttcgacacatctctgttt
SEQ ID NO: 49
SV40 NLS (Pro-Lys-Lys-Lys-Arg-Lys-Val)
SEQ ID NO: 50
GS linker (Gly-Gly-Gly-Gly-Ser)n, wherein n is an integer between 0 and 10
SEQ ID NO: 51
Gly-Gly-Gly-Gly-Gly
SEQ ID NO: 52
Gly-Gly-Ala-Gly-Gly
SEQ ID NO: 53
Gly-Gly-Gly-Gly-Ser-Ser-Ser
SEQ ID NO: 54
Gly-Gly-Gly-Gly-Ala-Ala-Ala
SEQ ID NO: 55
Polypeptide sequence of KRAB protein
RTLVTFKDVFVDFTREEWKLLDTAQQILYRNVMLENYKNLVSLGYQLTKPDVILRLEKGEEPWL
V
SEQ ID NO: 56
Polynucleotide sequence for KRAB
cggacactggtgaccttcaaggatgtgtttgtggacttcaccagggaggagtggaagctgctgg
acactgctcagcagatcctgtacagaaatgtgatgctggagaactataagaacctggtttcctt
gggttatcagcttactaagccagatgtgatcctccggttggagaagggagaagagccctggctg
gtg

### Sequences

1. A composition for treating leukemia, the composition comprising:
a Cas9 protein or a fusion protein, wherein the fusion protein comprises two heterologous polypeptide domains, wherein the first polypeptide domain comprises a Cas9 protein and the second polypeptide domain has an activity selected from the group consisting of transcription activation activity, transcription repression activity, transcription release factor activity, histone modification activity, nuclease activity, nucleic acid association activity, methylase activity, and demethylase activity; and
at least one guide RNA (gRNA) that targets the Cas9 protein to a regulatory element of a target gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GM PR.

2. The composition of claim 1, wherein the gRNA targets the Cas9 protein to a polynucleotide sequence comprising a sequence selected from SEQ ID NOs: 339-479.

3. The composition of claim 1, wherein the gRNA is encoded by a polynucleotide sequence comprising a sequence selected from SEQ ID NOs: 57-197 or comprises a polynucleotide sequence comprising a sequence selected from SEQ ID NOs: 198-338.

4. The composition of claim 1, wherein the composition inhibits cell viability, and wherein the target gene is selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1.

5-7. (canceled)

8. The composition of claim 1, wherein the composition increases cell viability, and wherein the target gene is selected from FADS3, RPAP1, SLC25A39, RP13-20L14.6, FOXA2, and GMPR.

9-12. (canceled)

13. The composition of claim 1, wherein the Cas9 protein comprises an amino acid sequence having at least 90% or greater identity to a sequence selected from SEQ ID NOs: 20-23, or any fragment thereof,
or wherein the Cas9 protein is encoded by a polynucleotide comprising a sequence having at least 90% or greater identity to a sequence selected from SEQ ID NOs: 24-26, or any fragment thereof,
or wherein the Cas9 protein comprises an amino acid sequence having one, two, three, four, five or more changes selected from amino acid substitutions, insertions, or deletions, relative to a sequence selected from SEQ ID NOs: 20-23, or any fragment thereof,
or wherein the Cas9 protein is encoded by a polynucleotide comprising a sequence having one, two, three, four, five or more changes selected from nucleotide substitutions, insertions, or deletions, relative to a sequence selected from SEQ ID NOs: 24-26, or any fragment thereof,
or wherein the Cas9 protein comprises the amino acid sequence of SEQ ID NO: 20 or 21 or 22 or 23, or any fragment thereof, or is encoded by a polynucleotide comprising a sequence selected from SEQ ID NOs: 24 or 25 or 26.

14-15. (canceled)

16. The composition of claim 1, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

17. The composition of claim 1, wherein the second polypeptide domain has transcription repression activity.

18. The composition of claim 17, wherein the second polypeptide domain comprises KRAB.

19-24. (canceled)

25. An isolated polynucleotide sequence comprising a sequence selected from SEQ ID NOs: 57-338.

26. An isolated polynucleotide sequence encoding the composition of claim 1.

27. (canceled)

28. A vector encoding the composition of claim 1.

29. A cell comprising the composition of claim 1.

30. A pharmaceutical composition comprising the composition of claim 1.

31. A method of treating leukemia in a subject, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the subject.

32-33. (canceled)

34. A method of modifying growth of a cell, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, IGBP1, FADS3, RPAP1, SLC25A39, RP13-20L14.6, FOXA2, and GMPR in the cell.

35. (canceled)

36. A method of decreasing cell fitness, the method comprising targeting a regulatory element of, or modifying the expression of, a gene selected from SCD, LDB1, NOLC1, CASP7, EIF3A, FAM45A, BNIP3, MASTL, AKR1E2, CRTAM, LMO2, LMO2, GAB2, GAB2, PGAM5, YARS2, KLHDC1, PDCD7, ZNF609, NR2F2, NR2F2-AS1, PLK1, ZG16B, CBFA2T3, MVD, SPATA33, SREBF1, CTD-2008P7.1, CCR10, HAP1, PTRF, STAT3, STAT5A, STAT5B, CAMKK1, RSAD1, XYLT2, ERN1, CARD14, KLF1, TNPO2, RASAL3, AC005256.1, GIPC3, MKNK2, PDCD5, CTC-273B12.10, CTD-3073N11.9, AC008440.5, SARS, SARS, RP5-1065J22.8, SARS, DFFA, KIAA2013, RP11-196G18.24, THEM4, SLAMF1, snoU13, PPP1R15B, RP5-1092A3.4, MEGF6, WRAP73, CDC20, TIE1, DNAJC11, BCL2L1, TPX2, OSBPL2, SS18L1, AP000265.1, IL10RB, MIS18A, MRPS6, AP001476.4, USP18, AC004463.6, ERCC3, SRBD1, BCYRN1, EPCAM, FOXN2, PNPT1, HK2, INO80B, GHRLOS, ATP6V1A, RP11-53616.2, DROSHA, PELO, PELO, RIOK2, PHACTR1, AHI1, MYB, MYB, ULBP1, FBXO5, HIST1H1D, HIST1H1T, HIST1H2AC, NFKBIL1, PPP1R10, XXbac-BPG252P9.10, ATP6V1G2, TUBB, DHX16, MICA, MICB, PPP1R10, RP11-140K17.3, FRS3, CDHR3, RP4-593H12.1, RP5-884M6.1, PSMG3, DDX56, MSRA, CDC26, RNF183, ENG, RP11-545E17.3, C9orf171, INPP5E, PTGDS, RAB33A, DUSP9, GATA1, GLOD5, HDAC6, PLP2, SUV39H1, WAS, PIM2, and IGBP1 in the cell.

37. (canceled)

38. The method of claim 36, wherein decreasing cell fitness comprises decreasing cell growth rate, decreasing cell growth duration, decreasing cell size, increasing cell death, or a combination thereof.

39. A method of increasing cell fitness, the method comprising targeting a regulatory element of, or modifying the expression of, a gene selected from FADS3, RPAP1, SLC25A39, RP13-20L14.6, FOXA2, and GMPR in the cell.

40. (canceled)

41. The method of claim 39, wherein increasing cell fitness comprises increasing cell growth rate, increasing cell growth duration, increasing cell size, or a combination thereof.


SEQUENCE LISTING (509 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 1368 aa, Streptococcus pyogenes [sequence omitted]

SEQ ID NO: 21 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 25 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 27 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 28 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 29 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 30 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 32 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 33 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 34 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 36 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 37 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 38 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 39 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 40 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 41 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 42 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 43 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 44 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 45 - protein, 378 aa, synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 46 - DNA, 1134 bp, synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 47 - protein, 522 aa, synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 48 - DNA, 1566 bp, synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 49 - protein, 7 aa, synthetic construct
PKKKRKV

SEQ ID NO: 50 - protein, 5 aa, synthetic construct
  REPEAT 1..5: note = 0 to 10
GGGGS

SEQ ID NO: 51 - protein, 5 aa, synthetic construct
GGGGG

SEQ ID NO: 52 - protein, 5 aa, synthetic construct
GGAGG

SEQ ID NO: 53 - protein, 7 aa, synthetic construct
GGGGSSS

SEQ ID NO: 54 - protein, 7 aa, synthetic construct
GGGGAAA

SEQ ID NO: 55 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 56 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 57 - DNA, 20 bp, synthetic construct
  source 1..20: mol_type = other DNA
ctcaagggagaaggttaatt

SEQ ID NO: 58 - DNA, 20 bp, synthetic construct
  source 1..20: mol_type = other DNA
ggcctaccagataaccagtt

SEQ ID NO: 59 - DNA, 20 bp, synthetic construct
  source 1..20: mol_type = other DNA
ggcctaccagataaccagtt

SEQ ID NO: 60 - DNA, 20 bp, synthetic construct
  source 1..20: mol_type = other DNA
tgcgtcacatgagaggaagt

SEQ ID NO: 61 - DNA, 20 bp, synthetic construct
  source 1..20: mol_type = other DNA
agtgacagtggatgccataa

SEQ ID NO: 62 - DNA, 20 bp, synthetic construct
  source 1..20: mol_type = other DNA
agtgacagtggatgccataa

SEQ ID NO: 63 - DNA, 20 bp, synthetic construct
  source 1..20: mol_type = other DNA
gagggggagcggggcgagag

SEQ ID NO: 64 - DNA, 20 bp, synthetic construct
  source 1..20: mol_type = other DNA
cgggagtggctgctcgcgga

SEQ ID NO: 65 - DNA, 20 bp, synthetic construct
  source 1..20: mol_type = other DNA
ggaggtgaggaaggagggaa

SEQ ID NO: 66 - DNA, 20 bp, synthetic construct
  source 1..20: mol_type = other DNA
gcagtgcctccggcgggggt

SEQ ID NO: 67 - DNA, 20 bp, synthetic construct
  source 1..20: mol_type = other DNA
tgattagggacagttccccg

SEQ ID NO: 68 - DNA, 20 bp, synthetic construct
  source 1..20: mol_type = other DNA
taactgttacatgaagacaa

SEQ ID NO: 69 - DNA, 20 bp, synthetic construct
  source 1..20: mol_type = other DNA
ggcgcccagaaaacctaggt

SEQ ID NO: 70 - DNA, 20 bp, synthetic construct
  source 1..20: mol_type = other DNA
tctatcttctgccctgactt

SEQ ID NO: 71 - DNA, 20 bp, synthetic construct
  source 1..20: mol_type = other DNA
aggggcgagcggagaggagg

SEQ ID NO: 72 - DNA, 20 bp, synthetic construct
  source 1..20: mol_type = other DNA
gcgcctcgcgttccttggta

SEQ ID NO: 73 - DNA, 20 bp, synthetic construct
  source 1..20: mol_type = other DNA
ttaagctagggagaatattg

SEQ ID NO: 74 - DNA, 20 bp, synthetic construct
  source 1..20: mol_type = other DNA
tccctctggggttgaggagg

SEQ ID NO: 75 - DNA, 20 bp, synthetic construct
  source 1..20: mol_type = other DNA
tccctctggggttgaggagg

SEQ ID NO: 76 - DNA, 20 bp, synthetic construct
  source 1..20: mol_type = other DNA
tgagaccagcctgggtgaca

SEQ ID NO: 77 - DNA, 20 bp, synthetic construct
  source 1..20: mol_type = other DNA
tgagaccagcctgggtgaca

SEQ ID NO: 78 - DNA, 20 bp, synthetic construct
  source 1..20: mol_type = other DNA
gggaactggaactgcctgcg

SEQ ID NO: 79 - DNA, 20 bp, synthetic construct
  source 1..20: mol_type = other DNA
ggcgaaagccgactcgaggg

SEQ ID NO: 80 - DNA, 20 bp, synthetic construct
  source 1..20: mol_type = other DNA
ccggtcagggatgttaggag

SEQ ID NO: 81 - DNA, 20 bp, synthetic construct
  source 1..20: mol_type = other DNA
ccggtcagggatgttaggag

SEQ ID NO: 82 - DNA, 20 bp, synthetic construct
  source 1..20: mol_type = other DNA
ccggtcagggatgttaggag

SEQ ID NO: 83 - DNA, 20 bp, synthetic construct
  source 1..20: mol_type = other DNA
tgctcctggaagccccaccc

SEQ ID NO: 84 - DNA, 20 bp, synthetic construct
  source 1..20: mol_type = other DNA
tgatagagctgggggacccg

SEQ ID NO: 85 - DNA, 20 bp, synthetic construct
  source 1..20: mol_type = other DNA
ttacagcaggaacaagactc

SEQ ID NO: 86 - DNA, 20 bp, synthetic construct
  source 1..20: mol_type = other DNA
ttacagcaggaacaagactc

SEQ ID NO: 87 - DNA, 20 bp, synthetic construct
  source 1..20: mol_type = other DNA
ttacagcaggaacaagactc

SEQ ID NO: 88 - DNA, 20 bp, synthetic construct
  source 1..20: mol_type = other DNA
ttacagcaggaacaagactc

SEQ ID NO: 89 - DNA, 20 bp, synthetic construct
  source 1..20: mol_type = other DNA
ttacagcaggaacaagactc

SEQ ID NO: 90 - DNA, 20 bp, synthetic construct
  source 1..20: mol_type = other DNA
ttacagcaggaacaagactc

SEQ ID NO: 91 - DNA, 20 bp, synthetic construct
  source 1..20: mol_type = other DNA
agggatggaccccagctcca

SEQ ID NO: 92 - DNA, 20 bp, synthetic construct
  source 1..20: mol_type = other DNA
actagcagaaggccctgaag

SEQ ID NO: 93 - DNA, 20 bp, synthetic construct
  source 1..20: mol_type = other DNA
actagcagaaggccctgaag

SEQ ID NO: 94 - DNA, 20 bp, synthetic construct
  source 1..20: mol_type = other DNA
agctggactgggccagagcg

SEQ ID NO: 95 - DNA, 20 bp, synthetic construct
  source 1..20: mol_type = other DNA
gcccagttgggggattcggg

SEQ ID NO: 96 - DNA, 20 bp, synthetic construct
  source 1..20: mol_type = other DNA
acgtggaggggcggctccgt

SEQ ID NO: 97 - DNA, 20 bp, synthetic construct
  source 1..20: mol_type = other DNA
acgtggaggggcggctccgt

SEQ ID NO: 98 - DNA, 20 bp, synthetic construct
  source 1..20: mol_type = other DNA
gattcctgcgggaaccgggg

SEQ ID NO: 99 - DNA, 20 bp, synthetic construct
  source 1..20: mol_type = other DNA
ggtcgacagcttgggtccct

SEQ ID NO: 100 - DNA, 20 bp, synthetic construct
  source 1..20: mol_type = other DNA
ggtcgacagcttgggtccct

SEQ ID NO: 101 - DNA, 20 bp, synthetic construct
  source 1..20: mol_type = other DNA
ggtcgacagcttgggtccct

SEQ ID NO: 102 - DNA, 20 bp, synthetic construct
  source 1..20: mol_type = other DNA
acttgagcctgggggtgtcc

SEQ ID NO: 103 - DNA, 20 bp, synthetic construct
  source 1..20: mol_type = other DNA
actaacccgctggccctccc

SEQ ID NO: 104 - DNA, 20 bp, synthetic construct
  source 1..20: mol_type = other DNA
tctgaggtgtgaccacacag

SEQ ID NO: 105 - DNA, 20 bp, synthetic construct
  source 1..20: mol_type = other DNA
gagtgaggggaggtggagag

SEQ ID NO: 106 - DNA, 20 bp, synthetic construct
  source 1..20: mol_type = other DNA
tcatcgacctagctcccaga

SEQ ID NO: 107 - DNA, 20 bp, synthetic construct
  source 1..20: mol_type = other DNA
gggagtggcacaccctgata

SEQ ID NO: 108 - DNA, 20 bp, synthetic construct
  source 1..20: mol_type = other DNA
tccctacacgggcaggagga

SEQ ID NO: 109 - DNA, 20 bp, synthetic construct
  source 1..20: mol_type = other DNA
tccctacacgggcaggagga

SEQ ID NO: 110 - DNA, 20 bp, synthetic construct
  source 1..20: mol_type = other DNA
gtccctcccgtggggctgat

SEQ ID NO: 111 - DNA, 20 bp, synthetic construct
  source 1..20: mol_type = other DNA
gtccctcccgtggggctgat

SEQ ID NO: 112 - DNA, 20 bp, synthetic construct
  source 1..20: mol_type = other DNA
tcatagtcacacccaggagg

SEQ ID NO: 113 - DNA, 20 bp, synthetic construct
  source 1..20: mol_type = other DNA
gaggcacagcctgctctctc

SEQ ID NO: 114 - DNA, 20 bp, synthetic construct
  source 1..20: mol_type = other DNA
ggggagctggggaggatgga

SEQ ID NO: 115 - DNA, 20 bp, synthetic construct
  source 1..20: mol_type = other DNA
ccgatgggaggaggcagggg

SEQ ID NO: 116 - DNA, 20 bp, synthetic construct
  source 1..20: mol_type = other DNA
agcgccctgagagccctgaa

SEQ ID NO: 117 - DNA, 20 bp, synthetic construct
  source 1..20: mol_type = other DNA
gctgtacagggcgagagaag

SEQ ID NO: 118 - DNA, 20 bp, synthetic construct
  source 1..20: mol_type = other DNA
tgtgaccggaccaccctcca

SEQ ID NO: 119 - DNA, 20 bp, synthetic construct
  source 1..20: mol_type = other DNA
tgtgaccggaccaccctcca

SEQ ID NO: 120 - DNA, 20 bp, synthetic construct
  source 1..20: mol_type = other DNA
gccagaacgtgaaccactgg

SEQ ID NO: 121 - DNA, 20 bp, synthetic construct
  source 1..20: mol_type = other DNA
gccagaacgtgaaccactgg

SEQ ID NO: 122 - DNA, 20 bp, synthetic construct
  source 1..20: mol_type = other DNA
ctgagacccaggaggtgctg

SEQ ID NO: 123 - DNA, 20 bp, synthetic construct
  source 1..20: mol_type = other DNA
aggcctagcagtgcgagtgt

SEQ ID NO: 124 - DNA, 20 bp, synthetic construct
  source 1..20: mol_type = other DNA
ggccacaggatgtcaagaaa

SEQ ID NO: 125 - DNA, 20 bp, synthetic construct
  source 1..20: mol_type = other DNA
ggagggcacccaggtcctca

SEQ ID NO: 126 - DNA, 20 bp, synthetic construct
  source 1..20: mol_type = other DNA
ggagggcacccaggtcctca

SEQ ID NO: 127 - DNA, 20 bp, synthetic construct
  source 1..20: mol_type = other DNA
gggcgtgagggggccaacca

SEQ ID NO: 128 - DNA, 20 bp, synthetic construct
  source 1..20: mol_type = other DNA
gggcgtgagggggccaacca

SEQ ID NO: 129 - DNA, 20 bp, synthetic construct
  source 1..20: mol_type = other DNA
gggcgtgagggggccaacca

SEQ ID NO: 130 - DNA, 20 bp, synthetic construct
  source 1..20: mol_type = other DNA
gggcgtgagggggccaacca

SEQ ID NO: 131 - DNA, 20 bp, synthetic construct
  source 1..20: mol_type = other DNA
agaggacagaggcaggaggg

SEQ ID NO: 132 - DNA, 20 bp, synthetic construct
  source 1..20: mol_type = other DNA
tcccctctagcggtaaggcc

SEQ ID NO: 133 - DNA, 20 bp, synthetic construct
  source 1..20: mol_type = other DNA
ctcatccatcggccatgtgc

SEQ ID NO: 134 - DNA, 20 bp, synthetic construct
  source 1..20: mol_type = other DNA
ttaacctggggggaacccac

SEQ ID NO: 135 - DNA, 20 bp, synthetic construct
  source 1..20: mol_type = other DNA
gcgctcagtaaccggaggaa

SEQ ID NO: 136 - DNA, 20 bp, synthetic construct
  source 1..20: mol_type = other DNA
ggagagctggggccacagct

SEQ ID NO: 137 - DNA, 20 bp, synthetic construct
  source 1..20: mol_type = other DNA
ggagagctggggccacagct

SEQ ID NO: 138 - DNA, 20 bp, synthetic construct
  source 1..20: mol_type = other DNA
gggcccagccagtcccaact

SEQ ID NO: 139 - DNA, 20 bp, synthetic construct
  source 1..20: mol_type = other DNA
gcccgaggagtgggacgtgg

SEQ ID NO: 140 - DNA, 20 bp, synthetic construct
  source 1..20: mol_type = other DNA
gtgggacctctccgattcac

SEQ ID NO: 141 - DNA, 20 bp, synthetic construct
  source 1..20: mol_type = other DNA
tcctcgagcccacccccgca

SEQ ID NO: 142 - DNA, 20 bp, synthetic construct
  source 1..20: mol_type = other DNA
gtccctatggaccagcacca

SEQ ID NO: 143 - DNA, 20 bp, synthetic construct
  source 1..20: mol_type = other DNA
ggaggtagcaaaaaccctgg

SEQ ID NO: 144 - DNA, 20 bp, synthetic construct
  source 1..20: mol_type = other DNA
ttcaggccatgaagggaagt

SEQ ID NO: 145 - DNA, 20 bp, synthetic construct
  source 1..20: mol_type = other DNA
actcccctccgagagccggg

SEQ ID NO: 146 - DNA, 20 bp, synthetic construct
  source 1..20: mol_type = other DNA
ctgccagcgggaactgtgta

SEQ ID NO: 147 - DNA, 20 bp, synthetic construct
  source 1..20: mol_type = other DNA
aggtgaacatccctaggaaa

SEQ ID NO: 148 - DNA, 20 bp, synthetic construct
  source 1..20: mol_type = other DNA
tcacagctggtcggaagctc

SEQ ID NO: 149 - DNA, 20 bp, synthetic construct
  source 1..20: mol_type = other DNA
gcatgcccgggaccagctgt

SEQ ID NO: 150 - DNA, 20 bp, synthetic construct
  source 1..20: mol_type = other DNA
ttagggcacttgagagactg

SEQ ID NO: 151 - DNA, 20 bp, synthetic construct
  source 1..20: mol_type = other DNA
ttagggcacttgagagactg

SEQ ID NO: 152 - DNA, 20 bp, synthetic construct
  source 1..20: mol_type = other DNA
ctgaaaccaaagcagtactt

SEQ ID NO: 153 - DNA, 20 bp, synthetic construct
  source 1..20: mol_type = other DNA
tgagagcaggcggagggaag

SEQ ID NO: 154 - DNA, 20 bp, synthetic construct
  source 1..20: mol_type = other DNA
gagggcggctgggtgtcggg

SEQ ID NO: 155 - DNA, 20 bp, synthetic construct
  source 1..20: mol_type = other DNA
ctgtgactcgcgtagcagag

SEQ ID NO: 156 - DNA, 20 bp, synthetic construct
  source 1..20: mol_type = other DNA
ctgtgactcgcgtagcagag

SEQ ID NO: 157 - DNA, 20 bp, synthetic construct
  source 1..20: mol_type = other DNA
ctgtgactcgcgtagcagag

SEQ ID NO: 158 - DNA, 20 bp, synthetic construct
  source 1..20: mol_type = other DNA
gggcggccctaagctcagga

SEQ ID NO: 159 - DNA, 20 bp, synthetic construct
  source 1..20: mol_type = other DNA
gggcggccctaagctcagga

SEQ ID NO: 160 - DNA, 20 bp, synthetic construct
  source 1..20: mol_type = other DNA
gggcggccctaagctcagga

SEQ ID NO: 161 - DNA, 20 bp, synthetic construct
  source 1..20: mol_type = other DNA
cccgacggtggttcacgaaa

SEQ ID NO: 162 - DNA, 20 bp, synthetic construct
  source 1..20: mol_type = other DNA
cccgacggtggttcacgaaa

SEQ ID NO: 163 - DNA, 20 bp, synthetic construct
  source 1..20: mol_type = other DNA
aggagcagcccgggaaccca

SEQ ID NO: 164 - DNA, 20 bp, synthetic construct
  source 1..20: mol_type = other DNA
aggagcagcccgggaaccca

SEQ ID NO: 165 - DNA, 20 bp, synthetic construct
  source 1..20: mol_type = other DNA
aggagcagcccgggaaccca

SEQ ID NO: 166 - DNA, 20 bp, synthetic construct
  source 1..20: mol_type = other DNA
aggagcagcccgggaaccca

SEQ ID NO: 167 - DNA, 20 bp, synthetic construct
  source 1..20: mol_type = other DNA
cgctgccggccagcgtcctc

SEQ ID NO: 168 - DNA, 20 bp, synthetic construct
  source 1..20: mol_type = other DNA
cgctaacagagccgccacat

SEQ ID NO: 169 - DNA, 20 bp, synthetic construct
  source 1..20: mol_type = other DNA
taagaagtcggcaggggcag

SEQ ID NO: 170 - DNA, 20 bp, synthetic construct
  source 1..20: mol_type = other DNA
taagaagtcggcaggggcag

SEQ ID NO: 171 - DNA, 20 bp, synthetic construct
  source 1..20: mol_type = other DNA
taagaagtcggcaggggcag

SEQ ID NO: 172 - DNA, 20 bp, synthetic construct
  source 1..20: mol_type = other DNA
ccagcgctggagggcagcgg

SEQ ID NO: 173 - DNA, 20 bp, synthetic construct
  source 1..20: mol_type = other DNA
cagccctcgcgtgtacctga

SEQ ID NO: 174 - DNA, 20 bp, synthetic construct
  source 1..20: mol_type = other DNA
tgcgaaaggcacaggatccc

SEQ ID NO: 175 - DNA, 20 bp, synthetic construct
  source 1..20: mol_type = other DNA
gtgctggctgtagaggttaa

SEQ ID NO: 176 - DNA, 20 bp, synthetic construct
  source 1..20: mol_type = other DNA
gtgctggctgtagaggttaa

SEQ ID NO: 177 - DNA, 20 bp, synthetic construct
  source 1..20: mol_type = other DNA
cctcacaacggggaggaaac

SEQ ID NO: 178 - DNA, 20 bp, synthetic construct
  source 1..20: mol_type = other DNA
gggcttgctgagcactcgcg

SEQ ID NO: 179 - DNA, 20 bp, synthetic construct
  source 1..20: mol_type = other DNA
gagcactgagaggagcgggg

SEQ ID NO: 180 - DNA, 20 bp, synthetic construct
  source 1..20: mol_type = other DNA
ccatgctcatgagcactgga

SEQ ID NO: 181 - DNA, 20 bp, synthetic construct
  source 1..20: mol_type = other DNA
ccatgctcatgagcactgga

SEQ ID NO: 182 - DNA, 20 bp, synthetic construct
  source 1..20: mol_type = other DNA
ggccgagcgccccaggtcgg

SEQ ID NO: 183 - DNA, 20 bp, synthetic construct
  source 1..20: mol_type = other DNA
tcagggatgggagaggagga

SEQ ID NO: 184 - DNA, 20 bp, synthetic construct
  source 1..20: mol_type = other DNA
tctaccggtaccctctcccc

SEQ ID NO: 185 - DNA, 20 bp, synthetic construct
  source 1..20: mol_type = other DNA
tctaccggtaccctctcccc

SEQ ID NO: 186 - DNA, 20 bp, synthetic construct
  source 1..20: mol_type = other DNA
tctaccggtaccctctcccc

SEQ ID NO: 187 - DNA, 20 bp, synthetic construct
  source 1..20: mol_type = other DNA
tctaccggtaccctctcccc

SEQ ID NO: 188 - DNA, 20 bp, synthetic construct
  source 1..20: mol_type = other DNA
tctaccggtaccctctcccc

SEQ ID NO: 189 - DNA, 20 bp, synthetic construct
  source 1..20: mol_type = other DNA
tctaccggtaccctctcccc

SEQ ID NO: 190 - DNA, 20 bp, synthetic construct
  source 1..20: mol_type = other DNA
tgagcctcagaggtatcctg

SEQ ID NO: 191 - DNA, 20 bp, synthetic construct
  source 1..20: mol_type = other DNA
tcgtaagatatcaaccatct

SEQ ID NO: 192 - DNA, 20 bp, synthetic construct
  source 1..20: mol_type = other DNA
gaactaggatcccacagggt

SEQ ID NO: 193 - DNA, 20 bp, synthetic construct
  source 1..20: mol_type = other DNA
gcttcctcctctccactcct

SEQ ID NO: 194 - DNA, 20 bp, synthetic construct
  source 1..20: mol_type = other DNA
caggtcctctcctatctctt

SEQ ID NO: 195 - DNA, 20 bp, synthetic construct
  source 1..20: mol_type = other DNA
aacagtttaatcaattagcg

SEQ ID NO: 196 - DNA, 20 bp, synthetic construct
  source 1..20: mol_type = other DNA
tactgagtcataccaatgtt

SEQ ID NO: 197 - DNA, 20 bp, synthetic construct
  source 1..20: mol_type = other DNA
tccatttcagtttataccag

SEQ ID NO: 198 - RNA, 20 nt, synthetic construct
  source 1..20: mol_type = other RNA
ctcaagggagaaggttaatt

SEQ ID NO: 199 - RNA, 20 nt, synthetic construct
  source 1..20: mol_type = other RNA
ggcctaccagataaccagtt

SEQ ID NO: 200 - RNA, 20 nt, synthetic construct
  source 1..20: mol_type = other RNA
ggcctaccagataaccagtt

SEQ ID NO: 201 - RNA, 20 nt, synthetic construct
  source 1..20: mol_type = other RNA
tgcgtcacatgagaggaagt

SEQ ID NO: 202 - RNA, 20 nt, synthetic construct
  source 1..20: mol_type = other RNA
agtgacagtggatgccataa

SEQ ID NO: 203 - RNA, 20 nt, synthetic construct
  source 1..20: mol_type = other RNA
agtgacagtggatgccataa

SEQ ID NO: 204 - RNA, 20 nt, synthetic construct
  source 1..20: mol_type = other RNA
gagggggagcggggcgagag

SEQ ID NO: 205 - RNA, 20 nt, synthetic construct
  source 1..20: mol_type = other RNA
cgggagtggctgctcgcgga

SEQ ID NO: 206 - RNA, 20 nt, synthetic construct
  source 1..20: mol_type = other RNA
ggaggtgaggaaggagggaa

SEQ ID NO: 207 - RNA, 20 nt, synthetic construct
  source 1..20: mol_type = other RNA
gcagtgcctccggcgggggt

SEQ ID NO: 208 - RNA, 20 nt, synthetic construct
  source 1..20: mol_type = other RNA
tgattagggacagttccccg

SEQ ID NO: 209 - RNA, 20 nt, synthetic construct
  source 1..20: mol_type = other RNA
taactgttacatgaagacaa

SEQ ID NO: 210 - RNA, 20 nt, synthetic construct
  source 1..20: mol_type = other RNA
ggcgcccagaaaacctaggt

SEQ ID NO: 211 - RNA, 20 nt, synthetic construct
  source 1..20: mol_type = other RNA
tctatcttctgccctgactt

SEQ ID NO: 212 - RNA, 20 nt, synthetic construct
  source 1..20: mol_type = other RNA
aggggcgagcggagaggagg

SEQ ID NO: 213 - RNA, 20 nt, synthetic construct
  source 1..20: mol_type = other RNA
gcgcctcgcgttccttggta

SEQ ID NO: 214 - RNA, 20 nt, synthetic construct
  source 1..20: mol_type = other RNA
ttaagctagggagaatattg

SEQ ID NO: 215 - RNA, 20 nt, synthetic construct
  source 1..20: mol_type = other RNA
tccctctggggttgaggagg

SEQ ID NO: 216 - RNA, 20 nt, synthetic construct
  source 1..20: mol_type = other RNA
tccctctggggttgaggagg

SEQ ID NO: 217 - RNA, 20 nt, synthetic construct
  source 1..20: mol_type = other RNA
tgagaccagcctgggtgaca

SEQ ID NO: 218 - RNA, 20 nt, synthetic construct
  source 1..20: mol_type = other RNA
tgagaccagcctgggtgaca

SEQ ID NO: 219 - RNA, 20 nt, synthetic construct
  source 1..20: mol_type = other RNA
gggaactggaactgcctgcg

SEQ ID NO: 220 - RNA, 20 nt, synthetic construct
  source 1..20: mol_type = other RNA
ggcgaaagccgactcgaggg

SEQ ID NO: 221 - RNA, 20 nt, synthetic construct
  source 1..20: mol_type = other RNA
ccggtcagggatgttaggag

SEQ ID NO: 222 - RNA, 20 nt, synthetic construct
  source 1..20: mol_type = other RNA
ccggtcagggatgttaggag

SEQ ID NO: 223 - RNA, 20 nt, synthetic construct
  source 1..20: mol_type = other RNA
ccggtcagggatgttaggag

SEQ ID NO: 224 - RNA, 20 nt, synthetic construct
  source 1..20: mol_type = other RNA
tgctcctggaagccccaccc

SEQ ID NO: 225 - RNA, 20 nt, synthetic construct
  source 1..20: mol_type = other RNA
tgatagagctgggggacccg

SEQ ID NO: 226 - RNA, 20 nt, synthetic construct
  source 1..20: mol_type = other RNA
ttacagcaggaacaagactc

SEQ ID NO: 227 - RNA, 20 nt, synthetic construct
  source 1..20: mol_type = other RNA
ttacagcaggaacaagactc

SEQ ID NO: 228 - RNA, 20 nt, synthetic construct
  source 1..20: mol_type = other RNA
ttacagcaggaacaagactc

SEQ ID NO: 229 - RNA, 20 nt, synthetic construct
  source 1..20: mol_type = other RNA
ttacagcaggaacaagactc

SEQ ID NO: 230 - RNA, 20 nt, synthetic construct
  source 1..20: mol_type = other RNA
ttacagcaggaacaagactc

SEQ ID NO: 231 - RNA, 20 nt, synthetic construct
  source 1..20: mol_type = other RNA
ttacagcaggaacaagactc

SEQ ID NO: 232 - RNA, 20 nt, synthetic construct
  source 1..20: mol_type = other RNA
agggatggaccccagctcca

SEQ ID NO: 233 - RNA, 20 nt, synthetic construct
  source 1..20: mol_type = other RNA
actagcagaaggccctgaag

SEQ ID NO: 234 - RNA, 20 nt, synthetic construct
  source 1..20: mol_type = other RNA
actagcagaaggccctgaag

SEQ ID NO: 235 - RNA, 20 nt, synthetic construct
  source 1..20: mol_type = other RNA
agctggactgggccagagcg

SEQ ID NO: 236 - RNA, 20 nt, synthetic construct
  source 1..20: mol_type = other RNA
gcccagttgggggattcggg

SEQ ID NO: 237 - RNA, 20 nt, synthetic construct
  source 1..20: mol_type = other RNA
acgtggaggggcggctccgt

SEQ ID NO: 238 - RNA, 20 nt, synthetic construct
  source 1..20: mol_type = other RNA
acgtggaggggcggctccgt

SEQ ID NO: 239 - RNA, 20 nt, synthetic construct
  source 1..20: mol_type = other RNA
gattcctgcgggaaccgggg

SEQ ID NO: 240 - RNA, 20 nt, synthetic construct
  source 1..20: mol_type = other RNA
ggtcgacagcttgggtccct

SEQ ID NO: 241 - RNA, 20 nt, synthetic construct
  source 1..20: mol_type = other RNA
ggtcgacagcttgggtccct

SEQ ID NO: 242 - RNA, 20 nt, synthetic construct
  source 1..20: mol_type = other RNA
ggtcgacagcttgggtccct

SEQ ID NO: 243 - RNA, 20 nt, synthetic construct
  source 1..20: mol_type = other RNA
acttgagcctgggggtgtcc

SEQ ID NO: 244 - RNA, 20 nt, synthetic construct
  source 1..20: mol_type = other RNA
actaacccgctggccctccc

SEQ ID NO: 245 - RNA, 20 nt, synthetic construct
  source 1..20: mol_type = other RNA
tctgaggtgtgaccacacag

SEQ ID NO: 246 - RNA, 20 nt, synthetic construct
  source 1..20: mol_type = other RNA
gagtgaggggaggtggagag

SEQ ID NO: 247 - RNA, 20 nt, synthetic construct
  source 1..20: mol_type = other RNA
tcatcgacctagctcccaga

SEQ ID NO: 248 - RNA, 20 nt, synthetic construct
  source 1..20: mol_type = other RNA
gggagtggcacaccctgata

SEQ ID NO: 249 - RNA, 20 nt, synthetic construct
  source 1..20: mol_type = other RNA
tccctacacgggcaggagga

SEQ ID NO: 250 - RNA, 20 nt, synthetic construct
  source 1..20: mol_type = other RNA
tccctacacgggcaggagga

SEQ ID NO: 251 - RNA, 20 nt, synthetic construct
  source 1..20: mol_type = other RNA
gtccctcccgtggggctgat

SEQ ID NO: 252 - RNA, 20 nt, synthetic construct
  source 1..20: mol_type = other RNA
gtccctcccgtggggctgat

SEQ ID NO: 253 - RNA, 20 nt, synthetic construct
  source 1..20: mol_type = other RNA
tcatagtcacacccaggagg

SEQ ID NO: 254 - RNA, 20 nt, synthetic construct
  source 1..20: mol_type = other RNA
gaggcacagcctgctctctc

SEQ ID NO: 255 - RNA, 20 nt, synthetic construct
  source 1..20: mol_type = other RNA
ggggagctggggaggatgga

SEQ ID NO: 256 - RNA, 20 nt, synthetic construct
  source 1..20: mol_type = other RNA
ccgatgggaggaggcagggg

SEQ ID NO: 257 - RNA, 20 nt, synthetic construct
  source 1..20: mol_type = other RNA
agcgccctgagagccctgaa

SEQ ID NO: 258 - RNA, 20 nt, synthetic construct
  source 1..20: mol_type = other RNA
gctgtacagggcgagagaag

SEQ ID NO: 259 - RNA, 20 nt, synthetic construct
  source 1..20: mol_type = other RNA
tgtgaccggaccaccctcca

SEQ ID NO: 260 - RNA, 20 nt, synthetic construct
  source 1..20: mol_type = other RNA
tgtgaccggaccaccctcca

SEQ ID NO: 261 - RNA, 20 nt, synthetic construct
  source 1..20: mol_type = other RNA
gccagaacgtgaaccactgg

SEQ ID NO: 262 - RNA, 20 nt, synthetic construct
  source 1..20: mol_type = other RNA
gccagaacgtgaaccactgg

SEQ ID NO: 263 - RNA, 20 nt, synthetic construct
  source 1..20: mol_type = other RNA
ctgagacccaggaggtgctg

SEQ ID NO: 264 - RNA, 20 nt, synthetic construct
  source 1..20: mol_type = other RNA
aggcctagcagtgcgagtgt

SEQ ID NO: 265 - RNA, 20 nt, synthetic construct
  source 1..20: mol_type = other RNA
ggccacaggatgtcaagaaa

SEQ ID NO: 266 - RNA, 20 nt, synthetic construct
  source 1..20: mol_type = other RNA
ggagggcacccaggtcctca

SEQ ID NO: 267 - RNA, 20 nt, synthetic construct
  source 1..20: mol_type = other RNA
ggagggcacccaggtcctca

SEQ ID NO: 268 - RNA, 20 nt, synthetic construct
  source 1..20: mol_type = other RNA
gggcgtgagggggccaacca

SEQ ID NO: 269 - RNA, 20 nt, synthetic construct
  source 1..20: mol_type = other RNA
gggcgtgagggggccaacca

SEQ ID NO: 270 - RNA, 20 nt, synthetic construct
  source 1..20: mol_type = other RNA
gggcgtgagggggccaacca

SEQ ID NO: 271 - RNA, 20 nt, synthetic construct
  source 1..20: mol_type = other RNA
gggcgtgagggggccaacca

SEQ ID NO: 272 - RNA, 20 nt, synthetic construct
  source 1..20: mol_type = other RNA
agaggacagaggcaggaggg

SEQ ID NO: 273 - RNA, 20 nt, synthetic construct
  source 1..20: mol_type = other RNA
tcccctctagcggtaaggcc

SEQ ID NO: 274 - RNA, 20 nt, synthetic construct
  source 1..20: mol_type = other RNA
ctcatccatcggccatgtgc

SEQ ID NO: 275 - RNA, 20 nt, synthetic construct
  source 1..20: mol_type = other RNA
ttaacctggggggaacccac

SEQ ID NO: 276 - RNA, 20 nt, synthetic construct
  source 1..20: mol_type = other RNA
gcgctcagtaaccggaggaa

SEQ ID NO: 277 - RNA, 20 nt, synthetic construct
  source 1..20: mol_type = other RNA
ggagagctggggccacagct

SEQ ID NO: 278 - RNA, 20 nt, synthetic construct
  source 1..20: mol_type = other RNA
ggagagctggggccacagct

SEQ ID NO: 279 - RNA, 20 nt, synthetic construct
  source 1..20: mol_type = other RNA
gggcccagccagtcccaact

SEQ ID NO: 280 - RNA, 20 nt, synthetic construct
  source 1..20: mol_type = other RNA
gcccgaggagtgggacgtgg

SEQ ID NO: 281 - RNA, 20 nt, synthetic construct
  source 1..20: mol_type = other RNA
gtgggacctctccgattcac

SEQ ID NO: 282 - RNA, 20 nt, synthetic construct
  source 1..20: mol_type = other RNA
tcctcgagcccacccccgca

SEQ ID NO: 283 - RNA, 20 nt, synthetic construct
  source 1..20: mol_type = other RNA
gtccctatggaccagcacca

SEQ ID NO: 284 - RNA, 20 nt, synthetic construct
  source 1..20: mol_type = other RNA
ggaggtagcaaaaaccctgg

SEQ ID NO: 285 - RNA, 20 nt, synthetic construct
  source 1..20: mol_type = other RNA
ttcaggccatgaagggaagt

SEQ ID NO: 286 - RNA, 20 nt, synthetic construct
  source 1..20: mol_type = other RNA
actcccctccgagagccggg

SEQ ID NO: 287 - RNA, 20 nt, synthetic construct
  source 1..20: mol_type = other RNA
ctgccagcgggaactgtgta

SEQ ID NO: 288 - RNA, 20 nt, synthetic construct
  source 1..20: mol_type = other RNA
aggtgaacatccctaggaaa

SEQ ID NO: 289 - RNA, 20 nt, synthetic construct
  source 1..20: mol_type = other RNA
tcacagctggtcggaagctc

SEQ ID NO: 290 - RNA, 20 nt, synthetic construct
  source 1..20: mol_type = other RNA
gcatgcccgggaccagctgt

SEQ ID NO: 291 - RNA, 20 nt, synthetic construct
  source 1..20: mol_type = other RNA
ttagggcacttgagagactg

SEQ ID NO: 292 - RNA, 20 nt, synthetic construct
  source 1..20: mol_type = other RNA
ttagggcacttgagagactg

SEQ ID NO: 293 - RNA, 20 nt, synthetic construct
  source 1..20: mol_type = other RNA
ctgaaaccaaagcagtactt

SEQ ID NO: 294 - RNA, 20 nt, synthetic construct
  source 1..20: mol_type = other RNA
tgagagcaggcggagggaag

SEQ ID NO: 295 - RNA, 20 nt, synthetic construct
  source 1..20: mol_type = other RNA
gagggcggctgggtgtcggg

SEQ ID NO: 296 - RNA, 20 nt, synthetic construct
  source 1..20: mol_type = other RNA
ctgtgactcgcgtagcagag

SEQ ID NO: 297 - RNA, 20 nt, synthetic construct
  source 1..20: mol_type = other RNA
ctgtgactcgcgtagcagag

SEQ ID NO: 298 - RNA, 20 nt, synthetic construct
  source 1..20: mol_type = other RNA
ctgtgactcgcgtagcagag

SEQ ID NO: 299 - RNA, 20 nt, synthetic construct
  source 1..20: mol_type = other RNA
gggcggccctaagctcagga

SEQ ID NO: 300 - RNA, 20 nt, synthetic construct
  source 1..20: mol_type = other RNA
gggcggccctaagctcagga

SEQ ID NO: 301 - RNA, 20 nt, synthetic construct
  source 1..20: mol_type = other RNA
gggcggccctaagctcagga

SEQ ID NO: 302 - RNA, 20 nt, synthetic construct
  source 1..20: mol_type = other RNA
cccgacggtggttcacgaaa

SEQ ID NO: 303 - RNA, 20 nt, synthetic construct
  source 1..20: mol_type = other RNA
cccgacggtggttcacgaaa

SEQ ID NO: 304 - RNA, 20 nt, synthetic construct
  source 1..20: mol_type = other RNA
aggagcagcccgggaaccca

SEQ ID NO: 305 - RNA, 20 nt, synthetic construct
  source 1..20: mol_type = other RNA
aggagcagcccgggaaccca

SEQ ID NO: 306 - RNA, 20 nt, synthetic construct
  source 1..20: mol_type = other RNA
aggagcagcccgggaaccca

SEQ ID NO: 307 - RNA, 20 nt, synthetic construct
  source 1..20: mol_type = other RNA
aggagcagcccgggaaccca

SEQ ID NO: 308 - RNA, 20 nt, synthetic construct
  source 1..20: mol_type = other RNA
cgctgccggccagcgtcctc

SEQ ID NO: 309 - RNA, 20 nt, synthetic construct
  source 1..20: mol_type = other RNA
cgctaacagagccgccacat

SEQ ID NO: 310 - RNA, 20 nt, synthetic construct
  source 1..20: mol_type = other RNA
taagaagtcggcaggggcag

SEQ ID NO: 311 - RNA, 20 nt, synthetic construct
  source 1..20: mol_type = other RNA
taagaagtcggcaggggcag

SEQ ID NO: 312 - RNA, 20 nt, synthetic construct
  source 1..20: mol_type = other RNA
taagaagtcggcaggggcag

SEQ ID NO: 313 - RNA, 20 nt, synthetic construct
  source 1..20: mol_type = other RNA
ccagcgctggagggcagcgg

SEQ ID NO: 314 - RNA, 20 nt, synthetic construct
  source 1..20: mol_type = other RNA
cagccctcgcgtgtacctga

SEQ ID NO: 315 - RNA, 20 nt, synthetic construct
  source 1..20: mol_type = other RNA
tgcgaaaggcacaggatccc

SEQ ID NO: 316 - RNA, 20 nt, synthetic construct
  source 1..20: mol_type = other RNA
gtgctggctgtagaggttaa

SEQ ID NO: 317 - RNA, 20 nt, synthetic construct
  source 1..20: mol_type = other RNA
gtgctggctgtagaggttaa

SEQ ID NO: 318 - RNA, 20 nt, synthetic construct
  source 1..20: mol_type = other RNA
cctcacaacggggaggaaac

SEQ ID NO: 319 - RNA, 20 nt, synthetic construct
  source 1..20: mol_type = other RNA
gggcttgctgagcactcgcg

SEQ ID NO: 320 - RNA, 20 nt, synthetic construct
  source 1..20: mol_type = other RNA
gagcactgagaggagcgggg

SEQ ID NO: 321 - RNA, 20 nt, synthetic construct
  source 1..20: mol_type = other RNA
ccatgctcatgagcactgga

SEQ ID NO: 322 - RNA, 20 nt, synthetic construct
  source 1..20: mol_type = other RNA
ccatgctcatgagcactgga

SEQ ID NO: 323 - RNA, 20 nt, synthetic construct
  source 1..20: mol_type = other RNA
ggccgagcgccccaggtcgg

SEQ ID NO: 324 - RNA, 20 nt, synthetic construct
  source 1..20: mol_type = other RNA
tcagggatgggagaggagga

SEQ ID NO: 325 - RNA, 20 nt, synthetic construct
  source 1..20: mol_type = other RNA
tctaccggtaccctctcccc

SEQ ID NO: 326 - RNA, 20 nt, synthetic construct
  source 1..20: mol_type = other RNA
tctaccggtaccctctcccc

SEQ ID NO: 327 - RNA, 20 nt, synthetic construct
  source 1..20: mol_type = other RNA
tctaccggtaccctctcccc

SEQ ID NO: 328 - RNA, 20 nt, synthetic construct
  source 1..20: mol_type = other RNA
tctaccggtaccctctcccc

SEQ ID NO: 329 - RNA, 20 nt, synthetic construct
  source 1..20: mol_type = other RNA
tctaccggtaccctctcccc

SEQ ID NO: 330 - RNA, 20 nt, synthetic construct
  source 1..20: mol_type = other RNA
tctaccggtaccctctcccc

SEQ ID NO: 331 - RNA, 20 nt, synthetic construct
  source 1..20: mol_type = other RNA
tgagcctcagaggtatcctg

SEQ ID NO: 332 - RNA, 20 nt, synthetic construct
  source 1..20: mol_type = other RNA
tcgtaagatatcaaccatct

SEQ ID NO: 333 - RNA, 20 nt, synthetic construct
  source 1..20: mol_type = other RNA
gaactaggatcccacagggt

SEQ ID NO: 334 - RNA, 20 nt, synthetic construct
  source 1..20: mol_type = other RNA
gcttcctcctctccactcct

SEQ ID NO: 335 - RNA, 20 nt, synthetic construct
  source 1..20: mol_type = other RNA
caggtcctctcctatctctt

SEQ ID NO: 336 - RNA, 20 nt, synthetic construct
  source 1..20: mol_type = other RNA
aacagtttaatcaattagcg

SEQ ID NO: 337 - RNA, 20 nt, synthetic construct
  source 1..20: mol_type = other RNA
tactgagtcataccaatgtt

SEQ ID NO: 338 - RNA, 20 nt, synthetic construct
  source 1..20: mol_type = other RNA
tccatttcagtttataccag

SEQ ID NO: 339 - DNA, 23 bp, synthetic construct
  source 1..23: mol_type = other DNA
ccactcaagggagaaggttaatt

SEQ ID NO: 340 - DNA, 23 bp, synthetic construct
  source 1..23: mol_type = other DNA
ggcctaccagataaccagttggg

SEQ ID NO: 341 - DNA, 23 bp, synthetic construct
  source 1..23: mol_type = other DNA
ggcctaccagataaccagttggg

SEQ ID NO: 342 - DNA, 23 bp, synthetic construct
  source 1..23: mol_type = other DNA
tgcgtcacatgagaggaagttgg

SEQ ID NO: 343 - DNA, 23 bp, synthetic construct
  source 1..23: mol_type = other DNA
agtgacagtggatgccataacgg

SEQ ID NO: 344 - DNA, 23 bp, synthetic construct
  source 1..23: mol_type = other DNA
agtgacagtggatgccataacgg

SEQ ID NO: 345 - DNA, 23 bp, synthetic construct
  source 1..23: mol_type = other DNA
cccgagggggagcggggcgagag

SEQ ID NO: 346 - DNA, 23 bp, synthetic construct
  source 1..23: mol_type = other DNA
cgggagtggctgctcgcggaggg

SEQ ID NO: 347 - DNA, 23 bp, synthetic construct
  source 1..23: mol_type = other DNA
ccaggaggtgaggaaggagggaa

SEQ ID NO: 348 - DNA, 23 bp, synthetic construct
  source 1..23: mol_type = other DNA
gcagtgcctccggcgggggtagg

SEQ ID NO: 349 - DNA, 23 bp, synthetic construct
  source 1..23: mol_type = other DNA
ccttgattagggacagttccccg

SEQ ID NO: 350 - DNA, 23 bp, synthetic construct
  source 1..23: mol_type = other DNA
ccctaactgttacatgaagacaa

SEQ ID NO: 351 - DNA, 23 bp, synthetic construct
  source 1..23: mol_type = other DNA
ggcgcccagaaaacctaggtggg

SEQ ID NO: 352 - DNA, 23 bp, synthetic construct
  source 1..23: mol_type = other DNA
ccatctatcttctgccctgactt

SEQ ID NO: 353 - DNA, 23 bp, synthetic construct
  source 1..23: mol_type = other DNA
cctaggggcgagcggagaggagg

SEQ ID NO: 354 - DNA, 23 bp, synthetic construct
  source 1..23: mol_type = other DNA
gcgcctcgcgttccttggtacgg

SEQ ID NO: 355 - DNA, 23 bp, synthetic construct
  source 1..23: mol_type = other DNA
ccattaagctagggagaatattg

SEQ ID NO: 356 - DNA, 23 bp, synthetic construct
  source 1..23: mol_type = other DNA
tccctctggggttgaggaggagg

SEQ ID NO: 357 - DNA, 23 bp, synthetic construct
  source 1..23: mol_type = other DNA
tccctctggggttgaggaggagg

SEQ ID NO: 358 - DNA, 23 bp, synthetic construct
  source 1..23: mol_type = other DNA
ccgtgagaccagcctgggtgaca

SEQ ID NO: 359 - DNA, 23 bp, synthetic construct
  source 1..23: mol_type = other DNA
ccgtgagaccagcctgggtgaca

SEQ ID NO: 360 - DNA, 23 bp, synthetic construct
  source 1..23: mol_type = other DNA
gggaactggaactgcctgcgggg

SEQ ID NO: 361 - DNA, 23 bp, synthetic construct
  source 1..23: mol_type = other DNA
ggcgaaagccgactcgagggtgg

SEQ ID NO: 362 - DNA, 23 bp, synthetic construct
  source 1..23: mol_type = other DNA
ccggtcagggatgttaggagcgg

SEQ ID NO: 363 - DNA, 23 bp, synthetic construct
  source 1..23: mol_type = other DNA
ccggtcagggatgttaggagcgg

SEQ ID NO: 364 - DNA, 23 bp, synthetic construct
  source 1..23: mol_type = other DNA
ccggtcagggatgttaggagcgg

SEQ ID NO: 365 - DNA, 23 bp, synthetic construct
  source 1..23: mol_type = other DNA
tgctcctggaagccccaccccgg

SEQ ID NO: 366 - DNA, 23 bp, synthetic construct
  source 1..23: mol_type = other DNA
ccctgatagagctgggggacccg

SEQ ID NO: 367 - DNA, 23 bp, synthetic construct
  source 1..23: mol_type = other DNA
ttacagcaggaacaagactcagg

SEQ ID NO: 368 - DNA, 23 bp, synthetic construct
  source 1..23: mol_type = other DNA
ttacagcaggaacaagactcagg

SEQ ID NO: 369 - DNA, 23 bp, synthetic construct
  source 1..23: mol_type = other DNA
ttacagcaggaacaagactcagg

SEQ ID NO: 370 - DNA, 23 bp, synthetic construct
  source 1..23: mol_type = other DNA
ttacagcaggaacaagactcagg

SEQ ID NO: 371 - DNA, 23 bp, synthetic construct
  source 1..23: mol_type = other DNA
ttacagcaggaacaagactcagg

SEQ ID NO: 372 - DNA, 23 bp, synthetic construct
  source 1..23: mol_type = other DNA
ttacagcaggaacaagactcagg

SEQ ID NO: 373 - DNA, 23 bp, synthetic construct
  source 1..23: mol_type = other DNA
agggatggaccccagctccaggg

SEQ ID NO: 374 - DNA, 23 bp, synthetic construct
  source 1..23: mol_type = other DNA
ccaactagcagaaggccctgaag

SEQ ID NO: 375 - DNA, 23 bp, synthetic construct
  source 1..23: mol_type = other DNA
ccaactagcagaaggccctgaag

SEQ ID NO: 376 - DNA, 23 bp, synthetic construct
  source 1..23: mol_type = other DNA
agctggactgggccagagcgggg

SEQ ID NO: 377 - DNA, 23 bp, synthetic construct
  source 1..23: mol_type = other DNA
ccagcccagttgggggattcggg

SEQ ID NO: 378 - DNA, 23 bp, synthetic construct
  source 1..23: mol_type = other DNA
acgtggaggggcggctccgtggg

SEQ ID NO: 379 - DNA, 23 bp, synthetic construct
  source 1..23: mol_type = other DNA
acgtggaggggcggctccgtggg

SEQ ID NO: 380 - DNA, 23 bp, synthetic construct
  source 1..23: mol_type = other DNA
gattcctgcgggaaccggggcgg

SEQ ID NO: 381 - DNA, 23 bp, synthetic construct
  source 1..23: mol_type = other DNA
ggtcgacagcttgggtccctcgg

SEQ ID NO: 382 - DNA, 23 bp, synthetic construct
  source 1..23: mol_type = other DNA
ggtcgacagcttgggtccctcgg

SEQ ID NO: 383 - DNA, 23 bp, synthetic construct
  source 1..23: mol_type = other DNA
ggtcgacagcttgggtccctcgg

SEQ ID NO: 384 - DNA, 23 bp, synthetic construct
  source 1..23: mol_type = other DNA
acttgagcctgggggtgtccagg

SEQ ID NO: 385 - DNA, 23 bp, synthetic construct
  source 1..23: mol_type = other DNA
cctactaacccgctggccctccc

SEQ ID NO: 386 - DNA, 23 bp, synthetic construct
  source 1..23: mol_type = other DNA
tctgaggtgtgaccacacagagg

SEQ ID NO: 387 - DNA, 23 bp, synthetic construct
  source 1..23: mol_type = other DNA
cctgagtgaggggaggtggagag

SEQ ID NO: 388 - DNA, 23 bp, synthetic construct
  source 1..23: mol_type = other DNA
ccatcatcgacctagctcccaga

SEQ ID NO: 389 - DNA, 23 bp, synthetic construct
  source 1..23: mol_type = other DNA
cctgggagtggcacaccctgata

SEQ ID NO: 390 - DNA, 23 bp, synthetic construct
  source 1..23: mol_type = other DNA
ccttccctacacgggcaggagga

SEQ ID NO: 391 - DNA, 23 bp, synthetic construct
  source 1..23: mol_type = other DNA
ccttccctacacgggcaggagga

SEQ ID NO: 392 - DNA, 23 bp, synthetic construct
  source 1..23: mol_type = other DNA
cccgtccctcccgtggggctgat

SEQ ID NO: 393 - DNA, 23 bp, synthetic construct
  source 1..23: mol_type = other DNA
cccgtccctcccgtggggctgat

SEQ ID NO: 394 - DNA, 23 bp, synthetic construct
  source 1..23: mol_type = other DNA
tcatagtcacacccaggaggtgg

SEQ ID NO: 395 - DNA, 23 bp, synthetic construct
  source 1..23: mol_type = other DNA
cctgaggcacagcctgctctctc

SEQ ID NO: 396 - DNA, 23 bp, synthetic construct
  source 1..23: mol_type = other DNA
ggggagctggggaggatggatgg

SEQ ID NO: 397 - DNA, 23 bp, synthetic construct
  source 1..23: mol_type = other DNA
ccgatgggaggaggcaggggtgg

SEQ ID NO: 398 - DNA, 23 bp, synthetic construct
  source 1..23: mol_type = other DNA
cccagcgccctgagagccctgaa

SEQ ID NO: 399 - DNA, 23 bp, synthetic construct
  source 1..23: mol_type = other DNA
ccggctgtacagggcgagagaag

SEQ ID NO: 400 - DNA, 23 bp, synthetic construct
  source 1..23: mol_type = other DNA
ccttgtgaccggaccaccctcca

SEQ ID NO: 401 - DNA, 23 bp, synthetic construct
  source 1..23: mol_type = other DNA
ccttgtgaccggaccaccctcca

SEQ ID NO: 402 - DNA, 23 bp, synthetic construct
  source 1..23: mol_type = other DNA
cctgccagaacgtgaaccactgg

SEQ ID NO: 403 - DNA, 23 bp, synthetic construct
  source 1..23: mol_type = other DNA
cctgccagaacgtgaaccactgg

SEQ ID NO: 404 - DNA, 23 bp, synthetic construct
  source 1..23: mol_type = other DNA
cccctgagacccaggaggtgctg

SEQ ID NO: 405 - DNA, 23 bp, synthetic construct
  source 1..23: mol_type = other DNA
aggcctagcagtgcgagtgtggg

SEQ ID NO: 406 - DNA, 23 bp, synthetic construct
  source 1..23: mol_type = other DNA
cctggccacaggatgtcaagaaa

SEQ ID NO: 407 - DNA, 23 bp, synthetic construct
  source 1..23: mol_type = other DNA
ggagggcacccaggtcctcatgg

SEQ ID NO: 408 - DNA, 23 bp, synthetic construct
  source 1..23: mol_type = other DNA
ggagggcacccaggtcctcatgg

SEQ ID NO: 409 - DNA, 23 bp, synthetic construct
  source 1..23: mol_type = other DNA
gggcgtgagggggccaaccatgg

SEQ ID NO: 410 - DNA, 23 bp, synthetic construct
  source 1..23: mol_type = other DNA
gggcgtgagggggccaaccatgg

SEQ ID NO: 411 - DNA, 23 bp, synthetic construct
  source 1..23: mol_type = other DNA
gggcgtgagggggccaaccatgg

SEQ ID NO: 412 - DNA, 23 bp, synthetic construct
  source 1..23: mol_type = other DNA
gggcgtgagggggccaaccatgg

SEQ ID NO: 413 - DNA, 23 bp, synthetic construct
  source 1..23: mol_type = other DNA
agaggacagaggcaggagggagg

SEQ ID NO: 414 - DNA, 23 bp, synthetic construct
  source 1..23: mol_type = other DNA
ccatcccctctagcggtaaggcc

SEQ ID NO: 415 - DNA, 23 bp, synthetic construct
  source 1..23: mol_type = other DNA
ctcatccatcggccatgtgcagg

SEQ ID NO: 416 - DNA, 23 bp, synthetic construct
  source 1..23: mol_type = other DNA
cctttaacctggggggaacccac

SEQ ID NO: 417 - DNA, 23 bp, synthetic construct
  source 1..23: mol_type = other DNA
cctgcgctcagtaaccggaggaa

SEQ ID NO: 418 - DNA, 23 bp, synthetic construct
  source 1..23: mol_type = other DNA
cccggagagctggggccacagct

SEQ ID NO: 419 - DNA, 23 bp, synthetic construct
  source 1..23: mol_type = other DNA
cccggagagctggggccacagct

SEQ ID NO: 420 - DNA, 23 bp, synthetic construct
  source 1..23: mol_type = other DNA
cccgggcccagccagtcccaact

SEQ ID NO: 421 - DNA, 23 bp, synthetic construct
  source 1..23: mol_type = other DNA
gcccgaggagtgggacgtggggg

SEQ ID NO: 422 - DNA, 23 bp, synthetic construct
  source 1..23: mol_type = other DNA
cctgtgggacctctccgattcac

SEQ ID NO: 423 - DNA, 23 bp, synthetic construct
  source 1..23: mol_type = other DNA
ccctcctcgagcccacccccgca

SEQ ID NO: 424 - DNA, 23 bp, synthetic construct
  source 1..23: mol_type = other DNA
gtccctatggaccagcaccaggg

SEQ ID NO: 425 - DNA, 23 bp, synthetic construct
  source 1..23: mol_type = other DNA
ggaggtagcaaaaaccctggagg

SEQ ID NO: 426 - DNA, 23 bp, synthetic construct
  source 1..23: mol_type = other DNA
ttcaggccatgaagggaagtggg

SEQ ID NO: 427 - DNA, 23 bp, synthetic construct
  source 1..23: mol_type = other DNA
actcccctccgagagccgggcgg

SEQ ID NO: 428 - DNA, 23 bp, synthetic construct
  source 1..23: mol_type = other DNA
ctgccagcgggaactgtgtaggg

SEQ ID NO: 429 - DNA, 23 bp, synthetic construct
  source 1..23: mol_type = other DNA
aggtgaacatccctaggaaaagg

SEQ ID NO: 430 - DNA, 23 bp, synthetic construct
  source 1..23: mol_type = other DNA
tcacagctggtcggaagctcagg

SEQ ID NO: 431 - DNA, 23 bp, synthetic construct
  source 1..23: mol_type = other DNA
cccgcatgcccgggaccagctgt

SEQ ID NO: 432 - DNA, 23 bp, synthetic construct
  source 1..23: mol_type = other DNA
ttagggcacttgagagactgggg

SEQ ID NO: 433 - DNA, 23 bp, synthetic construct
  source 1..23: mol_type = other DNA
ttagggcacttgagagactgggg

SEQ ID NO: 434 - DNA, 23 bp, synthetic construct
  source 1..23: mol_type = other DNA
ctgaaaccaaagcagtactttgg

SEQ ID NO: 435 - DNA, 23 bp, synthetic construct
  source 1..23: mol_type = other DNA
ccatgagagcaggcggagggaag

SEQ ID NO: 436 - DNA, 23 bp, synthetic construct
  source 1..23: mol_type = other DNA
cccgagggcggctgggtgtcggg

SEQ ID NO: 437 - DNA, 23 bp, synthetic construct
  source 1..23: mol_type = other DNA
ccactgtgactcgcgtagcagag

SEQ ID NO: 438 - DNA, 23 bp, synthetic construct
  source 1..23: mol_type = other DNA
ccactgtgactcgcgtagcagag

SEQ ID NO: 439 - DNA, 23 bp, synthetic construct
  source 1..23: mol_type = other DNA
ccactgtgactcgcgtagcagag

SEQ ID NO: 440 - DNA, 23 bp, synthetic construct
  source 1..23: mol_type = other DNA
gggcggccctaagctcaggatgg

SEQ ID NO: 441 - DNA, 23 bp, synthetic construct
  source 1..23: mol_type = other DNA
gggcggccctaagctcaggatgg

SEQ ID NO: 442 - DNA, 23 bp, synthetic construct
  source 1..23: mol_type = other DNA
gggcggccctaagctcaggatgg

SEQ ID NO: 443 - DNA, 23 bp, synthetic construct
  source 1..23: mol_type = other DNA
ccccccgacggtggttcacgaaa

SEQ ID NO: 444 - DNA, 23 bp, synthetic construct
  source 1..23: mol_type = other DNA
ccccccgacggtggttcacgaaa

SEQ ID NO: 445 - DNA, 23 bp, synthetic construct
  source 1..23: mol_type = other DNA
cccaggagcagcccgggaaccca

SEQ ID NO: 446 - DNA, 23 bp, synthetic construct
  source 1..23: mol_type = other DNA
cccaggagcagcccgggaaccca

SEQ ID NO: 447 - DNA, 23 bp, synthetic construct
  source 1..23: mol_type = other DNA
cccaggagcagcccgggaaccca

SEQ ID NO: 448 - DNA, 23 bp, synthetic construct
  source 1..23: mol_type = other DNA
cccaggagcagcccgggaaccca

SEQ ID NO: 449 - DNA, 23 bp, synthetic construct
  source 1..23: mol_type = other DNA
cgctgccggccagcgtcctctgg

SEQ ID NO: 450 - DNA, 23 bp, synthetic construct
  source 1..23: mol_type = other DNA
ccacgctaacagagccgccacat

SEQ ID NO: 451 - DNA, 23 bp, synthetic construct
  source 1..23: mol_type = other DNA
ccttaagaagtcggcaggggcag

SEQ ID NO: 452 - DNA, 23 bp, synthetic construct
  source 1..23: mol_type = other DNA
ccttaagaagtcggcaggggcag

SEQ ID NO: 453 - DNA, 23 bp, synthetic construct
  source 1..23: mol_type = other DNA
ccttaagaagtcggcaggggcag

SEQ ID NO: 454 - DNA, 23 bp, synthetic construct
  source 1..23: mol_type = other DNA
ccagcgctggagggcagcggggg

SEQ ID NO: 455 - DNA, 23 bp, synthetic construct
  source 1..23: mol_type = other DNA
cagccctcgcgtgtacctgaagg

SEQ ID NO: 456 - DNA, 23 bp, synthetic construct
  source 1..23: mol_type = other DNA
tgcgaaaggcacaggatccccgg

SEQ ID NO: 457 - DNA, 23 bp, synthetic construct
  source 1..23: mol_type = other DNA
gtgctggctgtagaggttaaagg

SEQ ID NO: 458 - DNA, 23 bp, synthetic construct
  source 1..23: mol_type = other DNA
gtgctggctgtagaggttaaagg

SEQ ID NO: 459 - DNA, 23 bp, synthetic construct
  source 1..23: mol_type = other DNA
ccacctcacaacggggaggaaac

SEQ ID NO: 460 - DNA, 23 bp, synthetic construct
  source 1..23: mol_type = other DNA
cccgggcttgctgagcactcgcg

SEQ ID NO: 461 - DNA, 23 bp, synthetic construct
  source 1..23: mol_type = other DNA
ccggagcactgagaggagcgggg

SEQ ID NO: 462 - DNA, 23 bp, synthetic construct
  source 1..23: mol_type = other DNA
cctccatgctcatgagcactgga

SEQ ID NO: 463 - DNA, 23 bp, synthetic construct
  source 1..23: mol_type = other DNA
cctccatgctcatgagcactgga

SEQ ID NO: 464 - DNA, 23 bp, synthetic construct
  source 1..23: mol_type = other DNA
cccggccgagcgccccaggtcgg

SEQ ID NO: 465 - DNA, 23 bp, synthetic construct
  source 1..23: mol_type = other DNA
tcagggatgggagaggaggaggg

SEQ ID NO: 466 - DNA, 23 bp, synthetic construct
  source 1..23: mol_type = other DNA
ccatctaccggtaccctctcccc

SEQ ID NO: 467 - DNA, 23 bp, synthetic construct
  source 1..23: mol_type = other DNA
ccatctaccggtaccctctcccc

SEQ ID NO: 468 - DNA, 23 bp, synthetic construct
  source 1..23: mol_type = other DNA
ccatctaccggtaccctctcccc

SEQ ID NO: 469 - DNA, 23 bp, synthetic construct
  source 1..23: mol_type = other DNA
ccatctaccggtaccctctcccc

SEQ ID NO: 470 - DNA, 23 bp, synthetic construct
  source 1..23: mol_type = other DNA
ccatctaccggtaccctctcccc

SEQ ID NO: 471 - DNA, 23 bp, synthetic construct
  source 1..23: mol_type = other DNA
ccatctaccggtaccctctcccc

SEQ ID NO: 472 - DNA, 23 bp, synthetic construct
  source 1..23: mol_type = other DNA
ccatgagcctcagaggtatcctg

SEQ ID NO: 473 - DNA, 23 bp, synthetic construct
  source 1..23: mol_type = other DNA
ccctcgtaagatatcaaccatct

SEQ ID NO: 474 - DNA, 23 bp, synthetic construct
  source 1..23: mol_type = other DNA
gaactaggatcccacagggttgg

SEQ ID NO: 475 - DNA, 23 bp, synthetic construct
  source 1..23: mol_type = other DNA
cccgcttcctcctctccactcct

SEQ ID NO: 476 - DNA, 23 bp, synthetic construct
  source 1..23: mol_type = other DNA
caggtcctctcctatctctttgg

SEQ ID NO: 477 - DNA, 23 bp, synthetic construct
  source 1..23: mol_type = other DNA
cctaacagtttaatcaattagcg

SEQ ID NO: 478 - DNA, 23 bp, synthetic construct
  source 1..23: mol_type = other DNA
ccgtactgagtcataccaatgtt

SEQ ID NO: 479 - DNA, 23 bp, synthetic construct
  source 1..23: mol_type = other DNA
ccttccatttcagtttataccag

SEQ ID NO: 480 - DNA, 60 bp, synthetic construct
  source 1..60: mol_type = other DNA
taacttgaaagtatttcgatttcttggctttatatatcttgtggaaaggacgaaacaccg

SEQ ID NO: 481 - DNA, 60 bp, synthetic construct
  source 1..60: mol_type = other DNA
gttgataacggactagccttatttaaacttgctatgctgtttccagcatagctcttaaac

SEQ ID NO: 482 - DNA, 52 bp, synthetic construct
  source 1..52: mol_type = other DNA
aatgatacggcgaccaccgagatctacacaatttcttgggtagtttgcagtt

SEQ ID NO: 483 - DNA, 45 bp, synthetic construct
  source 1..45: mol_type = other DNA
caagcagaagacggcatacgagatgactcggtgccactttttcaa

SEQ ID NO: 484 - DNA, 43 bp, synthetic construct
  source 1..43: mol_type = other DNA
gatttcttggctttatatatcttgtggaaaggacgaaacaccg

SEQ ID NO: 485 - DNA, 38 bp, synthetic construct
  source 1..38: mol_type = other DNA
gctagtccgttatcaacttgaaaaagtggcaccgagtc

SEQ ID NO: 486 - DNA, 20 bp, synthetic construct
  source 1..20: mol_type = other DNA
taactgttacatgaagacaa

SEQ ID NO: 487 - DNA, 20 bp, synthetic construct
  source 1..20: mol_type = other DNA
tgattagggacagttccccg

SEQ ID NO: 488 - DNA, 20 bp, synthetic construct
  source 1..20: mol_type = other DNA
cttgatcctgaaggcaacga

SEQ ID NO: 489 - DNA, 20 bp, synthetic construct
  source 1..20: mol_type = other DNA
gtgaggcaggagagagaggg

SEQ ID NO: 490 - DNA, 20 bp, synthetic construct
  source 1..20: mol_type = other DNA
caacgctcttggggacccgc

SEQ ID NO: 491 - DNA, 20 bp, synthetic construct
  source 1..20: mol_type = other DNA
cggccccctgggatcccctg

SEQ ID NO: 492 - DNA, 20 bp, synthetic construct
  source 1..20: mol_type = other DNA
cgggcgttagtccgaggagg

SEQ ID NO: 493 - DNA, 20 bp, synthetic construct
  source 1..20: mol_type = other DNA
tgccaaactggagaggccgg

SEQ ID NO: 494 - DNA, 20 bp, synthetic construct
  source 1..20: mol_type = other DNA
tgcaagggccaggcgaggtc

SEQ ID NO: 495 - DNA, 20 bp, synthetic construct
  source 1..20: mol_type = other DNA
gggcagataagggaatcagt

SEQ ID NO: 496 - DNA, 20 bp, synthetic construct
  source 1..20: mol_type = other DNA
ccgccagagggtgcactagc

SEQ ID NO: 497 - DNA, 20 bp, synthetic construct
  source 1..20: mol_type = other DNA
tccatttcagtttataccag

SEQ ID NO: 498 - DNA, 20 bp, synthetic construct
  source 1..20: mol_type = other DNA
tacgtcattctcggctgagc

SEQ ID NO: 499 - DNA, 20 bp, synthetic construct
  source 1..20: mol_type = other DNA
agccagtgaccaatgagacc

SEQ ID NO: 500 - DNA, 20 bp, synthetic construct
  source 1..20: mol_type = other DNA
cagctggaagggtcagaagt

SEQ ID NO: 501 - DNA, 20 bp, synthetic construct
  source 1..20: mol_type = other DNA
cgggacgcaggcctggcgta

SEQ ID NO: 502 - DNA, 20 bp, synthetic construct
  source 1..20: mol_type = other DNA
gaaactgattccgaaccagg

SEQ ID NO: 503 - DNA, 20 bp, synthetic construct
  source 1..20: mol_type = other DNA
gaggtttattgtgcccaatg

SEQ ID NO: 504 - DNA, 20 bp, synthetic construct
  source 1..20: mol_type = other DNA
ggtgactgaggcctacaggc

SEQ ID NO: 505 - DNA, 20 bp, synthetic construct
  source 1..20: mol_type = other DNA
aggaggaagactagctagcc

SEQ ID NO: 506 - DNA, 20 bp, synthetic construct
  source 1..20: mol_type = other DNA
agttcaggcttgctggtgag

SEQ ID NO: 507 - DNA, 20 bp, synthetic construct
  source 1..20: mol_type = other DNA
gaggtccctgtgttggctct

SEQ ID NO: 508 - DNA, 20 bp, synthetic construct
  source 1..20: mol_type = other DNA
aatatgacaggagtgtggtc

SEQ ID NO: 509 - DNA, 20 bp, synthetic construct
  source 1..20: mol_type = other DNA
cctcttcccgtccagcagta